(19) United States
(12) Patent Application Publication
(10) Pub. No.: US 2026/0265703 A1
(43) Pub. Date: Sep. 10, 2026

(54) DEGRON FUSION PROTEINS AND METHODS OF PRODUCTION AND USE THEREOF

(71) Applicant: BlueRock Therapeutics LP, Cambridge, MA (US)

(72) Inventors: Conor McAuliffe, West Orange, NJ (US); Chew-Li Soh, Brooklyn, NY (US); Mark Tomishima, Springfield, NJ (US)

(73) Assignee: BlueRock Therapeutics LP, Cambridge, MA (US)

(21) Appl. No.: 19/153,775

(22) PCT Filed: Feb. 5, 2024
(86) PCT No.: PCT/US2024/014395

§ 371 Date: Aug. 5, 2025

Related U.S. Application Data
(60) Provisional application No. 63/443,614, filed on Feb. 6, 2023.
(60) Provisional application No. 63/503,640, filed on May 22, 2023.

(51) Int. Cl. C12N 9/02; A61K 40/30; C07K 7/06; C07K 7/08; C07K 14/00; C12N 9/22; C12N 15/11; C12N 15/90

(52) U.S. Cl. CPC C12N 9/0008; A61K 40/30; C07K 7/06; C07K 7/08; C07K 14/001; C12N 9/222; C12N 15/11; C12N 15/907; C12Y 102/01012; C12N 2310/20

(57) ABSTRACT

The present disclosure relates in part to degron-based methods for controlling gene-edited therapeutic cells administered to patients, and targeting constructs encoding degrons, which are suitable for generating gene-edited target cells that can be eliminated following administration to a patient.

### 1. CROSS-REFERENCE TO RELATED APPLICATIONS

This application claims the priority benefit of U.S. provisional application No. 63/443,614, filed Feb. 6, 2023, and U.S. provisional application No. 63/503,640, filed May 22, 2023, the contents of which are incorporated herein in their entireties by reference thereto.

### 2. SEQUENCE LISTING

The instant application contains a Sequence Listing which has been submitted electronically in XML format and is hereby incorporated by reference in its entirety. Said XML Sequence Listing, created on Jan. 22, 2024, is named BRT-003WO_SL.xml and is 120,402 bytes in size.

### 3. BACKGROUND

Cell therapy provides great promise for the treatment of a variety of diseases and conditions, in some instances, by replacing or repairing defective or damaged cells or tissues with autologous or allogenic cells. Pluripotent stem cells, including induced pluripotent stem cells (iPSCs), which can be differentiated into cell types of interest and may be engineered to recombinantly express therapeutic polypeptides with desired therapeutic characteristics, are especially useful for cell therapy.

However, due to the nature and complexity of these “living drugs,” it would be advantageous to have the ability to remove the cell product after introduction into a patient in case of an adverse event, or as a safety measure after the cell product has performed its function. Therapeutic cell-induced adverse events require rapid and near complete elimination of the therapeutic cells. For instance, overzealous on-target effects, such as by immune-modulating cells, can lead to cytokine storms, associated with tumor lysis syndrome (TLS), cytokine release syndrome (CRS) or macrophage activation syndrome (MAS). Further, certain off-target effects can also arise, such as the cells of the cell therapy becoming cancerous. Thus, there is a need for controlled destruction of genetically engineered cells, when they need to be reduced in number or eliminated from the patients' bodies.

As a result, there is great interest in the development of methods that can eliminate therapeutic cells in the event that they trigger serious adverse events (SAEs) or become obsolete following treatment.

### 4. SUMMARY

The present disclosure provides stable, reliable “suicide genes” that can be used to eliminate therapeutic cells in the event that they trigger or may trigger serious adverse events (SAEs) or become obsolete following treatment.

In particular, the present disclosure provides compositions and methods useful for removing part or all of a transplanted cell therapy, by engineering into the cells for therapy an inducible apoptosis mechanism in the form of a fusion protein comprising an essential protein and a drug-inducible degron. The destruction of this fusion protein is activated by a drug (e.g., a clinically-approved small molecule drug such as an immunomodulatory imide drug (IMiD)).

The fusion protein (sometimes referred to as a “kill switch”) is typically expressed from the endogenous essential gene locus, thereby overcoming disadvantages of other kill switches such as transcriptional silencing or mutation of the transgene expressing a kill switch during cellular differentiation (e.g., to make a cell therapy product). Because the kill switches of the present disclosure incorporate an essential protein, cellular survival is dependent on the presence of the kill switch (and lack of induction of the degron). When a cell that expresses the fusion protein is exposed to a suitable condition (e.g., a drug), degradation of the fusion protein could induce apoptosis and provide better control of induced apoptosis, because: 1) an essential gene cannot be transcriptionally silenced without cell death, and 2) the kill switch itself (i.e., in the induced state) cannot be mutated without killing the cell.

Generally, fusion proteins of the disclosure comprise an essential polypeptide and one or more degrons, optionally connected via one or more peptide linkers. Fusion proteins are further described in Section 6.2 and numbered embodiments 1 to 56.

The present disclosure provides fusion proteins, which typically comprise an essential polypeptide or a fragment or derivative thereof. An essential polypeptide is a polypeptide encoded by an essential gene, null mutations of which are detrimental to the survival of affected cells. Therefore, degradation of an essential polypeptide via an inducible degron can be used to regulate the survival state of a target cell. Exemplary essential polypeptides are described in Section 6.2.1 and numbered embodiments 40 to 56.

The fusion proteins of the present disclosure further comprise one or more degrons. Broadly, a degron is a peptide sequence or protein element, e.g., a structural motif, a short amino acid sequence, etc., that regulates the degradation rate of a protein e.g., by targeting the protein for polyubiquitylation, and subsequently, degradation via proteasome. In some embodiments, the fusion protein comprises a drug inducible degron, whereby stability of the degron is controlled by the presence or absence of a small molecule that binds to the degron. Further details about degrons and exemplary degrons are described in Section 6.2.2 and numbered embodiments 2 to 27 and 35 to 39.

A fusion protein of the disclosure can further comprise an optional linker sequence between a degron sequence and an essential polypeptide sequence. In the case of fusion proteins with multiple degrons, individual degrons can be connected to one another via optional linkers. Section 6.2.3 and numbered embodiments 28 to 34 describe suitable optional linkers.

The present disclosure provides targeting constructs designed to generate a genomic sequence in a target cell that encodes a fusion protein under the control of expression regulatory elements. The targeting construct typically includes homology arms to direct the integration of the construct into an intended genomic locus in the target cell genome, e.g., an essential gene locus, wherein a sequence encoding a degron and an optional linker is flanked by two homology arms targeting the essential gene locus, so that the essential gene is modified to express a fusion protein comprising the essential polypeptide and the degron, optionally separated via a linker. Targeting constructs are further described in Section 6.3 and subsection 6.3.1 as well as numbered embodiments 57 to 157. Sections 6.3.2 and 6.3.3 describe integration sites and homology arms, respectively, for the targeting constructs of the disclosure.

The constructs and methods of the disclosure can also be used to generate a target cell to express both (a) a fusion protein comprising an essential polypeptide and a degron and (b) a recombinant polypeptide. A recombinant polypeptide can be expressed from a transgene, which can be introduced into a target cell via the same targeting construct or expression vector as the targeting construct or expression vector comprising the degron coding sequence. Section 6.4 and numbered embodiments 235 to 258 further describe and provide exemplary transgenes.

The disclosure further provides targeting constructs and recombinant target cell genomes, which can comprise a separator sequence between a degron coding sequence and the transgene to allow separate expression of polypeptides encoded by a single expression cassette. Exemplary separator sequences are described in Section 6.5.

The present disclosure also provides expression vectors encoding the fusion proteins of the disclosure, which typically comprise an expression cassette comprising a fusion polypeptide operably linked to a regulatory element such as a promoter and, optionally, a self-replication element. Further information about and examples of expression vectors are described in Section 6.6 and numbered embodiments 314 to 318.

The present disclosure further provides methods and systems for producing gene-edited target cells comprising nucleotide sequences encoding the fusion proteins of the disclosure. Further information about and examples of suitable methods and systems are described in Section 6.8 and 6.9 and numbered embodiments 158 to 177.

Examples of recombinant and gene edited target cells, e.g., comprising nucleotide sequences encoding the fusion proteins of the disclosure, are disclosed in, e.g., Section 6.7 and numbered embodiments 178 to 325.

The present disclosure further provides methods of treating patients with cell therapy, comprising administering a cell engineered to express a fusion protein comprising an essential protein and an inducible degron. If the subject experiences adverse effects of the cell therapy or is deemed to be at risk of adverse effects of the cell therapy, the cells can be eliminated in whole or in part through induction of the degron, e.g., by administration of the inducer of the degron to the subject. In some embodiments, the degron is a drug-inducible (e.g., an IMID-inducible) degron and the cells eliminated through administration of the drug (e.g. an IMiD). Further information about and examples of methods of the disclosure are described in Section 6.11 and numbered embodiments 327 to 366. For cell therapy, the cells may be formulated as a pharmaceutical composition, e.g., as described in Section 6.10 and numbered embodiment 326.

Additional features, advantages and applications of the fusion proteins, nucleic acids (targeting constructs, expression vectors), cells and methods of the disclosure are more particularly described below.

### 5. BRIEF DESCRIPTION OF THE FIGURES

FIGS. 1A-1D are cartoon illustrations of degron-essential polypeptide fusion proteins and their coding sequences. FIG. 1A represents a fusion protein comprising a degron polypeptide (D) linked to the N-terminus of an essential polypeptide, optionally via a linker (shown as a line connecting the degron and the essential polypeptide). FIG. 1B represents a fusion protein comprising a degron polypeptide (D) linked to the C-terminus of an essential polypeptide, optionally via a linker (shown as a line connecting the degron and the essential polypeptide). FIG. 1C is a diagram of a nucleic acid comprising from 5′ to 3′ an endogenous promoter of an essential gene, a transcription initiation site (represented by the arrow), and coding sequences of a degron, an optional linker, and an essential polypeptide, which when expressed, results in a fusion protein as depicted in FIG. 1A. FIG. 1D is a diagram of a nucleic acid comprising from 5′ to 3′, an endogenous promoter, a transcription initiation site (represented by the arrow), and coding sequences of an essential polypeptide, an optional linker, and a degron, which when expressed, results in a fusion protein as depicted in FIG. 1B.

FIGS. 2A-2D are schematic illustrations of exemplary targeting constructs and vectors that can be used to generate or introduce nucleic acids encoding the fusion proteins of the disclosure in (to) target cells. FIG. 2A represents a targeting construct with a degron coding sequence flanked by the first and second homology arms, wherein the degron coding sequence is connected on its 5′-end to the first homology arm via an optional linker coding sequence, with the homology arms configured such that integration of the targeting construct into the essential gene via recombination of the homology arms with the target genome results in the generation of a modified essential gene encoding the essential polypeptide fused at its C-terminus to the degron via the optional linker. FIG. 2B represents a targeting construct with a degron coding sequence flanked by the first and second homology arms, wherein the degron coding sequence is connected on its 3′-end to the second homology arm via an optional linker coding sequence, with the homology arms configured such that integration of the targeting construct into the essential gene via recombination of the homology arms with the target genome results in the generation of a modified essential gene encoding the essential polypeptide fused at its N-terminus to the degron via the optional linker. FIG. 2C represents a targeting construct similar to the targeting construct shown in FIG. 2A, but with two degron coding sequences that are connected to one another with a linker instead of a single degron coding sequence. FIG. 2D represents a targeting construct similar to the targeting construct shown in FIG. 2B but with two degron coding sequences that are connected to one another with a linker instead of a single degron coding sequence. Although the targeting constructs in FIGS. 2C and 2D have two degron coding sequences, the targeting constructs of the disclosure may comprise more than two degron coding sequences. In some embodiments, the degron coding sequences are connected to one another via linker sequences.

FIGS. 3A-3F are illustrations depicting the incorporation of targeting constructs at essential gene loci. FIG. 3A is a schematic illustration of monoallelic incorporation of a targeting construct depicted in FIG. 2A at an essential gene locus. FIG. 3B is a schematic illustration of bi-allelic incorporation of a targeting construct as depicted in FIG. 2A at an essential gene locus. FIG. 3C is a cartoon illustration depicting the mechanism of inducible-degron mediated degradation of an essential polypeptide and apoptosis in cells in whose genome the targeting construct of FIG. 2A is integrated. A similar effect can be achieved by introducing an extrachromosomal vector and, e.g., knocking out the essential gene at one or both alleles. FIG. 3D is a schematic illustration of monoallelic incorporation of a targeting construct depicted in FIG. 2B at an essential gene locus. FIG. 3E is a schematic illustration of bi-allelic incorporation of a targeting construct as depicted in FIG. 2B at an essential gene locus. FIG. 3F is a cartoon illustration depicting the mechanism of inducible-degron mediated degradation of an essential polypeptide and apoptosis in cells in whose genome the targeting construct of FIG. 2B is integrated. A similar effect can be achieved by introducing an extrachromosomal vector and, e.g., knocking out the essential gene at one or both alleles.

FIGS. 4A-4D are schematic illustrations of exemplary targeting constructs comprising a transgene in addition to a degron. FIG. 4A shows a construct targeting the 3′ end of the essential gene coding sequence, which comprises from 5′- to 3′, a first homology arm of an essential gene—an optional linker coding sequence—a degron coding sequence—an IRES coding sequence—a transgene—and a second homology arm of the essential gene. The homology arms configured such that integration of the targeting construct into the essential gene via recombination of the homology arms with the essential gene results in the generation of a modified essential gene encoding the essential polypeptide fused at its C-terminus to the degron via the optional linker, followed by the IRES and transgene. FIG. 4B shows a construct targeting the 5′ end of the essential gene coding sequence, which comprises from 5′- to 3′, a first homology arm of an essential gene—a transgene—an IRES coding sequence—a degron coding sequence—an optional linker coding sequence—and the second homology arm of the essential gene. The homology arms configured such that integration of the targeting construct into the essential gene via recombination of the homology arms with the essential gene results in the generation of a modified essential gene encoding the essential polypeptide fused at its N-terminus to the degron via the optional linker, preceded by the IRES and transgene. FIGS. 4C and 4D show targeting constructs similar to those in FIGS. 4A and 4B, respectively, but with two sets of degron sequences. Although FIGS. 4A-4D depict the degron coding sequences and transgene separated by an IRES coding sequence, they may also be separated by a sequence encoding a self-cleaving peptide, such as a 2A peptide, in frame with the degron and transgene sequences. Furthermore, although the targeting constructs in FIGS. 4C and 4D have two degron coding sequences, the targeting constructs of the disclosure may comprise more than two degron coding sequences. In some embodiments, the degron coding sequences are connected to one another via linker sequences.

FIGS. 5A-5D are illustrations depicting the incorporation of targeting constructs at target genomic loci and an exemplary effect of incorporation targeting constructs at essential genes. FIG. 5A is a schematic illustration of monoallelic incorporation of a targeting construct depicted in FIG. 4A at an essential gene locus. FIG. 5B is a schematic illustration of biallelic incorporation of a targeting construct depicted in FIG. 4A at an essential gene locus. FIG. 5C is a schematic illustration of monoallelic incorporation of a targeting construct depicted in FIG. 4B at an essential gene locus. FIG. 5D is a schematic illustration of biallelic incorporation of a targeting construct depicted in FIG. 4B at an essential gene locus.

FIGS. 6A-6E are illustrations depicting the incorporation of different targeting constructs at an essential gene's first and second allele loci. FIG. 6A is a schematic illustration of genomic integration of a first targeting construct comprising the coding sequences of an IRES and a transgene designed to be incorporated immediately 5′ of the endogenous STOP codon of the first allele of the essential gene and a second targeting construct, e.g., as depicted in FIG. 2A, comprising the coding sequences of a linker and a degron designed to be incorporated immediately 5′ of the endogenous STOP codon of the second allele of the essential gene. FIG. 6B is a schematic illustration of genomic integration of a first targeting construct comprising the coding sequences of a transgene and IRES designed to be incorporated immediately 3′ of the ATG initiation codon of the second allele of an essential gene and a second targeting construct, e.g., as depicted in FIG. 2A, comprising the coding sequences of a linker and a degron designed to be incorporated immediately 5′ of the endogenous STOP codon of the second allele of the essential gene. FIG. 6C is a schematic illustration of genomic integration of a first targeting construct comprising the coding sequences of an IRES and a transgene designed to be incorporated immediately 5′ of the endogenous STOP codon of the first allele of the essential gene and a second targeting construct, e.g., as depicted in FIG. 2B, comprising the coding sequences of an optional linker and a degron designed to be incorporated immediately 3′ of the ATG initiation codon of the second allele of the essential gene. FIG. 6D is a schematic illustration of genomic integration of a first targeting construct comprising the coding sequences of a linker and a degron designed to be incorporated immediately 3′ of the ATG initiation codon of the first allele of the essential gene; a second targeting construct comprising the coding sequences of IRES and a transgene designed to be incorporated immediately 5′ of the endogenous STOP codon of the first allele of the essential gene; and a third construct comprising the coding sequences of a linker and a degron designed to be incorporated immediately 3′ of the ATG initiation codon of the second allele of the essential gene. FIG. 6E is a schematic illustration of genomic integration of a first targeting construct comprising the coding sequences of a transgene and IRES designed to be incorporated immediately 3′ of the ATG initiation codon of the second allele of an essential gene; a second targeting construct, comprising the coding sequences of a linker and a degron designed to be incorporated immediately 5′ of the endogenous STOP codon of the second allele of the essential gene; and a third construct comprising the coding sequences of a linker and a degron designed to be incorporated immediately 3′ of the ATG initiation codon of the second allele of the essential gene.

FIGS. 7A-7L are schematic illustrations of exemplary targeting construct configurations, wherein the essential gene is GAPDH (FIGS. 7A-7J) or RPL13A (FIGS. 7K-7L). Therefore, a nucleic acid insert is flanked by GAPDH or RPL13A homology arms in each construct. The construct in FIG. 7A (e.g., a construct comprising the nucleotide sequence of SEQ ID NO: 1) has a nucleic acid insert, flanked by the left and right C-terminal GAPDH homology arms, designed to integrate the nucleic acid insert at the 3′ end of GAPDH locus, whereby the nucleic acid insert, in the N- to C-terminal direction, has a linker, i.e., linker 1 (GGS) and degron (SEQ ID NO: 3). The construct in FIG. 7B has a nucleic acid insert, flanked by the left and right C-terminal GAPDH homology arms, whereby the nucleic acid insert, in the N- to C-terminal direction, has linker 2 (SEQ ID NO:23) and degron (SEQ ID NO:3). The construct in FIG. 7C has a nucleic acid insert, flanked by the left and right C-terminal GAPDH homology arms, whereby the nucleic acid insert, in the N- to C-terminal direction, linker 3 (SEQ ID NO: 103) and degron (SEQ ID NO: 3). The construct in FIG. 7D (e.g., a construct comprising the nucleotide sequence of SEQ ID NO: 2) has a nucleic acid insert, flanked by the left and right C-terminal GAPDH homology arms, whereby the nucleic acid insert, in the N- to C-terminal direction, linker 4 (SEQ ID NO: 15) and superdegron (SEQ ID NO:4). The construct in FIG. 7E (e.g., a construct comprising the nucleotide sequence of SEQ ID NO:29) has a nucleic acid insert, flanked by the left and right N-terminal GAPDH homology arms, designed to integrate the nucleic acid insert at the 5′ end of GAPDH locus, whereby the nucleic acid insert, in the N- to C-terminal direction, has a degron (SEQ ID NO:3) and a linker, i.e., linker 1 (GGS). The construct in FIG. 7F (e.g., a construct comprising the nucleotide sequence of SEQ ID NO:30) has a nucleic acid insert, flanked by the left and right N-terminal GAPDH homology arms, whereby the nucleic acid insert, in the N- to C-terminal direction, has a superdegron (SEQ ID NO:4) and a linker, i.e., linker 4 (SEQ ID NO:15). The construct in FIG. 7G has a nucleic acid insert, flanked by the left and right C-terminal GAPDH homology arms, whereby the nucleic acid insert, in the N- to C-terminal direction, linker 1 (GGS), degron (SEQ ID NO:3), IRES, and GFP. The construct in FIG. 7H has a nucleic acid insert, flanked by the left and right C-terminal GAPDH homology arms, whereby the nucleic acid insert, in the N- to C-terminal direction, linker 2 (SEQ ID NO:23), degron (SEQ ID NO:3), IRES, and GFP. The construct in FIG. 7I has a nucleic acid insert, flanked by the left and right C-terminal GAPDH homology arms, whereby the nucleic acid insert, in the N- to C-terminal direction, linker 3 (SEQ ID NO:103), degron (SEQ ID NO:3), IRES, and GFP. The construct in FIG. 7J has a nucleic acid insert, flanked by the left and right C-terminal GAPDH homology arms, whereby the nucleic acid insert, in the N- to C-terminal direction, linker 4 (SEQ ID NO: 15), superdegron (SEQ ID NO:4), IRES, and GFP. The construct in FIG. 7K (e.g., a construct comprising the nucleotide sequence of SEQ ID NO: 14) has a nucleic acid insert, flanked by the left and right C-terminal RPL13A homology arms, designed to integrate the nucleic acid insert at the 3′ end of the RPL13A locus, whereby the nucleic acid insert, in the N- to C-terminal direction, has a linker, i.e. linker 1 (GGS) and degron (SEQ ID NO:3). The construct in FIG. 7L (e.g., a construct comprising the nucleotide sequence of SEQ ID NO: 17) has a nucleic acid insert, flanked by the left and right C-terminal RPL13A homology arms, designed to integrate the nucleic acid insert at the 3′ end of the RPL13A locus, whereby the nucleic acid insert, in the N- to C-terminal direction, has a linker, i.e. linker 4 (SEQ ID NO: 15) and superdegron (SEQ ID NO:4).

FIGS. 8A-8B demonstrate the effect of 3 UM pomalidomide (POM) treatment on survival of cells that have been edited with targeting constructs comprising a GFP marker and a fusion protein wherein a degron is linked to an essential gene. FIG. 8A shows the fractions of untreated iPSCs that have been gene-edited with the GFP and degron-comprising targeting construct as seen in FIG. 7G (GFP+ data points within the box) and unedited cells (data points outside the box). FIG. 8B shows the fractions of gene-edited and unedited iPSCs following a six-day POM treatment.

FIGS. 9A-9C show the effect of 1 μM POM treatment on survival of gene-edited iPSCs over time. FIG. 9A is a graph displaying the changes in the number of live cells/well at various time points depicted as a percentage of cells/well at the beginning (% of T0). FIG. 9B is a representative image of a gene-edited iPSC-containing well after 92 hours of POM treatment. FIG. 9C is a representative image of an untreated control well containing gene-edited iPSCs, monitored for 92 hours.

FIGS. 10A-10B show the effect of linker length on survival of a pool of iPSCs gene-edited with a targeting construct, wherein a degron or superdegron is linked to the essential gene, GAPDH, following treatment with 3 μM POM. FIG. 10A is a graph displaying the percentage of cells that are GFP-positive quantified using flow cytometry. FIG. 10B is a graph displaying the percentage of cells normalized to the untreated condition quantified using amplicon sequencing.

FIGS. 11A-11D demonstrate the activation of targeting constructs in homozygously gene-edited iPSCs, wherein a degron or superdegron is linked to GAPDH. FIG. 11A is a graph showing qPCR results assessing GAPDH expression following different durations of 3 μM POM treatment. FIG. 11B shows a western blot assessment of GAPDH protein levels in cells gene-edited with a degron linked to GAPDH. Lane 1: molecular weight marker; lane 2: untreated and untransfected parental cells; lane 3: untransfected parental cells treated with 3 UM POM for 24 hours; lane 4: untreated clone A cells transfected with degron 1; lane 5: clone A cells treated with 3 μM POM for 24 hours; lane 6: untreated clone B cells transfected with degron 1; lane 7: clone B cells treated with 3 UM POM for 24 hours; lane 8: untreated clone C cells transfected with degron 1; lane 9: clone C cells treated with 3 μM POM for 24 hours. FIG. 11C shows a western blot assessment of GAPDH protein levels in cells gene-edited with a superdegron linked to GAPDH. Lane 1: molecular weight marker; lane 2: untreated clone A cells transfected with a superdegron; lane 3: clone A cells treated with 3 μM POM for 24 hours. FIG. 11D is a graph that displays the growth kinetics of different iPSC lines gene-edited with a targeting construct comprising a degron or superdegron linked to GAPDH.

FIGS. 12A-12C show the effect of POM concentration on survival of iPSCs that were gene-edited with a targeting construct comprising a degron or superdegron linked to GAPDH. FIG. 12A shows representative images of unedited and gene-edited iPSCs after 5 days of 0.5 UM POM treatment. FIG. 12B is a graph displaying the differences in cell confluency over time, depicted as a percentage of cells/well at the beginning (% of TO) of cells gene-edited with a targeting construct comprising a degron linked to GAPDH, following treatment with different concentrations of POM ranging from 0.03125 to 10 μM. FIG. 12C is a graph displaying the differences in cell confluency depicted as a percentage of cells/well at the beginning (% of TO) of cells gene-edited with a targeting construct comprising a superdegron linked to GAPDH, following treatment with different concentrations of POM ranging from 0.03125 to 10 μM.

FIG. 13 is a cartoon diagram illustrating an assay that can be used to assess targeting construct activity in dopaminergic (DA) neurons differentiated from gene-edited iPSCs. See Kriks et al., 2011, Nature 480 (7378): 547-551 and U.S. Pat. No. 10,711,243, which are hereby incorporated by reference in their entireties.

FIG. 14 shows representative images of parental DA neurons and two lines of gene-edited DA neurons after 5 days of 0.13 μM POM treatment.

FIG. 15A-15D illustrate the effect of POM concentration on cell survival of DA neurons. FIG. 15A is a graph displaying the cell death percentage of unedited parental DA neurons following treatment with different POM concentrations for 5 days. FIG. 15B is a graph displaying the cell death percentage of DA neurons derived from iPSCs gene-edited with the targeting construct clone B, following 5-day treatment with POM concentrations ranging between 0.13 μM 1 μM. FIG. 15C is a graph displaying the cell death percentage of DA neurons derived from iPSCs gene-edited with the targeting construct clone C, following 5-day treatment with POM concentrations ranging between 0.13 μM-1 μM. FIG. 15D is a graph displaying the cell death percentage of DA neurons derived from iPSCs gene-edited with the targeting construct clone B, following 5-day treatment with POM concentrations ranging between 10 nM-100 nM.

FIGS. 16A-C illustrate the effect of an extended range of POM concentrations (0.01 μM-100 μM) on the survival of DA neurons differentiated from either unedited or gene-edited iPSCs. FIG. 16A is a graph displaying the cell death percentage of DA neurons differentiated from unedited parental iPSCs following treatment with different POM concentrations for 6 days. FIG. 16B is a graph displaying the cell death percentage of DA neurons derived from iPSCs gene-edited with the targeting construct comprising a linker that is three amino acids in length (3-aa linker) and a degron (Clone A), following 6-day treatment with POM concentrations ranging between 0.01 μM-100 μM. FIG. 16C is a graph showing the same treatment in another gene-edited iPSC clone containing the same targeting construct comprising a 3 aa linker and a degron (Clone C).

FIG. 17 is a cartoon diagram illustrating an assay that can be used to assess targeting construct activity in myeloid progenitor (MP) cells differentiated from gene-edited iPSCs. See Douvaras et al., 2017 Jun. 6; 8 (6): 1516-1524 and PCT publication nos. WO 2023/150089 A1 and WO 2017/152081 A1, which are hereby incorporated by reference in their entireties.

FIGS. 18A-D illustrate the effect of 1 μM POM on cell survival of myeloid progenitor (MP) cells differentiated from either unedited or gene-edited iPSCs with the targeting construct comprising a 3-aa linker and a degron. FIG. 18A is a graph displaying cell death percentage of MP cells differentiated from unedited parental iPSCs following treatment with or without POM.

FIGS. 18B-D are graphs displaying the cell death percentage of MP cells derived from three different iPSC clones-Clone A (FIG. 18B), Clone B (FIG. 18C), or Clone C (FIG. 18D) gene-edited with the targeting construct comprising a 3 aa linker and a degron, following 108 hours treatment with 1 μM POM or left untreated.

### 6. DETAILED DESCRIPTION

### 6.1. Definitions

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art.

Cell Therapy: The term “cell therapy” as used herein refers to a therapy in which cellular material is administered into a patient. The cellular material may be intact, living cells. For example, T cells capable of fighting cancer cells via cell-mediated immunity may be injected in the course of immunotherapy. Cell therapy is also called cellular therapy or cytotherapy.

Coding Sequence: The term “coding sequence” as used herein refers to the nucleic acid (RNA or DNA molecule) that comprise a nucleotide sequence which encodes a protein or a portion thereof (e.g., an essential polypeptide, a linker, or a degron component of a fusion protein of the disclosure). The coding sequence may be codon optimized for expression in a cell of interest.

Complement: The terms “complement” or “complementary” as used herein mean a nucleic acid can form Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

Degron: The term “degron” refers to a peptide sequence, protein element or portion of a protein involved in regulating the degradation rate of a protein. Degrons may include short amino acid sequences, structural motifs, and exposed amino acids (e.g., lysine or arginine). The stability of a fusion protein comprising an essential polypeptide and a degron sequence is controlled at least in part by the degron sequence. In some embodiments, a suitable degron is constitutive such that the degron exerts its influence on protein stability independent of external factors (e.g., the degron is not drug inducible, temperature inducible, etc.) whereas in other embodiments the degron is inducible (e.g., the degron can be turned on or off by a drug, light exposure, or temperature changes, etc.). In some embodiments, the degron provides the essential polypeptide, such as an essential polypeptide (e.g., GAPDH), to which it is fused with controllable stability. The fusion protein comprising the degron and the essential polypeptide can be maintained in “on” (or stable) condition until such time the cell expressing the fusion protein is to be eliminated, at which time the degron is induced and the fusion protein becoming unstable and eventually degraded, resulting in the killing of the cell. In some embodiments, the degron is drug-inducible, e.g., by an IMiD.

Electroporation: The term “electroporation” refers to the use of a transmembrane electric field pulse to induce microscopic pores in a biological membrane. These pores are commonly called “electropores” which allow macromolecules, ions, and water to pass from one side of the membrane to the other. Typically, electroporation has been used to introduce drugs, DNA, or other molecules into cells. Electroporation is the basis of nucleofection, which combines electrophoretic principles with cell-type specific reagents to transfer macromolecules directly into the nuclei of target cells.

Endonuclease: As used herein, term “endonuclease” refers to enzymes that cleave phosphodiester bonds within a nucleic acid chain. The nucleic acid may be double-stranded DNA (dsDNA), single-stranded DNA (ssDNA), RNA, double-stranded hybrids of DNA and RNA, and synthetic DNA (for example, containing bases other than A, C, G, and T). An endonuclease may cut a nucleic acid symmetrically, leaving “blunt” ends, or in positions that are not directly opposing, creating overhangs, which may be referred to as “sticky ends.” Sometimes, the term endonuclease is simply referred to as a “nuclease” for convenience.

Essential Gene: The term “essential gene” refers to a gene that is indispensable for the survival of a cell or organism. Null mutations of essential genes are detrimental to the survival of affected cells. Some essential genes are cell type or lineage specific, for example tumor-specific or neuronal-specific. Such lineage-specific essential genes are required for the survival of that cell type or lineage but not other cell types or, in some instances, the entire organism (see, e.g., Zhang et al., 2021, Translational Psychiatry. 11 (317)). An essential gene can also be a STEL gene.

Essential Protein, Essential Polypeptide: The terms “essential protein” and “essential polypeptide” are used interchangeably herein to refer to a polypeptide encoded by an essential gene, or a polypeptide having at least 85% (e.g., at least 90%, at least 95%, at least 96%, at least 97%, at least 98% or at least 99%) sequence identity thereto.

Gene-Edited Target Cell: As used herein, the term “gene-edited target cell” refers to a cell engineered to express a fusion protein of the disclosure (comprising an essential polypeptide sequence and a degron) via introduction of a targeting construct of the disclosure, or its descendants and progeny. Typically, the nucleotide sequence flanked by the homology arms of the targeting construct is integrated into the genome of the cell. A gene-edited target cell need not be of the same cell type as the cell into which the targeting construct was initially introduced. For example, the targeting construct may be introduced into a stem cell, such as an iPSC or a hESC, upon which the nucleotide sequence flanked by the homology arms of the targeting construct is integrated into the genome of the stem cell. The stem cell can then be differentiated to produce a differentiated cell type, for example any of the cell types disclosed in Section 6.7.1. Both the stem cell and the differentiated cell are referred to herein as a “gene-edited target cell”. In addition to encoding a fusion protein of the disclosure, the gene-edited target cell may include a transgene, e.g., as described in Section 6.4. In some embodiments, the fusion protein coding sequence and the transgene are inserted into the same essential gene. In some embodiments, both the fusion protein coding sequence and the transgene are positioned in the same allele of the essential gene (whether on a heterozygous or homozygous basis). In other embodiments, the fusion protein coding sequence and the transgene are positioned in different alleles of the essential gene. In yet other embodiments, the fusion protein coding sequence and the transgene are in different loci. Alternatively, one or both of the fusion protein coding sequence and the transgene are expressed from an extrachromosomal expression vector, e.g., in a cell in which the corresponding essential gene is knocked out at one or both alleles. In certain aspects, a gene-edited target cell has a single copy of a fusion protein coding sequence in one allele of the corresponding essential gene.

Guide RNA or gRNA: As used herein, the term “guide RNA” refers to a ribonucleic acid having a DNA-targeting sequence (also referred to as “spacer” or “DNA-targeting segment”) and a protein-binding sequence (also referred to as “protein-binding segment”). The DNA-targeting sequence has sufficient complementarity with a target DNA (e.g., genomic DNA) sequence, to hybridize with the target DNA sequence and direct sequence-specific binding of a nucleic acid-targeting complex to the target DNA sequence. The DNA-targeting sequence generally includes the “protospacer-like” sequence described herein. The protein-binding sequence interacts with a site-specific modifying enzyme (e.g., an endonuclease as described in Section 6.9.2). Site-specific cleavage of the target DNA occurs at locations determined by both (i) base pairing complementarity between the guide RNA and the target DNA; and (ii) a short motif (referred to as the protospacer adjacent motif (PAM)) in the target DNA. The protein-binding segment of a guide RNA includes, in part, two complementary stretches of nucleotides that hybridize to one another to form a double stranded RNA duplex (dsRNA duplex). In some embodiments, a guide RNA is a single-stranded guide RNA (sgRNA).

IMID: The term “IMiD” refers to immunomodulatory imide drugs and includes thalidomide and structural analogs of thalidomide that can act as immunomodulators. Examples of IMiDs include pomalidomide, thalidomide, lenalidomide, iberdomide, and avadomide.

iPSC: The term “induced pluripotent stem cell” or “iPSC” refers to a type of pluripotent stem cell artificially prepared from a non-pluripotent cell, such as an adult somatic cell, partially differentiated cell or terminally differentiated cell, such as a fibroblast, a cell of hematopoietic lineage, a myocyte, a neuron, an epidermal cell, or the like, by introducing or contacting the cell with one or more reprogramming factors. iPSCs can be derived from multiple different cell types, including terminally differentiated cells. iPSCs have an embryonic stem (ES) cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, Fox03, GDF3, Cyp26a1, TERT, and zfp42.

Examples of methods of generating and characterizing iPSCs may be found in, for example, US Patent Publication Nos. US20090047263, US20090068742, US20090191159, US20090227032, US20090246875, and US20090304646, and PCT patent publications WO2013177133 and WO2022204567, the disclosures of which are incorporated herein by reference. Generally, to generate iPSCs, somatic cells are provided with reprogramming factors (e.g., Oct4, SOX2, KLF4, MYC, Nanog, Lin28, etc.) known in the art to reprogram the somatic cells to become pluripotent stem cells.

Knockout: The term “knockout” and related terms (e.g., “knocking out”) refer to both a decrease in/partial ablation of gene expression of one or more genes or the complete ablation of gene expression of one or more genes, whether from one or both alleles. In some embodiments, the term knockout refers to a partial ablation of gene expression (e.g., a modification resulting in a decrease of at least 50%, at least 60% or at least 70% of gene expression in the absence of the modification) at one or both alleles. In other embodiments, the term knockout refers to complete ablation of gene expression at one or both alleles.

Linker or Linker Sequence: The terms “linker” or “linker sequence” as used in reference to a fusion protein, refers to a part that connects two or more domains, parts, or entities. In some embodiments, the linker may comprise an amino acid or a peptide. Generally, linkers have no specific biological activity other than to join or to preserve some minimum distance or other spatial relationship between the parts.

Nucleic Acid: The terms “nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

Nuclease: The terms “nuclease” and “endonuclease” are used interchangeably herein to mean an enzyme which possesses endonucleolytic catalytic activity for nucleic acid cleavage, as well as nuclease-inactivated variants thereof.

Nucleofection: The term “nucleofection” refers to an electroporation-based transfection method, which uses a combination of electrical parameters and cell-type specific reagents to transfer nucleic acids, proteins, or ribonucleoprotein complexes directly to the nuclei of target cells.

Operably linked: The term “operably linked” refers to a functional relationship between two or more peptide or polypeptide domains or nucleic acid (e.g., DNA) segments. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Polypeptide, peptide, and protein: The terms “polypeptide,” “peptide” and “protein” are used interchangeably herein to refer to polymers of amino acids of any length. The polymer may be linear or branched, it may comprise modified amino acids, and it may be interrupted by non-amino acids.

Pluripotent: As used herein, the term “pluripotent” or “pluripotency” refers to the capacity of a cell to self-renew and to differentiate into cells of any of the three germ layers: endoderm, mesoderm, or ectoderm. “Pluripotent stem cells” or “PSCs” include, for example, embryonic stem cells derived from the inner cell mass of a blastocyst or derived by somatic cell nuclear transfer, and iPSCs derived from non-pluripotent cells.

Recombinant Target Cell: As used herein, the term “recombinant target cell” refers to a cell which is engineered to express a fusion protein of the disclosure (comprising an essential polypeptide sequence and a degron), and includes the descendants and progeny of the target cell into which a targeting construct or expression vector of the disclosure was initially introduced. A recombinant target cell need not be of the same cell type as the cell into which the targeting construct or expression vector was initially introduced. For example, the cell initially engineered to express a fusion protein into the disclosure may be a stem cell, such as an iPSC or a hESC. The stem cell can then be differentiated to produce a differentiated cell type, for example any of the cell types disclosed in Section 6.7.1. Both the stem cell and the differentiated cell are referred to herein as a “recombinant target cell”. In addition to encoding a fusion protein of the disclosure, the recombinant target cell may include a transgene, e.g., as described in Section 6.4. In some embodiments, the fusion protein coding sequence and the transgene are located in the same gene, e.g., the essential gene encoding the essential polypeptide portion of the fusion polypeptide. In some embodiments, both the fusion protein coding sequence and the transgene are positioned in the same allele of the essential gene (whether on a heterozygous or homozygous basis). In other embodiments, the fusion protein coding sequence and the transgene are positioned in different alleles of the essential gene. Alternatively, one or both of the fusion protein coding sequence and the transgene are expressed from an extrachromosomal expression vector, e.g., in target cells in which the essential gene is knocked out at one or both loci. In certain aspects, a recombinant target cell has a single copy of a fusion protein coding sequence in one allele of the corresponding essential gene. In other aspects, a recombinant target cell has two or more copies of a fusion protein coding sequence, e.g., one or more copies at each allele of the corresponding essential gene.

Corresponding: The term “corresponding” as used herein in relation to a fusion protein of the disclosure and an essential gene, means that the fusion protein comprises the amino acid sequence of the essential protein encoded by the essential gene (or a fragment or variant of the essential protein).

Reprogramming Factor, Reprogramming Protein: As used herein, the term “reprogramming factor” or “reprogramming protein” refers to a protein, peptide, functional fragment of a protein or peptide, or a small molecule that, when overexpressed or otherwise introduced in a cell, alone or in combination with other proteins, peptides, functional fragments or proteins or peptides, or other small molecules, induces the cell to transition from one state of differentiation to another. In some embodiments, the reprogramming factor induces a somatic cell to transition from a differentiated state to a pluripotent state. The reprogramming factors used herein may be human proteins or modified versions thereof that retain the desired biological effects.

Ribonucleoprotein (RNP) complex: A “ribonucleoprotein complex,” or “ribonucleoprotein particle” as provided herein refers to a complex or particle including a nucleoprotein and a ribonucleic acid. A “nucleoprotein” as provided herein refers to a protein capable of binding a nucleic acid (e.g., RNA, DNA). Where the nucleoprotein binds a ribonucleic acid, it is referred to as “ribonucleoprotein.” The interaction between the ribonucleoprotein and the ribonucleic acid may be direct, e.g., by covalent bond, or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, the ribonucleoprotein includes an RNA-binding motif non-covalently bound to the ribonucleic acid. For example, positively charged aromatic amino acid residues (e.g., lysine residues) in the RNA-binding motif may form electrostatic interactions with the negative nucleic acid phosphate backbones of the RNA, thereby forming a ribonucleoprotein complex. In some embodiments, any one of the nucleases disclosed herein is in a RNP with a guide RNA.

STEL: The terms “sustained transgene expression locus” or “STEL” refer to a locus in the genome of a cell that enables persistent and stable expression of a transgene in that cell. STEL of the present disclosure include, without limitation, loci of robustly expressed endogenous genes, for instance certain housekeeping genes that are active in multiple cell types such as those involved in gene expression (e.g., transcription factors and histones), cellular metabolism (e.g., GAPDH), or cellular structures (e.g., actin), or those that encode ribosomal proteins (e.g., large or small ribosomal subunits, such as RPL13A, RPLP0 and RPL7). Additional examples of STEL include those that form ribonucleoprotein complex, focal adhesion, cell-substrate adherens junction, cell-substrate junction, cell anchoring, extracellular exosome, extracellular vesicle, intracellular organelle, or anchoring junction. Some of the proteins are involved in RNA binding, nucleic acid binding (e.g., rRNA or mRNA binding), or protein binding. A STEL gene can also be an essential gene (sometimes referred to herein as an “essential STEL gene”).

STEL Protein, STEL Polypeptide: The terms “STEL protein” and “STEL polypeptide” are used interchangeably herein to refer to a polypeptide encoded by a STEL gene, or a polypeptide having at least 85% (e.g., at least 90%, at least 95%, at least 96%, at least 97%, at least 98% or at least 99%) sequence identity thereto.

Subject: The terms “subject” or “patient” refer to an organism that is subjected to a procedure and/or treatment of the disclosure. The subject can include human and non-human animals. Non-human animals include all vertebrates, e.g., mammals and non-mammals, such as non-human primates, sheep, dog, cow, chickens, amphibians, and reptiles. In preferred embodiments, the subject is human.

Target Cell: The term “target cell” refers to a host cell into which is introduced (i) an expression vector or (ii) a targeting construct that following integration into the host cell genome results in the production of a recombinant nucleic acid encoding a fusion protein comprising an essential polypeptide, a degron and an optional linker. It should be understood that such terms are intended to refer not only to the particular subject cell but to the progeny of such a cell. Such progeny need not be identical to the parent cell into which the expression vector or targeting construct was initially introduced but include counterparts and progeny of the cell which carry the expression cassette or into which the targeting construct has integrated, as well as cells differentiated therefrom. Such counterparts and progeny are still included within the scope of the term “target cell” as used herein.

Targeting Construct: The term “targeting construct” refers to a recombinant nucleic acid molecule that can specifically interact with an essential gene locus. Recombination of the targeting construct and the target genomic locus leads to the modification of the essential gene, e.g., to modify an essential polypeptide to include a degron coding sequence and/or to introduce a transgene into the essential gene locus. Typically, a targeting construct comprises homology arms that allow integration of the targeting construct into a particular genetic locus, e.g., an essential gene.

Transfection: The term “transfection” refers to the introduction of nucleic acid molecules, such as DNA or RNA (e.g., mRNA) molecules, into cells, e.g., into eukaryotic cells. In the context of the present invention, the term “transfection” encompasses any method known to the skilled person for introducing nucleic acid molecules into cells, e.g., into eukaryotic cells, such as into mammalian cells. Such methods encompass, for example, electroporation, nucleofection, lipofection, e.g., based on cationic lipids and/or liposomes, calcium phosphate precipitation, nanoparticle-based transfection, virus-based transfection, or transfection based on cationic polymers, such as DEAE-dextran or polyethylenimine.

Vector: The term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. In some embodiments, a vector is a viral vector, e.g., a lentiviral vector, an adenoviral vector, or an adeno-associated virus (AAV) vector. Moreover, certain vectors are capable of directing the expression of nucleotide sequences to which they are operably linked. Such vectors are referred to herein as “expression vectors”.

### 6.2. Fusion Proteins

The present disclosure relates to fusion proteins comprising (i) an essential polypeptide and (ii) a degron, optionally connected via a peptide linker. In some embodiments, the degron is N-terminal to the essential polypeptide. In other embodiments, the degron is C-terminal to the essential polypeptide.

Nucleic acids encoding the fusion proteins of the disclosure may be introduced into a target cell, e.g., in the form of a vector as described in Section 6.6 or a targeting construct that results in integration of an exogenous nucleotide sequence encoding the fusion protein into an essential gene in the target cell genome.

In some embodiments, the nucleic acids encoding the fusion proteins of the disclosure can be generated in a target cell in situ by introduction of a targeting construct, e.g., as described in Section 6.3, that recombines with an essential gene in a target cell genome to create a modified essential gene that encodes a fusion protein of the disclosure comprising:
(i) an essential polypeptide, as described in Section 6.2.1;
(ii) a degron, as described in Section 6.2.2;
(iii) and an optional linker, as described in Section 6.2.3, connecting the essential polypeptide and the degron.

Thus, when the fusion protein is expressed as a result of integration of a targeting construct encoding a degron into the essential gene encoding the essential polypeptide, the essential gene is the essential gene of the targeting construct. When the fusion protein is expressed as a result of integration of a targeting construct encoding the essential polypeptide and the degron into a different locus than the essential gene encoding the essential polypeptide, then the other locus is the essential gene of the targeting construct.

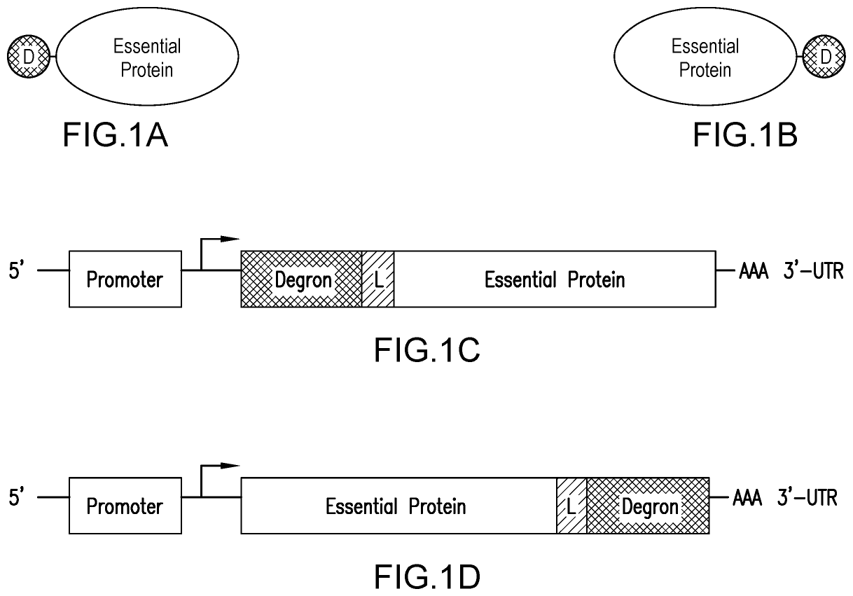

In some embodiments, the fusion protein is configured such that the degron is at the N-terminus of the essential polypeptide, e.g., as illustrated in FIG. 1A. In other embodiments, the fusion protein is configured such that the degron is at the C-terminus of the essential polypeptide, e.g., as illustrated in FIG. 1B.

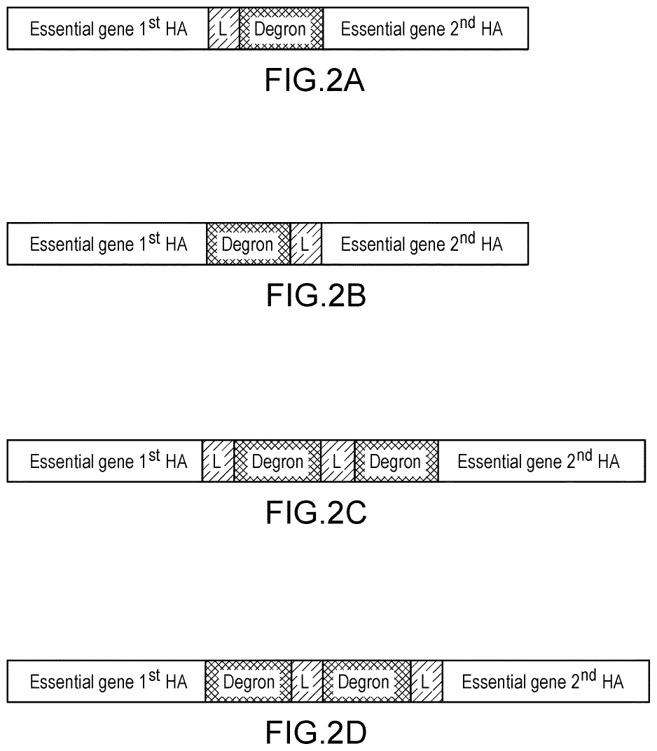

In some embodiments, the fusion protein comprises only one degron. In other embodiments, the fusion protein comprises two or more degrons. In some embodiments, the degrons are in tandem and separated by linkers. Fusion proteins comprising only one degron are produced following integration of the targeting constructs illustrated in FIG. 2A and FIG. 2B. Fusion protein comprising two degrons are produced following integration of the targeting constructs illustrated in FIG. 2C and FIG. 2D.

In some embodiments, fusion protein further comprises a self-cleaving peptide sequence, e.g., as described in Section 6.5, and a polypeptide encoded by a transgene, e.g., as described in Section 6.4. Such fusion proteins can be produced, by, e.g., incomplete processing of a self-cleaving peptide. Proper processing of a fusion protein with a self-cleaving peptide sequence at its N- or C-terminus will result in the fusion proteins comprising a few amino acid residues of the self-cleaving peptide. Fusion proteins comprising properly processed or incompletely processed self-cleaving peptide sequences (and in some embodiments polypeptide sequences encoded by transgenes) are encompassed by the term “fusion protein of the disclosure.”

Thus, in some embodiments, the fusion proteins of the disclosure lack self-cleaving peptide sequences. In other embodiments, the fusion proteins of the disclosure comprise properly processed self-cleaving peptide sequences (which may be single amino acid residues). In further embodiments, the fusion proteins of the disclosure comprise incompletely processed self-cleaving peptide sequences and, optionally, transgene-encoded polypeptide sequences.

In some embodiments, the degron is an inducible degron. Fusion of the essential polypeptide to the degron allows its stability to be controlled by induction of the degron. In the absence of an inducer, the degron is inactive and the essential polypeptide is stable. In the presence of an inducer, the degron is active and the essential polypeptide is unstable, leading to its destruction. Destruction of the essential polypeptide is detrimental to cell survival. Thus, the fusion proteins of the disclosure can be used to control cellular survival.

### 6.2.1. Essential Polypeptide

The fusion proteins of the disclosure typically comprise an essential polypeptide or a fragment or derivative thereof (all collectively referred to herein as “essential protein” or “essential polypeptide” for convenience). In some embodiments, the essential polypeptide is a STEL polypeptide. In other embodiments, the essential polypeptide is a non-STEL polypeptide.

In some embodiments, the essential polypeptide (whether a STEL or non-STEL polypeptide) is of a category of essential polypeptides identified below. In other embodiments, the essential polypeptide (whether a STEL or non-STEL polypeptide) is not from a category of essential polypeptides identified below (e.g., the category is the subject of a proviso).

Further, in some embodiments, the essential polypeptide (whether a STEL or non-STEL polypeptide) is one of the essential polypeptides identified below. In other embodiments, the essential polypeptide (whether a STEL or non-STEL polypeptide) is not one of the essential polypeptides identified below (e.g., the particular essential polypeptide is the subject of a proviso).

In some embodiments, the essential polypeptide is encoded by a gene associated with cellular metabolism, such as GAPDH. In various embodiments, the essential polypeptide has at least 85%, at least 90%, at least 95%, at least 96%, at least 97%, at least 98% or at least 99% sequence identity to a GAPDH polypeptide of SEQ ID NO: 100.

In some embodiments, an essential polypeptide is a ribosomal polypeptide (RPL), e.g., a polypeptide encoded by an RPL gene. Examples of RPL genes are RPL10, RPL13, RPS18, RPL3, RPLP1, RPL13A, RPL15, RPL41, RPL11, RPL32, RPL18A, RPL19, RPL28, RPL29, RPL9, RPL8, RPL6, RPL18, RPL7, RPL7A, RPL21, RPL37A, RPL12, RPL5, RPL34, RPL35A, RPL30, RPL24, RPL39, RPL37, RPL14, RPL27A, RPLP2, RPLP0, RPL23A, RPL26, RPL36, RPL35, RPL23, RPL4, and RPL22. In various embodiments, the essential polypeptide has at least 85%, at least 90%, at least 95%, at least 96%, at least 97%, at least 98% or at least 99% sequence identity to an RPL13A or RPLP0 polypeptide of SEQ ID NO: 101 or SEQ ID NO: 102, respectively.

In some embodiments, an essential polypeptide is a ribosomal polypeptide small subunit (RPS), e.g., a polypeptide encoded by an RPS gene. Examples of RPS genes are RPS2, RPS19, RPS14, RPS3A, RPS12, RPS3, RPS6, RPS23, RPS27A, RPS8, RPS4X, RPS7, RPS24, RPS27, RPS15A, RPS9, RPS28, RPS13, RPSA, RPS5, RPS16, RPS25, RPS15, RPS20, and RPS11.

In some embodiments, an essential polypeptide is a cytoskeletal protein, such as actin. Examples of actin-encoding genes are ACTG1 and ACTB.

In some embodiments, an essential polypeptide is a eukaryotic translation elongation factor, such as EEF1A1 and EEF2, or a eukaryotic translation initiation factor, such as EIF1.

In some embodiments, an essential polypeptide is a histone, such as a histone encoded by the genes H3F3A and H3F3B.

In other embodiments, an essential polypeptide is a STEL polypeptide selected from, FTH1, TPT1, PTMA, GNB2L1, NACA, YBX1, NPM1, FAU, UBA52, HSP90AB1, MYL6, SERF2, and SRP14.

In other embodiments, an essential polypeptide is a non-STEL polypeptide, such as HDAC3, DNMT1, NADH dehydrogenase, and PGK1.

The fusion proteins of the disclosure typically comprise the native essential polypeptide sequence, e.g., when the fusion protein coding sequence is constituted upon integration of a targeting construct into an essential gene locus.

The essential polypeptide sequence in the fusion protein may alternatively be a variant of a wild-type essential polypeptide sequence with substitutions, additions, and deletions, e.g., when expressed via an expression vector or from a target cell genome modified by a targeting construct encoding the entire fusion protein. Without being bound by theory, it is believed that recombinant expression of a fusion protein comprising an essential polypeptide and a degron can “poison” the native cellular essential polypeptide and result in its destabilization when the degron is activated, resulting in cell death even when the essential gene is intact. In various embodiments, the essential polypeptide sequence in the fusion protein has at least 80%, at least 85%, at least 90%, at least 95%, at least 97%, at least 98% or at least 99% sequence identity to the wild-type amino acid sequence of the essential polypeptide.

### 6.2.2. Degrons

The fusion proteins described herein comprise one or more peptide sequences that can serve as a “shutoff switch” or “kill switch” for elimination of target cells that have been engineered to express the fusion proteins of the disclosure. Such “kill switch” peptide sequences are herein referred to as degrons.

Generally, a degron is a peptide sequence or protein element that regulates the degradation rate of a protein, e.g., by targeting the protein for polyubiquitylation, and subsequently, degradation via proteasome. Degrons may include short amino acid sequences, structural motifs, and exposed amino acids (e.g., lysine or arginine).

The stability of a degron is controlled at least in part by the degron sequence. In some embodiments, a suitable degron is constitutive such that the degron exerts its influence on protein stability independent of experimental control (e.g., the degron is not drug inducible, temperature inducible, etc.). In some embodiments, the degron provides the essential polypeptide (e.g., GAPDH) to which it is fused with controllable stability such that the fusion protein can be turned “on” (e.g., stable) or “off” (e.g., unstable, degraded) depending on the desired conditions.

In some embodiments the degron is a drug inducible degron, whereby the presence or absence of drug can switch the protein from an “off” (e.g., unstable) state to an “on” (e.g., stable) state or vice versa. In some embodiments, the stability of the degron is controlled by the presence or absence of a small molecule that binds to the degron.

Examples of suitable degrons controlled by the presence or absence of a small molecule include, but are not limited to, degrons controlled by immunomodulatory imide drugs (IMiDs, e.g., pomalidomide, thalidomide, lenalidomide, iberdomide, avadomide, etc.), Shield-1, DHFR, and/or auxins. Other inducible degrons are temperature-sensitive degrons, light-inducible degrons, and degrons activated through expression of another protein, e.g., TEV protease. Non-limiting examples of suitable degrons are known in the art (e.g., Dohmen et al., Science, 1994. 263 (5151): 1273-1276; Schoeber et al., 2009, Am J Physiol Renal Physiol. 296 (1): F204-11; Chu et al., 2008, Bioorg Med Chem Lett. 18 (22): 5941-4; Kanemaki, 2012, Pflugers Arch. December 28; Yang et al., 2012, Mol Cell. 48 (4): 487-8; Barbour et al., 2013, Biosci Rep. 33 (1); and Greussing et al., 2012, J Vis Exp. (69), the contents of which are hereby incorporated by reference herein in their entireties).

In some embodiments, a fusion protein comprises an inducible degron sequence fused to an essential polypeptide. In some embodiments, the degron is a zinc finger degron that can be controlled with an IMiD, e.g., thalidomide, lenalidomide, pomalidomide, and/or analogs thereof. In some embodiments, the IMID-sensitive degron is an engineered degron, e.g., a superdegron, which possesses increased sensitivity to IMiDs and enables more efficient degradation of the essential polypeptide than the degradation achieved with the non-engineered degron.

Fusing a degron sequence to a polypeptide sequence can be used to produce a polypeptide with an off switch. For instance, fusion of an IMiD-sensitive degron to an essential polypeptide, such as GAPDH, results in a GAPDH-degron fusion protein, the expression of which can be switched off in the presence of an IMiD such as pomalidomide via targeted degradation of the GAPDH-degron fusion protein. The degradation of fusion protein comprising an essential polypeptide and a degron can result in apoptosis of cells engineered to express the fusion protein.

In some embodiments, a fusion protein of the disclosure serves as a kill switch to eliminate cells engineered to express the fusion protein. In some embodiments, a fusion protein of the disclosure allows elimination of cells in vitro, e.g., in the context of a functional screen as disclosed by Natsume and Kanemaki, 2017, Annu Rev Genet. 51:83-102, wherein it enables rapid control of expression of a protein of interest fused to a degron in order to determine whether the protein of interest is essential for cell viability. In other embodiments, a fusion protein of the disclosure allows elimination of cells in vivo, e.g., following gene therapy as disclosed in Section 6.11.

In some embodiments, the degron is an inducible degron.

In some embodiments, the degron is inducible by a small molecule, e.g., a drug.

In some embodiments, the degron is inducible by an IMID. Examples of IMID-inducible degron sequences are disclosed in Koduri et al., 2019, Proc. Nat'l Acad. Sci. USA 116 (7): 2539-2544, WO 2021/188286 A2, and WO 2019/089592 A1, the contents of each of which are incorporated herein in their entireties.

In some embodiments, the degron comprises or consists of the amino acid sequence RPFQCNQCGASFTQKGNLLRHIKLH (SEQ ID NO:3, from Koduri et al.), FNVLMVHKRSHTGERPLQCEICGFTCRQKGNLLRHIKLHTGEKPFKCHLCNYACQRRDAL (SEQ ID NO:4, corresponds to SEQ ID NO:42 of WO 2021/188286 A2), FNVLMVHKRSHTGERP (SEQ ID NO:5, corresponds to SEQ ID NO:97 of WO 2019/089592 A1), FNVLMVHRRSHTGERP (SEQ ID NO:6, corresponds to SEQ ID NO:100 of WO 2019/089592 A1), TGEKPFKCHLCNYACQRRDAL (SEQ ID NO:7, corresponds to SEQ ID NO: 102 of WO 2019/089592 A1), TGERPFRCHLCNYACQRRDAL (SEQ ID NO:8, corresponds to SEQ ID NO: 103 of WO 2019/089592 A1), FQCNQCGASFT (SEQ ID NO:9, corresponds to SEQ ID NO:528 of WO 2021/188286 A2), FQCPICGLVIK (SEQ ID NO: 10, corresponds to SEQ ID NO: 529 of WO 2021/188286 A2), LQCEICGFTCR (SEQ ID NO: 11, corresponds to SEQ ID NO: 530 of WO 2021/188286 A2), LQCEICGYQCR (SEQ ID NO: 12, corresponds to SEQ ID NO: 531 of WO 2021/188286 A2), or LQCEVCGFQCR (SEQ ID NO: 13, corresponds to SEQ ID NO: 532 of WO 2021/188286 A2).

In some embodiments, the degron is a superdegron. SEQ ID NO:4 is an example of a superdegron sequence.

In some embodiments, the degron is a SMASh (Small-Molecule-Assisted Shutoff) tag degron, which is a self-cleaving degron that can be stabilized upon treatment with a small molecule, e.g., a drug such as asunaprevir. In the absence of a protease inhibitor, the SMASh tag self-cleaves, and the protein is expressed at relatively normal levels. However, in the presence of the drug, the SMASh tag degron stays fused to the protein, inducing all newly synthetized fusion proteins to be rapidly degraded.

### 6.2.3. Linkers

The fusion proteins of the disclosure may comprise an optional linker sequence between the essential polypeptide sequence and the degron sequence.

Suitable linkers for use in the methods of the present disclosure are well known to those of skill in the art and include peptide linkers. In particular embodiments, the linker is used to separate the essential polypeptide and the degron by a distance sufficient to ensure that the essential polypeptide retains its required functional property. In some embodiments, peptide linker sequences adopt a flexible extended conformation and do not exhibit a propensity for developing an ordered secondary structure.

Typical amino acids in flexible peptide linkers include Gly, Asn and Ser. Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al., 1985, Gene 40:39-46; Murphy et al., 1986, Proc. Nat'l. Acad. Sci. USA 83:8258-62; U.S. Pat. Nos. 4,935,233; and 4,751,180, the contents of which are incorporated herein in their entireties.

Peptide linkers can be one amino acid sequence or repeats of one or more amino acid sequences. In some embodiments, a sequence can be used in repeats of 2. In some embodiments, a sequence can be used in repeats of 3. In some embodiments, a sequence can be used in repeats of 4. In some embodiments, a sequence can be used in repeats of 5 or more.

In some embodiments, the peptide linker is between 1 and 30 amino acids in length. In various aspects, the peptide linker is between 1 and 3 amino acids in length, between 3 and 8 amino acids in length, between 3 and 10 amino acids in length, between 5 and 15 amino acids in length, between 11 and 20 amino acids in length, between 15 and 25 amino acids in length, between 21 and 30 amino acids in length, or is a length range bounded by any pair of the forgoing values (e.g., between 3 and 15 amino acids in length, between 8 and 20 amino acids in length, between 25 and 30 amino acids in length, and so on and so forth).

In some embodiments, the linker is a “short” linker of up to 15 amino acids, e.g., 1, 2, 3, 4, 5, 6, 7, 8, 9, 10, 11, 12, 13, 14 or 15 amino acids in length, or is a length range bounded by any pair of the forgoing values (e.g., between 1 and 3 amino acids, between 1 and 12 amino acids in length, between 2 and 12 amino acids in length, between 1 and 10 amino acids in length, and so on and so forth)

Non-limiting examples of linker sequences are set forth in Table 1 below.

TABLE 1

| Linker Sequence | SEQ ID NO |
|---|---|
| GGS | N/A |
| GSGSGSGSGG | 15 |
| GGGS | 16 |
| GSG | N/A |
| GGSG | 18 |
| GGGS | 19 |
| GGGGS | 20 |
| (GGS)3 | 21 |
| (GGGGS)2 | 22 |
| (GGGGS)3 | 23 |
| PAPAP | 35 |
| AEAAAKEAAAKA | 36 |
| A(EAAAK)4ALEA(EAAAK)4A | 37 |
| (GGS)9 | 103 |

6.3. Targeting Constructs 6.3.1. Targeting Constructs for Fusion Protein Expression

The present disclosure provides a targeting construct designed to generate a genomic sequence in a target cell that encodes a fusion protein as described herein, e.g., an essential polypeptide as described in Section 6.2.1, a degron and, optionally, a linker sequence, under the control of expression regulatory elements. The targeting construct typically includes homology arms to direct the integration of the construct into an intended genomic locus in the target cell genome.

The targeting constructs of the disclosure can include the entire coding sequence of the fusion protein, for integration of the full fusion protein coding sequence into genomic locus in the target cell genome. Depending on the site of integration, the targeting construct may further include expression regulatory sequences, for example a promoter sequence, or utilize expression regulatory sequences within the target cell genome at the intended integration site.

In some embodiments, the targeting constructs do not include the entire coding sequence of the fusion protein, but only include the degron coding sequence and the optional linker sequence in addition to homology arms. The homology arms may or may not include essential polypeptide coding sequences, as the coding sequence for the entire fusion protein (continuous or with intron sequences) is constituted upon integration of the targeting construct into the target cell genome. The homology arms may be designed to recombine with the essential gene and/or flanking sequences.

In some embodiments, a targeting construct of the disclosure comprises:
(i) a first homology arm as described in Section 6.3.3, corresponding to a 5′ target sequence comprising a first region of homology to an essential gene as described in Section 6.3.2 or its flanking sequence;
(ii) a nucleotide sequence encoding a degron, e.g., an inducible degron such as a drug-inducible degron (“degron coding sequence”) as described in Section 6.2.2 and optionally a nucleotide sequence encoding a linker 5′ or 3′ to the degron coding sequence;
(iii) a second homology arm as described in Section 6.3.3, corresponding to a 3′ target sequence comprising a second region of homology to an essential gene as described in Section 6.3.2 or its flanking sequence;
   wherein the targeting construct is configured such that upon its recombination with the target genomic locus, the essential gene is modified such to encode a fusion protein as described in Section 6.2 comprising the essential polypeptide, the degron and, optionally, a linker.

In certain aspects, the targeting construct further comprises a transgene as described in Section 6.4, e.g., between the degron sequence and the second homology arm. In certain other aspects, the targeting construct of the disclosure further comprises separator sequences as described in Section 6.5, wherein the fusion protein coding sequence and transgene are connected via a separator sequence, e.g., a nucleotide sequence encoding an internal ribosome entry site (IRES) or a self-cleaving peptide.

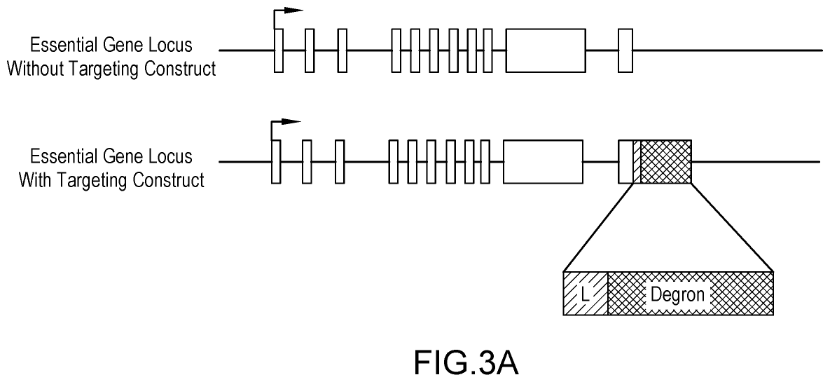

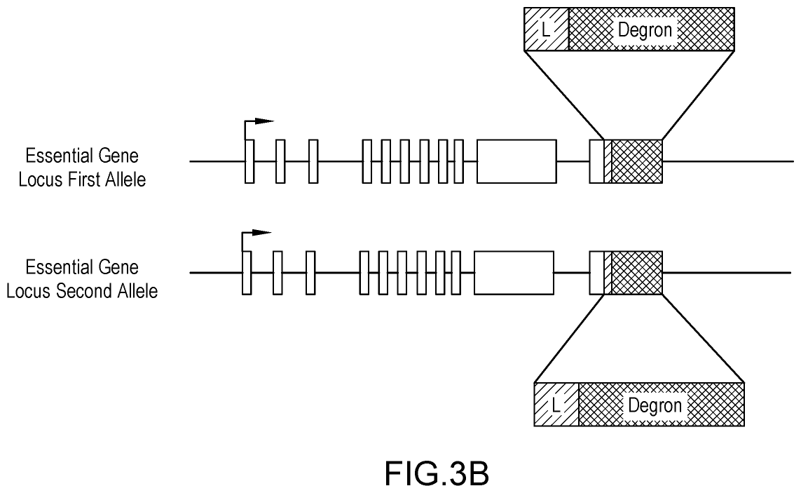

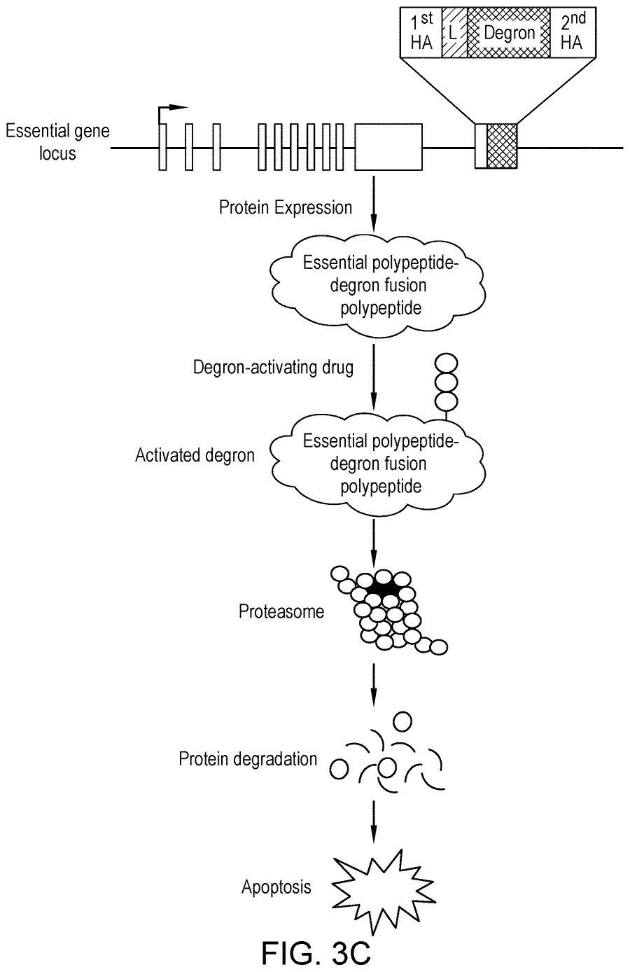

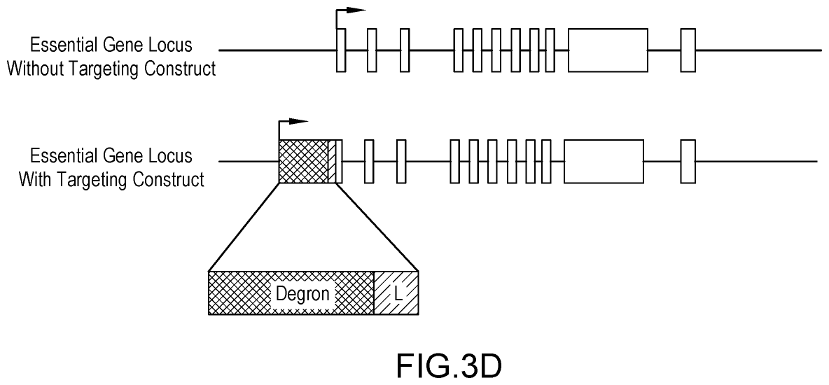

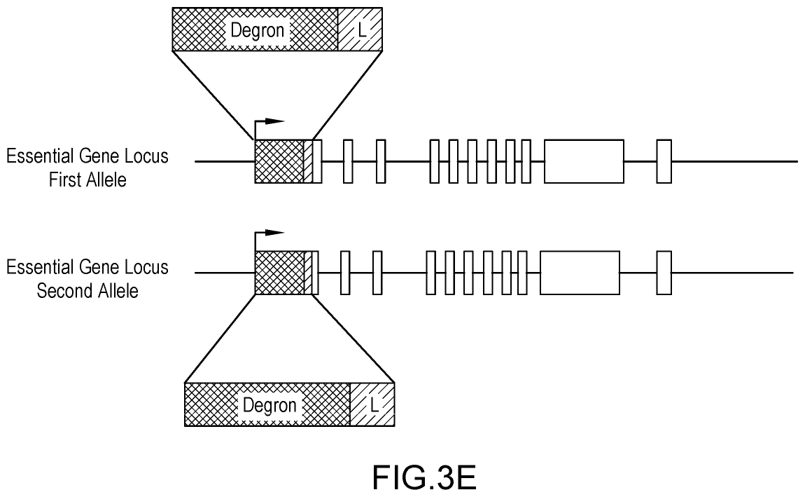

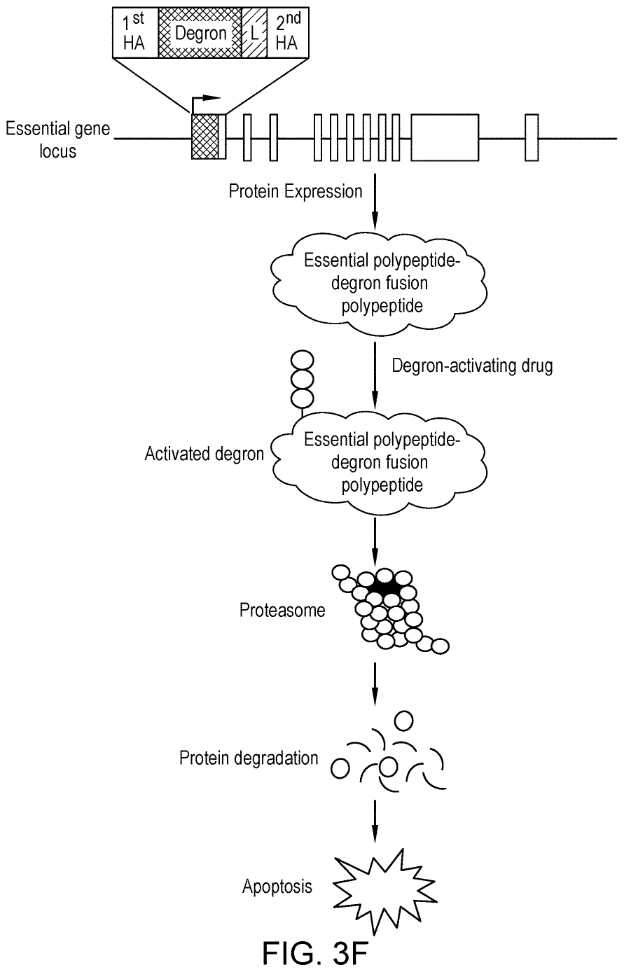

Exemplary configurations of targeting constructs lacking transgenes are presented in FIG. 1 and FIG. 3.

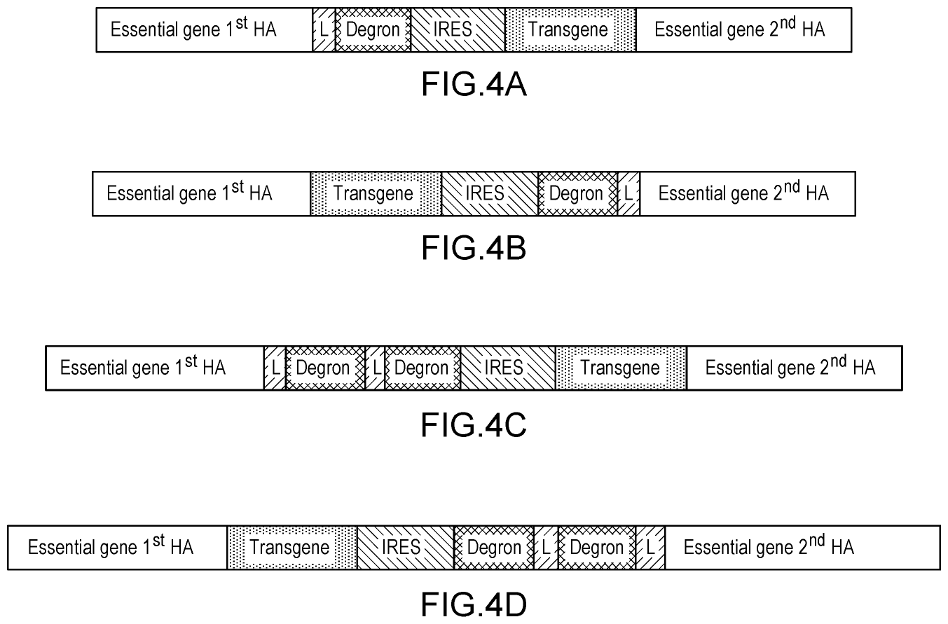

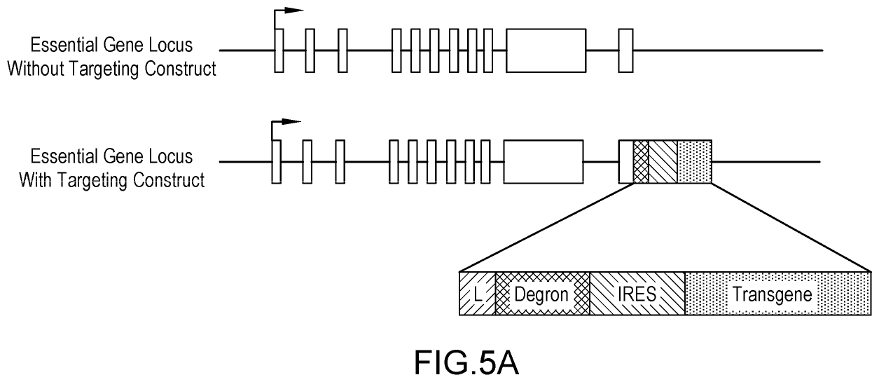

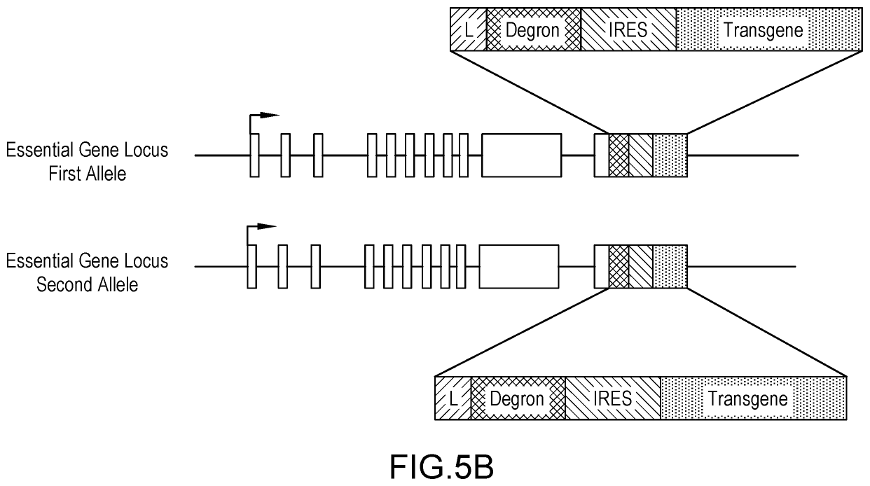

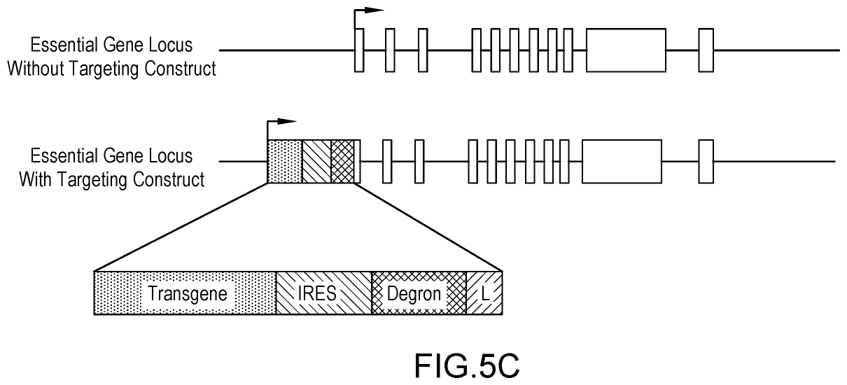

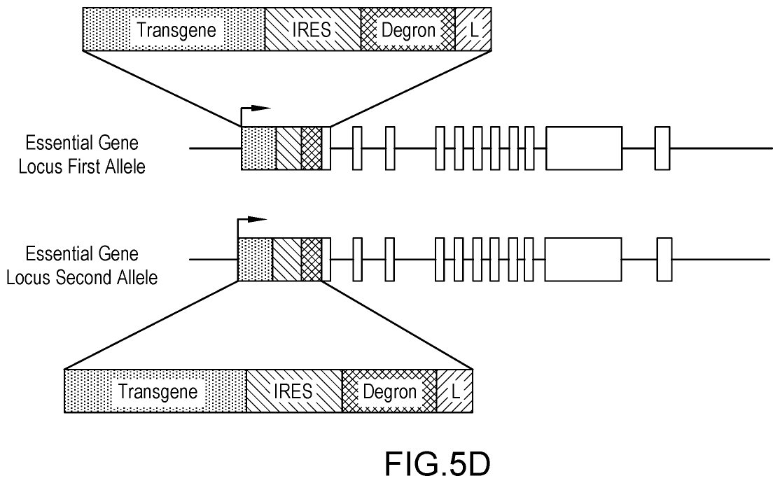

Exemplary configurations of targeting constructs comprising transgenes are depicted in FIG. 4, and exemplary configurations targeting construct integration into essential gene loci are depicted in FIGS. 5A through 5D. As depicted in FIGS. 5A through 5D, the targeting construct may be integrated 5′ of the essential protein coding sequence or 3′ of the essential protein coding sequence, and cells can be selected that are either heterozygous or homozygous for essential gene modification.

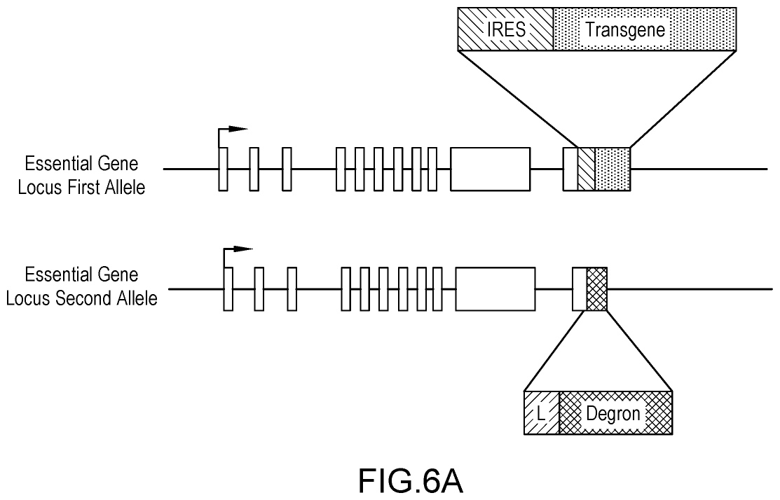

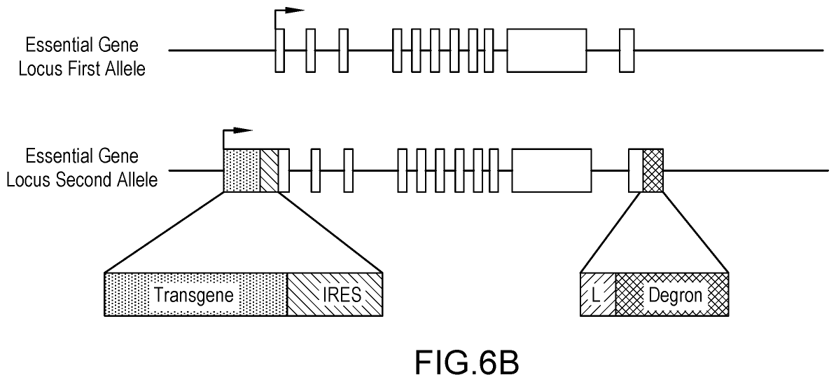

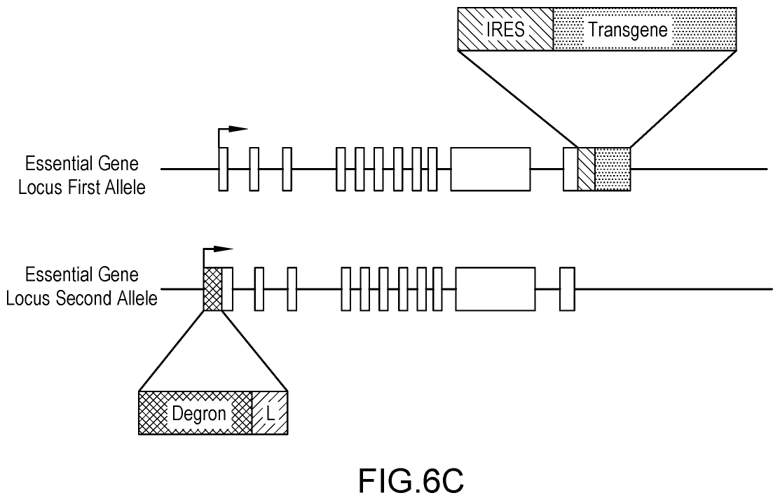

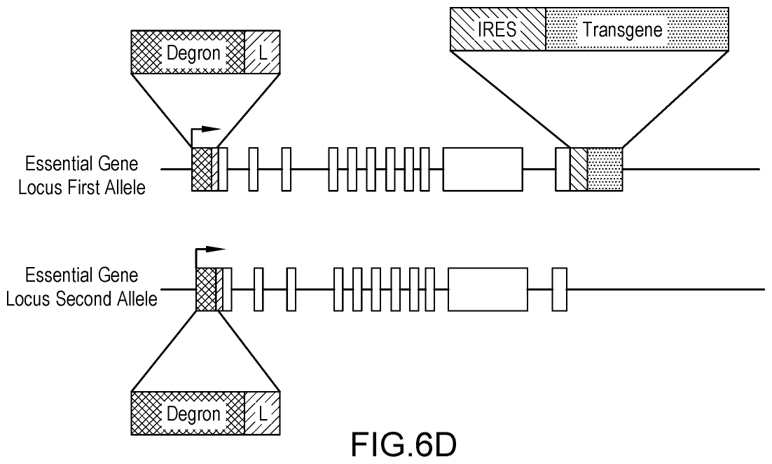

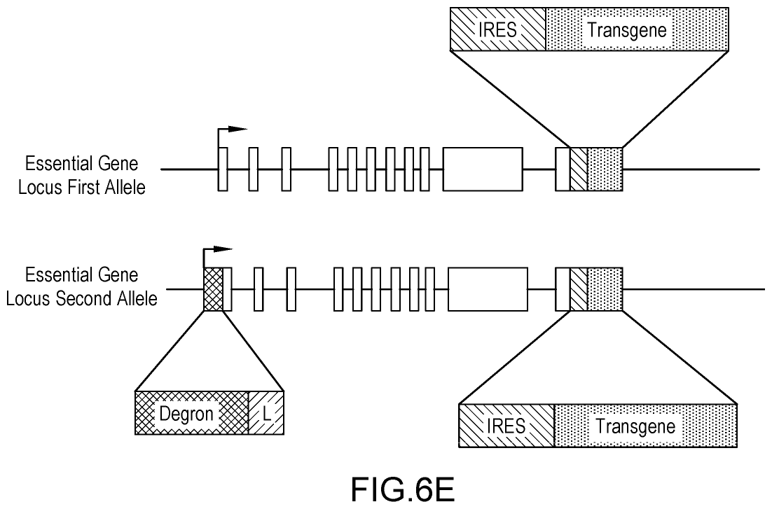

As an alternative (or in addition) to incorporating a transgene within a targeting construct comprising a degron coding sequence, first targeting construct comprising the degron coding sequence can be introduced into a cell with a second targeting construct that lacks the degron but comprises the transgene. The targeting constructs may both be targeted to the same gene, followed by selection of heterozygous cells as depicted in FIGS. 6A through 6F, or they may be targeted to different loci, such that the transgene and the fusion protein comprising the essential polypeptide and the degron are expressed from separate genes. In some embodiments, the first targeting construct and the second targeting construct are both introduced into a STEL locus, as described in PCT application WO 2021/072329 A1. In some embodiments, the first and second targeting constructs are introduced into the same STEL locus, e.g., the GAPDH locus. In other embodiments, the first and second targeting constructs are introduced into different STEL loci, e.g., a GAPDH locus and another STEL locus. In some embodiments, the first targeting construct (i.e., the targeting construct comprising the degron coding sequence) is introduced into the GAPDH locus and the second targeting construct (i.e., the targeting construct comprising the transgene) is introduced into a different STEL locus. In other embodiments, the second targeting construct (i.e., the targeting construct comprising the transgene) is introduced into the GAPDH locus and the first targeting construct (i.e., the targeting construct comprising the degron coding sequence) is introduced into a different STEL locus. In yet other embodiments, the first targeting construct (i.e., the targeting construct comprising the degron coding sequence) is introduced into an essential gene that is not a STEL gene and the second targeting construct (i.e., the targeting construct comprising the transgene) is introduced into a STEL locus.

Nucleic acid sequences for example constructs are set forth in Table 2.

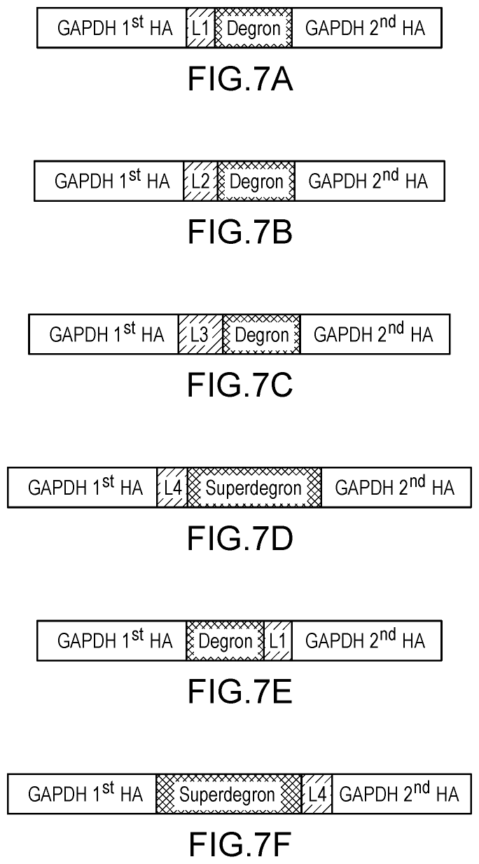

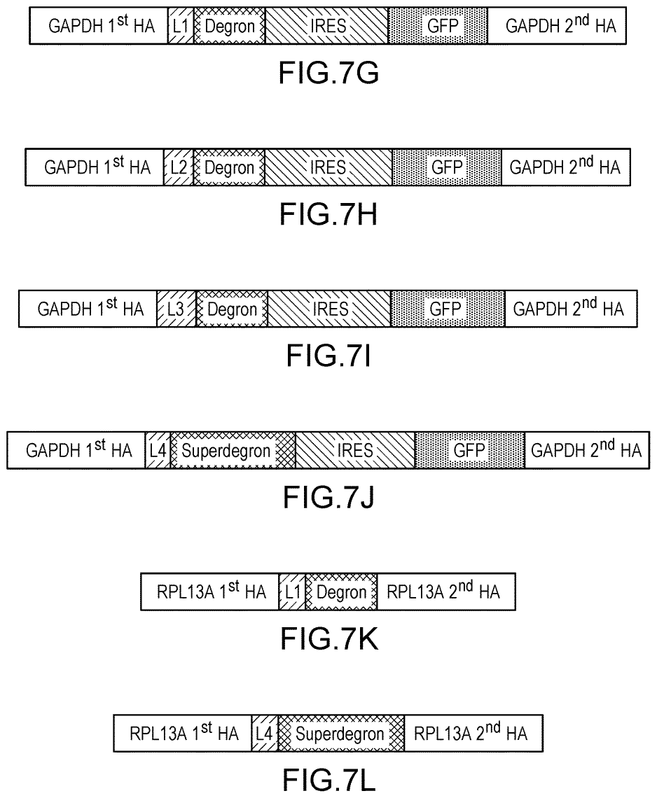

TABLE 2

| Targeting | | Corresponding | SEQ ID |
|---|---|---|---|
| Construct | Sequence | Schematic | NO |
| GAPDH C- | TTGGTATCGTGGAAGGACTCATGGTATGAGAGCTGG | FIG. 7A | 1 |
| terminal LHA- | GGAATGGGACTGAGGCTCCCACCTTTCTCATCCAAG | | |
| linker 1- | ACTGGCTCCTCCCTGCCGGGGCTGCGTGCAACCCT | | |
| degron- | GGGGTTGGGGGTTCTGGGGACTGGCTTTCCCATAA | | |
| stop codon- | TTTCCTTTCAAGGTGGGGAGGGAGGTAGAGGGGTG | | |
| GAPDH C- | ATGTGGGGAGTACGCTGCAGGGCCTCACTCCTTTTG | | |
| terminal RHA | CAGACCACAGTCCATGCCATCACTGCCACCCAGAAG | | |
| | ACTGTGGATGGCCCCTCCGGGAAACTGTGGCGTGA | | |
| | TGGCCGCGGGGCTCTCCAGAACATCATCCCTGCCT | | |
| | CTACTGGCGCTGCCAAGGCTGTGGGCAAGGTCATC | | |
| | CCTGAGCTGAACGGGAAGCTCACTGGCATGGCCTT | | |
| | CCGTGTCCCCACTGCCAACGTGTCAGTGGTGGACC | | |
| | TGACCTGCCGTCTAGAAAAACCTGCCAAATATGATG | | |
| | ACATCAAGAAGGTGGTGAAGCAGGCGTCGGAGGGC | | |
| | CCCCTCAAGGGCATCCTGGGCTACACTGAGCACCA | | |
| | GGTGGTCTCCTCTGACTTCAACAGCGACACCCACTC | | |
| | CTCCACCTTTGACGCTGGGGCTGGCATTGCCCTCAA | | |
| | CGACCACTTTGTCAAGCTCATTTCCTGGTATGTGGC | | |
| | TGGGGCCAGAGACTGGCTCTTAAAAAGTGCAGGGT | | |
| | CTGGCGCCCTCTGGTGGCTGGCTCAGAAAAAGGGC | | |
| | CCTGACAACTCTTTACATCTTCTAGGTATGACAACGA | | |
| | ATTTGGCTACAGCAACAGGGTGGTGGACCTCATGGC | | |
| | CCACATGGCCTCCAAGGAGGGCGGGTCTCGCCCAT | | |
| | TCCAGTGTAATCAGTGTGGGGCATCTTTTACTCAGA | | |
| | AAGGTAACCTCCTCCGCCACATTAAACTGCACTAAG | | |
| | ACCCCTGGACCACCAGCCAAAGCAAGAGCACAAGA | | |
| | GGAAGAGAGAGACCCTCACTGCTGGGGAGTCCCTG | | |
| | CCACACTCAGTCCCCCACCACACTGAATCTCCCCTC | | |
| | CTCACAGTTGCCATGTAGACCCCTTGAAGAGGGGAG | | |
| | GGGCCTAGGGAGCCGCACCTTGTCATGTACCATCAA | | |
| | TAAAGTACCCTGTGCTCAACCAGTTACTTGTCCTGTC | | |
| | TTATTCTAGGGTCTGGGGCAGAGGGGAGGGAAGCT | | |
| | GGGCTTGTGTCAAGGTGAGACATTCTTGCTGGGGA | | |
| | GGGACCTGGTATGTTCTCCTCAGACTGAGGGTAGG | | |
| | GCCTCCAAACAGCCTTGCTTGCTTCGAGAACCATTT | | |
| | GCTTCCCGCTCAGACGTCTTGAGTGCTACAGGAAGC | | |
| | TGGCACCACTACTTCAGAGAACAAGGCCTTTTCCTC | | |
| | TCCTCGCTCCAGTCCTAGGCTATCTGCTGTTGGCCA | | |
| | AACATGGAAGAAGCTATTCTGTGGGCAGCCCCAGG | | |
| | GAGGCTGACAGGTGGAGGAAGTCAGGGCTCGCACT | | |
| | GGGCTCTGACGCTGACTGGTTAGTGGAGCTCAGCC | | |
| | TGGAGCTGAGCTGCAGCGGGCAATTCCAGCTTGGC | | |
| | CTCCGCAGCTGTGAGGTCTTGAGCACGTGCTCTATT | | |
| | GCTTTCTGTGCCCTCGTGTCTTATCTGAGGACATCG | | |
| | TGGCCAGCCCCTAAGGTCTTCAAGCAGGATTCATCT | | |
| | AGGTAAACCAAGTACCTAAAACCATGCCCAAGGCGG | | |
| | TAAGGACTATATAATGTTTAAAAATCGGTAAAAATGC | | |
| | CCACCTCGCATAGT | | |
| GAPDH C- | TTGGTATCGTGGAAGGACTCATGGTATGAGAGCTGG | FIG. 7D | 2 |
| terminal LHA- | GGAATGGGACTGAGGCTCCCACCTTTCTCATCCAAG | | |
| linker 4- | ACTGGCTCCTCCCTGCCGGGGCTGCGTGCAACCCT | | |
| superdegron- | GGGGTTGGGGGTTCTGGGGACTGGCTTTCCCATAA | | |
| stop codon- | TTTCCTTTCAAGGTGGGGAGGGAGGTAGAGGGGTG | | |
| GAPDH C- | ATGTGGGGAGTACGCTGCAGGGCCTCACTCCTTTTG | | |
| terminal RHA | CAGACCACAGTCCATGCCATCACTGCCACCCAGAAG | | |
| | ACTGTGGATGGCCCCTCCGGGAAACTGTGGCGTGA | | |
| | TGGCCGCGGGGCTCTCCAGAACATCATCCCTGCCT | | |
| | CTACTGGCGCTGCCAAGGCTGTGGGCAAGGTCATC | | |
| | CCTGAGCTGAACGGGAAGCTCACTGGCATGGCCTT | | |
| | CCGTGTCCCCACTGCCAACGTGTCAGTGGTGGACC | | |
| | TGACCTGCCGTCTAGAAAAACCTGCCAAATATGATG | | |
| | ACATCAAGAAGGTGGTGAAGCAGGCGTCGGAGGGC | | |
| | CCCCTCAAGGGCATCCTGGGCTACACTGAGCACCA | | |
| | GGTGGTCTCCTCTGACTTCAACAGCGACACCCACTC | | |
| | CTCCACCTTTGACGCTGGGGCTGGCATTGCCCTCAA | | |
| | CGACCACTTTGTCAAGCTCATTTCCTGGTATGTGGC | | |
| | TGGGGCCAGAGACTGGCTCTTAAAAAGTGCAGGGT | | |
| | CTGGCGCCCTCTGGTGGCTGGCTCAGAAAAAGGGC | | |
| | CCTGACAACTCTTTACATCTTCTAGGTATGACAACGA | | |
| | ATTTGGCTACAGCAACAGGGTGGTGGACCTCATGGC | | |
| | CCACATGGCCTCCAAGGAGGGCTCAGGTAGCGGAA | | |
| | GCGGATCAGGTGGATTCAATGTACTGATGGTCCATA | | |
| | AACGGAGTCACACTGGCGAGCGCCCGCTCCAATGT | | |
| | GAAATCTGCGGGTTCACGTGTCGGCAGAAGGGCAA | | |
| | CCTCCTCCGGCATATCAAGCTGCACACGGGTGAAAA | | |
| | ACCGTTTAAGTGCCATCTCTGCAATTACGCCTGTCA | | |
| | GAGAAGAGATGCTTTGTAAGACCCCTGGACCACCAG | | |
| | CCAAAGCAAGAGCACAAGAGGAAGAGAGAGACCCT | | |
| | CACTGCTGGGGAGTCCCTGCCACACTCAGTCCCCC | | |
| | ACCACACTGAATCTCCCCTCCTCACAGTTGCCATGT | | |
| | AGACCCCTTGAAGAGGGGAGGGGCCTAGGGAGCC | | |
| | GCACCTTGTCATGTACCATCAATAAAGTACCCTGTG | | |
| | CTCAACCAGTTACTTGTCCTGTCTTATTCTAGGGTCT | | |
| | GGGGCAGAGGGGAGGGAAGCTGGGCTTGTGTCAA | | |
| | GGTGAGACATTCTTGCTGGGGAGGGACCTGGTATG | | |
| | TTCTCCTCAGACTGAGGGTAGGGCCTCCAAACAGCC | | |
| | TTGCTTGCTTCGAGAACCATTTGCTTCCCGCTCAGA | | |
| | CGTCTTGAGTGCTACAGGAAGCTGGCACCACTACTT | | |
| | CAGAGAACAAGGCCTTTTCCTCTCCTCGCTCCAGTC | | |
| | CTAGGCTATCTGCTGTTGGCCAAACATGGAAGAAGC | | |
| | TATTCTGTGGGCAGCCCCAGGGAGGCTGACAGGTG | | |
| | GAGGAAGTCAGGGCTCGCACTGGGCTCTGACGCTG | | |
| | ACTGGTTAGTGGAGCTCAGCCTGGAGCTGAGCTGC | | |
| | AGCGGGCAATTCCAGCTTGGCCTCCGCAGCTGTGA | | |
| | GGTCTTGAGCACGTGCTCTATTGCTTTCTGTGCCCT | | |
| | CGTGTCTTATCTGAGGACATCGTGGCCAGCCCCTAA | | |
| | GGTCTTCAAGCAGGATTCATCTAGGTAAACCAAGTA | | |
| | CCTAAAACCATGCCCAAGGCGGTAAGGACTATATAA | | |
| | TGTTTAAAAATCGGTAAAAATGCCCACCTCGCATAGT | | |
| GAPDH N- | GGAGAAGTTCCCCAACTTTCCCGCCTCTCAGCCTTT | FIG. 7E | 29 |
| terminal LHA- | GAAAGAAAGAAAGGGGAGGGGGCAGGCCGCGTGC | | |
| start codon-- | AGCCGCGAGCGGTGCTGGGCTCCGGCTCCAATTCC | | |
| degron- | CCATCTCAGTCGTTCCCAAAGTCCTCCTGTTTCATCC | | |
| linker 1- | AAGCGTGTAAGGGTCCCCGTCCTTGACTCCCTAGTG | | |
| GAPDH N- | TCCTGCTGCCCACAGTCCAGTCCTGGGAACCAGCA | | |
| terminal RHA | CCGATCACCTCCCATCGGGCCAATCTCAGTCCCTTC | | |
| | CCCCCTACGTCGGGGCCCACACGCTCGGTGCGTGC | | |
| | CCAGTTGAACCAGGCGGCTGCGGAAAAAAAAAAGC | | |
| | GGGGAGAAAGTAGGGCCCGGCTACTAGCGGTTTTA | | |
| | CGGGCGCACGTAGCTCAGGCCTCAAGACCTTGGGC | | |
| | TGGGACTGGCTGAGCCTGGCGGGAGGCGGGGTCC | | |
| | GAGTCACCGCCTGCCGCCGCGCCCCCGGTTTCTAT | | |
| | AAATTGAGCCCGCAGCCTCCCGCTTCGCTCTCTGCT | | |
| | CCTCCTGTTCGACAGTCAGCCGCATCTTCTTTTGCG | | |
| | TCGCCAGGTGAAGACGGGCGGAGAGAAACCCGGGA | | |
| | GGCTAGGGACGGCCTGAAGGCGGCAGGGGCGGGC | | |
| | GCAGGCCGGATGTGTTCGCGCCGCTGCGGGGTGG | | |
| | GCCCGGGCGGCCTCCGCATTGCAGGGGCGGGCGG | | |
| | AGGACGTGATGCGGCGCGGGCTGGGCATGGAGGC | | |
| | CTGGTGGGGGAGGGGAGGGGAGGCGTGTGTGTCG | | |
| | GCCGGGGCCACTAGGCGCTCACTGTTCTCTCCCTC | | |
| | CGCGCAGCCGAGCCACATCGCTCAGACACCATGCG | | |
| | CCCATTCCAGTGTAATCAGTGTGGGGCATCTTTTACT | | |
| | CAGAAAGGTAACCTCCTCCGCCACATTAAACTGCAC | | |
| | GGCGGGTCTGGGAAGGTGAAGGTCGGAGTCAACGG | | |
| | GTGAGTTCGCGAATGGCTGGGGGGCCCTGGGCTGC | | |
| | GACCGCCCCCGAACCGCGTCTACGAGCCTTGCGGG | | |
| | CTCCGGGTCTTAGCAGTCGTATGGGGGCAGGGTAG | | |
| | CTGTTCCCCGCAAGGAGAGCTCAAGGTCAGCGCTC | | |
| | GGACCTGGCGGAGCCCCGCACCCAGGCTGTGGCG | | |
| | CCCTGTGCAGCTCCGCCCTTGCGGCGCCATCTGCC | | |
| | CGGAGCCTCCTTCCCCTAGTCCCCAGAAACAGGAG | | |
| | GTCCCTACTCCCGCCCGAGATCCCGACCCGGACCC | | |
| | CTAGGTGGGGGACGCTTTCTTTCCTTTCGCGCTCTG | | |
| | CGGGGTCACGTGTCGCAGAGGAGCCCCTCCCCCAC | | |
| | GGCCTCCGGCACCGCAGGCCCCGGGATGCTAGTGC | | |
| | GCAGCGGGTGCATCCCTGTCCGGATGCTGCGCCTG | | |
| | CGGTAGAGCGGCCGCCATGTTGCAACCGGGAAGGA | | |
| | AATGAATGGGCAGCCGTTAGGAAAGCCTGCCGGTG | | |
| | ACTAACCCTGCGCTCCTGCCTCGATGGGTGGAGTC | | |
| | GCGTGTGGCGGGGAAGTCAGGTGGAGCGAGGCTA | | |
| | GCTGGCCCGATTTCTCCTCCGGGTGATGCTTTTCCT | | |
| | AGATTATTCTCTGGTAAATCAAAGAAGTGGGTTTATG | | |
| | GAGGTCCTCTTGTGTCCCCTCCCCGCAGAGGTGTG | | |
| | GTGGCTGTGGCATGGTGCCAAGCCGGGAGAAGCTG | | |
| | AGTCATGGGTAGTTGGAAAAGGACATTTCCACCGCA | | |
| | AAATGGCCCCTCTGGTGGTGGCCCCTTCCTGCAGC | | |
| | G | | |
| GAPDH N- | GGAGAAGTTCCCCAACTTTCCCGCCTCTCAGCCTTT | FIG. 7F | 30 |
| terminal LHA- | GAAAGAAAGAAAGGGGAGGGGGCAGGCCGCGTGC | | |
| start codon- | AGCCGCGAGCGGTGCTGGGCTCCGGCTCCAATTCC | | |
| superdegron- | CCATCTCAGTCGTTCCCAAAGTCCTCCTGTTTCATCC | | |
| linker 4- | AAGCGTGTAAGGGTCCCCGTCCTTGACTCCCTAGTG | | |
| GAPDH N- | TCCTGCTGCCCACAGTCCAGTCCTGGGAACCAGCA | | |
| terminal RHA | CCGATCACCTCCCATCGGGCCAATCTCAGTCCCTTC | | |
| | CCCCCTACGTCGGGGCCCACACGCTCGGTGCGTGC | | |
| | CCAGTTGAACCAGGCGGCTGCGGAAAAAAAAAAGC | | |
| | GGGGAGAAAGTAGGGCCCGGCTACTAGCGGTTTTA | | |
| | CGGGCGCACGTAGCTCAGGCCTCAAGACCTTGGGC | | |
| | TGGGACTGGCTGAGCCTGGCGGGAGGCGGGGTCC | | |
| | GAGTCACCGCCTGCCGCCGCGCCCCCGGTTTCTAT | | |
| | AAATTGAGCCCGCAGCCTCCCGCTTCGCTCTCTGCT | | |
| | CCTCCTGTTCGACAGTCAGCCGCATCTTCTTTTGCG | | |
| | TCGCCAGGTGAAGACGGGCGGAGAGAAACCCGGGA | | |
| | GGCTAGGGACGGCCTGAAGGCGGCAGGGGCGGGC | | |
| | GCAGGCCGGATGTGTTCGCGCCGCTGCGGGGTGG | | |
| | GCCCGGGCGGCCTCCGCATTGCAGGGGCGGGCGG | | |
| | AGGACGTGATGCGGCGCGGGCTGGGCATGGAGGC | | |
| | CTGGTGGGGGAGGGGAGGGGAGGCGTGTGTGTCG | | |
| | GCCGGGGCCACTAGGCGCTCACTGTTCTCTCCCTC | | |
| | CGCGCAGCCGAGCCACATCGCTCAGACACCATGTT | | |
| | CAATGTACTGATGGTCCATAAACGGAGTCACACTGG | | |
| | CGAGCGCCCGCTCCAATGTGAAATCTGCGGGTTCA | | |
| | CGTGTCGGCAGAAGGGCAACCTCCTCCGGCATATC | | |
| | AAGCTGCACACGGGTGAAAAACCGTTTAAGTGCCAT | | |
| | CTCTGCAATTACGCCTGTCAGAGAAGAGATGCTTTG | | |
| | GGCTCAGGTAGCGGAAGCGGATCAGGTGGAGGGA | | |
| | AGGTGAAGGTCGGAGTCAACGGGTGAGTTCGCGAA | | |
| | TGGCTGGGGGGCCCTGGGCTGCGACCGCCCCCGA | | |
| | ACCGCGTCTACGAGCCTTGCGGGCTCCGGGTCTTA | | |
| | GCAGTCGTATGGGGGCAGGGTAGCTGTTCCCCGCA | | |
| | AGGAGAGCTCAAGGTCAGCGCTCGGACCTGGCGGA | | |
| | GCCCCGCACCCAGGCTGTGGCGCCCTGTGCAGCTC | | |
| | CGCCCTTGCGGCGCCATCTGCCCGGAGCCTCCTTC | | |
| | CCCTAGTCCCCAGAAACAGGAGGTCCCTACTCCCG | | |
| | CCCGAGATCCCGACCCGGACCCCTAGGTGGGGGAC | | |
| | GCTTTCTTTCCTTTCGCGCTCTGCGGGGTCACGTGT | | |
| | CGCAGAGGAGCCCCTCCCCCACGGCCTCCGGCACC | | |
| | GCAGGCCCCGGGATGCTAGTGCGCAGCGGGTGCAT | | |
| | CCCTGTCCGGATGCTGCGCCTGCGGTAGAGCGGCC | | |
| | GCCATGTTGCAACCGGGAAGGAAATGAATGGGCAG | | |
| | CCGTTAGGAAAGCCTGCCGGTGACTAACCCTGCGC | | |
| | TCCTGCCTCGATGGGTGGAGTCGCGTGTGGCGGGG | | |
| | AAGTCAGGTGGAGCGAGGCTAGCTGGCCCGATTTC | | |
| | TCCTCCGGGTGATGCTTTTCCTAGATTATTCTCTGGT | | |
| | AAATCAAAGAAGTGGGTTTATGGAGGTCCTCTTGTG | | |
| | TCCCCTCCCCGCAGAGGTGTGGTGGCTGTGGCATG | | |
| | GTGCCAAGCCGGGAGAAGCTGAGTCATGGGTAGTT | | |
| | GGAAAAGGACATTTCCACCGCAAAATGGCCCCTCTG | | |
| | GTGGTGGCCCCTTCCTGCAGCG | | |
| RPL13A N- | CACAGGAATCCAAATCTCTAAGCCTAGAATGGGCAA | N/A | 31 |
| terminal LHA- | GGCTCCGGGGCTCACGCCGGTAATCCCAGCACTTT | | |
| start codon- | GGGAGGCCGAAACGGGAGGATCGCTTGAGCCCAG | | |
| degron- | GAGTTCGAGATCAGCCTGGCCAACATGTCAGACTCC | | |
| linker 1- | CCCCGTTCCCGGGCCACAACAAAATATATATATATAT | | |
| RPL13A N- | ATATATTAGCCAGGCATGGTGGCGCGCCCCTGTAGT | | |
| terminal RHA | CCAAGTTACTCGAGAGGCTGAGGCAGGAGGATCCC | | |
| | TTGAGCCCAGGAAGTGGAGGCTGCGGTGAGCCATG | | |
| | ATCGCCCCACAGCACTCTGGTGTGGACAACACAGC | | |
| | GAGACCCTGTCTCACAAAATAAAGTAAGCCCGGACT | | |
| | GAGTGCGGAAAGGCGGGCCTGGCGGGTCTGGTCTC | | |
| | CCCATGCGGGCCACCAGAGGCCCTGCAGCCTTCAG | | |
| | TCGCTTGAAGGGGTAATGGCGCTTCCACTCACAAAC | | |
| | ATGGCGGACAGAGCGTGTGAACGAGATGAACAGCC | | |
| | CCTCAAAAATATGGCCGCCGAGGCTGGACGGCCGT | | |
| | GCCCCAGCAGCACCGCCTCCGCGCCCCACGTGATC | | |
| | TCTCGCCGGGCACAGCGCTGACCGCGGAGGTCCAA | | |
| | CCGGAAGAATGTCCGGATTGGACATTCGGAAGAGG | | |
| | GCCCGCCTTCCCTGGGGAATCTCTGCGCACGCGCA | | |
| | GAACGCTTCGACCAATGAAAACACAGGAAGCCGTCC | | |
| | GCGCAACCGCGTTGCGTCACTTCTGCCGCCCCTGTT | | |
| | TCAAGGGATAAGAAACCCTGCGACAAAACCTCCTCC | | |
| | TTTTCCAAGCGGCTGCCGAAGATGCGCCCATTCCAG | | |
| | TGTAATCAGTGTGGGGCATCTTTTACTCAGAAAGGT | | |
| | AACCTCCTCCGCCACATTAAACTGCACGGCGGGTCT | | |
| | GCGGAGGTGCAGGTATGGGCTCCGCGCGGGCCGG | | |
| | GGCGGCAAGGGGCCGGGTGGGATCCAGGCCGGAA | | |
| | TGGGGTCGCACCCTCTCTGTCTTGCATGTTTTGCGG | | |
| | AGCTTAACATCCATAATGAAGCAAAATGGAAGTCTTT | | |
| | TGGGATAAGGGGAATCTGTAGGAAATGCCGCGGCT | | |
| | CTCTTTAGGCCTAGGGTTGGCTACAATGTGGCATTT | | |
| | CCTTCTCGAGGCGAGGGTGATAGAGCTTCCAGCAC | | |
| | AGGACAGGTATTTTGCAACTAGATTTTGCTTACCTTG | | |
| | AACTCGGGTAGTGGGTGGGGGCCCTGGGGTAGAGT | | |
| | CTTGGCCGAATTGGACCCTTGTGTGTCCTCAGCGAT | | |
| | GAGGAACACGCGGAAGTGTGGCCGGGCGGTCTCG | | |
| | GGGCAATGAGAAACTGAGTTTTGGCCAAAATGGAAG | | |
| | GTATTTTTATCCCGCTCAGTGCGCGGGCTACTTTGA | | |
| | TTTCACGTGGAGGTGTTAATCGGGGTCCCCTGCAGC | | |
| | TCCGGTTCTTTTAATTCTCAATGCCTTTCTGCGGGGG | | |
| | GTCGAGAGCTGTTGGCTGCGGGGCACTCTGGAATG | | |
| | AACTCGTTTTCCGTCGTTTTGGGGATTGGCGATTGT | | |
| | CCTGAGGCGTGTTACTGGAAGTCGAGAGCTGTGAT | | |
| | GAATGGTTCCACAAGGATGGGACATCTTTTGGGCCT | | |
| | GACACCTGGGGGCCCTTTCCCTGGCTCCTGGTGTA | | |
| | GCTACTTAAGTATAAAGGAGGGATTGGCCCTGGAAA | | |
| | GGTTTCTTTGTGAGTAGTCTGTTGTTGGGGCGGGTG | | |
| | ACATTTCAGAGCCAGGC | | |
| RPL13A N- | CACAGGAATCCAAATCTCTAAGCCTAGAATGGGCAA | N/A | 32 |
| terminal LHA- | GGCTCCGGGGCTCACGCCGGTAATCCCAGCACTTT | | |
| start codon- | GGGAGGCCGAAACGGGAGGATCGCTTGAGCCCAG | | |
| superdegron- | GAGTTCGAGATCAGCCTGGCCAACATGTCAGACTCC | | |
| linker 4- | CCCCGTTCCCGGGCCACAACAAAATATATATATATAT | | |
| RPL13A N- | ATATATTAGCCAGGCATGGTGGCGCGCCCCTGTAGT | | |
| terminal RHA | CCAAGTTACTCGAGAGGCTGAGGCAGGAGGATCCC | | |
| | TTGAGCCCAGGAAGTGGAGGCTGCGGTGAGCCATG | | |
| | ATCGCCCCACAGCACTCTGGTGTGGACAACACAGC | | |
| | GAGACCCTGTCTCACAAAATAAAGTAAGCCCGGACT | | |
| | GAGTGCGGAAAGGCGGGCCTGGCGGGTCTGGTCTC | | |
| | CCCATGCGGGCCACCAGAGGCCCTGCAGCCTTCAG | | |
| | TCGCTTGAAGGGGTAATGGCGCTTCCACTCACAAAC | | |
| | ATGGCGGACAGAGCGTGTGAACGAGATGAACAGCC | | |
| | CCTCAAAAATATGGCCGCCGAGGCTGGACGGCCGT | | |
| | GCCCCAGCAGCACCGCCTCCGCGCCCCACGTGATC | | |
| | TCTCGCCGGGCACAGCGCTGACCGCGGAGGTCCAA | | |
| | CCGGAAGAATGTCCGGATTGGACATTCGGAAGAGG | | |
| | GCCCGCCTTCCCTGGGGAATCTCTGCGCACGCGCA | | |
| | GAACGCTTCGACCAATGAAAACACAGGAAGCCGTCC | | |
| | GCGCAACCGCGTTGCGTCACTTCTGCCGCCCCTGTT | | |
| | TCAAGGGATAAGAAACCCTGCGACAAAACCTCCTCC | | |
| | TTTTCCAAGCGGCTGCCGAAGATGTTCAATGTACTG | | |
| | ATGGTCCATAAACGGAGTCACACTGGCGAGCGCCC | | |
| | GCTCCAATGTGAAATCTGCGGGTTCACGTGTCGGCA | | |
| | GAAGGGCAACCTCCTCCGGCATATCAAGCTGCACAC | | |
| | GGGTGAAAAACCGTTTAAGTGCCATCTCTGCAATTA | | |
| | CGCCTGTCAGAGAAGAGATGCTTTGGGCTCAGGTA | | |
| | GCGGAAGCGGATCAGGTGGAGCGGAGGTGCAGGT | | |
| | ATGGGCTCCGCGCGGGCCGGGGCGGCAAGGGGCC | | |
| | GGGTGGGATCCAGGCCGGAATGGGGTCGCACCCTC | | |
| | TCTGTCTTGCATGTTTTGCGGAGCTTAACATCCATAA | | |
| | TGAAGCAAAATGGAAGTCTTTTGGGATAAGGGGAAT | | |
| | CTGTAGGAAATGCCGCGGCTCTCTTTAGGCCTAGGG | | |
| | TTGGCTACAATGTGGCATTTCCTTCTCGAGGCGAGG | | |
| | GTGATAGAGCTTCCAGCACAGGACAGGTATTTTGCA | | |
| | ACTAGATTTTGCTTACCTTGAACTCGGGTAGTGGGT | | |
| | GGGGGCCCTGGGGTAGAGTCTTGGCCGAATTGGAC | | |
| | CCTTGTGTGTCCTCAGCGATGAGGAACACGCGGAA | | |
| | GTGTGGCCGGGCGGTCTCGGGGCAATGAGAAACTG | | |
| | AGTTTTGGCCAAAATGGAAGGTATTTTTATCCCGCTC | | |
| | AGTGCGCGGGCTACTTTGATTTCACGTGGAGGTGTT | | |
| | AATCGGGGTCCCCTGCAGCTCCGGTTCTTTTAATTC | | |
| | TCAATGCCTTTCTGCGGGGGGTCGAGAGCTGTTGG | | |
| | CTGCGGGGCACTCTGGAATGAACTCGTTTTCCGTCG | | |
| | TTTTGGGGATTGGCGATTGTCCTGAGGCGTGTTACT | | |
| | GGAAGTCGAGAGCTGTGATGAATGGTTCCACAAGGA | | |
| | TGGGACATCTTTTGGGCCTGACACCTGGGGGCCCTT | | |
| | TCCCTGGCTCCTGGTGTAGCTACTTAAGTATAAAGG | | |
| | AGGGATTGGCCCTGGAAAGGTTTCTTTGTGAGTAGT | | |
| | CTGTTGTTGGGGCGGGTGACATTTCAGAGCCAGGC | | |
| RPLP0 N- | GCGTGAACCCGGGAGGCGGAGCTTGCAGTCAGCCG | N/A | 33 |
| terminal LHA- | AGATGGCGCCACTGCGCTCCAGCCTGGGCGACAGA | | |
| start codon- | GCGACACTCCGTCTCAAAAAAAAAAAAAAAAAGAGA | | |
| degron- | AACCATTCTGTTTCTAAGCATGTGCAATTATGTCTCA | | |
| linker 1- | GCTCCACGTCATACCTTTTAGTTTGCTGAGCTCGCC | | |
| RPLP0 N- | AGGTGGCACAGGGAACCGGGACTCCGAATTCGCAG | | |
| terminal RHA | CTCCAGCTCCAGCTCCAGCCCAGCAGGTGGCAGCA | | |
| | GCTCAGAGCAAGCTCCTCCAACGCGAGGCAGCGCC | | |
| | TTCCTTCGCGACCCTACTTAAAGGCGGCTTTCCGTT | | |
| | GGCTGGAGTGGCAGCGATATTATCCAATGGTTGCCT | | |
| | GTATTCGTTCAGCTTTGTCTGACGGGCGATGGCGCA | | |
| | GCCAATAGACAGGAGCGCTATCCGCGGTTTCTGATT | | |
| | GGCTACTTTGTTCGCATTATAAAAGGCACGCGCGGG | | |
| | CGCGAGGCCCTTCTCTCGCCAGGCGTCCTCGTGGA | | |
| | AGGTTCGTGTGCTAGTTAGATGGGCGCCAGGGGTC | | |
| | GCCGGCGGGAAGCATGGAGGGGTCTTTGGGGGCC | | |
| | TTTGGGAACATGGAGTCCTATTCTGTTCCGCCTGGG | | |
| | GCCTCGGTGGCGGCTTGCACGCCCCGAGATGACGG | | |
| | CCGCTGCCCTAGGCAGGGCCGGCGGGCGATTGCG | | |
| | CGTGTCCTGCTCCTCTTAGGCCCGGGACCGCGGGA | | |
| | TGGGTGTCGGCGTGACCAGGCCTGAGCTCCCTGTC | | |
| | TCTCCTCAGTGACATCGTCTTTAAACCCTGCGTGGC | | |
| | AATCCCTGACGCACCGCCGTGATGCGCCCATTCCA | | |
| | GTGTAATCAGTGTGGGGCATCTTTTACTCAGAAAGG | | |
| | TAACCTCCTCCGCCACATTAAACTGCACGGCGGGTC | | |
| | TCCCAGGGAAGACAGGGCGACCTGGAAGTCCAACT | | |
| | ACTTCCTTAAGATCATCGTAAGTGCAGGGTGGGTCG | | |
| | CCTCTGCTCTTCATGTTGCCCCAGCGCAAATAGGGA | | |
| | CAGTCAGCTGCTATGTGCTGAGGGTCTACTCACACC | | |
| | GGCTACTGAATTAGGCCATTTTTGGGAAAATACTGTT | | |
| | TAGTTAACAATTTCCTGAGATAGGTCCCTTCTGTTGC | | |
| | AGATAAACGGGCTCAGGCAAGTTAAGTGGGTCCTAA | | |
| | GATGACAGCATTCGTATCCAGGTCTGTCTGGCTTCT | | |
| | AAAAGAGCGCGCTTTATACTTTTTTTTTTTTAAACGG | | |
| | AGTCTCGCTCTGTTGTCCAGGCTGGAGTGCAGTGG | | |
| | CTTGATCTTGGCTTACTGCAACCTCCGCTTCCCAGG | | |
| | TTCAAGCGATTCTCCTGCTTCAGCCTCCCGAGTAGC | | |
| | TGGGATTACAGGGGCGCGACACCACACCCAGCTAA | | |
| | TTTTTGTATTTTCAATAGAGACAGGGATTCACCATGT | | |
| | TGGCCAGGATGGTCTCGATCTCTTGACTTCATGATC | | |
| | CACCTTCCTCGGCCTCCCTAAGTGCTAGGATTACAG | | |
| | GTGTGAGCCACCGCGCCCGGCCGCACATTACAGTT | | |
| | TTACTCCATTTTTGAGAAGGTCTGAGGTCAGGAATG | | |
| | ACTCATTGTAAGGAAGCAAGCTGTTGCATATTTAGGA | | |
| | CCTGTCAGCCAAGAAACTTAAGTGTCCAATGACTTTT | | |
| | GGATGGCCTTTGAGGTGGGTCTCCCTTGCCCAGAAA | | |
| | TGCTGACTGTACTGCGCTTAAGCAATTAGGCAGCAT | | |
| | TGTAGAG | | |
| RPLP0 N- | GCGTGAACCCGGGAGGCGGAGCTTGCAGTCAGCCG | N/A | 34 |
| terminal LHA- | AGATGGCGCCACTGCGCTCCAGCCTGGGCGACAGA | | |
| start codon- | GCGACACTCCGTCTCAAAAAAAAAAAAAAAAAGAGA | | |
| superdegron- | AACCATTCTGTTTCTAAGCATGTGCAATTATGTCTCA | | |
| linker 4- | GCTCCACGTCATACCTTTTAGTTTGCTGAGCTCGCC | | |
| RPLP0 N- | AGGTGGCACAGGGAACCGGGACTCCGAATTCGCAG | | |
| terminal RHA | CTCCAGCTCCAGCTCCAGCCCAGCAGGTGGCAGCA | | |
| | GCTCAGAGCAAGCTCCTCCAACGCGAGGCAGCGCC | | |
| | TTCCTTCGCGACCCTACTTAAAGGCGGCTTTCCGTT | | |
| | GGCTGGAGTGGCAGCGATATTATCCAATGGTTGCCT | | |
| | GTATTCGTTCAGCTTTGTCTGACGGGCGATGGCGCA | | |
| | GCCAATAGACAGGAGCGCTATCCGCGGTTTCTGATT | | |
| | GGCTACTTTGTTCGCATTATAAAAGGCACGCGCGGG | | |
| | CGCGAGGCCCTTCTCTCGCCAGGCGTCCTCGTGGA | | |
| | AGGTTCGTGTGCTAGTTAGATGGGCGCCAGGGGTC | | |
| | GCCGGCGGGAAGCATGGAGGGGTCTTTGGGGGCC | | |
| | TTTGGGAACATGGAGTCCTATTCTGTTCCGCCTGGG | | |
| | GCCTCGGTGGCGGCTTGCACGCCCCGAGATGACGG | | |
| | CCGCTGCCCTAGGCAGGGCCGGCGGGCGATTGCG | | |
| | CGTGTCCTGCTCCTCTTAGGCCCGGGACCGCGGGA | | |
| | TGGGTGTCGGCGTGACCAGGCCTGAGCTCCCTGTC | | |
| | TCTCCTCAGTGACATCGTCTTTAAACCCTGCGTGGC | | |
| | AATCCCTGACGCACCGCCGTGATGTTCAATGTACTG | | |
| | ATGGTCCATAAACGGAGTCACACTGGCGAGCGCCC | | |
| | GCTCCAATGTGAAATCTGCGGGTTCACGTGTCGGCA | | |
| | GAAGGGCAACCTCCTCCGGCATATCAAGCTGCACAC | | |
| | GGGTGAAAAACCGTTTAAGTGCCATCTCTGCAATTA | | |
| | CGCCTGTCAGAGAAGAGATGCTTTGGGCTCAGGTA | | |
| | GCGGAAGCGGATCAGGTGGACCCAGGGAAGACAG | | |
| | GGCGACCTGGAAGTCCAACTACTTCCTTAAGATCAT | | |
| | CGTAAGTGCAGGGTGGGTCGCCTCTGCTCTTCATGT | | |
| | TGCCCCAGCGCAAATAGGGACAGTCAGCTGCTATGT | | |
| | GCTGAGGGTCTACTCACACCGGCTACTGAATTAGGC | | |
| | CATTTTTGGGAAAATACTGTTTAGTTAACAATTTCCT | | |
| | GAGATAGGTCCCTTCTGTTGCAGATAAACGGGCTCA | | |
| | GGCAAGTTAAGTGGGTCCTAAGATGACAGCATTCGT | | |
| | ATCCAGGTCTGTCTGGCTTCTAAAAGAGCGCGCTTT | | |
| | ATACTTTTTTTTTTTTAAACGGAGTCTCGCTCTGTTGT | | |
| | CCAGGCTGGAGTGCAGTGGCTTGATCTTGGCTTACT | | |
| | GCAACCTCCGCTTCCCAGGTTCAAGCGATTCTCCTG | | |
| | CTTCAGCCTCCCGAGTAGCTGGGATTACAGGGGCG | | |
| | CGACACCACACCCAGCTAATTTTTGTATTTTCAATAG | | |
| | AGACAGGGATTCACCATGTTGGCCAGGATGGTCTCG | | |
| | ATCTCTTGACTTCATGATCCACCTTCCTCGGCCTCCC | | |
| | TAAGTGCTAGGATTACAGGTGTGAGCCACCGCGCC | | |
| | CGGCCGCACATTACAGTTTTACTCCATTTTTGAGAAG | | |
| | GTCTGAGGTCAGGAATGACTCATTGTAAGGAAGCAA | | |
| | GCTGTTGCATATTTAGGACCTGTCAGCCAAGAAACT | | |
| | TAAGTGTCCAATGACTTTTGGATGGCCTTTGAGGTG | | |
| | GGTCTCCCTTGCCCAGAAATGCTGACTGTACTGCGC | | |
| | TTAAGCAATTAGGCAGCATTGTAGAG | | |
| RPL13A C- | TCTTAAGCCCCTCTCTTTCTCTAACAGAAAAAGCGGA | FIG. 7K | 14 |
| terminal LHA- | TGGTGGTTCCTGCTGCCCTCAAGGTCGTGCGTCTGA | | |
| linker 1- | AGCCTACAAGAAAGGTGAGTCCCAGCTTACGCTGCA | | |
| degron- | CCATCTACTTGGGAGATTTCAGGCCTGCTGAGGGAC | | |
| stop codon- | CTGGGGACCTGGAGCCTGGCAGATGATGTCCTTATC | | |
| RPL13A C- | TCACGATGGTCTGCGGATGTCCCTGTGGGAATGGC | | |
| terminal RHA | GACAATGCCAATGGCTTAGCTGATGCCAGGAGGCTT | | |
| | GGGTGGGTGCTTTTCTAACAGGCCTGCAGAGAACA | | |
| | GTTGCATTATGATATGCCCAGCTGTCAGTCACCTCC | | |
| | CAGCTCTCAACAGCTCCGGCTCTTCAGGGTGTGGG | | |
| | GGCTTAGATATCCTTACAACTTCATTTGTTCACCCCC | | |
| | CCCCCCCCCCCCCGCAGTTTGCCTATCTGGGGCGC | | |
| | CTGGCTCACGAGGTTGGCTGGAAGTACCAGGCAGT | | |
| | GACAGCCACCCTGGAGGAGAAGAGGAAAGAGAAAG | | |
| | CCAAGATCCACTACCGGAAGAAGAAACAGCTCATGG | | |
| | TGAGGCCAGGGGCTGGTGCTGAGGGGGGCATCTCA | | |
| | CTCCTGGACAGGCCTGGCAGGTGCCTTGCTCACAG | | |
| | AGTACTCTTAACTGGCAAAGGACCAGCCGGGGTTG | | |
| | GGGTGGGATGCAGTCCATGTAATGAGGGCAATGCA | | |
| | ACCCCTCCTGACCACCACCACCTGCACTTATTCTTG | | |
| | GCAGAGGCTACGCAAACAAGCCGAGAAGAACGTGG | | |
| | AGAAGAAAATTGACAAATACACAGAGGTCCTCAAGA | | |
| | CCCACGGACTCCTGGTCGGCGGGTCTCGCCCATTC | | |
| | CAGTGTAATCAGTGTGGGGCATCTTTTACTCAGAAA | | |
| | GGTAACCTCCTCCGCCACATTAAACTGCACTAAGCC | | |
| | CAATAAAGACTGTTAATTCCTCATGCGTTGCCTGCCC | | |
| | TTCCTCCATTGTTGCCCTGGAATGTACGGGACCCAG | | |
| | GGGCAGCAGCAGTCCAGGTGCCACAGGCAGCCCTG | | |
| | GGACATAGGAAGCTGGGAGCAAGGAAAGGGTCTTA | | |
| | GTCACTGCCTCCCGAAGTTGCTTGAAAGCACTCGGA | | |
| | GAATTGTGCAGGTGTCATTTATCTATGACCAATAGGA | | |
| | AGAGCAACCAGTTACTATGAGTGAAAGGGAGCCAGA | | |
| | AGACTGATTGGAGGGCCCTATCTTGTGAGTGGGGC | | |
| | ATCTGTTGGACTTTCCACCTGGTCATATACTCTGCAG | | |
| | CTGTTAGAATGTGCAAGCACTTGGGGACAGCATGAG | | |
| | CTTGCTGTTGTACACAGGGTATTTCTAGAAGCAGAA | | |
| | ATAGACTGGGAAGATGCACAACCAAGGGGTTACAG | | |
| | GCATCGCCCATGCTCCTCACCTGTATTTTGTAATCAG | | |
| | AAATAAATTGCTTTTAAAGAAATCTGGCGTCTTTGCA | | |
| | CTGTGTCTGCTGTGGAGGCAGGCCCCTGGCAAATG | | |
| | GGGGGTGAGGAGCTTGAAGAGGGTAGAATGGGCTG | | |
| | TGCTAATATACAGAATATATGTAACTTGCTATAAATTG | | |
| | AATGATCCTTTATAGACACCGTTTACAAACCAAAGAC | | |
| | ATAAAATGTGGCCAGCAGTGCCTGGTGCTTCCTAGT | | |
| | TAATGTAAAGCTGTCTCATTCTAATTCAGCTGCAAAG | | |
| | TATGGACCCATGCCCTGCTGCCAGGCTGCTGTAGTC | | |
| | CCGGCGGTCTGTAGAGACTAGCATTTTGCAAATGAT | | |
| | AA | | |
| RPL13A C- | TCTTAAGCCCCTCTCTTTCTCTAACAGAAAAAGCGGA | FIG. 7L | 17 |
| terminal LHA- | TGGTGGTTCCTGCTGCCCTCAAGGTCGTGCGTCTGA | | |
| linker 4- | AGCCTACAAGAAAGGTGAGTCCCAGCTTACGCTGCA | | |
| superdegron- | CCATCTACTTGGGAGATTTCAGGCCTGCTGAGGGAC | | |
| stop codon- | CTGGGGACCTGGAGCCTGGCAGATGATGTCCTTATC | | |
| RPL13A C- | TCACGATGGTCTGCGGATGTCCCTGTGGGAATGGC | | |
| terminal RHA | GACAATGCCAATGGCTTAGCTGATGCCAGGAGGCTT | | |
| | GGGTGGGTGCTTTTCTAACAGGCCTGCAGAGAACA | | |
| | GTTGCATTATGATATGCCCAGCTGTCAGTCACCTCC | | |
| | CAGCTCTCAACAGCTCCGGCTCTTCAGGGTGTGGG | | |
| | GGCTTAGATATCCTTACAACTTCATTTGTTCACCCCC | | |
| | CCCCCCCCCCCCCGCAGTTTGCCTATCTGGGGCGC | | |
| | CTGGCTCACGAGGTTGGCTGGAAGTACCAGGCAGT | | |
| | GACAGCCACCCTGGAGGAGAAGAGGAAAGAGAAAG | | |
| | CCAAGATCCACTACCGGAAGAAGAAACAGCTCATGG | | |
| | TGAGGCCAGGGGCTGGTGCTGAGGGGGGCATCTCA | | |
| | CTCCTGGACAGGCCTGGCAGGTGCCTTGCTCACAG | | |
| | AGTACTCTTAACTGGCAAAGGACCAGCCGGGGTTG | | |
| | GGGTGGGATGCAGTCCATGTAATGAGGGCAATGCA | | |
| | ACCCCTCCTGACCACCACCACCTGCACTTATTCTTG | | |
| | GCAGAGGCTACGCAAACAAGCCGAGAAGAACGTGG | | |
| | AGAAGAAAATTGACAAATACACAGAGGTCCTCAAGA | | |
| | CCCACGGACTCCTGGTCGGCTCAGGTAGCGGAAGC | | |
| | GGATCAGGTGGATTCAATGTACTGATGGTCCATAAA | | |
| | CGGAGTCACACTGGCGAGCGCCCGCTCCAATGTGA | | |
| | AATCTGCGGGTTCACGTGTCGGCAGAAGGGCAACC | | |
| | TCCTCCGGCATATCAAGCTGCACACGGGTGAAAAAC | | |
| | CGTTTAAGTGCCATCTCTGCAATTACGCCTGTCAGA | | |
| | GAAGAGATGCTTTGTAAGCCCAATAAAGACTGTTAAT | | |
| | TCCTCATGCGTTGCCTGCCCTTCCTCCATTGTTGCC | | |
| | CTGGAATGTACGGGACCCAGGGGCAGCAGCAGTCC | | |
| | AGGTGCCACAGGCAGCCCTGGGACATAGGAAGCTG | | |
| | GGAGCAAGGAAAGGGTCTTAGTCACTGCCTCCCGA | | |
| | AGTTGCTTGAAAGCACTCGGAGAATTGTGCAGGTGT | | |
| | CATTTATCTATGACCAATAGGAAGAGCAACCAGTTAC | | |
| | TATGAGTGAAAGGGAGCCAGAAGACTGATTGGAGG | | |
| | GCCCTATCTTGTGAGTGGGGCATCTGTTGGACTTTC | | |
| | CACCTGGTCATATACTCTGCAGCTGTTAGAATGTGC | | |
| | AAGCACTTGGGGACAGCATGAGCTTGCTGTTGTACA | | |
| | CAGGGTATTTCTAGAAGCAGAAATAGACTGGGAAGA | | |
| | TGCACAACCAAGGGGTTACAGGCATCGCCCATGCTC | | |
| | CTCACCTGTATTTTGTAATCAGAAATAAATTGCTTTTA | | |
| | AAGAAATCTGGCGTCTTTGCACTGTGTCTGCTGTGG | | |
| | AGGCAGGCCCCTGGCAAATGGGGGGTGAGGAGCTT | | |
| | GAAGAGGGTAGAATGGGCTGTGCTAATATACAGAAT | | |
| | ATATGTAACTTGCTATAAATTGAATGATCCTTTATAGA | | |
| | CACCGTTTACAAACCAAAGACATAAAATGTGGCCAG | | |
| | CAGTGCCTGGTGCTTCCTAGTTAATGTAAAGCTGTC | | |
| | TCATTCTAATTCAGCTGCAAAGTATGGACCCATGCC | | |
| | CTGCTGCCAGGCTGCTGTAGTCCCGGCGGTCTGTA | | |
| | GAGACTAGCATTTTGCAAATGATAA | | |
| RPLP0 C- | CTGAGCTGCCAACCTGGCAATTATTGTCTGCTAAGG | N/A | 104 |
| terminal LHA- | GTTCTCTTTATTCACCCTTACTTGGACTTCCTTTCCT | | |
| linker 1- | GTAGGGAATCTCACGTAAAATGAAATCTTCCCTCCC | | |
| degron- | CCAGGGTGTCCGCAATGTTGCCAGTGTCTGTCTGCA | | |
| stop codon- | GATTGGCTACCCAACTGTTGCATCAGTACCCCATTC | | |
| RPLP0 C- | TATCATCAACGGGTACAAACGAGTCCTGGCCTTGTC | | |
| terminal RHA | TGTGGAGACGGATTACACCTTCCCACTTGCTGAAAA | | |
| | GGTAAAAGGATCCCACCAGGACCACAGTGGGCCTG | | |
| | ACTGTGACAAATTAGCAGGGTGATGTGGCCTTCTAC | | |
| | CTTACTGCTTTTATAGTTGTATTTTATATAGCAGATAA | | |
| | TTTTGTGAGGGGATATTTGAGAGGTTGGGAGGCAGG | | |
| | GAAGGCGTTTCTCACTTGAGAAATGACAAGAGACCC | | |
| | AAAGAGGGGGTTAATGGGCAAGAGCTGGGCCTTAG | | |
| | GAACCCTGCCTCACTAGGCCATACCCAAGCTGTCCT | | |
| | GCTTGGGCTGCTTCTGACAGGAAAGGCTTCACACG | | |
| | GACTTTGATATTGTTGGTCCTTAAACTCTACCAAGGC | | |
| | AGGAGGGTGGTGGGTAATAGAGGAGTGTGGATGAC | | |
| | CATTTTGACCACTTCCCCCCTCCTTTCAGGTCAAGG | | |
| | CCTTCTTGGCTGATCCATCTGCCTTTGTGGCTGCTG | | |
| | CCCCTGTGGCTGCTGCCACCACAGCTGCTCCTGCT | | |
| | GCTGCTGCAGCCCCAGCTAAGGTTGAAGCCAAGGA | | |
| | AGAGTCGGAGGAGTCGGACGAGGATATGGGATTTG | | |
| | GTCTCTTTGACGGCGGGTCTCGCCCATTCCAGTGTA | | |
| | ATCAGTGTGGGGCATCTTTTACTCAGAAAGGTAACC | | |
| | TCCTCCGCCACATTAAACTGCACTAATCACCAAAAAG | | |
| | CAACGAACTTAGCCAGTTTTATTTGCAAAACAAGGAA | | |
| | ATAAATGCTTACTTCTTTAAAAAGTCTCTTGACTCTTA | | |
| | ATTTTGTAATTTTTTTTCCTTTTTGACACAGGGTCTGG | | |
| | CTGTTGCCCAGGCTGGAGTGTGGTGGTGTAATCATA | | |
| | ACTCACTGCACCCTTGAACTCCTGGGATCAAGGGAT | | |
| | CCTCGTATCTCAGCCTCCCAAGTAGCTGGGACTACA | | |
| | GGCACACACCATGACACTCAGCTACTAATTTTTAAAT | | |
| | TTTTTTTTTGTAGAGATGTTGCACAAGCTGGTCTCAA | | |
| | ATTCCTGGCCTCAAGGAATCCTGCCTCAGCCTCCCA | | |
| | AAGTGCTAGGATTACAGGCTTGAGCCACCATGTGCC | | |
| | TGGCCCTTAATTTTGAGGTTTATAGTGCCATATGCTA | | |
| | GAAACGAAAGCCATGGTAAAACCAGAGCTTTGTATT | | |
| | TAGGTGTTGATGTTTGGGTATCTAAATGAAGCTACCA | | |
| | ATCAAACATCCTATACAGTTTTCTAGACACAGTTGTA | | |
| | ACTATTACACTAGAATTACTGTTTCTATGGCTGCTGC | | |
| | ATACTTGGAGTAGGTTTAGTGTCAGCTGAGATAGGC | | |
| | ACCTGGTGGATGCTGGGGCCAGTCCCCTAGAGTAA | | |
| | AGTTTTTCAAACTGGGTGGTGCTCCAACTCGGTGGT | | |
| | AACCAATTTATATTTTCGAGATAGTCTCAAATATATTT | | |
| | GAGACTGGGGTGCAGTGGCTTGGACTTGGCTCACT | | |
| | GCAACCTCCGCCTCCTGGGTTCAAGTGATTCTCCTG | | |
| | CCTCAGCCTCCCAAGTAGCTGC | | |
| RPLP0 C- | CTGAGCTGCCAACCTGGCAATTATTGTCTGCTAAGG | N/A | 105 |
| terminal LHA- | GTTCTCTTTATTCACCCTTACTTGGACTTCCTTTCCT | | |
| linker 1- | GTAGGGAATCTCACGTAAAATGAAATCTTCCCTCCC | | |
| superdegron- | CCAGGGTGTCCGCAATGTTGCCAGTGTCTGTCTGCA | | |
| stop codon- | GATTGGCTACCCAACTGTTGCATCAGTACCCCATTC | | |
| RPLP0 C- | TATCATCAACGGGTACAAACGAGTCCTGGCCTTGTC | | |
| terminal RHA | TGTGGAGACGGATTACACCTTCCCACTTGCTGAAAA | | |
| | GGTAAAAGGATCCCACCAGGACCACAGTGGGCCTG | | |
| | ACTGTGACAAATTAGCAGGGTGATGTGGCCTTCTAC | | |
| | CTTACTGCTTTTATAGTTGTATTTTATATAGCAGATAA | | |
| | TTTTGTGAGGGGATATTTGAGAGGTTGGGAGGCAGG | | |
| | GAAGGCGTTTCTCACTTGAGAAATGACAAGAGACCC | | |
| | AAAGAGGGGGTTAATGGGCAAGAGCTGGGCCTTAG | | |
| | GAACCCTGCCTCACTAGGCCATACCCAAGCTGTCCT | | |
| | GCTTGGGCTGCTTCTGACAGGAAAGGCTTCACACG | | |
| | GACTTTGATATTGTTGGTCCTTAAACTCTACCAAGGC | | |
| | AGGAGGGTGGTGGGTAATAGAGGAGTGTGGATGAC | | |
| | CATTTTGACCACTTCCCCCCTCCTTTCAGGTCAAGG | | |
| | CCTTCTTGGCTGATCCATCTGCCTTTGTGGCTGCTG | | |
| | CCCCTGTGGCTGCTGCCACCACAGCTGCTCCTGCT | | |
| | GCTGCTGCAGCCCCAGCTAAGGTTGAAGCCAAGGA | | |
| | AGAGTCGGAGGAGTCGGACGAGGATATGGGATTTG | | |
| | GTCTCTTTGACGGCTCAGGTAGCGGAAGCGGATCA | | |
| | GGTGGATTCAATGTACTGATGGTCCATAAACGGAGT | | |
| | CACACTGGCGAGCGCCCGCTCCAATGTGAAATCTG | | |
| | CGGGTTCACGTGTCGGCAGAAGGGCAACCTCCTCC | | |
| | GGCATATCAAGCTGCACACGGGTGAAAAACCGTTTA | | |
| | AGTGCCATCTCTGCAATTACGCCTGTCAGAGAAGAG | | |
| | ATGCTTTGTAATCACCAAAAAGCAACGAACTTAGCCA | | |
| | GTTTTATTTGCAAAACAAGGAAATAAATGCTTACTTC | | |
| | TTTAAAAAGTCTCTTGACTCTTAATTTTGTAATTTTTTT | | |
| | TCCTTTTTGACACAGGGTCTGGCTGTTGCCCAGGCT | | |
| | GGAGTGTGGTGGTGTAATCATAACTCACTGCACCCT | | |
| | TGAACTCCTGGGATCAAGGGATCCTCGTATCTCAGC | | |
| | CTCCCAAGTAGCTGGGACTACAGGCACACACCATGA | | |
| | CACTCAGCTACTAATTTTTAAATTTTTTTTTTGTAGAG | | |
| | ATGTTGCACAAGCTGGTCTCAAATTCCTGGCCTCAA | | |
| | GGAATCCTGCCTCAGCCTCCCAAAGTGCTAGGATTA | | |
| | CAGGCTTGAGCCACCATGTGCCTGGCCCTTAATTTT | | |
| | GAGGTTTATAGTGCCATATGCTAGAAACGAAAGCCA | | |
| | TGGTAAAACCAGAGCTTTGTATTTAGGTGTTGATGTT | | |
| | TGGGTATCTAAATGAAGCTACCAATCAAACATCCTAT | | |
| | ACAGTTTTCTAGACACAGTTGTAACTATTACACTAGA | | |
| | ATTACTGTTTCTATGGCTGCTGCATACTTGGAGTAGG | | |
| | TTTAGTGTCAGCTGAGATAGGCACCTGGTGGATGCT | | |
| | GGGGCCAGTCCCCTAGAGTAAAGTTTTTCAAACTGG | | |
| | GTGGTGCTCCAACTCGGTGGTAACCAATTTATATTTT | | |
| | CGAGATAGTCTCAAATATATTTGAGACTGGGGTGCA | | |
| | GTGGCTTGGACTTGGCTCACTGCAACCTCCGCCTCC | | |
| | TGGGTTCAAGTGATTCTCCTGCCTCAGCCTCCCAAG | | |
| | TAGCTGC | | |

Methods of Introducing Targeting Constructs into Target Cells are Described in Sections 6.8 and 6.9.

In some embodiments, a target cell is modified to include only one copy of a nucleotide sequence encoding a fusion protein comprising an essential polypeptide and a degron. For example, when a targeting construct is integrated into a host cell genome, an engineered cell is selected that only contains the targeting construct insert at one allele of the essential gene. In some embodiments, the second allele is engineered to incorporate a transgene, e.g., by virtue of introducing a second targeting construct comprising the transgene as well as the targeting construct comprising the degron coding sequence.

In other embodiments, a target cell is modified to include two copies of the nucleotide sequence encoding the fusion protein comprising the essential polypeptide and the degron. For example, when a targeting construct is integrated into a host cell genome, an engineered cell is selected that contains the targeting construct insert at both alleles of the essential gene.

### 6.3.2. Integration Site

Targeting constructs that are intended for integration into a target cell genome typically comprise a heterologous sequence that is not present in the target cell genome, e.g., a degron coding sequence or a transgene.

In some embodiments, a degron sequence is introduced into an essential gene, so that the essential gene is modified to express a fusion protein comprising the essential polypeptide and the degron, optionally separated via a linker. One of skill in the art would readily appreciate that the term “essential gene” is not limited to STEL genes. However, due to robust expression from STEL gene loci, in some embodiments, particularly when used to co-localize a transgene into the genome of a host cell, the essential gene into which the degron is introduced is also a STEL gene.

In some embodiments, the degron sequence is introduced into an essential gene (whether a STEL or non-STEL gene) of a functional category of essential genes identified below. In other embodiments, the degron sequence is introduced into an essential gene (whether a STEL or non-STEL gene) not from a category of essential genes identified below (e.g., the category is the subject of a proviso).

In some embodiments, the degron sequence is introduced into an individual essential gene (whether a STEL or non-STEL gene) selected from the individual essential genes identified below. In other embodiments, the degron sequence is introduced into an essential gene (whether a STEL or non-STEL gene) that is not one of the essential genes identified below (e.g., the particular essential gene is the subject of a proviso).

In some embodiments, the essential gene is the GAPDH gene.

Where the essential gene is a STEL gene, a transgene may also be introduced into the STEL locus so that the transgene can be expressed from a locus of sustained expression. In some embodiments, the transgene is introduced into the GAPDH gene.

The degron coding sequence and the transgene can be introduced into the same STEL gene, whether via a single targeting construct, as depicted in FIGS. 4 and 5, or via different targeting constructs, as depicted in FIG. 6. Target cells can be selected that are homozygous or heterozygous for both the degron coding sequence and the transgene.

The targeting constructs typically include one or more regions that are homologous to regions of DNA within or near (e.g., flanking or adjoining) a target sequence. These homologous regions are referred to here as “homology arms.” For ease of reference, the homology arms are referred to herein as first and second (i.e., 5′ and 3′, upstream and downstream, or left and right) homology arms. This terminology relates to the relative position of the homology arms to the nucleic acid insert within the targeting construct. The first and second homology arms correspond to regions within the target genomic locus, which are referred to herein as “first region of homology” and “second region of homology,” respectively.

In some embodiments, the targeting construct comprises homology arms that target integration of a heterologous sequence into an essential gene locus, whereby integration of a heterologous sequence introduces degron coding sequence in-frame with an essential polypeptide coding sequence, connected directly or via a linker sequence. Thus, following integration, the essential gene locus is engineered to express a fusion polypeptide of the disclosure.

In some embodiments, the essential gene is active in multiple cell types such as a gene involved in gene expression (e.g., transcription factors and/or histones), cellular metabolism (e.g., glyceraldehyde 3-phosphate dehydrogenase (GAPDH)), or cellular structures (e.g., actin), or encodes a ribosomal protein (e.g., large or small ribosomal subunits, such as RPL13A, RPLP0 and/or RPL7).

Additional examples of essential genes include those that are involved in one or more of glycolysis, ribonucleoprotein complex formation, focal adhesion, cell-substrate adherens junction, cell-substrate junction, cell anchoring, extracellular exosome, extracellular vesicle, intracellular organelle, or anchoring junction. Some of the proteins are involved in RNA binding, nucleic acid binding (e.g., rRNA or mRNA binding), or protein binding.

In some embodiments, an essential gene is robustly and consistently expressed in the pluripotent state as well as during differentiation (e.g., as examined by single-cell RNA sequencing (scRNAseq) analysis). For example, the expression level of the endogenous gene does not change (e.g., decrease) by more than 50%, more than 40%, more than 35%, more than 30%, more than 25%, more than 20%, more than 15%, more than 10%, or more than 5% over five or more, ten or more, or 15 or more passages or as the cell state changes (e.g., state of pluripotency and/or differentiation).

In some embodiments, an essential gene is a gene that is associated with cellular metabolism, such as GAPDH.

In some embodiments, an essential gene is a ribosomal protein gene or ribosomal protein gene locus, such as an RPL or RPS gene locus. Examples of RPL genes are RPL10, RPL13, RPS18, RPL3, RPLP1, RPL13A, RPL15, RPL41, RPL11, RPL32, RPL18A, RPL19, RPL28, RPL29, RPL9, RPL8, RPL6, RPL18, RPL7, RPL7A, RPL21, RPL37A, RPL 12, RPL5, RPL34, RPL35A, RPL30, RPL24, RPL39, RPL37, RPL14, RPL27A, RPLP2, RPLP0, RPL23A, RPL26, RPL36, RPL35, RPL23, RPL4, and RPL22. Examples of RPS genes are RPS2, RPS 19, RPS 14, RPS3A, RPS 12, RPS3, RPS6, RPS23, RPS27A, RPS8, RPS4X, RPS7, RPS24, RPS27, RPS15A, RPS9, RPS28, RPS 13, RPSA, RPS5, RPS16, RPS25, RPS15, RPS20, and RPS11.

In some embodiments, an essential gene encodes a cytoskeletal protein, such as actin. Examples of actin genes are ACTG1 and ACTB.

In some embodiments, the essential gene encodes a eukaryotic translation elongation factor, such as EEF1A1 and EEF2, or a eukaryotic translation initiation factor, such as EIF1.

In some embodiments, an essential gene encodes a histone, such as H3F3A and H3F3B.

In other embodiments, an essential gene is a STEL gene selected from FTH1, TPT1, PTMA, GNB2L1, NACA, YBX1, NPM1, FAU, UBA52, HSP90AB1, MYL6, SERF2, and SRP14.

In other embodiments, an essential gene is a non-STEL gene, such as HDAC3, DNMT1, NADH dehydrogenase, and PGK1.

### 6.3.3. Homology Arms

The current disclosure provides a targeting construct comprising a first homology arm that corresponds to a first region of homology, a nucleic acid insert, and a second homology arm that corresponds to a second region of homology.

A homology arm and a target sequence “correspond” or are “corresponding” to one another when the two regions share a sufficient level of sequence identity to one another to act as substrates for a homologous recombination reaction, whereby the homology arms are suitable for directing recombination of a nucleic acid insert with a desired genomic locus to facilitate genomic integration and/or replacement of endogenous sequence.

The term “homology” includes DNA sequences that are either identical or share sequence identity to a corresponding sequence. The sequence identity between a given target sequence and the corresponding homology arm found in the exogenous donor nucleic acid can be any degree of sequence identity that allows for homologous recombination to occur. For example, the amount of sequence identity shared by the homology arm of the exogenous donor nucleic acid (or a fragment thereof) and the target sequence (or a fragment thereof) can be at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity, such that the sequences undergo homologous recombination. Moreover, a corresponding region of homology between the homology arm and the corresponding target sequence can be of any length that is sufficient to promote homologous recombination. In some targeting vectors, the intended mutation in the target genomic locus is included in an insert nucleic acid flanked by the homology arms.

In some embodiments, the first homology arm is between 50 to 250 nucleotides in length. In some embodiments, the first homology arm is between 50-2000 nucleotides in length. In some embodiments, the first homology arm is between 50-1500 nucleotides in length. In some embodiments, the first homology arm is between 50-1000 nucleotides in length. In some embodiments, the first homology arm is between 50-500 nucleotides in length. In some embodiments, the first homology arm is between 150 to 250 nucleotides in length. In some embodiments, the first homology arm is 2000 nucleotides or less in length. In some embodiments, the first homology arm is 1500 nucleotides or less in length. In some embodiments, the first homology arm is 1000 nucleotides or less in length. In some embodiments, the first homology arm is 700 nucleotides or less in length. In some embodiments, the first homology arm is 650 nucleotides or less in length. In some embodiments, the first homology arm is 600 nucleotides or less in length. In some embodiments, the first homology arm is 550 nucleotides or less in length. In some embodiments, the first homology arm is 500 nucleotides or less in length. In some embodiments, the first homology arm is 400 nucleotides or less in length. In some embodiments, the first homology arm is 300 nucleotides or less in length. In some embodiments, the first homology arm is 250 nucleotides or less in length. In some embodiments, the first homology arm is 200 nucleotides or less in length. In some embodiments, the first homology arm is 150 nucleotides or less in length. In some embodiments, the first homology arm is less than 100 nucleotides in length. In some embodiments, the first homology arm is 50 nucleotides in length or less. In some embodiments, the first homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In some embodiments, the first homology arm is at least 20 nucleotides in length. In some embodiments, the first homology arm is at least 40 nucleotides in length. In some embodiments, the first homology arm is at least 50 nucleotides in length. In some embodiments, the first homology arm is at least 70 nucleotides in length. In some embodiments, the first homology arm is at least 100 nucleotides in length. In some embodiments, the first homology arm is at least 200 nucleotides in length. In some embodiments, the first homology arm is at least 300 nucleotides in length. In some embodiments, the first homology arm is at least 400 nucleotides in length. In some embodiments, the first homology arm is at least 500 nucleotides in length. In some embodiments, the first homology arm is at least 600 nucleotides in length. In some embodiments, the first homology arm is at least 700 nucleotides in length. In some embodiments, the first homology arm is at least 1000 nucleotides in length. In some embodiments, the first homology arm is at least 1500 nucleotides in length. In some embodiments, the first homology arm is at least 2000 nucleotides in length. In some embodiments, the first homology arm is about 20 nucleotides in length. In some embodiments, the first homology arm is about 40 nucleotides in length. In some embodiments, the first homology arm is 250 nucleotides in length or less. In some embodiments, the first homology arm is about 100 nucleotides in length. In some embodiments, the first homology arm is about 200 nucleotides in length.

In some embodiments, the second homology arm is between 50 to 250 nucleotides in length. In some embodiments, the second homology arm is between 50-2000 nucleotides in length. In some embodiments, the second homology arm is between 50-1500 nucleotides in length. In some embodiments, the second homology arm is between 50-1000 nucleotides in length. In some embodiments, the second homology arm is between 50-500 nucleotides in length. In some embodiments, the second homology arm is between 150 to 250 nucleotides in length. In some embodiments, the second homology arm is 2000 nucleotides or less in length. In some embodiments, the second homology arm is 1500 nucleotides or less in length. In some embodiments, the second homology arm is 1000 nucleotides or less in length. In some embodiments, the second homology arm is 700 nucleotides or less in length. In some embodiments, the second homology arm is 650 nucleotides or less in length. In some embodiments, the second homology arm is 600 nucleotides or less in length. In some embodiments, the second homology arm is 550 nucleotides or less in length. In some embodiments, the second homology arm is 500 nucleotides or less in length. In some embodiments, the second homology arm is 400 nucleotides or less in length. In some embodiments, the second homology arm is 300 nucleotides or less in length. In some embodiments, the second homology arm is 200 nucleotides in length or less. In some embodiments, the second homology arm is 150 nucleotides in length or less. In some embodiments, the second homology arm is 100 nucleotides in length or less. In some embodiments, the second homology arm is 50 nucleotides in length or less. In some embodiments, the second homology arm is 250, 200, 190, 180, 170, 160, 150, 140, 130, 120, 110, 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, or 20 nucleotides in length. In some embodiments, the second homology arm is at least 20 nucleotides in length. In some embodiments, the second homology arm is at least 40 nucleotides in length. In some embodiments, the second homology arm is at least 50 nucleotides in length. In some embodiments, the second homology arm is at least 70 nucleotides in length. In some embodiments, the second homology arm is at least 100 nucleotides in length. In some embodiments, the second homology arm is at least 200 nucleotides in length. In some embodiments, the second homology arm is at least 300 nucleotides in length. In some embodiments, the second homology arm is at least 400 nucleotides in length. In some embodiments, the second homology arm is at least 500 nucleotides in length. In some embodiments, the second homology arm is at least 600 nucleotides in length. In some embodiments, the second homology arm is at least 700 nucleotides in length. In some embodiments, the second homology arm is at least 1000 nucleotides in length. In some embodiments, the second homology arm is at least 1500 nucleotides in length. In some embodiments, the second homology arm is at least 2000 nucleotides in length. In some embodiments, the second homology arm is about 20 nucleotides in length. In some embodiments, the second homology arm is about 40 nucleotides in length. In some embodiments, the second homology arm is 250 nucleotides in length or less. In some embodiments, the second homology arm is about 100 nucleotides in length. In some embodiments, the second homology arm is about 200 nucleotides in length.

The first and second homology arms can be of the same length or can differ in length. In some embodiments, the first and second homology arms are amplified to allow for the quantitative assessment of gene editing events, such as targeted integration, at a target nucleic acid. In some embodiments, the quantitative assessment of the gene editing events may rely on the amplification of both the 5′ junction and 3′ junction at the site of targeted integration by amplifying the whole or a part of the homology arm using a single pair of PCR primers in a single amplification reaction. Accordingly, although the length of the first and second homology arms may differ, the length of each homology arm should be capable of amplification (e.g., using PCR), as desired. Moreover, when amplification of both the first and second homology arms, the difference in lengths of the first and second homology arms in a single PCR reaction is desired, the length difference between the first and second homology arms should allow for PCR amplification using a single pair of PCR primers.

In some embodiments, the length of the first and second homology arms does not differ by more than 75 nucleotides. Thus, in some embodiments, when the first and second homology arms differ in length, the length difference between the homology arms is less than 70, 60, 50, 40, 30, 20, 10, 9, 8, 7, 6, 5, 4, 3, 2, 1 nucleotides or base pairs. In some embodiments, the first and second homology arms differ in length by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 nucleotides. In some embodiments, the length difference between the first and second homology arms is less than 70, 60, 50, 40, 30, 20, 10, 9, 8, 7, 6, 5, 4, 3, 2, 1 base pairs. In some embodiments, the first and second homology arms differ in length by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 base pairs.

Homology arms are capable of directing recombination of a nucleic acid insert with a desired target genomic locus to facilitate genomic integration and/or replacement of endogenous sequence. Regardless of the format used, a donor template can be designed to avoid undesirable sequences. In certain embodiments, one or both homology arms can be shortened to avoid overlap with certain sequence repeat elements, e.g., Alu repeats, LINE elements, etc.

### 6.4. Transgenes

In some embodiments, the constructs and methods of the disclosure are designed to engineer a target cell to express both (a) a fusion protein comprising an essential polypeptide and a degron and (b) a recombinant polypeptide. In some embodiments, the recombinant polypeptide is expressed from a transgene.

In some embodiments, the transgene is introduced into a target cell via the same targeting construct or expression vector as the targeting construct or expression vector comprising the degron coding sequence. The transgene may be positioned 5′ or 3′ to the degron coding sequence. Illustrative embodiments of suitable targeting constructs comprising both a transgene and a degron coding sequence are shown in FIG. 4.

In some embodiments, a transgene may be expressed from a different allele of the essential gene that has been modified to express a fusion protein comprising an essential polypeptide and a degron, as depicted in FIGS. 6A and 6C. In some embodiments, the essential gene is a STEL gene. In some embodiments, a transgene is located in a gene/locus that is different from the essential gene to which a degron as described herein is fused.

In some embodiments, the transgene and the fusion protein are expressed from separate loci. In some embodiments, the separate loci are both STEL loci, e.g., a GAPDH locus and another STEL locus. In other embodiments, the separate loci are both non-STEL loci. In yet other embodiments, one of the loci is a STEL (e.g., GAPDH) locus and the other locus is a non-STEL locus.

In yet further embodiments, the transgene is expressed from an expression vector and the fusion protein is expressed from a genomic locus (e.g., a STEL locus).

In yet further embodiments, the fusion protein is expressed from an expression vector and the transgene is expressed from a genomic locus (e.g., a STEL locus).

In some embodiments, the transgene encodes a reporter protein, such as a fluorescent protein (e.g., green fluorescent protein, red fluorescent protein, cyan fluorescent protein, yellow fluorescent protein, blue fluorescent protein, DsRed, mCherry, mKate2, and tdTomato) and an enzyme (e.g., luciferase and lacZ). A reporter protein may aid the tracking of therapeutic cells once they are implanted to a patient.

In some embodiments, the transgene encodes a therapeutic molecule, such as a therapeutic nucleotide or a therapeutic polypeptide or protein.

In some embodiments, the therapeutic molecule encoded by the transgene is a therapeutic nucleotide, e.g., an oligonucleotide (e.g., a miRNA, gapmers, a steric block ON, an antagomir, a small interfering RNA (siRNA), a micro-RNA mimic, a splice switching ON, or an aptamer).

In some embodiments, the therapeutic transgene is a miRNA or other small interfering nucleic acid that can regulate gene expression via RNA transcript cleavage/degradation or translational repression of an mRNA. Nonlimiting examples of miRNA genes or other small interfering nucleic acids that may be used as therapeutic transgenes include hsa-let-7a, hsa-let-7a *. hsa-let-7b, hsa-let-7b*, hsa-let-7c, hsa-let-7c*, hsa-let-7d, hsa-let-7d*, hsa-let-7e, hsa-let-7e*, hsa-let-7f, hsa-let-7f-1*, hsa-let-7f-2*, hsa-let-7g, hsa-let-7g*, hsa-let-71, hsa-let-71*, hsa-miR-1, hsa-miR-100, hsa-miR-100*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b*, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR- 124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-127-3p, hsa-miR-1274a, hsa-miR-1274b, hsa-miR-1275, hsa-miR-127-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, hsa-miR-136*, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hsa-miR-145*, hsa-miR-146a, hsa-miR-146a*, hsa-miR-146b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-17*, hsa-miR-181a, hsa-miR-181a*, hsa-miR-181a-2*, hsa-miR-181b, hsa-miR-181c, hsa-miR-181c*, hsa-miR-181d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR- 19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-296-1*, hsa-miR-296-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302e, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30e, hsa-miR-30e*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-337-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-361-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a+, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-431*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518c*, hsa-miR-518d-3p, hsa-miR-518d-5p, hsa-miR-518e, hsa-miR-518e*, hsa-miR-518f, hsa-miR-518f*, hsa-miR-519a, hsa-miR-519b-3p, hsa-miR-519c-3p, hsa-miR-519d, hsa-miR-519e, hsa-miR-519e*, hsa-miR-520a-3p, hsa-miR-520a-5p, hsa-miR-520b, hsa-miR-520c-3p, hsa-miR-520d-3p, hsa-miR-520d-5p, hsa-miR-520e, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b*, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-5486-5p, hsa-miR-548c-3p, hsa-miR-548c-5p, hsa-miR-548d-3p, hsa-miR-548d-5p, hsa-miR-548e, hsa-miR-548f, hsa-miR-548g, hsa-miR-548h, hsa-miR-548i, hsa-miR-548j, hsa-miR-548k, hsa-miR-5481, hsa-miR-548m, hsa-miR-548n, hsa-miR-5480, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-miR-598, hsa-miR-599, hsa-miR-600, hsa-miR-601, hsa-miR-602, hsa-miR-603, hsa-miR- 604, hsa-miR-605, hsa-miR-606, hsa-miR-607, hsa-miR-608, hsa-miR-609, hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96*, hsa-miR-98, hsa-miR-99a, hsa-miR-99a*, hsa-miR-99b, and hsa-miR-99b*.

In some embodiments, a transgene encodes a therapeutic protein or polypeptide. A therapeutic protein or polypeptide may be introducing a protein or peptide that is absent in a patient. A therapeutic protein or polypeptide may also be replacing a protein that is deficient or abnormal (e.g., a having a mutation) in a patient such as those associated with rare or orphan diseases. Examples of such rare diseases may include spinal muscular atrophy (SMA), Huntingdon's Disease, Rett Syndrome (e.g., methyl-CpG-binding protein 2 (MeCP2); UniProtKB-P51608), Amyotrophic Lateral Sclerosis (ALS), Duchenne Type Muscular dystrophy, Friedrichs Ataxia (e.g., frataxin), progranulin (PRGN) (associated with non-Alzheimer's cerebral degenerations, including, frontotemporal dementia (FTD), progressive non-fluent aphasia (PNFA) and semantic dementia), among others.

Therapeutic proteins and polypeptides that replace absent, deficient, or abnormal proteins in a patient may also target familial hypercholesterolemia, muscular dystrophy, mucopolysaccaridoses, cystic fibrosis, diabetes, and blood coagulation disorders. Nonlimiting examples of therapeutic proteins and polypeptides that replace absent, deficient, or abnormal proteins include insulin, growth hormone, coagulation factors, albumin, H-protein, T-protein, dystonin, neurofilament light chain (NEFL), and various enzymes that can be used for enzyme replacement therapy, such as lactase, lipase, amylase, adenosine deaminase, β-glucocerebrosidase carbamoyl synthetase I, ornithine transcarbamylase (OTC), arginosuccinate synthetase, arginosuccinate lyase (ASL) for treatment of argunosuccinate lyase deficiency, arginase, fumarylacetate hydrolase, phenylalanine hydroxylase, alpha-1 antitrypsin, rhesus alpha-fetoprotein (AFP), rhesus chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, beta-glucosidase, pyruvate carboxylate, hepatic phosphorylase, phosphorylase kinase, and glycine decarboxylase, α-L-iduronidase (IDUA), iduronate-2-sulfatase (IDS), sulfamidase, N-acetylgalactosamine-6-sulfate sulfatase (GALNS), arylsulfatase B, hyaluronidase, β-glucuronidase, phosphoenolpyruvate-carboxykinase (PEPCK), cyclin-dependent kinase-like 5 (CDKL5), galactose-phosphate uridyl transferase, branched chain alpha-ketoacid dehydrogenase, fumarylacetoacetate hydrolase, methylmalonyl-CoA mutase, argininosuccinic acid synthetase, lecithin-cholesterol acyltransferase, hypoxanthine guanine phosphoribosyl transferase, biotimidase, α-galactosidase A, hexosaminidase, ceramidase, aspartylglucosaminidase, and α-fucosidase

In some embodiments, the therapeutic protein or polypeptide can be used to augment an existing pathway. Some nonlimiting examples of augmenting therapeutic proteins and polypeptides include peptide hormones that are used to treat hormonal deficiencies or infertility, growth and differentiation factors, and proteins to treat hematopoietic deficiencies, hemotherapy-induced anemia, or myelodysplastic syndrome.

Nonlimiting examples of hormones and growth and differentiation factors that can be used as therapeutic proteins or polypeptides include glucagon, glucagon-like peptide-1 (GLP1), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO) (including, e.g., human, canine or feline epo), connective tissue growth factor (CTGF), neutrophic factors including, e.g., basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

In some embodiments, the therapeutic protein or polypeptide can be used to provide a novel function or activity to endogenous proteins or introduce nonendogenous proteins with novel functions or activity. Some nonlimiting examples are proteins and peptides used for enzymatic degradation of macromolecules, such as papain, collagenase, hyaluronidase, botulinum toxin type A and type B; as well as proteins and peptides used for enzymatic degradation of small molecule metabolites, such as L-asparaginase, Peg-asparaginase, and rasburicase. Other examples may include chimeric or hybrid polypeptides having a non-naturally occurring amino acid sequence containing insertions, deletions, or amino acid substitutions. For example, single-chain engineered immunoglobulins could be useful in certain immunocompromised patients. Further examples of non-naturally occurring gene sequences may include antisense molecules and catalytic nucleic acids, such as ribozymes, which could be used to reduce overexpression of a target.

In some embodiments, the therapeutic nucleic acid, protein, or polypeptide can be used to interfere with a molecule or organism. Nonlimiting examples include those that are used to treat an infection or various forms of cancer, such as proteins and peptides that are produced exclusively or at higher levels in hyperproliferative cells as compared to normal cells, e.g., polypeptides encoded by oncogenes myb, myc, fyn, and the translocation gene bcr/abl, ras, src, P53, neu, trk and EGRF. The following is a nonlimiting list of exemplary genes known to be associated with the development of cancer (e.g., oncogenes and tumor suppressors) which can be targeted by a therapeutic transgene: AARS, ABCB1, ABCC4, ABI2, ABL1, ABL2, ACK1, ACP2, ACY1, ADSL, AK1, AKR1C2, AKT1, ALB, ANPEP, ANXA5, ANXA7, AP2M1, APC, ARHGAP5, ARHGEF5, ARID4A, ASNS, ATF4, ATM, ATP5B, ATP50, AXL, BARD1, BAX, BCL2, BHLHB2, BLMH, BRAF, BRCA1, BRCA2, BTK, CANX, CAP1, CAPN1, CAPNS1, CAV1, CBFB, CBLB, CCL2, CCND1, CCND2, CCND3, CCNE1, CCT5, CCYR61, CD24, CD44, CD59, CDC20, CDC25, CDC25A, CDC25B, CDC2L5, CDK10, CDK4, CDK5, CDK9, CDKL1, CDKN1A, CDKN1B, CDKN1C, CDKN2A, CDKN2B, CDKN2D, CEBPG, CENPC1, CGRRF1, CHAF1A, CIB1, CKMT1, CLK1, CLK2, CLK3, CLNS1A, CLTC, COL1A1, COL6A3, COX6C, COX7A2, CRAT, CRHR1, CSF1R, CSK, CSNK1G2, CTNNA1, CTNNB1, CTPS, CTSC, CTSD, CUL1, CYR61, DCC, DCN, DDX10, DEK, DHCR7, DHRS2, DHX8, DLG3, DVL1, DVL3, E2F1, E2F3, E2F5, EGFR, EGR1, EIF5, EPHA2, ERBB2, ERBB3, ERBB4, ERCC3, ETV1, ETV3, ETV6, F2R, FASTK, FBN1, FBN2, FES, FGFR1, FGR, FKBP8, FN1, FOS, FOSL1, FOSL2, FOXG1A, FOXO1A, FRAP1, FRZB, FTL, FZD2, FZD5, FZD9, G22P1, GAS6, GCN5L2, GDF15, GNA13, GNAS, GNB2, GNB2L1, GPR39, GRB2, GSK3A, GSPT1, GTF21, HDAC1, HDGF, HMMR, HPRT1, HRB, HSPA4, HSPA5, HSPA8, HSPB1, HSPH1, HYAL1, HYOU1, ICAM1, ID1, ID2, IDUA, IER3, IFITM1, IGF1R, IGF2R, IGFBP3, IGFBP4, IGFBP5, IL1B, ILK, ING1, IRF3, ITGA3, ITGA6, ITGB4, JAK1, JARID1A, JUN, JUNB, JUND, K-ALPHA-1, KIT, KITLG, KLK10, KPNA2, KRAS2, KRT18, KRT2A, KRT9, LAMB1, LAMP2, LCK, LCN2, LEP, LITAF, LRPAP1, LTF, LYN, LZTR1, MADH1, MAP2K2, MAP3K8, MAPK12, MAPK13, MAPKAPK3, MAPRE1, MARS, MAS1, MCC, MCM2, MCM4, MDM2, MDM4, MET, MGST1, MICB, MLLT3, MME, MMP1, MMP14, MMP17, MMP2, MNDA, MSH2, MSH6, MT3, MYB, MYBL1, MYBL2, MYC, MYCL1, MYCN, MYD88, MYL9, MYLK, NEO1, NF1, NF2, NFKB1, NFKB2, NFSF7, NID, NINE, NMBR, NME1, NME2, NME3, NOTCH1, NOTCH2, NOTCH4, NPM1, NQO1, NR1D1, NR2F1, NR2F6, NRAS, NRG1, NSEP1, OSM, PA2G4, PABPC1, PCNA, PCTK1, PCTK2, PCTK3, PDGFA, PDGFB, PDGFRA, PDPK1, PEA15, PFDN4, PFDN5, PGAM1, PHB, PIK3CA, PIK3CB, PIK3CG, PIM1, PKM2, PKMYT1, PLK2, PPARD, PPARG, PPIH, PPP1CA, PPP2R5A, PRDX2, PRDX4, PRKAR1A, PRKCBP1, PRNP, PRSS15, PSMA1, PTCH, PTEN, PTGS1, PTMA, PTN, PTPRN, RAB5A, RAC1, RAD50, RAF1, RALBP1, RAP1A, RARA, RARB, RASGRF1, RB1, RBBP4, RBL2, REA, REL, RELA, RELB, RET, RFC2, RGS19, RHOA, RHOB, RHOC, RHOD, RIPK1, RPN2, RPS6 KB1, RRM1, SARS, SELENBP1, SEMA3C, SEMA4D, SEPP1, SERPINH1, SFN, SFPQ, SFRS7, SHB, SHH, SIAH2, SIVA, SIVA TP53, SKI, SKIL, SLC16A1, SLC1A4, SLC20A1, SMO, sphingomyelin phosphodiesterase 1 (SMPD1), SNAI2, SND1, SNRPB2, SOCS1, SOCS3, SOD1, SORT1, SPINT2, SPRY2, SRC, SRPX, STAT1, STAT2, STAT3, STAT5B, STC1, TAF1, TBL3, TBRG4, TCF1, TCF7L2, TFAP2C, TFDP1, TFDP2, TGFA, TGFB1, TGFBI, TGFBR2, TGFBR3, THBS1, TIE, TIMP1, TIMP3, TJP1, TK1, TLE1, TNF, TNFRSF10A, TNFRSF10B, TNFRSF1A, TNFRSF1B, TNFRSF6, TNFSF7, TNK1, TOB1, TP53, TP53BP2, TP5313, TP73, TPBG, TPT1, TRADD, TRAM1, TRRAP, TSG101, TUFM, TXNRD1, TYRO3, UBC, UBE2L6, UCHL1, USP7, VDAC1, VEGF, VHL, VIL2, WEE1, WNT1, WNT2, WNT2B, WNT3, WNT5A, WT1, XRCC1, YES1, YWHAB, YWHAZ, ZAP70, and ZNF9.

In some embodiments, a therapeutic transgene may be an apoptosis modulator. Nonlimiting examples of apoptosis modulators include RPS27A, ABL1, AKT1, APAF1, BAD, BAG1, BAG3, BAG4, BAK1, BAX, BCL10, BCL2, BCL2A1, BCL2L1, BCL2L10, BCL2L11, BCL2L12, BCL2L13, BCL2L2, BCLAF1, BFAR, BID, BIK, NAIP, BIRC2, BIRC3, XIAP, BIRC5, BIRC6, BIRC7, BIRC8, BNIP1, BNIP2, BNIP3, BNIP3L, BOK, BRAF, CARD10, CARD11, NLRC4, CARD14, NOD2, NOD1, CARD6, CARDS, CARDS, CASP1, CASP10, CASP14, CASP2, CASP3, CASP4, CASP5, CASP6, CASP7, CASP8, CASP9, CFLAR, CIDEA, CIDEB, CRADD, DAPK1, DAPK2, DFFA, DFFB, FADD, GADD45A, GDNF, HRK, IGF1R, LTA, LTBR, MCL1, NOL3, PYCARD, RIPK1, RIPK2, TNF, TNFRSF10A, TNFRSF10B, TNFRSF10C, TNFRSF10D, TNFRSF11B, TNFRSF12A, TNFRSF14, TNFRSF19, TNFRSF1A, TNFRSF1B, TNFRSF21, TNFRSF25, CD40, FAS, TNFRSF6B, CD27, TNFRSF9, TNFSF10, TNFSF14, TNFSF18, CD40LG, FASLG, CD70, TNFSF8, TNFSF9, TP53, TP53BP2, TP73, TP63, TRADD, TRAF1, TRAF2, TRAF3, TRAF4, and TRAF5.

In some embodiments, the therapeutic protein or polypeptide can be used to deliver other compounds or proteins, such as a radionuclide, cytotoxic drug, or an effector protein to a target tissue or organ.

In some other embodiments, therapeutic proteins and polypeptides include those which may be useful for treating individuals suffering from autoimmune diseases and disorders by conferring a broad based protective immune response against targets that are associated with autoimmunity including cell receptors and cells which produce “self”-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease, and ulcerative colitis.

In some embodiments, a therapeutic protein is a receptor or a ligand for a receptor. Nonlimiting examples of receptors include any one of the receptors for hormones, growth factors, cytokines, lymphokines, regulatory proteins and immune system proteins, and receptors for cholesterol regulation and/or lipid modulation, including the low-density lipoprotein (LDL) receptor, high density lipoprotein (HDL) receptor, the very low-density lipoprotein (VLDL) receptor, and scavenger receptors. In some embodiments, the therapeutic protein is a member of the steroid hormone receptor superfamily including glucocorticoid receptors and estrogen receptors, Vitamin D receptors, and other nuclear receptors.

Therapeutic proteins and polypeptides also include complement regulatory proteins such as complement regulatory proteins, membrane cofactor protein (MCP), decay accelerating factor (DAF), CR1, CF2, CD59, and C1 esterase inhibitor (C1-INH). In some embodiments, a therapeutic protein may be a noncovalent binder other than a mAb, Fc fusion protein, or polyclonal immunoglobulins (for examples, see Table 4 of Dimitrov, 2012, Methods Mol Biol. 899:1-26, which is incorporated herein by reference).

In some embodiments, the transgene is a therapeutic protein that can be used to treat lysosomal storage disorders. In some embodiments, the therapeutic protein is a lysosomal enzyme, such as alpha-L-iduronidase, arylsulfatase A, beta-glucocerebrosidase, acid sphingomyelinase, alpha-galactosidase or beta galactosidase.

In some embodiments, the transgene is a therapeutic protein that can be used to treat hemophilia or other genetic blood disorders. In some embodiments, the therapeutic polypeptide is Factor VIII and Factor IX. In some embodiments, the therapeutic transgene comprises first 57 base pairs of the Factor VIII heavy chain which encodes the 10 amino acid signal sequence, as well as the human growth hormone (hGH) polyadenylation sequence. In alternative embodiments, the therapeutic transgene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor Vlll heavy chain and light chain are provided in a single minigene separated by 42 nucleic acids coding for 14 amino acids of the B domain (see U.S. Pat. No. 6,200,560).

In some embodiments, a therapeutic protein may be an immune system associated protein or polypeptide, such as an antibody, a Fab fragment, an immunoglobulin light chain, an immunoglobulin heavy chain, a Fc fusion protein, an immunoadhesin, an interferon, a lymphokine, an immunomodulating agent, e.g., a cytokine or cytokine receptor, or an interleukin or interleukin receptor. Immune system regulating therapeutic proteins and polypeptides may include, without limitation, thrombopoietin (TPO), interleukins IL-1 through IL-36 (including, e.g., human interleukins IL-1, IL-1a, IL-1B, IL-2, IL-3, IL-4, IL-6, IL-8, IL-10, IL-12, IL-11, IL-12, IL-13, IL-15, IL-18, IL-21, IL-23, IL-27, IL-31, IL-35), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Accordingly, in some embodiments, the transgene may comprise a nucleic acid encoding a pro-inflammatory agent or an immunosuppressive agent. For example, in some embodiments, the transgene may comprise a nucleic acid encoding one of IL-1Ra, IL-1β, IL-6, IL-10, IL-12, IL-15, GM-CSF, IFN-α, IFN-β, IFN-γ, TNF-α, CCL2, CCL5, CXCL9, CXCL10, CXCL12, TGFβ, or CSF-1. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules.

In some other embodiments, the immunomodulatory therapeutic protein is a human leukocyte antigen (“HLA”) polypeptide, including but not limited to an HLA-class Ib polypeptide. In some embodiments, the HLA polypeptide is HLA-E, HLA-F, or an isoform of HLA-G (e.g., HLA-G1, -G2, -G3, -G4, -G5, -G6, or -G7). Other suitable immunomodulatory polypeptides include but are not limited to CD47, PD-L1, CTLA-4, M-CSF, TGF-β1, IFN-γ, and various isoforms thereof.

In some embodiments, the transgene is a therapeutic polypeptide comprising an antibody or antigen-binding fragment thereof, e.g., an scFv.

In some embodiments, the therapeutic protein or polypeptide is a bone morphogenetic protein, an engineered protein scaffold, a serum protein, a globular protein, a defensive protein, a membrane or membrane-bound protein, a channel (e.g., an ion-exchange channel), a signaling protein, a regulatory protein, a transport protein, a sensory protein, a motor protein, a storage protein, a structural protein, or a thrombolytic protein.

In some embodiments, a therapeutic protein is a transcription factor such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, and a GATA-box binding protein, e.g., GATA-3, and the forkhead family of winged helix proteins.

In some embodiments, the transgene is a therapeutic polypeptide that binds to a pathogenic polypeptide, e.g., tau, alpha-synuclein, or beta-amyloid polypeptide.

In some embodiments, the transgene is a therapeutic polypeptide which targets cancer cells. In some embodiments, the therapeutic polypeptide is a chimeric antigen receptor, which bind to a tumor-associated antigen, such as CD19 or CD20.

In some embodiments, the therapeutic polypeptide is a T cell receptor (TCR) or an antigen-binding fragment thereof, e.g., a recombinant TCR. In some embodiments, the recombinant TCR can bind to an antigen of interest, e.g., an antigen selected from, but not limited to, CD279, CD2, CD95, CD152, CD223, CD272, TIM3, KIR, A2aR, SIRPa, CD200, CD200R, CD300, LPA5, NY-ESO, PD1, PDL1, or MAGE-A3/A6.

In some embodiments, the TCR or antigen-binding fragment thereof can bind to a viral antigen, e.g., an antigen from hepatitis A, hepatitis B, hepatitis C (HCV), human papilloma virus (HPV) (e.g., HPV-16 (such as HPV-16 E6 or HPV-16 E7), HPV-18, HPV-31, HPV-33, or HPV-35), Epstein-Barr virus (EBV), human herpes virus 8 (HHV-8), human T-cell leukemia virus-1 (HTLV-1), human T-cell leukemia virus-2 (HTLV-2) or a cytomegalovirus (CMV).

In some embodiments, a therapeutic protein or polypeptide may be a neutralizing antibody against a viral pathogen. Such anti-viral antibodies may include anti-influenza antibodies directed against one or more of Influenza A, Influenza B, and Influenza C. Other target pathogenic viruses include arenaviruses (including funin, machupo, and Lassa), filoviruses (including Marburg and Ebola), hantaviruses, picornoviridae (including rhinoviruses, echovirus), coronaviruses, paramyxovirus, morbillivirus, respiratory synctial virus, togavirus, coxsackievirus, JC virus, parvovirus B19, parainfluenza, adenoviruses, reoviruses, and variola (Variola major (Smallpox)) and Vaccinia (Cowpox) from the poxvirus family, and varicella-zoster (pseudorabies). For instance, in some embodiments, the therapeutic protein may be an anti-ebola antibody, e.g., 2G4, 4G7, 13C6, an anti-influenza antibody, e.g., FI6, CR8033, or an anti-RSV antibody, e.g., palivizumab, motavizumab.

In some embodiments, the therapeutic protein may be a neutralizing antibody construct against a bacterial pathogen. In one embodiment, the neutralizing antibody construct is directed against the bacteria itself. In another embodiment, the neutralizing antibody construct is directed against a toxin produced by the bacteria, such as the causative agent of anthrax, a toxin produced by Bacillius anthracis. Examples of airborne bacterial pathogens include, e.g., Neisseria meningitidis (meningitis), Klebsiella pneumonia (pneumonia), Pseudomonas aeruginosa (pneumonia), Pseudomonas pseudomallei (pneumonia), Pseudomonas mallei (pneumonia), Acinetobacter (pneumonia), Moraxella catarrhalis, Moraxella lacunata, Alkaligenes, Cardiobacterium, Haemophilus influenzae (flu), Haemophilus parainfluenzae, Bordetella pertussis (whooping cough), Francisella tularensis (pneumonia/fever), Legionella pneumoniae (Legionnaires disease), Chlamydia psittaci (pneumonia), Chlamydia pneumoniae (pneumonia), Mycobacterium tuberculosis (tuberculosis (TB)), Mycobacterium kansasii (TB), Mycobacterium avium (pneumonia), Nocardia asteroides (pneumonia), Bacillus anthracis (anthrax), Staphylococcus aureus (pneumonia), Streptococcus pyogenes (scarlet fever), Streptococcus pneumoniae (pneumonia), Corynebacteria diphtheria (diphtheria), Mycoplasma pneumoniae (pneumonia).

The therapeutic proteins may be antibodies against other infectious agents such as parasites or by fungi, including, e.g., Aspergillus species, Absidia corymbifera, Rhixpus stolonifer, Mucor plumbeaus, Cryptococcus neoformans, Histoplasm capsulatum, Blastomyces dermatitidis, Coccidioides immitis, Penicillium species, Micropolyspora faeni, Thermoactinomyces vulgaris, Alternaria alternate, Cladosporium species, Helminthosporium, and Stachybotrys species.

Additionally, in some embodiments, the transgene encodes a cell lineage commitment factor. For example, in some embodiments, the transgene encodes a gene product that, when expressed, promotes differentiation of a cell towards a more specialized cell type. For example, in some embodiments, the transgene encodes a lineage commitment factor that promotes differentiation of the cell towards a fibroblast, a hematopoietic cell, a neuron, a glial cell, an oligodendrocyte, a muscle cell, a bone cell, a hepatocyte, a pancreatic cell, a myeloid cell or a myeloid progenitor cell, a microglial cell or a microglial progenitor cell, a T cell, e.g., a CD4+ T cell, such as, a Treg. For example, in some embodiments, the transgene comprises CD4, CD25, ThPOK, FOXP3, CD45RA, CD62L, HELIOS, GITR, IKAROS, CTLA4, GATA3, TOX, ETS1, TCF7, LEF1, RORA, TNFR2, EOS, IRF5, SATB1, GATA1, or C-MYB.

### 6.5. Separator Sequences

The targeting constructs and recombinant target cell genomes described herein can also comprise a separator sequence between a degron coding sequence and the transgene. Such separator sequences can allow separate expression of polypeptides encoded by a single expression cassette.

In some embodiments, the separator sequence is an internal ribosome entry site (IRES), which allows the transgene to be translated separately from the fusion protein comprising the sequences of the essential polypeptide and the degron.

In some embodiments, the separator sequence is a self-cleaving peptide, associated with ribosomal skipping during translation, in which the ribosomes skip the peptide bond between a C-terminal Gly and Pro, resulting in the production of two separate polypeptides, i.e., the transgene and the fusion protein comprising the sequences of the essential polypeptide and the degron. In further embodiments, polypeptide coding sequences comprising a fusion protein in an expression cassette may be separated by translation-skipping sequences (i.e., in-frame coding sequences for a self-cleaving peptide), such that translation of the mRNA transcript from the polycistronic cassette will result in separate proteins. A self-cleaving peptide causes ribosomal skipping during translation. Examples of self-cleaving peptides are 2A peptides, which are viral derived peptides with a typical length of 18-22 amino acids. 2A peptides include T2A, P2A, E2A, F2A, and PQR (Lo et al., 2015, Cell Reports 13:2634-2644). By way of example, P2A is a peptide of 19 amino acids; after the cleavage, a few amino acid residues from the P2A are left on the upstream polypeptide and a proline is left at the beginning of the second polypeptide. 2A residues left on the fusion protein and the polypeptide encoded by the transgene are not believed to affect their functionality.

### 6.6. Expression Vectors

The present disclosure provides expression vectors encoding the fusion proteins of the disclosure. An expression vector typically comprises an expression cassette comprising a fusion polypeptide as described in Section 6.2 operably linked to a regulatory element such as a promoter and, optionally, a self-replication element.

Without being bound by theory, it is believed that recombinant expression of a fusion protein comprising an essential polypeptide and a degron can “poison” the native cellular protein and result in its destabilization when the degron is activated, resulting in cell death even when the essential gene is intact and not modified by recombination with a targeting construct of the disclosure. Thus, the fusion proteins of the disclosure can be expressed by an expression cassette that is not integrated into an essential gene. In some embodiments, the expression cassette is part of an extrachromosomal vector. In other embodiments, the expression cassette is integrated into the target cell genome without modifying the sequence of the native essential polypeptide.

The expression vector may be a viral genome, a single-stranded RNA or DNA, or double-stranded DNA, e.g., a plasmid.

Expression vectors can include other coding or non-coding elements. For example, an expression cassette can be delivered as part of a viral genome (e.g., in an AAV, adenoviral, Sendai virus, or lentiviral genome) that includes certain genomic backbone elements (e.g., inverted terminal repeats, in the case of an AAV genome).

In some embodiments, an expression vector a circular plasmid that has not been linearized.

In some embodiments, an expression vector is a circular plasmid that has been linearized.

In some embodiments, an expression vector is a viral genome. Viral genomes provide a rich source of vectors that can be used for the efficient delivery of an exogenous nucleic acid into the genome of a target cell (e.g., a mammalian cell, such as a human cell). Viral genomes are particularly useful vectors for delivery of exogenous nucleic acids because the nucleic acids contained within such genomes are typically incorporated into the genome of a target cell by generalized or specialized transduction. These processes occur as part of the natural viral replication cycle, and do not require added proteins or reagents in order to induce gene integration. Examples of viral vectors include AAV, retrovirus, adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering exogenous nucleic acids include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses. Other examples of vectors are described, for example, in U.S. Pat. No. 5,801,030.

In some embodiments, the expression vectors are recombinant adeno-associated viral vectors (“rAAV vectors”). rAAV vectors useful in the invention are recombinant nucleic acid constructs that include (1) an expression cassette (e.g., a nucleic acid encoding a fusion protein comprising an essential polypeptide and a degron, optionally connected via a linker) and (2) viral nucleic acids that facilitate expression of the fusion protein. The viral nucleic acids may include those sequences of AAV that are required in cis for replication and packaging (e.g., functional ITRs) of the DNA into a virion. Useful rAAV vectors have one or more of the AAV WT genes deleted in whole or in part, but retain functional flanking ITR sequences. The AAV ITRs may be of any serotype suitable for a particular application. Methods for using rAAV vectors are described, for example, in Tal et al., 2000, J. Biomed. Sci. 7:279-291, and Monahan and Samulski, 2000, Gene Delivery 7:24-30. rAAVs can be derived from any suitable serotype, including AAV1, 2, 3, 4, 5, 6, 7, 8 and 9.

In some embodiments, the expression cassette comprises a transgene, e.g., a transgene encoding a therapeutic polypeptide, in addition to the fusion protein coding sequence.

The transgene and the fusion protein may be expressed from a common promoter. In some embodiments, the transgene is separated from the fusion protein coding sequence by a translation-skipping sequence (for example, an in-frame coding sequences for a self-cleaving peptide), such that translation of the mRNA transcript from the polycistronic cassette will result in separate polypeptides, i.e., the fusion protein comprising the essential polypeptide and the degron and the polypeptide encoded by the transgene. Self-cleaving peptides are described in Section 6.5.

Alternatively, the expression cassette can be a polycistronic expression cassette with the fusion protein coding sequence and the transgene separated by an internal ribosome entry site (IRES) in the mRNA.

### 6.7. Target Cells

In some embodiments, a targeting construct or expression vector is introduced into target cells or populations of target cells. Methods for introducing proteins and nucleic acids to target cells are described further in Section 6.9.

The target cells and target cell populations of the disclosure can be cells engineered to express a fusion protein comprising an essential polypeptide and a degron and optionally a transgene. A cell population can comprise, for example, a population in which at least 1%, at least 5%, at least 10%, at least 15%, at least 20%, at least 30%, at least 40%, at least 50%, at least 60%, or at least 70% of the cells have been engineered to express a fusion protein comprising the essential polypeptide and degron.

In some embodiments, the methods of the disclosure may be employed to express a fusion protein in mitotic or post-mitotic target cells in vivo and/or ex vivo and/or in vitro (e.g., to produce engineered target cells that can be reintroduced into an individual).

Any type of cell may be of interest (e.g., a stem cell, e.g., a human embryonic stem cell (hESC), an induced pluripotent stem cell (iPSC), a germ cell; a somatic cell, e.g., a fibroblast, a hematopoietic cell, a neuron, a glial cell, an oligodendrocyte, a muscle cell, a bone cell, a hepatocyte, a pancreatic cell, a myeloid cell or a myeloid progenitor cell, e.g., a primitive myeloid progenitor cell, a microglial cell or a microglial progenitor cell, a T cell, e.g., a CD4+ T cell, such as, a Treg; an in vitro or in vivo embryonic cell of an embryo at any stage, e.g., a 1-cell, 2-cell, 4-cell, 8-cell, etc. stage zebrafish embryo; etc.). Cells may be from established cell lines, or they may be primary cells, where “primary cells”, “primary cell lines”, and “primary cultures” are used interchangeably herein to refer to cells and cells cultures that have been derived from a subject and allowed to grow in vitro for a limited number of passages, e.g., splittings, of the culture. For example, primary cultures include cultures that may have been passaged 0 times, 1 time, 2 times, 4 times, 5 times, 10 times, or 15 times, but not enough times to go through the crisis stage. Primary cell lines can be maintained for fewer than 10 passages in vitro. Target cells are, in some embodiments, unicellular organisms, or are grown in culture. Preferably, the target cells are of human origin. In some embodiments, a target cell is an autologous cell in the context of cell therapy. In some embodiments, a target cell is an allogeneic cell in the context of a cell therapy.

If the cells are primary cells, such cells may be harvested from an individual by any suitable method. For example, leukocytes may be suitably harvested by apheresis, leukocytapheresis, density gradient separation, etc., while cells from tissues such as skin, muscle, bone marrow, spleen, liver, pancreas, lung, intestine, stomach, etc. are most suitably harvested by biopsy. An appropriate solution may be used for dispersion or suspension of the harvested cells. Such solution will generally be a balanced salt solution, e.g., normal saline, phosphate-buffered saline (PBS), Hank's balanced salt solution, etc., suitably supplemented with fetal calf serum or other naturally occurring factors, in conjunction with an acceptable buffer at low concentration, e.g., from 5-25 mM. Suitable buffers include HEPES, phosphate buffers, lactate buffers, etc. The cells may be used immediately, or they may be stored, frozen, for long periods of time, being thawed and capable of being reused. In such cases, the cells will generally be frozen in 10% dimethyl sulfoxide (DMSO), 50% serum, 40% buffered medium, or some other such solution as is commonly used in the art to preserve cells at such freezing temperatures and thawed in a manner as commonly known in the art for thawing frozen cultured cells.

Methods of introducing heterologous nucleic acids into target cells, e.g., to engineer the target cells to express a fusion protein of the disclosure and optionally a transgene, are disclosed in Sections 6.8 and 6.9.

In some embodiments, a target cell is engineered to incorporate a single copy of a coding sequence encoding a fusion protein comprising an essential polypeptide and a degron. In other embodiments, a target cell is engineered to incorporate two copies of a coding sequence encoding a fusion protein comprising an essential polypeptide and a degron. In some embodiments, each allele of an essential gene locus is modified to express a fusion protein comprising an essential polypeptide and a degron. In yet other embodiments, a target cell is engineered to express multiple copies of a coding sequence encoding a fusion protein comprising an essential polypeptide and a degron, wherein two or more copies are integrated at each allele of the corresponding essential gene locus. As set forth in Section 6.2, in each of the foregoing embodiments, the fusion protein can comprise one, two or more degrons in tandem. In some embodiments, the degrons separated by linkers.

In some embodiments, the single copy is achieved by integrating a targeting construct into a single allele of an essential gene. The degron coding sequence may be positioned 5′ or 3′ to the essential polypeptide coding sequence, such that the fusion protein can include the degron(s) at its N- or C-terminus. The essential polypeptide and the degron(s) may be separated by a linker. The target cell may further be engineered to express a transgene. The transgene may be expressed from the same allele of the essential gene that has been modified to encode a fusion protein, from the opposite allele, from another genomic locus altogether, or from an extrachromosomal expression vector. In some embodiments, the degron coding sequence(s) and the transgene are both introduced into the same allele or different alleles of the same essential STEL gene, e.g., into the GAPDH locus.

In some embodiments, the two copies achieved by integrating a targeting construct into both alleles of an essential gene. The degron coding sequence(s) may be positioned 5′ or 3′ to the essential polypeptide coding sequence, such that the fusion protein can include the degron(s) at its N- or C-terminus. The essential polypeptide and the degron(s) may be separated by a linker. The target cell may further be engineered to express a transgene. The transgene may be expressed from the same alleles of the essential gene that has been modified to encode a fusion protein, from another genomic locus, or from an extrachromosomal expression vector. In some embodiments, the degron coding sequence(s) and the transgene are both introduced into the same allele of an essential STEL gene, e.g., into the GAPDH locus.

In some embodiments, a target cell of the disclosure of the disclosure comprises a single allele of an essential gene into which a targeting construct comprising a degron, e.g., a targeting construct according to any one of FIG. 2A, 2B, 2C, or 2D, is integrated. Optionally, the target cell comprises a transgene integrated into one allele or both alleles of a different gene locus.

In some embodiments, a target cell of the disclosure of the disclosure comprises two alleles of an essential gene into which a targeting construct comprising a degron, e.g., a targeting construct according to any one of FIG. 2A, 2B, 2C, or 2D, is integrated. Optionally, the target cell comprises a transgene integrated into one allele or both alleles of a different gene locus.

In some embodiments, a target cell of the disclosure comprises two essential gene loci configured as illustrated in FIG. 3A. In some embodiments, a target cell of the disclosure comprises two essential gene loci configured as illustrated in FIG. 3B. In some embodiments, a target cell of the disclosure comprises two essential gene loci configured as illustrated in FIG. 3D. In some embodiments, a target cell of the disclosure comprises two essential gene loci configured as illustrated in FIG. 3E. In each of the foregoing embodiments, the target cell optionally comprises a transgene integrated into one allele or both alleles of a different gene locus.

In some embodiments, a target cell of the disclosure of the disclosure comprises one allele of an essential gene into which a targeting construct comprising a degron and a transgene, e.g., a targeting construct according to any one of FIG. 4A, 4B, 4C, or 4D, is integrated. In some embodiments, a target cell of the disclosure of the disclosure comprises two alleles of an essential gene into which a targeting construct comprising a degron and a transgene, e.g., a targeting construct according to any one of FIG. 4A, 4B, 4C, or 4D, is integrated.

In some embodiments, a target cell of the disclosure of the disclosure comprises one allele of an essential gene into which a targeting construct comprising a degron and a transgene, e.g., a targeting construct according to any one of FIG. 7A, 7B, 7C, 7D, 7E, 7F, 7G, 7H, 7I, 7J, 7K or 7L, is integrated. In some embodiments, a target cell of the disclosure of the disclosure comprises two alleles of an essential gene into which a targeting construct comprising a degron and a transgene, e.g., a targeting construct according to any one of FIGS. FIG. 7A, 7B, 7C, 7D, 7E, 7F, 7G, 7H, 7I, 7J, 7K or 7L, is integrated.

In some embodiments, a target cell of the disclosure comprises two essential gene loci configured as illustrated in FIG. 5A. In some embodiments, a target cell of the disclosure comprises two essential gene loci configured as illustrated in FIG. 5B. In some embodiments, a target cell of the disclosure comprises two essential gene loci configured as illustrated in FIG. 5C. In some embodiments, a target cell of the disclosure comprises two essential gene loci configured as illustrated in FIG. 5D. In some embodiments, a target cell of the disclosure comprises two essential gene loci configured as illustrated in FIG. 6A. In some embodiments, a target cell of the disclosure comprises two essential gene loci configured as illustrated in FIG. 6B. In some embodiments, a target cell of the disclosure comprises two essential gene loci configured as illustrated in FIG. 6C. In some embodiments, a target cell of the disclosure comprises two essential gene loci configured as illustrated in FIG. 6D.

### 6.7.1. Stem Cells

In some embodiments, the target cells that are engineered to express a fusion protein of the disclosure and, optionally, a transgene are stem cells, particularly pluripotent stem cells (PSCs) such as induced pluripotent stem cells (iPSCs) or human embryonic stem cells (hESCs), which are the starting point for the potential generation of large numbers of a specific cell type that can be delivered for regenerative medicine in patients with many different diseases.

Suitable methods of introducing heterologous nucleic acids into stem cells, e.g., to engineer the stem cells to express a fusion protein of the disclosure and optionally a transgene, are disclosed in Sections 6.8 and 6.9.

Following engineering to a PSC to express a fusion protein of the disclosure and, optionally, a transgene, the PSC can be differentiated into a cell type of interest for cell therapy.

The recombinant PSCs can be differentiated into cells suitable for therapy, including the cells in the endoderm (e.g., lung, thyroid, or pancreatic cells, or progenitors thereof), ectoderm (e.g., skin, neuronal, or pigment cells, or progenitors thereof) and mesoderm (e.g., cardiac cells, skeletal muscle cells, red blood cells, smooth muscle cells, or progenitors or precursors thereof) lineages.

In some embodiments, the recombinant PSCs are differentiated into cells in the endoderm (e.g., lung, thyroid, or pancreatic cells, or progenitors or precursors thereof), ectoderm (e.g., skin, neuronal, or pigment cells, or progenitors or precursors thereof) or mesoderm (e.g., cardiac cells, skeletal muscle cells, red blood cells, smooth muscle cells, or progenitors or precursors thereof) lineages.

In some embodiments, a recombinant PSC of the disclosure is differentiated into a cardiac cell. In various embodiments, the cardiac cell is a cardiac progenitor cell or a mature or immature (atrial or ventricular) cardiomyocyte. In other embodiments, the cardiac cell is a cardiac endothelial cell or a nodal cell.

In some embodiments, a recombinant PSC of the disclosure is differentiated into a human immune cell, optionally selected from a T cell, a T cell expressing a chimeric antigen receptor (CAR) or recombinant TCR, a regulatory T cell, a myeloid cell, a dendritic cell, and/or a macrophage (e.g., an immunosuppressive macrophage), or a progenitor or precursor thereof. In some embodiments, a recombinant PSC of the disclosure is differentiated into a myeloid progenitor cell, e.g., as described in WO 2023/150089 A1, the contents of which are incorporated by reference in their entireties herein.

In some embodiments, a recombinant PSC of the disclosure is differentiated into an oligodendrocyte progenitor cell or precursor cell, or an oligodendrocyte.

In some embodiments, a recombinant PSC of the disclosure is differentiated into a neural lineage cell, for example a neural crest cells, an astrocyte, a dopaminergic neuron progenitor cell, a dopaminergic neuron cells, a midbrain dopaminergic neuron progenitor cell, a midbrain dopaminergic neuron, an authentic midbrain dopamine (DA) neuron, a dopaminergic neuron precursor cell, a floor plate midbrain progenitor cell, a floor plate midbrain DA neuron, or a progenitor or precursor thereof.

In some embodiments, a recombinant PSC of the disclosure is differentiated into a cell of the ocular system, such as a photoreceptor cell, a photoreceptor progenitor or precursor cell, a retinal pigmented epithelium cell or a progenitor or precursor thereof, a neural retinal cell or a progenitor or precursor thereof. In other embodiments, an unedited PSC is differentiated into a cell of the ocular system, which is then recombinant with a targeting construct of the disclosure.

In further embodiments, a recombinant PSC of the disclosure is differentiated into a microglial cell or a microglial progenitor or precursor cell.

In further embodiments, a recombinant PSC of the disclosure is differentiated into a cell in the human metabolic system, optionally selected from a hepatocyte, a cholangiocyte, and a pancreatic beta cell, or a progenitor or precursor thereof.

In further embodiments, a recombinant PSC of the disclosure is differentiated into an enteric progenitor or precursor cell or an enteric cell.

### 6.7.2. Differentiated Cells

In various embodiments, a cell at any stage of differentiation is engineered to express a fusion protein of the disclosure and optionally a transgene.

Suitable methods of introducing heterologous nucleic acids into differentiated cells, e.g., to engineer the differentiated cells to express a fusion protein of the disclosure and optionally a transgene, are disclosed in Sections 6.8 and 6.9.

Exemplary differentiated cell types that can be engineered to express a fusion protein of the disclosure include the cells in the endoderm (e.g., lung, thyroid, or pancreatic cells, or progenitors thereof), ectoderm (e.g., skin, neuronal, or pigment cells, or progenitors or precursors thereof) and mesoderm (e.g., cardiac cells, skeletal muscle cells, red blood cells, smooth muscle cells, or progenitors or precursors thereof) lineages. Alternatively, PSCs can be differentiated into cells in these lineages and then recombinant with a targeting construct of the disclosure.

In some embodiments, a cardiac cell is engineered to express a fusion protein of the disclosure. In some embodiments, the cardiac cell is a cardiac progenitor cell or a mature or immature (atrial or ventricular) cardiomyocyte. In other embodiments, the cardiac cell is a cardiac endothelial cell or a nodal cell.

In some embodiments, a human immune cell is engineered to express a fusion protein of the disclosure. The human immune cell is optionally selected from a T cell, a T cell expressing a chimeric antigen receptor (CAR) or recombinant TCR, a regulatory T cell, a myeloid cell, a dendritic cell, and/or a macrophage (e.g., an immunosuppressive macrophage), or a progenitor or precursor thereof. In some embodiments, a myeloid progenitor cell is engineered to express a fusion protein of the disclosure following differentiation from a PSC, e.g., as described in WO 2023/150089 A1, the contents of which are incorporated by reference in their entireties herein.

In some embodiments, an oligodendrocyte progenitor cell or precursor cell or an oligodendrocyte is engineered to express a fusion protein of the disclosure.

In some embodiments, a neural lineage cell is engineered to express a fusion protein of the disclosure. In various embodiments, the neural lineage cell is a neural crest cell, an astrocyte, a dopaminergic neuron progenitor cell, a dopaminergic neuron cell, a midbrain dopaminergic neuron progenitor cell, a midbrain dopaminergic neuron, an authentic midbrain dopamine (DA) neuron, a dopaminergic neuron precursor cell, a floor plate midbrain progenitor cell, a floor plate midbrain DA neuron, or a progenitor or precursor thereof.

In some embodiments, a cell of the ocular system is engineered to express a fusion protein of the disclosure. In various embodiments, the cell of the ocular system is a photoreceptor cell, a photoreceptor progenitor or precursor cell, a retinal pigmented epithelium cell or a progenitor or precursor thereof, a neural retinal cell or a progenitor or precursor thereof.

In further embodiments, a microglial cell or a microglial progenitor or precursor cell is engineered to express a fusion protein of the disclosure.

In further embodiments, a cell in the human metabolic system is engineered to express a fusion protein of the disclosure. In various embodiments, the cell in the human metabolic system is optionally selected from a hepatocyte, a cholangiocyte, and a pancreatic beta cell, or a progenitor or precursor thereof.

In further embodiments, an enteric progenitor or precursor cell or an enteric cell is engineered to express a fusion protein of the disclosure.

Any of the foregoing differentiated cell types can differentiated from PSCs prior to engineering them to express a fusion protein of the disclosure.

### 6.8. Methods for the Delivery of Exogenous Nucleic Acids to Target Cells

The targeting constructs and expression vectors of the disclosure are delivered to a target cell, thereby generating a recombinant target cell that comprises a nucleic acid encoding a fusion protein comprising an essential polypeptide, a degron and an optional linker. The nucleic acid may be integrated into the target cell genome, e.g., when a targeting construct is used, or remain extrachromosomal, e.g., when an extrachromosomal expression vector is used.

Exemplary methods of introducing the targeting constructs and expression vectors are described below.

Techniques that can be used to introduce a nucleic acid, such as a targeting construct or expression vector of the disclosure, into a target cell are known in the art. For example, electroporation can be used to permeabilize mammalian cells (e.g., human target cells) by the application of an electrostatic potential to the cell of interest. Mammalian cells, such as human cells, subjected to an external electric field in this manner are subsequently predisposed to the uptake of exogenous nucleic acids. Electroporation of mammalian cells is described in detail, e.g., in Chu et al., 1987, Nucleic Acids Research 15:131. A similar technique, Nucleofection™ utilizes an applied electric field in order to stimulate the uptake of exogenous nucleic acids into the nucleus of a eukaryotic cell. Nucleofection™ and protocols useful for performing this technique are described in detail, e.g., in Distler et al., 2005, Experimental Dermatology 14:315, as well as in US 2010/03171 14.

Additional techniques useful for the transfection of target cells include the squeeze-poration methodology. This technique induces the rapid mechanical deformation of cells in order to stimulate the uptake of exogenous DNA through membranous pores that form in response to the applied stress. This technology is advantageous in that a vector is not required for delivery of nucleic acids into a cell, such as a human target cell. Squeeze-poration is described in detail, e.g., in Sharei et al., 2013, Journal of Visualized Experiments 81: e50980.

Lipofection represents another technique useful for transfection of target cells. This method involves the loading of nucleic acids into a liposome, which often presents cationic functional groups, such as quaternary or protonated amines, towards the liposome exterior. This promotes electrostatic interactions between the liposome and a cell due to the anionic nature of the cell membrane, which ultimately leads to uptake of the exogenous nucleic acids, for example, by direct fusion of the liposome with the cell membrane or by endocytosis of the complex. Lipofection is described in detail, for example, in U.S. Pat. No. 7,442,386. Similar techniques that exploit ionic interactions with the cell membrane to provoke the uptake of foreign nucleic acids include contacting a cell with a cationic polymer-nucleic acid complex. Exemplary cationic molecules that associate with nucleic acids so as to impart a positive charge favorable for interaction with the cell membrane are activated dendrimers (described, e.g., in Dennig, 2003, Topics in Current Chemistry 228:227 and diethylaminoethyl (DEAE)-dextran, the use of which as a transfection agent is described in detail, for example, in Gulick et al., 1997, Current Protocols in Molecular Biology 40:1:9.2:9.2.1. Magnetic beads are another tool that can be used to transfect target cells in a mild and efficient manner, as this methodology utilizes an applied magnetic field in order to direct the uptake of nucleic acids. This technology is described in detail, for example, in US 2010/0227406.

Another useful tool for inducing the uptake of exogenous nucleic acids by target cells is laserfection, a technique that involves exposing a cell to electromagnetic radiation of a particular wavelength in order to gently permeabilize the cells and allow nucleic acids to penetrate the cell membrane. This technique is described in detail, e.g., in Rhodes et al., 2007, Methods in Cell Biology 82:309.

Microvesicles represent another potential vehicle that can be used to introduce a nucleic acid, such as a targeting construct as disclosed herein, into the genome of a target cell. For example, microvesicles that have been induced by the co-overexpression of the glycoprotein VSV-G with, e.g., a genome-modifying protein, such as a nuclease, can be used to efficiently deliver proteins into a cell that subsequently catalyze the site-specific cleavage of an endogenous nucleic acid sequence so as to prepare the genome of the cell for the covalent incorporation of a nucleic acid of interest, such as a gene or regulatory sequence. The use of such vesicles, also referred to as Gesicles, for the genetic modification of eukaryotic cells is described in detail, e.g., in Quinn et al., 2015, Genetic Modification of Target Cells by Direct Delivery of Active Protein (at Abstract). In: Methylation changes in early embryonic genes in cancer (Abstract), in: Proceedings of the 18th Annual Meeting of the American Society of Gene and Cell Therapy, Abstract No. 122.

In addition to the above, a variety of tools have been developed that can be used for the incorporation of a gene of interest into a target cell, such as a human cell. One such method that can be used for incorporating transgenes or polynucleotides encoding fusion proteins into target cells involves the use of transposons. Transposons are polynucleotides that encode transposase enzymes and contain a polynucleotide sequence or gene of interest flanked by 5′ and 3′ excision sites. Once a transposon has been delivered into a cell, expression of the transposase gene commences and results in active enzymes that cleave the gene of interest from the transposon. This activity is mediated by the site-specific recognition of transposon excision sites by the transposase. In some instances, these excision sites may be terminal repeats or inverted terminal repeats. Once excised from the transposon, the gene of interest can be integrated into the genome of a mammalian cell by transposase-catalyzed cleavage of similar excision sites that exist within the nuclear genome of the cell. This allows the gene of interest to be inserted into the cleaved nuclear DNA at the complementary excision sites, and subsequent covalent ligation of the phosphodiester bonds that join the gene of interest to the DNA of the mammalian cell genome completes the incorporation process. In certain cases, the transposon may be a retrotransposon, such that the gene encoding the essential gene is first transcribed to an RNA product and then reverse-transcribed to DNA before incorporation in the mammalian cell genome. Exemplary transposon systems are the piggybac transposon (described in detail in, e.g., WO 2010/085699) and the sleeping beauty transposon (described in detail in, e.g., US 2005/01 12764).

Other tools for the integration of exogenous nucleic acids into the genome of a target cell are based on nuclease-based gene editing (or genome editing), for example the CRISPR/Cas system, zinc finger nucleases (ZFNs) and transcription activator-like effector nucleases (TALENs). Exemplary CRISPR/Cas gene-editing approaches are disclosed in Section 6.9. The use of ZFNs and TALENs in genome editing applications is described, e.g., in Urnov et al., 2010, Nature Reviews Genetics 11:636 and in Joung et al., 2013, Nature Reviews Molecular Cell Biology 14:49.

Additional genome editing techniques that can be used to incorporate nucleic acids comprising transgenes or encoding fusion proteins into the genome of a target cell include the use of ARCUS™ meganucleases that can be rationally designed so as to site-specifically cleave genomic DNA. The use of these enzymes for the incorporation of transgenes or nucleic acids encoding fusion proteins into the genome of a mammalian cell is advantageous in view of the defined structure-activity relationships that have been established for such enzymes. Single chain meganucleases can be modified at certain amino acid positions in order to create nucleases that selectively cleave DNA at desired locations, enabling the site-specific incorporation of an essential gene into the nuclear DNA of a target cell. These single-chain nucleases have been described extensively in, for example, U.S. Pat. Nos. 8,021,867 and 8,445,251.

### 6.9 Exemplary Gene Editing Approaches

### 6.9.1. Endonuclease Systems

The targeting construct of the present disclosure can be incorporated into a target cell with an endonuclease system.

An endonuclease system may comprise:
(i) a targeting construct as described in Section 6.3;
(ii) an endonuclease enzyme as described in Section 6.9.2 or nucleic acid encoding the endonuclease enzyme;
(iii) a guide RNA as described in Section 6.9.3 or a nucleic acid encoding the guide RNA.

In some embodiments, an endonuclease system comprises:
(i) a targeting construct as described in Section 6.3;
(ii) an endonuclease enzyme as described in Section 6.9.2;
(iii) a guide RNA as described in Section 6.9.3.

The endonuclease system may be delivered into a target cell in the form of a ribonucleoprotein complex, as described in Section 6.9.4.

### 6.9.2. Endonucleases

The targeting constructs of the disclosure may be incorporated into a specific target genomic locus by facilitating homologous recombination at DNA breaks generated by a suitable endonuclease.

In some embodiments, the endonuclease is a CRISPR-associated endonuclease, such as a Cas endonuclease, selected from, without limitation, a type II, type IV, or type V Cas protein.

In some embodiments, the endonuclease is a Cas protein, including but not limited to, Cas1, Cas1B, Cas2, Cas3, Cas4, Cas5, Cas6, Cas7, Cas8, Cas9, Cas12a (e.g., Cpf1) or Cas12b, homologs thereof, or modified versions thereof, e.g., truncated versions or variants of a wildtype Cas protein with a nuclease activity.

In some embodiments, the Cas endonuclease is a Cpf1 (Cas12a) endonuclease, or a variant, derivative, or fragment thereof, such as, for example, Cpf1 derived from Francisella novicida U112 (FnCpf1), Acidaminococcus sp. BV3L6 (AsCpf1, including improved variants such as enAsCpf1), Lachnospiraceae bacterium ND2006 (LbCpf1), Lachnospiraceae bacterium MA2020 (Lb2Cpf1), Lachnospiraceae bacterium MC2017 (Lb3Cpf1), Moraxella bovoculi 237 (MbCpf1), or Prevotella disiens (PdCpf1).

In some embodiments, the Cas endonuclease is a Cas9 protein or a variant, derivative, or fragment thereof. In some embodiments, the Cas9 protein is SaCas9, SpCas9, SpCas9n, Cas9-HF, Cas9-H840A, Fokl-dCas9, or D10A nickase.

In some embodiments, the Cas endonuclease is a Type V RNA programmable nuclease, as disclosed in WO 2022/258753 A1, the contents of which are incorporated by reference herein in their entireties.

In some embodiments, the Cas endonuclease is a MAD nuclease, such as MAD7 nuclease, as disclosed in U.S. Pat. No. 10,337,028, the contents of which are incorporated by reference herein in their entireties.

In some aspects, the targeting constructs may be incorporated into target genomic loci using non-CRISPR endonucleases, including but not limited to, Transcription Activator-Like Effector Nucleases (TALENs), zinc finger nuclease (ZFNs) homing endonucleases, sequence-specific endonucleases, or meganucleases.

Non-limiting examples of suitable endonucleases are set forth in Table 3.

TABLE 3

| | | SEQ ID |
|---|---|---|
| Enzyme | Sequence | NO |
| Cas12a | MTQFEGFTNLYQVSKTLRFELIPQGKTLKHIQEQGFIEEDKARNDHYK | 24 |
| | ELKPIIDRIYKTYADQCLQLVQLDWENLSAAIDSYRKEKTEETRNALIEE | |
| | QATYRNAIHDYFIGRTDNLTDAINKRHAEIYKGLFKAELFNGKVLKQLG | |
| | TVTTTEHENALLRSFDKFTTYFSGFYENRKNVFSAEDISTAIPHRIVQD | |
| | NFPKFKENCHIFTRLITAVPSLREHFENVKKAIGIFVSTSIEEVFSFPFY | |
| | NQLLTQTQIDLYNQLLGGISREAGTEKIKGLNEVLNLAIQKNDETAHIIA | |
| | SLPHRFIPLFKQILSDRNTLSFILEEFKSDEEVIQSFCKYKTLLRNENVL | |
| | ETAEALFNELNSIDLTHIFISHKKLETISSALCDHWDTLRNALYERRISE | |
| | LTGKITKSAKEKVQRSLKHEDINLQEIISAAGKELSEAFKQKTSEILSHA | |
| | HAALDQPLPTTLKKQEEKEILKSQLDSLLGLYHLLDWFAVDESNEVDP | |
| | EFSARLTGIKLEMEPSLSFYNKARNYATKKPYSVEKFKLNFQMPTLAS | |
| | GWDVNKEKNNGAILFVKNGLYYLGIMPKQKGRYKALSFEPTEKTSEG | |
| | FDKMYYDYFPDAAKMIPKCSTQLKAVTAHFQTHTTPILLSNNFIEPLEI | |
| | TKEIYDLNNPEKEPKKFQTAYAKKTGDQKGYREALCKWIDFTRDFLSK | |
| | YTKTTSIDLSSLRPSSQYKDLGEYYAELNPLLYHISFQRIAEKEIMDAV | |
| | ETGKLYLFQIYNKDFAKGHHGKPNLHTLYWTGLFSPENLAKTSIKLNG | |
| | QAELFYRPKSRMKRMAHRLGEKMLNKKLKDQKTPIPDTLYQELYDYV | |
| | NHRLSHDLSDEARALLPNVITKEVSHEIIKDRRFTSDKFFFHVPITLNY | |
| | QAANSPSKFNQRVNAYLKEHPETPIIGIDRGERNLIYITVIDSTGKILEQ | |
| | RSLNTIQQFDYQKKLDNREKERVAARQAWSVVGTIKDLKQGYLSQVI | |
| | HEIVDLMIHYQAVVVLENLNFGFKSKRTGIAEKAVYQQFEKMLIDKLNC | |
| | LVLKDYPAEKVGGVLNPYQLTDQFTSFAKMGTQSGFLFYVPAPYTSKI | |
| | DPLTGFVDPFVWKTIKNHESRKHFLEGFDFLHYDVKTGDFILHFKMNR | |
| | NLSFQRGLPGFMPAWDIVFEKNETQFDAKGTPFIAGKRIVPVIENHRF | |
| | TGRYRDLYPANELIALLEEKGIVFRDGSNILPKLLENDDSHAIDTMVALI | |
| | RSVLQMRNSNAATGEDYINSPVRDLNGVCFDSRFQNPEWPMDADA | |
| | NGAYHIALKGQLLLNHLKESKDLKLQNGISNQDWLAYIQELRN | |
| Cas12a | MTQFEGFTNLYQVSKTLRFELIPQGKTLKHIQEQGFIEEDKARNDHYK | 25 |
| variant 1 | ELKPIIDRIYKTYADQCLQLVQLDWENLSAAIDSYRKEKTEETRNALIEE | |
| | QATYRNAIHDYFIGRTDNLTDAINKRHAEIYKGLFKAELFNGKVLKQLG | |
| | TVTTTEHENALLRSFDKFTTYFSGFYENRKNVFSAEDISTAIPHRIVQD | |
| | NFPKFKENCHIFTRLITAVPSLREHFENVKKAIGIFVSTSIEEVFSFPFY | |
| | NQLLTQTQIDLYNQLLGGISREAGTEKIKGLNEVLNLAIQKNDETAHIIA | |
| | SLPHRFIPLFKQILSDRNTLSFILEEFKSDEEVIQSFCKYKTLLRNENVL | |
| | ETAEALFNELNSIDLTHIFISHKKLETISSALCDHWDTLRNALYERRISE | |
| | LTGKITKSAKEKVQRSLKHEDINLQEIISAAGKELSEAFKQKTSEILSHA | |
| | HAALDQPLPTTLKKQEEKEILKSQLDSLLGLYHLLDWFAVDESNEVDP | |
| | EFSARLTGIKLEMEPSLSFYNKARNYATKKPYSVEKFKLNFQRPTLAS | |
| | GWDVNKEKNNGAILFVKNGLYYLGIMPKQKGRYKALSFEPTEKTSEG | |
| | FDKMYYDYFPDAAKMIPKCSTQLKAVTAHFQTHTTPILLSNNFIEPLEI | |
| | TKEIYDLNNPEKEPKKFQTAYAKKTGDQKGYREALCKWIDFTRDFLSK | |
| | YTKTTSIDLSSLRPSSQYKDLGEYYAELNPLLYHISFQRIAEKEIMDAV | |
| | ETGKLYLFQIYNKDFAKGHHGKPNLHTLYWTGLFSPENLAKTSIKLNG | |
| | QAELFYRPKSRMKRMAARLGEKMLNKKLKDQKTPIPDTLYQELYDYV | |
| | NHRLSHDLSDEARALLPNVITKEVSHEIIKDRRFTSDKFLFHVPITLNYQ | |
| | AANSPSKFNQRVNAYLKEHPETPIIGIDRGERNLIYITVIDSTGKILEQR | |
| | SLNTIQQFDYQKKLDNREKERVAARQAWSVVGTIKDLKQGYLSQVIH | |
| | EIVDLMIHYQAVVVLENLNFGFKSKRTGIAEKAVYQQFEKMLIDKLNCL | |
| | VLKDYPAEKVGGVLNPYQLTDQFTSFAKMGTQSGFLFYVPAPYTSKI | |
| | DPLTGFVDPFVWKTIKNHESRKHFLEGFDFLHYDVKTGDFILHFKMNR | |
| | NLSFQRGLPGFMPAWDIVFEKNETQFDAKGTPFIAGKRIVPVIENHRF | |
| | TGRYRDLYPANELIALLEEKGIVFRDGSNILPKLLENDDSHAIDTMVALI | |
| | RSVLQMRNSNAATGEDYINSPVRDLNGVCFDSRFQNPEWPMDADA | |
| | NGAYHIALKGQLLLNHLKESKDLKLQNGISNQDWLAYIQELRNGRSSD | |
| | DEATADSQHAAPPKKKRKV | |
| B-GEn.1 | MTIRSMKLKLKIYSGRSAPQLRQGLWRLHRLLNEGTAYYMDWLVHM | 26 |
| | RQEALPGKSKEEIRAELERRVRQQQEKNGVQNDQVPMDEVLSALRQ | |
| | LYELLVPSAVNNSGDAQTLSRKFLSPLVDPNSEGGKGTSNAGAKPG | |
| | WRKKQEAGDPSWEKDYERWLKRKQADPTAEILGKLETAGLKPLFPL | |
| | YTNEVKDIRWMPLTSKQYVRNWDRDMFQQAIEHLLSWETWNRKVN | |
| | EERAKLKETVRRFEEQHLANGKDWLSPLQAYEANREQALRDMAISPS | |
| | DRFRITRRQIKGWSELYERWNKLAPTASVEAYMQEVRHVQKKLGGT | |
| | FGDADLYRFLAKPENVHIWRDHQERLHYYAAYNDLHKRLMSAKEQA | |
| | AFTLPDPVAHPLWVRFDARDGNLFTYILQADSSKQRSRRYVNFSRFL | |
| | WPVEDGYFEETENVKVELALSKQFYRQVIVHDNPTGKQKITFQDYSS | |
| | KEILEGHLGGAKLQLDRNFLRKSGRDFETGDFGPAFLNVVLDLKPKQ | |
| | EVKNGRLQSPLGQALLVKSRPNDIPKVYGYKPDALAAWLEQASGEES | |
| | LGSESLRQGFRVMSIDLGVRSAAAISVFSVKGEKTREGDKVCYPVGE | |
| | TGLFAVHDRSFLLRLPGESSEKRVNVERDKRKTERMQIRYHIRTLARV | |
| | LRLANKATPMDRIKAVQDVLNDIESTRFMNDHDHHVYNHALETLRTY | |
| | APDHQGIWEEQVIAAHRQLEHHVGVIVGEWRKNWGKDRRGTVGLS | |
| | MDNIEELDEMRRLLISWSRRARYPREAKPFQVNESNPVHLLRHLQNL | |
| | KEDRLKQLANLIVMTALGYVYDSKEKKWKAAYPACQLILFEDLQRYRF | |
| | HLDRSARENSQLMKWAHRSIPKYVWMQGEPYGLQIGDVWAGFTSR | |
| | YHAKTGAPGIRCKALTEKDFQQGRLLESLVAEGMFTLQEVGTLKPGDI | |
| | VPAEGGELFVTLADDSGDRIVITHADINAAQNVQKRFWLANSERFRVA | |
| | CRSVQIASQECFIPSSESVAKKMGKGVFVRDFSFHKDMEVYHWNNQ | |
| | VKLTAKNVPTDHSDDLQDLQDYQAILEEARESSSSYKTLFRDPSGFFF | |
| | PDDVWVPQNIYWREVKKTITALLRKRIMST | |
| B-GEn.2 | MPIRSFKLKLVTHNGDSTYMDKLRRGLWKTHVIINRGIAYYMNTLALM | 27 |
| | RQEPYGSKSREEVRLDLLSTLREQQRRNNWSEQTGTDDELLSLSRR | |
| | VYELLVPSAIGEKGDAQMLSRKFLSPLVDPNSEGGRGTAKSGRKPR | |
| | WKKMMEEGHPDWEKEKEKDAAKKAEDPTASILADLEAVGLLPLFPLF | |
| | SDEQKEIRWLPKKKRQFVRTWDRDMFQQALERMLSWESWNRRVAE | |
| | EYLKLQAQRDEVYAKYLEDAGSWLNDLQTFEKQREEELAEVSFEPNS | |
| | EYLITRRQIRGWKEVYEKWSKTSENASQEQLWRMVADVQTAMAGAF | |
| | GDPKVYQFLSQPKHHHIWREHPNRLFYYSKYNEVREKLNRAKKQAA | |
| | FTLPDPVEHPLWTRFDARGGNIHDYEISKVGKQYHVTFSSLILPEAQS | |
| | WVEIENVTVGIGNSLQLKRQIRLDGYADKKQKVKYYDYSSRFELTGVL | |
| | GGAKIQFDRKHLKKAAHRLAEGETGPIFLNVVVDVEPFLEVKNGRLRT | |
| | PLGQVLQVNTRDWPKVVDYKAKELSVLMENTQIGNENGVSTIEAGM | |
| | RIMSIDLGQRTAAAVSIFEVISKKPDEKETKLFYPIADTDLYAVHRRSLL | |
| | LRLPGEEISSKKMIEKRKERARIRSLVRYQIRLLSEVLRLHTQGTAEQR | |
| | RFKLDELLVSIQKKLELDQSEWISELEKLFDYIDESAEKWKEALVVAHR | |
| | TLEPIVVEAVRNWKKSLSKENKDRRRIAGISIWSIEELEETRKLLIAWSK | |
| | HSREPGIPKRLEKEETFAPEHLQHIQNVKDDRLKQMANLFVMTALGY | |
| | KYDEGNKRWVEAYPACQVILFEDLSRYRFALDRPRRENNRLMKWAH | |
| | RSIPRLTYMQAELFGIQVGDVYSAYTSRFHAKTGAPGIRCHALTEADL | |
| | QSNSYVVNQLIKDKFIQDNQTEILKAGQIVPWQGGELFVTFADRSGAS | |
| | LAVIHADINAAQNLQKRFWQHNSEVFRVPCKVVKGGLVPVYEKMRKL | |
| | FGKGLFVNIDDPESKEVYRWEHSTKMKSKTTPVDLESEDIDHEELSD | |
| | EWEDMQEGYKTLLRDPSGFFWSSDSWIPQKDFWIRVKSRIGKSLRE | |
| | QIR | |
| B.- | MPIRSFKLKLVTHNGDSTYMDKLRRGLWKTHVIINRGIAYYMNTLALM | 28 |
| GEn.1.2 | RQEPYGSKSREEVRLDLLSTLREQQRRNNWSEQTGTDDELLSLSRR | |
| | VYELLVPSAIGEKGDAQMLSRKFLSPLVDPNSEGGRGTAKSGRKPR | |
| | WKKMMEEGHPDWEKEKEKDAAKKAEDPTASILADLEAVGLLPLFPLF | |
| | SDEQKEIRWLPKKKRQFVRTWDRDMFQQALERMLSWESWNRRVAE | |
| | EYQKLQAQRDEVYAKYLEDAGSWLNDLQTFEKQREEELAEVSFEPN | |
| | SEYLITRRQIRGWKEVYEKWSKTSENASQEQLWRMVADVQTAMAGA | |
| | FGDPKVYQFLSQPKHHHIWREHPNRLFYYSKYNEVREKLNRAKKQA | |
| | AFTLPDPVEHPLWTRFDARGGNIHDYEISKVGKQYHVTFSSLILPEAQ | |
| | SWVEIENVTVGIGNSLQLKRQIRLDGYADKKQKVKYYDYSSRFELTGV | |
| | LGGAKIQFDRKHLKKAAHRLAEGETGPIFLNVVVDVEPFLEVKNGRLR | |
| | TPLGQVLQVNTRDWPKVVDYKAKELSVLMENTQIGNENGVSTIEAGM | |
| | RIMSIDLGQRTAAAVSIFEVISKKPDEKETKLFYPIADTDLYAVHRRSLL | |
| | LRLPGEEISSKKMIEKRKERARIRSLVRYQIRLLSEVLRLHTQGTAEQR | |
| | RFKLDELLVSIQRKLELDQSEWISELEKLFDYIDESAEKWKEALVVAHR | |
| | TLEPIVVEAVRNWKKSLSKENKDRRRIAGISIWSIEELEETRKLLIAWSK | |
| | HSREPGIPKRLEKEETFAPEHLQHIQNVKDDRLKQMANLFVMTALGY | |
| | KYDEGNKRWVEAYPACQVILFEDLSRYRFALDRPRRENNRLMKWAH | |
| | RSIPRLTYMQAELFGIQVGDVYSAYTSRFHAKTGAPGIRCHALTEADL | |
| | QSNSYVVNQLIKDKFIQDNQTEILKAGQIVPWQGGELFVTFADRSGAS | |
| | LAVIHADINAAQNLQKRFWQHNSEVFRVPCKVVKGGLVPVYEKMRKL | |
| | FGKGLFVNIDDPESKEVYRWEHSTKMKSKTTPVDLESEDIEHEELSD | |
| | EWEDMQEGYKTLLRDPSGFFWSSDSWIPQKDFWIRVKSRIGKSLRE | |
| | QIR | |

6.9.3. gRNAs

The systems, compositions, and methods described herein in some embodiments employ a genome-targeting nucleic acid, such as an RNA molecule, that can direct the activities of the Cas polypeptide to a specific target sequence within a target nucleic acid. Such RNA molecules are referred to as “guide RNA” or “gRNA” herein.

A guide RNA has at least a spacer sequence that can hybridize to a target nucleic acid sequence of interest and a CRISPR repeat sequence (such a CRISPR repeat sequence is also referred to as a “tracr mate sequence”). In Type II systems, the gRNA also has a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-specific polypeptide such that the guide RNA and site-direct polypeptide form a complex. The genome-targeting nucleic acid provides target specificity to the complex by virtue of its association with the site-specific polypeptide. The genome-targeting nucleic acid thus directs the activity of the site-specific polypeptide.

In some embodiments, the genome-targeting nucleic acid is a double-molecule guide RNA, which has two strands of RNA. The first strand has in the 5′ to 3′ direction, an optional spacer extension sequence, a spacer sequence and a minimum CRISPR repeat sequence. The second strand has a minimum tracrRNA sequence (complementary to the minimum CRISPR repeat sequence), a 3′ tracrRNA sequence and an optional tracrRNA extension sequence.

In some embodiments, the guide RNA is a single guide RNA (sgRNA). A single-molecule guide RNA (sgRNA) in a Type II system has, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may have elements that contribute additional functionality (e.g., stability) to the guide RNA. The single-molecule guide linker links the minimum CRISPR repeat and the minimum tracrRNA sequence to form a hairpin structure. The optional tracrRNA extension has one or more hairpins. A single-molecule guide RNA (sgRNA) in a Type V system has, in the 5′ to 3′ direction, a minimum CRISPR repeat sequence and a spacer sequence. Or alternatively, a single-molecule guide RNA (sgRNA) in a Type V system has, in the 5′ to 3′ direction, optional tracr extension sequence, a tracr RNA sequence, a single molecule guide linker, a minimum CRISPR repeat sequence, a spacer sequence, and an optional spacer extension sequence.

Modifications of guide RNAs can be used to enhance the formation or stability of the CRISPR-Cas genome editing complex comprising guide RNAs and a Cas endonuclease. Modifications of guide RNAs can also or alternatively be used to enhance the initiation, stability, or kinetics of interactions between the genome editing complex with the target sequence in the genome, which can be used for example to enhance on-target activity. Modifications of guide RNAs can also or alternatively be used to enhance specificity, e.g., the relative rates of genome editing at the on-target site as compared to effects at other (off-target) sites. Modifications can also or alternatively used to increase the stability of a guide RNA, e.g., by increasing its resistance to degradation by ribonucleases (RNases) present in a cell, thereby causing its half-life in the cell to be increased.

Exemplary Cpf1 guide RNA sequences that target the essential gene GAPDH are listed in Table 4.

TABLE 4

| gRNA Sequence | GAPDH locus | SEQ ID NO |
|---|---|---|
| UGAGCCAGCCACCAGAGGGCG | Intron 8 | 38 |
| AUCUUCUAGGUAUGACAACGA | Intron 8/Exon 9 | 39 |
| GCUACAGCAACAGGGUGGUGG | Exon 9 | 40 |
| CCAUAAUUUCCUUUCAAGGUG | Intron 7 | 41 |
| CUUUCAAGGUGGGGAGGGAGG | Intron 7 | 42 |
| AAGGUGGGGAGGGAGGUAGAG | Intron 7 | 43 |
| GCAGACCACAGUCCAUGCCAU | Exon 8 | 44 |
| CAGACCACAGUCCAUGCCAUC | Exon 8 | 45 |
| CCGGAGGGGCCAUCCACAGUC | Exon 8 | 46 |
| UAGACGGCAGGUCAGGUCCAC | Exon 8 | 47 |
| CUAGACGGCAGGUCAGGUCCA | Exon 8 | 48 |
| UCUAGACGGCAGGUCAGGUCC | Exon 8 | 49 |
| GCAGGUUUUUCUAGACGGCAG | Exon 8 | 50 |
| UCAAGCUCAUUUCCUGGUAUG | Exon 8 | 51 |
| CUGGUAUGUGGCUGGGGCCAG | Exon 8/Intron 8 | 52 |
| AGAGCCAGUCUCUGGCCCCAG | Intron 8 | 53 |
| AAGAGCCAGUCUCUGGCCCCA | Intron 8 | 54 |
| UAAGAGCCAGUCUCUGGCCCC | Intron 8 | 55 |
| CUGAGCCAGCCACCAGAGGGC | Intron 8 | 56 |
| UCUGAGCCAGCCACCAGAGGG | Intron 8 | 57 |
| CAUCUUCUAGGUAUGACAACG | Exon 9 | 58 |
| UUGAUGGUACAUGACAAGGUG | 1 kb downstream | 59 |
| GAGGCCCUACCCUCAGUCUGA | 1 kb downstream | 60 |
| CCUCUCCUCGCUCCAGUCCUA | 1 kb downstream | 61 |
| CUCUCCUCGCUCCAGUCCUAG | 1 kb downstream | 62 |
| GCCAACAGCAGAUAGCCUAGG | 1 kb downstream | 63 |
| UGUGCCCUCGUGUCUUAUCUG | 1 kb downstream | 64 |
| CCUAGAUGAAUCCUGCUUGAA | 1 kb downstream | 65 |
| GGUACUUGGUUUACCUAGAUG | 1 kb downstream | 66 |
| AGGUACUUGGUUUACCUAGAU | 1 kb downstream | 67 |
| AAACAUUAUAUAGUCCUUACC | 1 kb downstream | 68 |
| UAAACAUUAUAUAGUCCUUAC | 1 kb downstream | 69 |
| CCGAUUUUUAAACAUUAUAUA | 1 kb downstream | 70 |
| ACCGAUUUUUAAACAUUAUAU | 1 kb downstream | 71 |
| UACCGAUUUUUAAACAUUAUA | 1 kb downstream | 72 |
| AAAAUCGGUAAAAAUGCCCAC | 1 kb downstream | 73 |
| GAGGAAGAUGAACUGAGAUGU | 1 kb downstream | 74 |
| AGGAAGAUGAACUGAGAUGUG | 1 kb downstream | 75 |

Exemplary Cas9 guide RNA sequences that target the essential STEL gene GAPDH are listed in Table 5.

TABLE 5
GAPDH Cas9 gRNA

| gRNA Sequence | GAPDH locus | SEQ ID NO |
|---|---|---|
| CUUCCUCUUGUGCUCUUGCU | 3′ UTR | 76 |
| CCUCCAAGGAGUAAGACCCC | 3′ UTR | 77 |
| CAUGGCCCACAUGGCCUCCA | Exon 9 | 78 |
| AGCCCCAGCAAGAGCACAAG | 3′ UTR | 79 |

Exemplary Cas9 guide RNA sequences that target the essential STEL gene RPL13A are listed in Table 6.

TABLE 6
RPL13A Cas9 sgRNA

| gRNA Sequence | RPL13A locus | SEQ ID NO |
|---|---|---|
| GGAAGGGCAGGCAACGCAUG | 3′ UTR | 80 |
| GGCUCAGACCAGGAGUCCGU | Exon 8 CDS | 81 |
| CCUCAAGACCCACGGACUCC | Exon 8 CDS | 82 |
| GUCUUUAUUGGGCUCAGACC | Exon 8 CDS | 83 |
| ACAUUCCAGGGCAACAAUGG | 3′ UTR | 84 |
| UUGGCAGAGGCUACGGAAAC | Exon 8 CDS | 85 |
| CUUAUUCUUGGCAGAGGCUA | Exon 8 CDS | 86 |

Exemplary Cas9 guide RNA sequences that target the essential STEL gene RPLP0 are listed in Table 7.

TABLE 7
RPLP0 Cas9 sgRNA

| gRNA Sequence | RPLP0 locus | SEQ ID NO |
|---|---|---|
| GCAAAUAAAACUGGCUAAGU | 3′ UTR | 87 |
| UUACUUCUUUAAAAAGUCUC | 3′ UTR | 88 |
| UUUGCAAAACAAGGAAAUAA | 3′ UTR | 89 |

Exemplary Cpf1 guide RNA sequences that target the essential STEL gene RPLP0 are listed in Table 8.

TABLE 8
RPLP0 Cpf1 sgRNA

| gRNA Sequence | RPLP0 locus | SEQ ID NO |
|---|---|---|
| UUUCCUUGUUUUGCAAAUAAAAC | 3′ UTR | 90 |
| CAAAUAAAACUGGCUAAGUUGGU | 3′ UTR | 91 |
| CUUGUUUUGCAAAUAAAACUGGC | 3′ UTR | 92 |
| GUGAUUAGUCAAAGAGACCAAAU | 3′ UTR | 93 |
| ACUAAUCACCAAAAAGCAACCAA | 3′ UTR | 94 |
| AGGUCAAGGCCUUCUUGGCUG | Exon 8 CDS | 95 |
| CAAAUAAAACUGGCUAAGUUG | 3′ UTR | 96 |

Exemplary Cas9 guide RNA sequences that target the essential STEL gene RPL7 are listed in Table 9.

TABLE 9
RPL7 Cas9 sgRNA

| gRNA Sequence | RPL7 locus | SEQ ID NO |
|---|---|---|
| ACUCAACCUUUCUCAGGAUG | 3′ UTR | 97 |
| ACAUUAACUCAACCUUUCUC | 3′ UTR | 98 |
| AGUUAAUGUUAGAGUAUAAG | 3′ UTR | 99 |

### 6.9.4. Ribonucleoprotein (RNP) Complexes

In some embodiments, the endonucleases are delivered into target cells in a composition known as a ribonucleoprotein or RNP complex. An RNP complex is assembled by combining an endonuclease with a ribonucleic acid.

In some embodiments, the ribonucleoprotein complex comprises a Cas endonuclease, complexed with a suitable ribonucleic acid. In some embodiments, the ribonucleic acid is a gRNA or an sgRNA, which are described further in Section 6.9.3.

One of the most common techniques for delivery of RNPs is electroporation, which generates pores in the cell membrane, allowing for entry of the RNP into the cytoplasm. Further, electroporation can be combined with cell-type specific reagents in a technique known as nucleofection, which forms pores in the nuclear membrane, allowing for entry of a DNA template. In some embodiments, an RNP complex is delivered into target cells via nucleofection.

### 6.9.5. Methods of Gene Editing

In some embodiments, the methods of the disclosure include introducing targeting constructs into a target cell (or a population of target cells). The targeting construct of the present disclosure can be incorporated into a target cell with an endonuclease system, wherein the endonuclease system can be introduced into a host or target cell by any of a variety of well-known methods and any known method. For instance, the endonuclease system of the disclosure may be delivered into a target cell via one or more vectors encoding the endonuclease system or in the form of a ribonucleoprotein complex.

Suitable methods include, e.g., viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv Drug Deliv Rev. 2012 Sep. 13. pii: 50169-409X (12) 00283-9. doi: 10.1016/j.addr.2012.09.023), and the like, including but not limiting to exosome delivery. Nucleic acids may also be delivered by non-viral delivery vehicles including, but not limited to, nanoparticles, liposomes, ribonucleoproteins, positively charged peptides, small molecule RNA-conjugates, aptamer-RNA chimeras, and RNA-fusion protein complexes. Some exemplary non-viral delivery vehicles are described in Peer and Lieberman, 2011, Gene Therapy, 18:1127-1133.

In some embodiments, an endonuclease system comprises a ribonucleoprotein complex (e.g., a Cas endonuclease and an sgRNA), for example as described in Section 6.9.4, and can be delivered to target cells through nucleofection, electroporation, or similar methods.

As an alternative to RNP delivery, the endonuclease system may be delivered into a target cell in nucleic acid form via a delivery vector, e.g., a viral delivery vector.

Suitable nucleic acids comprising nucleotide sequences encoding a Cas endonuclease and/or a guide RNA include expression vectors. In some embodiments, the expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, etc. Suitable expression vectors include, but are not limited to, viral vectors (e.g. viral vectors based on vaccinia virus; poliovirus; adenovirus (see, e.g. Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:10881097, 1999; WO 94/12649, WO 93/03769; WO 93/19191; WO 94/28938; WO 95/11984 and WO 95/00655); adeno-associated virus (see, e.g. Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683-690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Viral. (1988) 166:154-165; and Flotte et al., PNAS (1993) 90:10613-10617); SV40; herpes simplex virus; human immunodeficiency virus (see, e.g. Miyoshi et al., PNAS 94:10319 23, 1997; Takahashi et al., J Virol 73:7812 7816, 1999); a retroviral vector (e.g. Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

In addition to encoding the endonuclease and guide RNA, the nucleic acid vector may further comprise the targeting construct of the disclosure. Alternatively, the targeting construct may be introduced into the target cell on a separate nucleic acid molecule.

The target cells are then grown in conditions under which gene editing occurs. Without being bound by theory, it is believed that the endonuclease cleaves the target cell genome as guided by the guide RNA, allowing the first and second homology arms of the targeting construct to recombine with the target cell genome, which results in integration of the nucleotide sequence flanked by the homology arms of the targeting construct into the genome of the target cell.

In some embodiments, a target cell (e.g., a cell comprising a target DNA locus that is targeted by the targeting construct) is in vitro. In some embodiments, a target cell is in vivo.

### 6.10. Pharmaceutical Compositions

Also disclosed herein are pharmaceutical formulations and medications comprising recombinant cells engineered to express a fusion protein of the disclosure and, optionally, a transgene, together with a pharmaceutically acceptable excipient.

Suitable excipients include, but are not limited to, salts, diluents, (e.g., Tris-HCl, acetate, phosphate), preservatives (e.g., Thimerosal, benzyl alcohol, parabens), binders, fillers, solubilizers, disintegrants, sorbents, solvents, pH modifying agents, antioxidants, anti-infective agents, suspending agents, wetting agents, viscosity modifiers, tonicity agents, stabilizing agents, and other components and combinations thereof. Suitable pharmaceutically acceptable excipients can be selected from materials which are generally recognized as safe (GRAS) and may be administered to an individual without causing undesirable biological side effects or unwanted interactions. Suitable excipients and their formulations are described in Remington's Pharmaceutical Sciences, 16th ed. 1980, Mack Publishing Co. In addition, such compositions can be complexed with polyethylene glycol (PEG), metal ions, or incorporated into polymeric compounds such as polyacetic acid, polyglycolic acid, hydrogels, etc., or incorporated into liposomes, microemulsions, micelles, unilamellar or multilamellar vesicles, erythrocyte ghosts or spheroblasts. Suitable dosage forms for administration, e.g., parenteral administration, include solutions, suspensions, and emulsions.

The components of the pharmaceutical formulation can be dissolved or suspended in a suitable solvent such as, for example, water, Ringer's solution, phosphate buffered saline (PBS), or isotonic sodium chloride. The formulation may also be a sterile solution, suspension, or emulsion in a nontoxic, parenterally acceptable diluent or solvent such as 1,3-butanediol.

In some cases, formulations can include one or more tonicity agents to adjust the isotonic range of the formulation. Suitable tonicity agents are well known in the art and include glycerin, mannitol, sorbitol, sodium chloride, and other electrolytes. In some cases, the formulations can be buffered with an effective amount of buffer necessary to maintain a pH suitable for parenteral administration. Suitable buffers are well known by those skilled in the art and some examples of useful buffers are acetate, borate, carbonate, citrate, and phosphate buffers.

In some embodiments, the formulation can be distributed or packaged in a liquid form, or alternatively, as a solid, obtained, for example by lyophilization of a suitable liquid formulation, which can be reconstituted with an appropriate carrier or diluent prior to administration. In some embodiments, the formulations can comprise a guide RNA and a Type II Cas protein in a pharmaceutically effective amount sufficient to edit a gene in a cell. The pharmaceutical compositions can be formulated for medical and/or veterinary use.

### 6.11. Methods of Treatment

The recombinant target cells of the disclosure and pharmaceutical compositions can be introduced into an individual for treatment. For instance, a therapeutic cell of the disclosure may be used to treat genetic ailments by grafting cells that express a functional transgene into the affected tissue or organ of a subject. A recombinant target cell can also be used to treat tissue injury, trauma, aging-related cell damages, or tissue or organ damages associated with exposure to certain environmental factors or other conditions, by replacing dead, injured, damaged, or dysfunctional cells in an affected tissue, organ, or bodily system.

The recombinant target calls can be autologous to the subject or allogeneic to the subject.

The recombinant target cells described herein may be provided in a pharmaceutical composition containing the cells and a pharmaceutically acceptable carrier. The pharmaceutically acceptable carrier may be cell culture medium that optionally does not contain any animal-derived component. For storage and transportation, the cells may be cryopreserved at <−70° C. (e.g., on dry ice or in liquid nitrogen). Prior to use, the cells may be thawed, and diluted in a sterile cell medium that is supportive of the cell type of interest.

The recombinant target cells may be administered into the patient systemically (e.g., through intravenous injection or infusion), or locally (e.g., through direct injection to a local tissue, e.g., the heart, the brain, and a site of damaged tissue). Various methods are known in the art for administering cells into a patient's tissue or organs, including, without limitation, intracoronary administration, intramyocardial administration, transendocardial administration, or intracranial administration.

A therapeutically effective number of recombinant target cells are administered to the patient. As used herein, the term “therapeutically effective” refers to a number of cells or amount of pharmaceutical composition that is sufficient, when administered to a human subject suffering from or susceptible to a disease, disorder, and/or condition, to treat, prevent, and/or delay the onset or progression of the symptom(s) of the disease, disorder, and/or condition. It will be appreciated by those of ordinary skill in the art that a therapeutically effective amount is typically administered via a dosing regimen comprising at least one-unit dose. In some embodiments, at least 10³ (e.g., at least 10⁴, at least 10⁵, at least 10⁶, at least 10⁷, at least 10⁸, at least 10⁹, at least 10¹⁰, at least 10¹¹, or at least 10¹²) cells are administered to a subject at a time in one or more sites. In some embodiments, 10³-10¹⁸ (e.g., 10³-10⁴, 10³-10⁵, 10³-10⁶, 10³-10⁷, 10³-10⁸, 10³-10⁹, 10³-10¹⁰, 10³-10¹¹, 10³-10¹², 10⁶-10⁷, 10⁶-10⁸, 10⁶-10⁹, 10⁶-10¹⁰, 10⁶-10¹¹, 10⁶-10¹², 10⁹-10¹⁰, 10⁹-10¹¹, 10⁹-10¹²) cells are administered to a subject at a time in one or more sites. In some embodiments, more than 10¹² (e.g., more than 10¹², more than 10¹³, more than 10¹⁴, more than 10¹⁵, more than 10¹⁶, more than 10¹⁷, more than 10¹⁸ or more) cells are administered to a subject at a time at one or more sites.

In some embodiments, a method of treatment comprises selective killing of recombinant target cells that have been grafted into a subject. Recombinant target cells can be selected against by induction of the degron, such as by administering the subject with a drug, such as an IMID, that activates the degron, which leads to degradation of degron-comprising fusion proteins. The degradation of a fusion protein comprising an essential polypeptide causes apoptosis, and therefore, selective killing of that cell.

Selective killing of target cells may be carried out, e.g., if adverse events, such as adverse events resulting from overexpression of a therapeutic polypeptide, occur. Selective killing of recombinant target cells may also be carried out if the treatment goals are accomplished and the recombinant target cells are no longer needed for therapy. Further, selective killing of recombinant target cells may be used to eradicate a graft completely, such as when a graft causes serious side effects, such as a cytokine storm, excessive (systemic) inflammation, tumor formation, graft vs. host disease, organ damage, or other health problems to the subject.

The selective killing of target cells can be induced any time following administration of target cells into a subject, e.g., one hour to one year (or longer) after the administration of the target cells.

In various embodiments, selective killing of target cells is induced in a subject one hour to one day after the administration of the target cells, one day to one week after the administration of the target cells, one week to two weeks after the administration of the cells, two weeks to one month after the administration of the target cells, one month to three months after the administration of the target cells, three months to one year after the administration of the target cells, or any time range bounded by two of the foregoing embodiments (e.g., two weeks to three months after the administration of the target cells).

Accordingly, the present disclosure provides therapeutic methods in which a subject who previously received cell therapy with a recombinant target cell as described herein, comprising administering to the subject an effective amount of a degron-inducing agent. In some embodiments, the subject previously received recombinant target cells engineered to express a fusion protein comprising an essential polypeptide and an IMID-inducible degron, and the methods comprise administering to the subject an IMiD in an amount effective to selectively kill the recombinant target cells. Exemplary IMiDs include, but are not limited to, pomalidomide, thalidomide, lenalidomide, iberdomide, and avadomide. In various embodiments, the IMiD is administered if the subject experiences a cytokine storm, excessive (systemic) inflammation, tumor formation, graft vs. host disease, or another health problem caused by the cell therapy.

### 7. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below. Unless otherwise specified, features of any of the concepts, aspects and/or embodiments described in the detailed description above are applicable mutatis mutandis to any of the following numbered embodiments.

1. A fusion protein comprising:
(a) an essential polypeptide;
(b) a degron; and
(c) optionally; a linker.

2. The fusion protein of embodiment 1, wherein the degron is N-terminal to the essential polypeptide.

3. The fusion protein of embodiment 1, wherein the degron is C-terminal to the essential polypeptide.

4. The fusion protein of any one of embodiments 1 to 3, wherein the degron is an inducible degron.

5. The fusion protein of embodiment 4, wherein the degron is drug-inducible, temperature-sensitive, light-inducible, or activated by a polypeptide (optionally wherein the polypeptide is TEV protease).

6. The fusion protein of embodiment 5, wherein the degron is drug-inducible.

7. The fusion protein of embodiment 6, wherein the drug is an immunomodulatory drug (IMID).

8. The fusion protein of embodiment 7, wherein the IMiD is lenalidomide, iberdomide, thalidomide, avadomide or pomalidomide.

9. The fusion protein of embodiment 8, wherein the IMiD is iberdomide.

10. The fusion protein of embodiment 8, wherein the IMID is avadomide.

11. The fusion protein of embodiment 8, wherein the IMID is thalidomide.

12. The fusion protein of embodiment 8, wherein the IMiD is lenalidomide.

13. The fusion protein of embodiment 8, wherein the IMiD is pomalidomide.

14. The fusion protein of any one of embodiments 1 to 13, wherein the degron comprises or consists of the amino acid sequence RPFQCNQCGASFTQKGNLLRHIKLH (SEQ ID NO:3), FNVLMVHKRSHTGERPLQCEICGFTCRQKGNLLRHIKLHTGEKPFKCHLCNYACQRRDAL (SEQ ID NO: 4, corresponds to SEQ ID NO:42 of WO 2021/188286 A2), FNVLMVHKRSHTGERP (SEQ ID NO: 5, corresponds to SEQ ID NO:97 of WO 2019/089592 A1), FNVLMVHRRSHTGERP (SEQ ID NO: 6, corresponds to SEQ ID NO: 100 of WO 2019/089592 A1), TGEKPFKCHLCNYACQRRDAL (SEQ ID NO:7, corresponds to SEQ ID NO:102 of WO 2019/089592 A1), TGERPFRCHLCNYACQRRDAL (SEQ ID NO:8, corresponds to SEQ ID NO: 103 of WO 2019/089592 A1), FQCNQCGASFT (SEQ ID NO:9, corresponds to SEQ ID NO:528 of WO 2021/188286 A2), FQCPICGLVIK (SEQ ID NO:10, corresponds to SEQ ID NO:529 of WO 2021/188286 A2), LQCEICGFTCR (SEQ ID NO: 11, corresponds to SEQ ID NO:530 of WO 2021/188286 A2), LQCEICGYQCR (SEQ ID NO:12, corresponds to SEQ ID NO:531 of WO 2021/188286 A2), or LQCEVCGFQCR (SEQ ID NO:13, corresponds to SEQ ID NO:532 of WO 2021/188286 A2).

15. The fusion protein of embodiment 14, wherein the degron comprises or consists of the amino acid sequence of SEQ ID NO:3.

16. The fusion protein of embodiment 14, wherein the degron comprises or consists of the amino acid sequence of SEQ ID NO:4.

17. The fusion protein of embodiment 14, wherein the degron comprises or consists of the amino acid sequence of SEQ ID NO:5.

18. The fusion protein of embodiment 14, wherein the degron comprises or consists of the amino acid sequence of SEQ ID NO:6.

19. The fusion protein of embodiment 14, wherein the degron comprises or consists of the amino acid sequence of SEQ ID NO:7.

20. The fusion protein of embodiment 14, wherein the degron comprises or consists of the amino acid sequence of SEQ ID NO:8.

21. The fusion protein of embodiment 14, wherein the degron comprises or consists of the amino acid sequence of SEQ ID NO:9.

22. The fusion protein of embodiment 14, wherein the degron comprises or consists of the amino acid sequence of SEQ ID NO: 10.

23. The fusion protein of embodiment 14, wherein the degron comprises or consists of the amino acid sequence of SEQ ID NO:11.

24. The fusion protein of embodiment 14, wherein the degron comprises or consists of the amino acid sequence of SEQ ID NO:12.

25. The fusion protein of embodiment 14, wherein the degron comprises or consists of the amino acid sequence of SEQ ID NO:13.

26. The fusion protein of any one of embodiments 1 to 25, wherein the degron is a superdegron.

27. The fusion protein of embodiment 6, wherein the degron is a SMASh (Small-Molecule-Assisted Shutoff) tag degron.

28. The fusion protein of any one of embodiments 1 to 27, which comprises a linker sequence between the essential polypeptide and the degron.

29. The fusion protein of embodiment 27, wherein the linker is 1 to 30 amino acids in length.

30. The fusion protein of embodiment 29, wherein the linker is 1, 2, 3, 4, 5, 6, 7, 8, 9, 10, 11, 12, 13, 14, or 15 amino acids in length.

31. The fusion protein of embodiment 29 or embodiment 30, wherein the linker is between 1 and 12 amino acids in length, between 2 and 12 amino acids in length, or between 1 and 10 amino acids in length.

32. The fusion protein of embodiment 29 or embodiment 30, wherein the linker is 3 to 10 amino acids in length.

33. The fusion protein of embodiment 29 or embodiment 30, wherein the linker is 11 to 20 amino acids in length.

34. The fusion protein of embodiment 29 or embodiment 30, wherein the linker is 21 to 30 amino acids in length.

35. The fusion protein of any one of embodiments 1 to 34, which comprises a plurality of degrons.

36. The fusion protein of embodiment 35, wherein the fusion protein comprises two or more degrons.

37. The fusion protein of embodiment 36, wherein the degrons are in tandem.

38. The fusion protein of embodiment 37, wherein each pair of degrons is separated by a linker, optionally wherein (a) the linker is a linker described in Section 6.2.3 and/or (b) all the linkers separating the pairs of degrons are the same.

39. The fusion protein of any one of embodiments 35 to 38, wherein the plurality of degrons are inducible in the same manner, optionally wherein the plurality of degrons are the same.

40. The fusion protein of any one of embodiments 1 to 39, wherein the essential polypeptide is:
(a) a STEL polypeptide; or
(b) a non-STEL polypeptide.

41. The fusion protein of any one of embodiments 1 to 40, wherein the essential polypeptide is involved in one or more of: glycolysis, ribonucleopolypeptide complex formation, focal adhesion, cell-substrate adherens junction, cell-substrate junction, cell anchoring, extracellular exosome, extracellular vesicle, intracellular organelle, anchoring junction, RNA binding, nucleic acid binding (e.g., rRNA or mRNA binding), and polypeptide binding.

42. The fusion protein of any one of embodiments 1 to 41, wherein the essential polypeptide (a) is a ribosomal polypeptide or (b) is not a ribosomal polypeptide.

43. The fusion protein of embodiment 42, wherein the essential polypeptide is a ribosomal polypeptide, optionally wherein the ribosomal polypeptide is RPL13A, RPLP0, RPL10, RPL13, RPSJ8, RPL3, RPLP1, RPL15, RPL41, RPL11, RPL32, RPL18 A, RPL19, RPL28, RPL29, RPL30, RPL24, RPL39, RPL37, RPL14, RPL27A, RPLP2, RPL23A, RPL26, RPL36, RPL35, RPL23, RPL4, or RPL22.

44. The fusion protein of any one of embodiments 1 to 41, wherein the essential polypeptide (a) is a ribosomal polypeptide small subunit (RPS) or (b) is not a ribosomal polypeptide small subunit (RPS).

45. The fusion protein of embodiment 44, wherein the essential polypeptide is a ribosomal polypeptide small subunit (RPS), optionally wherein the ribosomal polypeptide small subunit (RPS) is RPS2, RPS19, RPS14, RPS3A, RPS12, RPS3, RPS6, RPS23, RPS27A, RPS8, RPS4X, RPS7, RPS24, RPS27, RPS15A, RPS9, RPS28, RPS13, RPSA, RPS5, RPS 16, RPS25, RPS15, RPS20, or RPS11.

46. The fusion protein of any one of embodiments 1 to 41, wherein the essential polypeptide (a) is an actin polypeptide or (b) is not an actin polypeptide.

47. The fusion protein of embodiment 46, wherein the essential polypeptide is an actin polypeptide, optionally wherein the actin polypeptide is ACTG1 or ACTB.

48. The fusion protein of any one of embodiments 1 to 41, wherein the essential polypeptide (a) is a eukaryotic translation factor or (b) is not a eukaryotic translation factor.

49. The fusion protein of embodiment 48, wherein the essential polypeptide is a eukaryotic translation factor, optionally wherein the eukaryotic translation factor is EEF1A1, EEF2, or EIF1.

50. The fusion protein of any one of embodiments 1 to 41, wherein the essential polypeptide (a) is a histone or (b) is not a histone.

51. The fusion protein of embodiment 50, wherein the essential polypeptide is a histone, optionally wherein the histone is H3F3A or H3F3B.

52. The fusion protein of any one of embodiments 1 to 41, wherein the essential polypeptide is (a) selected from FTH1, TPT1, GAPDH, PTMA, GNB2L1, NACA, YBX1, NPM1, FAU, UBA52, HSP90AB1, MYL6, SERF2, SRP14, RPL13A, RPL7, or RPLP0 or (b) is not FTH1, TPT1, PTMA, GNB2L1, NACA, YBX1, NPM1, FAU, UBA52, HSP90AB1, MYL6, SERF2, or SRP14.

53. The fusion protein of embodiment 52, wherein the essential polypeptide is GAPDH.

54. The fusion protein of embodiment 52, wherein the essential polypeptide is RPL13A.

55. The fusion protein of embodiment 52, wherein the essential polypeptide is RPL7.

56. The fusion protein of embodiment 52, wherein the essential polypeptide is RPLP0.

57. A targeting construct comprising:
(a) a first homology arm corresponding to a 5′ target sequence comprising a first region of homology to an essential gene encoding an essential polypeptide in a target genomic locus;
(b) a nucleotide sequence encoding a degron (“degron coding sequence”);
(c) a second homology arm corresponding to a 3′ target sequence comprising second region of homology to the essential gene in the target genomic locus
wherein the targeting construct is configured such that upon its recombination with the target genomic locus, the essential gene is modified such to encode a fusion protein comprising the essential polypeptide and the degron, optionally wherein the fusion protein has one or more features as defined in any one of embodiments 1 to 56 or in Section 6.2.

58. The targeting construct of embodiment 57, wherein the targeting construct is configured such that upon its recombination with the target genomic locus, the essential gene is modified such to encode a fusion protein comprising the degron at the C-terminus of the essential polypeptide.

59. The targeting construct of embodiment 57, wherein the targeting construct is configured such that upon its recombination with the target genomic locus, the essential gene is modified such to encode a fusion protein comprising the degron at the N-terminus of the essential polypeptide.

60. The targeting construct of any one of embodiments 57 to 59, wherein the degron is an inducible degron.

61. The targeting construct of embodiment 60, wherein the degron is drug-inducible, temperature-sensitive, light-inducible, or activated by a polypeptide (optionally wherein the polypeptide is TEV protease).

62. The targeting construct of embodiment 61, wherein the degron is drug-inducible.

63. The targeting construct of embodiment 62, wherein the drug is an immunomodulatory drug (IMiD).

64. The targeting construct of embodiment 63, wherein the IMiD is lenalidomide, iberdomide, thalidomide, avadomide or pomalidomide.

65. The targeting construct of embodiment 63, wherein the IMiD is iberdomide.

66 The targeting construct of embodiment 63, wherein the IMiD is avadomide.

67 The targeting construct of embodiment 63, wherein the IMiD is thalidomide.

68. The targeting construct of embodiment 63, wherein the IMiD is lenalidomide.

69 The targeting construct of embodiment 63, wherein the IMiD is pomalidomide.

70. The targeting construct of any one of embodiments 57 to 69, wherein the degron comprises or consists of the amino acid sequence RPFQCNQCGASFTQKGNLLRHIKLH (SEQ ID NO: 3), FNVLMVHKRSHTGERPLQCEICGFTCRQKGNLLRHIKLHTGEKPFKCHLCNYACQRRDAL (SEQ ID NO:4, corresponds to SEQ ID NO:42 of WO 2021/188286 A2), FNVLMVHKRSHTGERP (SEQ ID NO:5, corresponds to SEQ ID NO:97 of WO 2019/089592 A1), FNVLMVHRRSHTGERP (SEQ ID NO:6, corresponds to SEQ ID NO:100 of WO 2019/089592 A1), TGEKPFKCHLCNYACQRRDAL (SEQ ID NO:7, corresponds to SEQ ID NO: 102 of WO 2019/089592 A1), TGERPFRCHLCNYACQRRDAL (SEQ ID NO:8, corresponds to SEQ ID NO:103 of WO 2019/089592 A1), FQCNQCGASFT (SEQ ID NO:9, corresponds to SEQ ID NO:528 of WO 2021/188286 A2), FQCPICGLVIK (SEQ ID NO:10, corresponds to SEQ ID NO:529 of WO 2021/188286 A2), LQCEICGFTCR (SEQ ID NO:11, corresponds to SEQ ID NO:530 of WO 2021/188286 A2), LQCEICGYQCR (SEQ ID NO:12, corresponds to SEQ ID NO:531 of WO 2021/188286 A2), or LQCEVCGFQCR (SEQ ID NO:13, corresponds to SEQ ID NO:532 of WO 2021/188286 A2).

71. The targeting construct of embodiment 70, wherein the degron comprises or consists of the amino acid sequence of SEQ ID NO:3.

72. The targeting construct of embodiment 70, wherein the degron comprises or consists of the amino acid sequence of SEQ ID NO:4.

73. The targeting construct of embodiment 70, wherein the degron comprises or consists of the amino acid sequence of SEQ ID NO:5.

74. The targeting construct of embodiment 70, wherein the degron comprises or consists of the amino acid sequence of SEQ ID NO:6.

75. The targeting construct of embodiment 70, wherein the degron comprises or consists of the amino acid sequence of SEQ ID NO:7.

76. The targeting construct of embodiment 70, wherein the degron comprises or consists of the amino acid sequence of SEQ ID NO:8.

77. The targeting construct of embodiment 70, wherein the degron comprises or consists of the amino acid sequence of SEQ ID NO:9.

78. The targeting construct of embodiment 70, wherein the degron comprises or consists of the amino acid sequence of SEQ ID NO:10.

79. The targeting construct of embodiment 70, wherein the degron comprises or consists of the amino acid sequence of SEQ ID NO: 11.

80. The targeting construct of embodiment 70, wherein the degron comprises or consists of the amino acid sequence of SEQ ID NO:12.

81. The targeting construct of embodiment 70, wherein the degron comprises or consists of the amino acid sequence of SEQ ID NO: 13.

82. The targeting construct of any one of embodiments 57 to 81, wherein the degron is a superdegron.

83. The targeting construct of embodiment 62, wherein the degron is a SMASh (Small-Molecule-Assisted Shutoff) tag degron.

84. The targeting construct of any one of embodiments 57 to 83, wherein the fusion protein comprises a linker sequence between the essential polypeptide and the degron.

85. The targeting construct of embodiment 82, wherein the linker is 1 to 30 amino acids in length.

86. The targeting construct of embodiment 85, wherein the linker is 1, 2, 3, 4, 5, 6, 7, 8, 9, 10, 11, 12, 13, 14, or 15 amino acids in length.

87. The targeting construct of embodiment 85, wherein the linker is between 1 and 12 amino acids in length, between 2 and 12 amino acids in length, or between 1 and 10 amino acids in length.

88. The targeting construct of embodiment 85, wherein the linker is 3 to 10 amino acids in length.

89. The targeting construct of embodiment 85, wherein the linker is 11 to 20 amino acids in length.

90. The targeting construct of embodiment 85, wherein the linker is 21 to 30 amino acids in length.

91. The targeting construct of any one of embodiments 57 to 90, wherein the fusion protein comprises a plurality of degrons.

92. The targeting construct of embodiment 91, wherein the fusion protein comprises two or more degrons.

93. The targeting construct of embodiment 92, wherein the degrons are in tandem.

94. The targeting construct of embodiment 93, wherein each pair of degrons is separated by a linker, optionally wherein (a) the linker is a linker described in Section 6.2.3 and/or (b) all the linkers separating the pairs of degrons are the same.

95. The targeting construct of any one of embodiments 91 to 94, wherein the plurality of degrons are inducible in the same manner, optionally wherein the plurality of degrons are the same.

96. The targeting construct of any one of embodiments 57 to 95, wherein the essential gene is:
(a) a STEL gene; or
(b) a non-STEL gene.

97. The targeting construct of any one of embodiments 57 to 96, wherein the essential gene encodes a polypeptide involved in one or more of: glycolysis, ribonucleopolypeptide complex formation, focal adhesion, cell-substrate adherens junction, cell-substrate junction, cell anchoring, extracellular exosome, extracellular vesicle, intracellular organelle, anchoring junction, RNA binding, nucleic acid binding (e.g., rRNA or mRNA binding), and polypeptide binding.

98. The targeting construct of any one of embodiments 57 to 97, wherein the essential gene (a) encodes a ribosomal polypeptide or (b) does not encode a ribosomal polypeptide.

99. The targeting construct of embodiment 98, wherein the essential gene encodes a ribosomal polypeptide, optionally wherein the ribosomal polypeptide is RPL13A, RPLP0, RPL10, RPL13, RPSJ8, RPL3, RPLP1, RPL15, RPL41, RPL11, RPL32, RPL18 A, RPL19, RPL28, RPL29, RPL30, RPL24, RPL39, RPL37, RPL14, RPL27A, RPLP2, RPL23A, RPL26, RPL36, RPL35, RPL23, RPL4, or RPL22.

100. The targeting construct of any one of embodiments 57 to 98, wherein the essential gene (a) encodes a ribosomal polypeptide small subunit (RPS) or (b) does not encode a ribosomal polypeptide small subunit (RPS).

101. The targeting construct of embodiment 100, wherein the essential gene encodes a ribosomal polypeptide small subunit (RPS), optionally wherein the ribosomal polypeptide small subunit (RPS) is RPS2, RPS19, RPS14, RPS3A, RPS12, RPS3, RPS6, RPS23, RPS27A, RPS8, RPS4X, RPS7, RPS24, RPS27, RPS15A, RPS9, RPS28, RPS13, RPSA, RPS5, RPS 16, RPS25, RPS15, RPS20, or RPS11.

102. The targeting construct of any one of embodiments 57 to 97, wherein the essential gene (a) encodes an actin polypeptide or (b) does not encode an actin polypeptide.

103. The targeting construct of embodiment 102, wherein the essential gene encodes an actin polypeptide, optionally wherein the actin polypeptide is ACTG1 or ACTB.

104. The targeting construct of any one of embodiments 57 to 97, wherein the essential gene (a) encodes a eukaryotic translation factor or (b) does not encode a eukaryotic translation factor.

105. The targeting construct of embodiment 104, wherein the essential gene encodes a eukaryotic translation factor, optionally wherein the eukaryotic translation factor is EEF1A1, EEF2, or EIF1.

106. The targeting construct of any one of embodiments 57 to 97, wherein the essential gene (a) encodes a histone or (b) does not encode a histone.

107. The targeting construct of embodiment 106, wherein the essential gene encodes a histone, optionally wherein the histone is H3F3A or H3F3B.

108. The targeting construct of any one of embodiments 57 to 97, wherein the essential gene is (a) selected from FTH1, TPT1, GAPDH, PTMA, GNB2L1, NACA, YBX1, NPM1, FAU, UBA52, HSP90AB1, MYL6, SERF2, SRP14, RPL13A, RPL7, or RPLP0 or (b) is not FTH1, TPT1, PTMA, GNB2L1, NACA, YBX1, NPM1, FAU, UBA52, HSP90AB1, MYL6, SERF2, or SRP14.

109. The targeting construct of embodiment 108, wherein the essential gene is GAPDH.

110. The targeting construct of embodiment 108, wherein the essential gene is RPL13A.

111. The targeting construct of embodiment 108, wherein the essential gene is RPL7.

112. The targeting construct of embodiment 108, wherein the essential gene is RPLP0.

113. The targeting construct of any one of embodiments 57 to 112, wherein the first homology arm and second homology arm are each 500 to 1,500 nucleotides in length. 114. The targeting construct of embodiment 113, wherein the first homology arm and second homology arm are each 600 to 1200 nucleotides in length or 700 to 1000 nucleotides in length.

115. The targeting construct of any one of embodiments 57 to 114, wherein the length difference between the first and second homology arms, if any, is less than 75 nucleotides.

116. The targeting construct of embodiment 115, wherein the length difference between the first and second homology arms, if any, is less than 50 nucleotides.

117. The targeting construct of any one of embodiments 57 to 116, wherein the targeting construct further comprises a transgene between the degron coding sequence and the second homology arm.

118. The targeting construct of embodiment 113, wherein the transgene is linked to the nucleotide sequence encoding the fusion protein (“fusion protein coding sequence”).

119. The targeting construct of embodiment 118, wherein the fusion protein coding sequence and transgene are connected via a nucleotide sequence encoding an internal ribosome entry site (“IRES”).

120. The targeting construct of embodiment 118, wherein the fusion protein coding sequence and transgene are connected in frame via a nucleotide sequence encoding a self-cleaving peptide (the “self-cleaving peptide coding sequence”).

121. The targeting construct of embodiment 120, wherein the self-cleaving peptide is a 2A peptide.

122. The targeting construct of embodiment 120 or embodiment 121, wherein the self-cleaving peptide is T2A, P2A, E2A, F2A, or PQR.

123. The targeting construct of any one of embodiments 57 to 122, wherein the transgene (a) encodes a therapeutic polypeptide; and/or
(b) is a transgene described in Section 6.4.

124. The targeting construct of embodiment 123, wherein the therapeutic polypeptide is a lysosomal enzyme.

125. The targeting construct of embodiment 124, wherein the lysosomal enzyme is alpha-L-iduronidase, arylsulfatase A, beta-glucocerebrosidase, acid sphingomyelinase, alpha-galactosidase or beta-galactosidase.

126. The targeting construct of embodiment 123, wherein the therapeutic polypeptide is a polypeptide whose deficiency is associated with hemophilia.

127. The targeting construct of embodiment 126, wherein the therapeutic polypeptide is Factor VIII or Factor IX.

128. The targeting construct of embodiment 123, wherein the therapeutic polypeptide is an immunomodulatory polypeptide.

129. The targeting construct of embodiment 128, wherein the immunomodulatory polypeptide is a human leukocyte antigen (“HLA”) polypeptide.

130. The targeting construct of embodiment 129, wherein the HLA polypeptide is a HLA-class Ib polypeptide.

131. The targeting construct of embodiment 129 or embodiment 130, wherein the HLA polypeptide is HLA-E, HLA-F or an isoform of HLA-G (e.g., HLA-G1, -G2, -G3, -G4, -G5, -G6, or -G7).

132. The targeting construct of embodiment 128, wherein the immunomodulatory polypeptide is a cytokine.

133. The targeting construct of embodiment 132, wherein the cytokine is IL-1, IL-1α, IL-1β, IL-2, IL-3, IL-4, IL-6, IL-8, IL-10, IL-12, IL-11, IL-12, IL-13, IL-15, IL-18, IL-21, IL-23, IL-27, IL-31, or IL-35.

134. The targeting construct of embodiment 123, wherein the therapeutic polypeptide is or comprises an antibody or antigen-binding fragment thereof.

135. The targeting construct of embodiment 134, wherein the therapeutic polypeptide binds to a pathogenic polypeptide.

136. The targeting construct of embodiment 135, wherein the pathogenic polypeptide is tau, alpha-synuclein, or beta-amyloid polypeptide.

137. The targeting construct of embodiment 134, wherein the therapeutic polypeptide binds to cancer cells.

138. The targeting construct of embodiment 137, wherein the therapeutic polypeptide is a chimeric antigen receptor.

139. The targeting construct of embodiment 137 or embodiment 138, wherein the therapeutic polypeptide binds to a tumor-associated antigen.

140. The targeting construct of embodiment 139, wherein the tumor-associated antigen is CD19 or CD20.

141. The targeting construct of any one of embodiments 57 to 140, which is a vector.

142. The targeting construct of embodiment 141, wherein the vector is a viral vector.

143. The targeting construct of embodiment 142, wherein the viral vector is an AAV vector, a retroviral vector or a lentiviral vector.

144. The targeting construct of embodiment 141, wherein the vector is a DNA vector.

145. The targeting construct of embodiment 141, wherein the vector is an RNA vector.

146. The targeting construct of any of embodiments 57 to 145, which comprises a nucleotide sequence configured as shown in FIG. 7A, optionally wherein the nucleotide sequence comprises the nucleotide sequence of SEQ ID NO:1.

147. The targeting construct of any of embodiments 57 to 145, which comprises a nucleotide sequence configured as shown in FIG. 7B.

148. The targeting construct of any of embodiments 57 to 145, which comprises a nucleotide sequence configured as shown in FIG. 7C.

149. The targeting construct of any of embodiments 57 to 145, which comprises a nucleotide sequence configured as shown in FIG. 7D, optionally wherein the nucleotide sequence comprises the nucleotide sequence of SEQ ID NO:2.

150. The targeting construct of any of embodiments 57 to 145, which comprises a nucleotide sequence configured as shown in FIG. 7E, optionally wherein the nucleotide sequence comprises the nucleotide sequence of SEQ ID NO:29.

151. The targeting construct of any of embodiments 57 to 145, which comprises a nucleotide sequence configured as shown in FIG. 7F, optionally wherein the nucleotide sequence comprises the nucleotide sequence of SEQ ID NO:30.

152. The targeting construct of any of embodiments 57 to 145, which comprises a nucleotide sequence configured as shown in FIG. 7G.

153. The targeting construct of any of embodiments 57 to 145, which comprises a nucleotide sequence configured as shown in FIG. 7H.

154. The targeting construct of any of embodiments 57 to 145, which comprises a nucleotide sequence configured as shown in FIG. 7I.

155. The targeting construct of any of embodiments 57 to 145, which comprises a nucleotide sequence configured as shown in FIG. 7J.

156. The targeting construct of any of embodiments 57 to 145, which comprises a nucleotide sequence configured as shown in FIG. 7K, optionally wherein the nucleotide sequence comprises the nucleotide sequence of SEQ ID NO: 14.

157. The targeting construct of any of embodiments 57 to 145, which comprises a nucleotide sequence configured as shown in FIG. 7L optionally wherein the nucleotide sequence comprises the nucleotide sequence of SEQ ID NO: 17.

158. A system comprising:
(a) the targeting construct of any one of embodiments 57 to 157;
(b) a CRISPR-associated endonuclease (“Cas polypeptide”) or a nucleic acid encoding a Cas polypeptide; and
(c) a guide RNA (“gRNA”) comprising a scaffold for binding the Cas polypeptide and a spacer sequence corresponding to the essential gene, or a nucleic acid encoding the gRNA.

159. The system of embodiment 158, wherein the guide RNA is a single guide RNA (“sgRNA”).

160. The system of embodiment 158 or embodiment 159, which comprises the Cas polypeptide and gRNA.

161. The system of any one of embodiments 158 to 160, which is in the form of a ribonucleoprotein particle (“RNP”).

162. A method of producing a gene-edited target cell, comprising:
(a) introducing the system of any one of embodiments 158 to 161 into a target cell, optionally wherein the target cell is as defined in Section 6.7; and
(b) culturing the target cell under conditions in which gene editing occurs, thereby producing gene-edited target cell.

163. The method of embodiment 162, wherein the target cell is a stem cell or a cell differentiated from a stem cell.

164. The method of embodiment 162 or embodiment 163, wherein the target cell is a stem cell.

165. The method of embodiment 164, wherein the stem cell is a human embryonic stem cell, an induced pluripotent stem cell (“iPSC”) or a cell differentiated therefrom.

166. The method of any one of embodiments 162 to 164, wherein the target cell is:
(a) a human immune cell, optionally selected from a T cell, a T cell expressing a chimeric antigen receptor (CAR) or recombinant TCR, a regulatory T cell, a myeloid cell, a dendritic cell, and a macrophage (e.g., an immunosuppressive macrophage);
(b) a cell in the human nervous system, optionally selected from dopaminergic neuron, a microglial cell, an oligodendrocyte, an astrocyte, a cortical neuron, a spinal or oculomotor neuron, an enteric neuron, a Placode-derived cell, a Schwann cell, and a trigeminal or sensory neuron;
(c) a cell in the human cardiovascular system, optionally selected from a cardiomyocyte, an endothelial cell, and a nodal cell;
(d) a cell in the human metabolic system, optionally selected from a hepatocyte, a cholangiocyte, and a pancreatic beta cell,
(e) a cell in the human ocular system, optionally selected from a retinal pigment epithelial cell, a photoreceptor cone cell, a photoreceptor rod cell, a bipolar cell, or a ganglion cell, or
(f) a progenitor or precursor of any one of the aforementioned cells.

167. The method of any one of embodiments 162 to 166, wherein the gene-edited target cell is of ectoderm lineage, optionally wherein the gene-edited target cell is a neuron or a progenitor or precursor thereof.

168. The method of any one of embodiments 162 to 166, wherein the gene-edited target cell is of mesoderm lineage, optionally wherein the gene-edited target cell is a cardiomyocyte or a progenitor or precursor thereof.

169. The method of any one of embodiments 162 to 166, wherein the gene-edited target cell is of endoderm lineage, optionally wherein the gene-edited cell is a lung, thyroid, or pancreatic cell, or a progenitor or precursor thereof.

170. The method of any one of embodiments 162 to 166, wherein the gene-edited target cell is a cardiac cell, a cardiac progenitor cell or a mature or immature (atrial or ventricular) cardiomyocyte, a cardiac endothelial cell, a nodal cell or a progenitor or precursor thereof.

171. The method of any one of embodiments 162 to 166, wherein the gene-edited target cell is a T cell, a CAR-T cell, a recombinant TCR-expressing T-cell, a regulatory T cell, a myeloid cell, a dendritic cell, and/or a macrophage, or a progenitor or precursor thereof.

172. The method of any one of embodiments 162 to 166, wherein the gene-edited target cell is an oligodendrocyte or a progenitor or precursor thereof.

173. The method of any one of embodiments 162 to 166, wherein the gene-edited target cell is a neural crest cell, an astrocyte, a dopaminergic neuron, or a progenitor or precursor thereof.

174. The method of any one of embodiments 162 to 166, wherein the gene-edited target cell is a photoreceptor cell, a retinal pigmented epithelium cell, a neural retinal cell, or a progenitor or precursor thereof.

175. The method of any one of embodiments 162 to 166, wherein the gene-edited target cell is a microglial cell or a microglial progenitor or precursor cell.

176. The method of any one of embodiments 162 to 166, wherein the gene-edited target cell is a hepatocyte, a cholangiocyte, and a pancreatic beta cell, or a progenitor or precursor thereof.

177. The method of any one of embodiments 162 to 166, wherein the gene-edited target cell is enteric cell or an enteric progenitor or precursor cell.

178. A gene-edited target cell obtained or obtainable by the method of any one of embodiments 162 to 177.

179. A gene-edited target cell comprising an essential gene that encodes a fusion protein comprising:
(a) a degron;
(b) an essential polypeptide encoded by an essential gene as defined in any one of embodiments 96 to 112.

180. The gene-edited target cell of embodiment 179, wherein the degron is at the C-terminus of the essential polypeptide.

181. The gene-edited target cell of embodiment 179, wherein the degron is at the N-terminus of the essential polypeptide.

182. The gene-edited target cell of any one of embodiments 179 to 181, wherein the degron is an inducible degron.

183. The gene-edited target cell of embodiment 182, wherein the degron is drug-inducible, temperature-sensitive, light-inducible, or activated by a polypeptide (optionally wherein the polypeptide is TEV protease).

184. The gene-edited target cell of embodiment 183, wherein the degron is drug-inducible.

185. The gene-edited target cell of embodiment 184, wherein the drug is an immunomodulatory drug (IMiD).

186. The gene-edited target cell of embodiment 185, wherein the IMiD is lenalidomide, iberdomide, thalidomide, avadomide or pomalidomide.

187. The gene-edited target cell of embodiment 185, wherein the IMiD is iberdomide.

188. The gene-edited target cell of embodiment 185, wherein the IMiD is avadomide.

189. The gene-edited target cell of embodiment 185, wherein the IMiD is thalidomide.

190. The gene-edited target cell of embodiment 185, wherein the IMID is lenalidomide.

191. The gene-edited target cell of embodiment 185, wherein the IMiD is pomalidomide.

192. The gene-edited target cell of any one of embodiments 179 to 191, wherein the degron comprises or consists of the amino acid sequence RPFQCNQCGASFTQKGNLLRHIKLH (SEQ ID NO:3), FNVLMVHKRSHTGERPLQCEICGFTCRQKGNLLRHIKLHTGEKPFKCHLCNYACQRRDAL (SEQ ID NO: 4, corresponds to SEQ ID NO:42 of WO 2021/188286 A2), FNVLMVHKRSHTGERP (SEQ ID NO: 5, corresponds to SEQ ID NO:97 of WO 2019/089592 A1), FNVLMVHRRSHTGERP (SEQ ID NO: 6, corresponds to SEQ ID NO: 100 of WO 2019/089592 A1), TGEKPFKCHLCNYACQRRDAL (SEQ ID NO:7, corresponds to SEQ ID NO:102 of WO 2019/089592 A1),

TGERPFRCHLCNYACQRRDAL (SEQ ID NO:8, corresponds to SEQ ID NO: 103 of WO 2019/089592 A1), FQCNQCGASFT (SEQ ID NO:9, corresponds to SEQ ID NO:528 of WO 2021/188286 A2), FQCPICGLVIK (SEQ ID NO:10, corresponds to SEQ ID NO:529 of WO 2021/188286 A2), LQCEICGFTCR (SEQ ID NO: 11, corresponds to SEQ ID NO:530 of WO 2021/188286 A2), LQCEICGYQCR (SEQ ID NO:12, corresponds to SEQ ID NO:531 of WO 2021/188286 A2), or LQCEVCGFQCR (SEQ ID NO:13, corresponds to SEQ ID NO:532 of WO 2021/188286 A2).

193. The gene-edited target cell of claim 192, wherein the degron comprises or consists of the amino acid sequence of SEQ ID NO:3.

194. The gene-edited target cell of claim 192, wherein the degron comprises or consists of the amino acid sequence of SEQ ID NO:4.

195. The gene-edited target cell of claim 192, wherein the degron comprises or consists of the amino acid sequence of SEQ ID NO:5.

196. The gene-edited target cell of claim 192, wherein the degron comprises or consists of the amino acid sequence of SEQ ID NO:6.

197. The gene-edited target cell of claim 192, wherein the degron comprises or consists of the amino acid sequence of SEQ ID NO:7.

198. The gene-edited target cell of claim 192, wherein the degron comprises or consists of the amino acid sequence of SEQ ID NO:8.

199. The gene-edited target cell of claim 192, wherein the degron comprises or consists of the amino acid sequence of SEQ ID NO:9.

200. The gene-edited target cell of claim 192, wherein the degron comprises or consists of the amino acid sequence of SEQ ID NO: 10.

201. The gene-edited target cell of claim 192, wherein the degron comprises or consists of the amino acid sequence of SEQ ID NO:11.

202. The gene-edited target cell of claim 192, wherein the degron comprises or consists of the amino acid sequence of SEQ ID NO:12.

203. The gene-edited target cell of claim 192, wherein the degron comprises or consists of the amino acid sequence of SEQ ID NO:13.

204. The gene-edited target cell of any one of embodiments 179 to 203, wherein the degron is a superdegron.

205. The gene-edited target cell of embodiment 184, wherein the degron is a SMASh (Small-Molecule-Assisted Shutoff) tag degron.

206. The gene-edited target cell of any one of embodiments 179 to 205, wherein the fusion protein comprises a linker sequence between the essential polypeptide and the degron.

207. The gene-edited target cell of embodiment 205, wherein the linker is 1 to 30 amino acids in length.

208. The gene-edited target cell of embodiment 207, wherein the linker is 1, 2, 3, 4, 5, 6, 7, 8, 9, 10, 11, 12, 13, 14, or 15 amino acids in length.

209. The gene-edited target cell of embodiment 207 or embodiment 208, wherein the linker is between 1 and 12 amino acids in length, between 2 and 12 amino acids in length, or between 1 and 10 amino acids in length.

210. The gene-edited target cell of embodiment 207 or embodiment 208, wherein the linker is 3 to 10 amino acids in length.

211. The gene-edited target cell of embodiment 207 or embodiment 208, wherein the linker is 11 to 20 amino acids in length.

212. The gene-edited target cell of embodiment 207 or embodiment 208, wherein the linker is 21 to 30 amino acids in length.

213. The gene-edited target cell of any one of embodiments 179 to 212, wherein the fusion protein comprises a plurality of degrons.

214. The gene-edited target cell of embodiment 213, wherein the fusion protein comprises two or more degrons.

215. The gene-edited target cell of embodiment 214, wherein the degrons are in tandem.

216. The gene-edited target cell of embodiment 215 wherein each pair of degrons is separated by a linker, optionally wherein (a) the linker is a linker described in Section 6.2.3 and/or (b) all the linkers separating the pairs of degrons are the same.

217. The gene-edited target cell of any one of embodiments 213 to 216, wherein the plurality of degrons are inducible in the same manner, optionally wherein the plurality of degrons are the same.

218. The gene-edited target cell of any one of embodiments 179 to 217, wherein the essential gene is:
(a) a STEL gene; or
(b) a non-STEL gene.

219. The gene-edited target cell of any one of embodiments 179 to 218, wherein the essential gene encodes a polypeptide involved in one or more of: glycolysis, ribonucleopolypeptide complex formation, focal adhesion, cell-substrate adherens junction, cell-substrate junction, cell anchoring, extracellular exosome, extracellular vesicle, intracellular organelle, anchoring junction, RNA binding, nucleic acid binding (e.g., rRNA or mRNA binding), and polypeptide binding.

220. The gene-edited target cell of any one of embodiments 179 to 219, wherein the essential gene (a) encodes a ribosomal polypeptide or (b) does not encode a ribosomal polypeptide.

221. The gene-edited target cell of embodiment 220, wherein the essential gene encodes a ribosomal polypeptide, optionally wherein the ribosomal polypeptide is RPL13A, RPLP0, RPL10, RPL13, RPSJ8, RPL3, RPLP1, RPL15, RPL41, RPL11, RPL32, RPL18 A, RPL19, RPL28, RPL29, RPL9, RPL8, RPL6, RPL18, RPL7, RPL7A, RPL21, RPL37A, RPL 12, RPL5, RPL34, RPL35A, RPL30, RPL24, RPL39, RPL37, RPL14, RPL27A, RPLP2, RPL23A, RPL26, RPL36, RPL35, RPL23, RPL4, or RPL22.

222. The gene-edited target cell of gene-edited target cell of any one of embodiments 179 to 219 wherein the essential gene (a) encodes a ribosomal polypeptide small subunit (RPS) or (b) does not encode a ribosomal polypeptide small subunit (RPS).

223. The gene-edited target cell of embodiment 222, wherein the essential gene encodes a ribosomal polypeptide small subunit (RPS), optionally wherein the ribosomal polypeptide small subunit (RPS) is RPS2, RPS19, RPS14, RPS3A, RPS12, RPS3, RPS6, RPS23, RPS27A, RPS8, RPS4X, RPS7, RPS24, RPS27, RPS15A, RPS9, RPS28, RPS13, RPSA, RPS5, RPS 16, RPS25, RPS15, RPS20, or RPS11.

224. The gene-edited target cell of any one of embodiments 179 to 219, wherein the essential gene (a) encodes an actin polypeptide or (b) does not encode an actin polypeptide.

225. The gene-edited target cell of embodiment 224, wherein the essential gene encodes an actin polypeptide, optionally wherein the actin polypeptide is ACTG1 or ACTB.

226. The gene-edited target cell of any one of embodiments 179 to 219, wherein the essential gene (a) encodes a eukaryotic translation factor or (b) does not encode a eukaryotic translation factor.

227. The gene-edited target cell of embodiment 226, wherein the essential gene encodes a eukaryotic translation factor, optionally wherein the eukaryotic translation factor is EEF1A1, EEF2, or EIF1.

228. The gene-edited target cell of any one of embodiments 179 to 219, wherein the essential gene (a) encodes a histone or (b) does not encode a histone.

229. The gene-edited target cell of embodiment 228, wherein the essential gene encodes a histone, optionally wherein the histone is H3F3A or H3F3B.

230. The gene-edited target cell of any one of embodiments 179 to 219, wherein the essential gene is (a) selected from FTH1, TPT1, GAPDH, PTMA, GNB2L1, NACA, YBX1, NPM1, FAU, UBA52, HSP90AB1, MYL6, SERF2, SRP14, RPL13A, RPL7, or RPLP0 or (b) is not FTH1, TPT1, PTMA, GNB2L1, NACA, YBX1, NPM1, FAU, UBA52, HSP90AB1, MYL6, SERF2, or SRP14.

231. The gene-edited target cell of embodiment 230, wherein the essential gene is GAPDH.

232. The gene-edited target cell of embodiment 230, wherein the essential gene is RPL13A.

233. The gene-edited target cell of embodiment 230, wherein the essential gene is RPL7.

234. The gene-edited target cell of embodiment 230, wherein the essential gene is RPLP0.

235. The gene-edited target cell of any one of embodiments 179 to 234, in which the essential further comprises a transgene.

236. The gene-edited target cell of embodiment 235, wherein the transgene is linked to the nucleotide sequence encoding the fusion protein (“fusion protein coding sequence”) via a nucleotide sequence encoding an internal ribosome entry site (“IRES”).

237. The gene-edited target cell of embodiment 235, wherein the transgene is linked to the nucleotide sequence encoding the fusion protein (“fusion protein coding sequence”) via a nucleotide sequence encoding a self-cleaving peptide (the “self-cleaving peptide coding sequence”).

238. The gene-edited target cell of embodiment 237, wherein the self-cleaving peptide is a 2A peptide.

239. The gene-edited target cell of embodiment 237 or 238, wherein the self-cleaving peptide is T2A, P2A, E2A, F2A, or PQR.

240. The gene-edited target cell of any one of embodiments 179 to 239, wherein the transgene
(a) encodes a therapeutic polypeptide; and/or
(b) is a transgene described in Section 6.4.

241. The gene-edited target cell of embodiment 240, wherein the therapeutic polypeptide is a lysosomal enzyme.

242. The gene-edited target cell of embodiment 241, wherein the lysosomal enzyme is alpha-L-iduronidase, arylsulfatase A, beta-glucocerebrosidase, acid sphingomyelinase, alpha-galactosidase or beta-galactosidase.

243. The gene-edited target cell of embodiment 240, wherein the therapeutic polypeptide is a polypeptide whose deficiency is associated with hemophilia.

244. The gene-edited target cell of embodiment 243, wherein the therapeutic polypeptide is Factor VIII or Factor IX.

245. The gene-edited target cell of embodiment 240, wherein the therapeutic polypeptide is an immunomodulatory polypeptide.

246. The gene-edited target cell of embodiment 245, wherein the immunomodulatory polypeptide is a human leukocyte antigen (“HLA”) polypeptide.

247. The gene-edited target cell of embodiment 246, wherein the HLA polypeptide is a HLA-class Ib polypeptide.

248. The gene-edited target cell of embodiment 246 or embodiment 247, wherein the HLA polypeptide is HLA-E, HLA-F or an isoform of HLA-G (e.g., HLA-G1, -G2, -G3, -G4, -G5, -G6, or -G7).

249. The gene-edited target cell of embodiment 245, wherein the immunomodulatory polypeptide is a cytokine or cytokine receptor.

250. The gene-edited target cell of embodiment 245 or embodiment 249, wherein immunomodulatory polypeptide is a cytokine is the cytokine is:

(a) IL-1, IL-1a, IL-1B, IL-2, IL-3, IL-4, IL-6, IL-8, IL-10, IL-12, IL-11, IL-12, IL-13, IL-15, IL-18, IL-21, IL-23, IL-27, IL-31, or IL-35; or
(b) IL-1B, IL-6, IL-10, IL-12, IL-15, GM-CSF, IFN-α, IFN-β, IFN-γ, TNF-α, CCL2, CCL5, CXCL9, CXCL10, CXCL12, TGFβ, or CSF-1.

251. The gene-edited target cell of embodiment 245 or embodiment 249, wherein the immunomodulatory polypeptide is IL-1Ra.

252. The gene-edited target cell of embodiment 240, wherein the therapeutic polypeptide is or comprises an antibody or antigen-binding fragment thereof.

253. The gene-edited target cell of embodiment 252, wherein the therapeutic polypeptide binds to a pathogenic polypeptide.

254. The gene-edited target cell of embodiment 253, wherein the pathogenic polypeptide is tau, alpha-synuclein, or beta-amyloid polypeptide.

255. The gene-edited target cell of embodiment 240, wherein the therapeutic polypeptide binds to cancer cells.

256. The gene-edited target cell of embodiment 255, wherein the therapeutic polypeptide is a chimeric antigen receptor.

257. The gene-edited target cell of embodiment 255 or embodiment 256, wherein the therapeutic polypeptide binds to a tumor-associated antigen.

258. The gene-edited target cell of embodiment 257 wherein the tumor-associated antigen is CD19 or CD20.

259. A recombinant cell engineered to express the fusion protein of any one of embodiments 1 to 56, optionally wherein the recombinant cell is a gene-edited target cell according to any one of embodiments 178 to 258.

260. The recombinant cell of embodiment 259, which is obtainable by integration of a targeting construct depicted in FIG. 2A into a single allele of an essential gene locus.

261. The recombinant cell of embodiment 259, which is obtainable by integration of a targeting construct depicted in FIG. 2A into both alleles of an essential gene locus.

262. The recombinant cell of embodiment 259, which is obtainable by integration of a targeting construct depicted in FIG. 2B into a single allele of an essential gene locus.

263. The recombinant cell of embodiment 259, which is obtainable by integration of a targeting construct depicted in FIG. 2B into both alleles of an essential gene locus.

264. The recombinant cell of embodiment 259, which is obtainable by integration of a targeting construct depicted in FIG. 2C into a single allele of an essential gene locus.

265. The recombinant cell of embodiment 259, which is obtainable by integration of a targeting construct depicted in FIG. 2C into both alleles of an essential gene locus.

266. The recombinant cell of embodiment 259, which is obtainable by integration of a targeting construct depicted in FIG. 2D into a single allele of an essential gene locus.

267. The recombinant cell of embodiment 259, which is obtainable by integration of a targeting construct depicted in FIG. 2D into both alleles of an essential gene locus.

268. The recombinant cell of embodiment 259, which is obtainable by integration of a targeting construct depicted in FIG. 4A into a single allele of an essential gene locus.

269. The recombinant cell of embodiment 259, which is obtainable by integration of a targeting construct depicted in FIG. 4A into both alleles of an essential gene locus.

270. The recombinant cell of embodiment 259, which is obtainable by integration of a targeting construct depicted in FIG. 4B into a single allele of an essential gene locus.

271. The recombinant cell of embodiment 259, which is obtainable by integration of a targeting construct depicted in FIG. 4B into both alleles of an essential gene locus.

272. The recombinant cell of embodiment 259, which is obtainable by integration of a targeting construct depicted in FIG. 4C into a single allele of an essential gene locus.

273. The recombinant cell of embodiment 259, which is obtainable by integration of a targeting construct depicted in FIG. 4C into both alleles of an essential gene locus.

274. The recombinant cell of embodiment 259, which is obtainable by integration of a targeting construct depicted in FIG. 4D into a single allele of an essential gene locus.

275. The recombinant cell of embodiment 259, which is obtainable by integration of a targeting construct depicted in FIG. 4D into both alleles of an essential gene locus.

276. The recombinant cell of embodiment 259, which is obtainable by integration of a targeting construct depicted in FIG. 7A, e.g., a targeting construct comprising the nucleotide sequence of SEQ ID NO:1, into a single allele of an essential gene locus.

277. The recombinant cell of embodiment 259, which is obtainable by integration of a targeting construct depicted in FIG. 7A, e.g., a targeting construct comprising the nucleotide sequence of SEQ ID NO: 1, into both alleles of an essential gene locus.

278. The recombinant cell of embodiment 259, which is obtainable by integration of a targeting construct depicted in FIG. 7B into a single allele of an essential gene locus.

279. The recombinant cell of embodiment 259, which is obtainable by integration of a targeting construct depicted in FIG. 7B into both alleles of an essential gene locus.

280. The recombinant cell of embodiment 259, which is obtainable by integration of a targeting construct depicted in FIG. 7C into a single allele of an essential gene locus.

281. The recombinant cell of embodiment 259, which is obtainable by integration of a targeting construct depicted in FIG. 7C into both alleles of an essential gene locus.

282. The recombinant cell of embodiment 259, which is obtainable by integration of a targeting construct depicted in FIG. 7D, e.g., a targeting construct comprising the nucleotide sequence of SEQ ID NO:2, into a single allele of an essential gene locus.

283. The recombinant cell of embodiment 259, which is obtainable by integration of a targeting construct depicted in FIG. 7D, e.g., a targeting construct comprising the nucleotide sequence of SEQ ID NO:2, into both alleles of an essential gene locus.

284. The recombinant cell of embodiment 259, which is obtainable by integration of a targeting construct depicted in FIG. 7E, e.g., a targeting construct comprising the nucleotide sequence of SEQ ID NO:29, into a single allele of an essential gene locus.

285. The recombinant cell of embodiment 259, which is obtainable by integration of a targeting construct depicted in FIG. 7E, e.g., a targeting construct comprising the nucleotide sequence of SEQ ID NO:29, into both alleles of an essential gene locus.

286. The recombinant cell of embodiment 259, which is obtainable by integration of a targeting construct depicted in FIG. 7F, e.g., a targeting construct comprising the nucleotide sequence of SEQ ID NO:30, into a single allele of an essential gene locus.

287. The recombinant cell of embodiment 259, which is obtainable by integration of a targeting construct depicted in FIG. 7F, e.g., a targeting construct comprising the nucleotide sequence of SEQ ID NO:30, into both alleles of an essential gene locus.

288. The recombinant cell of embodiment 259, which is obtainable by integration of a targeting construct depicted in FIG. 7G into a single allele of an essential gene locus.

289. The recombinant cell of embodiment 259, which is obtainable by integration of a targeting construct depicted in FIG. 7G into both alleles of an essential gene locus.

290. The recombinant cell of embodiment 259, which is obtainable by integration of a targeting construct depicted in FIG. 7H into a single allele of an essential gene locus.

291. The recombinant cell of embodiment 259, which is obtainable by integration of a targeting construct depicted in FIG. 7H into both alleles of an essential gene locus.

292. The recombinant cell of embodiment 259, which is obtainable by integration of a targeting construct depicted in FIG. 7I into a single allele of an essential gene locus.

293. The recombinant cell of embodiment 259, which is obtainable by integration of a targeting construct depicted in FIG. 7I into both alleles of an essential gene locus.

294. The recombinant cell of embodiment 259, which is obtainable by integration of a targeting construct depicted in FIG. 7J into a single allele of an essential gene locus.

295. The recombinant cell of embodiment 259, which is obtainable by integration of a targeting construct depicted in FIG. 7J into both alleles of an essential gene locus.

296. The recombinant cell of embodiment 259, which is obtainable by integration of a targeting construct depicted in FIG. 7K, e.g., a targeting construct comprising the nucleotide sequence of SEQ ID NO: 14, into a single allele of an essential gene locus.

297. The recombinant cell of embodiment 259, which is obtainable by integration of a targeting construct depicted in FIG. 7K, e.g., a targeting construct comprising the nucleotide sequence of SEQ ID NO:14, into both alleles of an essential gene locus.

298. The recombinant cell of embodiment 259, which is obtainable by integration of a targeting construct depicted in FIG. 7L, e.g., a targeting construct comprising the nucleotide sequence of SEQ ID NO:17, into a single allele of an essential gene locus.

299. The recombinant cell of embodiment 259, which is obtainable by integration of a targeting construct depicted in FIG. 7L, e.g., a targeting construct comprising the nucleotide sequence of SEQ ID NO: 17, into both alleles of an essential gene locus.

300. The recombinant cell of embodiment 259, which has an engineered essential gene having the configuration depicted in FIG. 3A.

301. The recombinant cell of embodiment 259, which has an engineered essential gene having the configuration depicted in FIG. 3B.

302. The recombinant cell of embodiment 259, which has an engineered essential gene having the configuration depicted in FIG. 3D.

303. The recombinant cell of embodiment 259, which has an engineered essential gene having the configuration depicted in FIG. 3E.

304. The recombinant cell of embodiment 259, which has an engineered essential gene having the configuration depicted in FIG. 5A.

305. The recombinant cell of embodiment 259, which has an engineered essential gene having the configuration depicted in FIG. 5B.

306. The recombinant cell of embodiment 259, which has an engineered essential gene having the configuration depicted in FIG. 5C.

307. The recombinant cell of embodiment 259, which has an engineered essential gene having the configuration depicted in FIG. 5D.

308. The recombinant cell of embodiment 259, which has an engineered essential gene having the configuration depicted in FIG. 6A.

309. The recombinant cell of embodiment 259, which has an engineered essential gene having the configuration depicted in FIG. 6B.

310. The recombinant cell of embodiment 259, which has an engineered essential gene having the configuration depicted in FIG. 6C.

311. The recombinant cell of embodiment 259, which has an engineered essential gene having the configuration depicted in FIG. 6D.

312. The recombinant cell of embodiment 259, which has an engineered essential gene having the configuration depicted in FIG. 6E.

313. The recombinant cell of any one of embodiments 259 to 312, which is further engineered to express a transgene from a locus other than the essential gene.

314. The recombinant cell of embodiment 259, which comprises an expression vector comprising a nucleotide sequence encoding the fusion protein of any one of embodiments 1 to 56.

315. The recombinant cell of embodiment 314, wherein the vector is a viral vector.

316. The recombinant cell of embodiment 315, wherein the viral vector is an AAV vector, a retroviral vector or a lentiviral vector.

317. The recombinant cell of embodiment 314, wherein the vector is a DNA vector.

318. The recombinant cell of embodiment 314, wherein the vector is an RNA vector.

319. The recombinant cell of any one of embodiments 314 to 318, in which the corresponding essential gene is knocked out at a single allele.

320. The recombinant cell of any one of embodiments 314 to 318, in which the corresponding essential gene is knocked out at both alleles.

321. The recombinant cell of any one of embodiments 314 to 320, which is also engineered to express a transgene.

322. The recombinant cell of embodiment 321, wherein the transgene is not expressed from the corresponding essential gene locus.

323. The recombinant cell of embodiment 321, wherein the transgene is expressed from the corresponding essential gene locus.

324. The recombinant cell of any one of embodiments 321 to 323, wherein the transgene is as defined in Section 6.4.

325. The recombinant cell of any one of embodiments 259 to 324, wherein the cell is a defined in Section 6.7.

326. A pharmaceutical composition comprising the gene-edited target cell of any one of embodiments 178 to 258 or the recombinant cell of any one of embodiments 259 to325 and a pharmaceutically acceptable excipient.

327. Use of the gene-edited target cell of any one of embodiments 178 to 258 or the recombinant cell of any one of embodiments 259 to 325, for the manufacture of a medicament for treating a patient in need thereof, optionally wherein the gene-edited target cell or the recombinant cell is (a) autologous to the patient or (b) allogeneic to the patient.

328. The gene-edited target cell of any one of embodiments 178 to 258, the recombinant cell of any one of embodiments 259 to 325, or the pharmaceutical composition of embodiment 326, for use in treating a patient in need thereof, optionally wherein the gene-edited target cell or the recombinant cell is, or the pharmaceutical comprises cells that are: (a) autologous to the patient or (b) allogeneic to the patient.

329. A method of treating a subject with a cell therapy, comprising administering to a subject in need thereof the gene-edited target cell of any one of embodiments 178 to 258, the recombinant cell of any one of embodiments 259 to 325, or the pharmaceutical composition of embodiment 326, optionally wherein optionally wherein the gene-edited target cell or the recombinant cell is, or the pharmaceutical comprises cells that are: (a) autologous to the patient or (b) allogeneic to the patient.

330. The method of embodiment 329, which further comprises administering to the subject an inducer of the degron.

331. The method of embodiment 330, wherein the subject has developed an adverse effect of the cell therapy.

332. The method of embodiment 330, wherein the subject is at risk of developing an adverse effect of the cell therapy.

333. The method of embodiment 331 or embodiment 332, wherein the adverse effect is a cytokine storm or cytokine release syndrome (CRS).

334. The method of embodiment 331 or embodiment 332, wherein the adverse effect is excessive (systemic) inflammation.

335. The method of embodiment 331 or embodiment 332, wherein the adverse effect is tumor formation.

336. The method of embodiment 331 or embodiment 332, wherein the adverse effect is tumor lysis syndrome.

337. The method of embodiment 331 or embodiment 332, wherein the adverse effect comprises aberrantly functioning neurons and/or dyskinesia.

338. The method of embodiment 331 or embodiment 332, wherein the adverse effect comprises aberrantly functioning cardiomyocytes and/or cardiac arrhythmia.

339. The method of embodiment 331 or embodiment 332, wherein the adverse effect is macrophage activation syndrome (MAS).

340. The method of embodiment 331 or embodiment 332, wherein the adverse effect is graft-vs-host disease.

341. The method of any one of embodiments 330 to 340, wherein the degron is a drug-inducible degron.

342. The method of embodiment 341, wherein the drug is an immunomodulatory drug (IMiD).

343. The method of embodiment 342, wherein the IMiD is lenalidomide, iberdomide, thalidomide, avadomide or pomalidomide.

344. The method of embodiment 342, wherein the IMiD is iberdomide.

345. The method of embodiment 342, wherein the IMiD is avadomide.

346. The method of embodiment 342, wherein the IMID is thalidomide.

347. The method of embodiment 342, wherein the IMiD is lenalidomide.

348. The method of embodiment 342, wherein the IMID is pomalidomide.

349. A method of reducing engineered cells in, or eliminating engineered cells from, a subject who previously received cell therapy with a gene-edited target cell of any one of embodiments 178 to 258, the recombinant cell of any one of embodiments 259 to 325, or the pharmaceutical composition of embodiment 326, comprising administering to the subject an inducer of the degron, optionally wherein the cell therapy comprises or consists of cells that are (a) autologous to the subject or (b) allogeneic to the subject.

350. The method of embodiment 349, wherein the subject experienced or is at risk of an adverse effect of the cell therapy.

351. The method of embodiment 350, wherein the adverse effect is a cytokine storm or cytokine release syndrome (CRS).

352. The method of embodiment 350, wherein the adverse effect is excessive (systemic) inflammation.

353. The method of embodiment 350, wherein the adverse effect is tumor formation.

354. The method of embodiment 350, wherein the adverse effect is tumor lysis syndrome. 355. The method of embodiment 350, wherein the adverse effect comprises aberrantly functioning neurons and/or dyskinesia.

356. The method of embodiment 350, wherein the adverse effect comprises aberrantly functioning cardiomyocytes and/or cardiac arrhythmia.

357. The method of embodiment 350, wherein the adverse effect is macrophage activation syndrome (MAS).

358. The method of embodiment 350, wherein the adverse effect is graft-vs-host disease.

359. The method of any one of embodiments 349 to 351, wherein the degron is a drug-inducible degron.

360. The method of embodiment 359, wherein the drug is an immunomodulatory drug (IMiD).

361. The method of embodiment 360, wherein the IMiD is lenalidomide, iberdomide, thalidomide, avadomide or pomalidomide.

362. The method of embodiment 360, wherein the IMID is iberdomide.

363. The method of embodiment 360, wherein the IMID is avadomide.

364. The method of embodiment 360, wherein the IMID is thalidomide.

365. The method of embodiment 360, wherein the IMiD is lenalidomide.

366. The method of embodiment 360, wherein the IMiD is pomalidomide.

### 8. EXAMPLES

### 8.1. Example 1: Design and Generation of Targeting Constructs

Targeting constructs were designed to comprise a linker and a degron sequence flanked by GAPDH homology arms, as depicted in FIGS. 7A-7D. Additional targeting constructs were designed to comprise a linker, a degron sequence, an IRES sequence and a GFP transgene sequence flanked by GAPDH homology arms as depicted in FIGS. 7G-7J. The homology arms were designed to enable integration of the construct in the endogenous GAPDH locus, immediately 5′ of the GAPDH endogenous STOP codon. Desired sequences were sent to GenScript (Piscataway, NJ) for de novo gene synthesis via on-site oligo design, oligo synthesis and gene assembly. Amplified fragments were ligated into a pUC57-Kan cloning vector, transformed into bacteria, and the resultant plasmid comprising the targeted construct was isolated.

### 8.2. Example 2: Selective Targeting of Gene-Edited iPSCs

Functionality of the kill switch was tested in iPSCs using the targeting construct depicted in FIG. 7G, which comprises a linker, a degron sequence, an IRES, and a GFP transgene sequence flanked by GAPDH homology arms. On the day of Nucleofection, iPSCs were harvested and resuspended in Lonza P3 primary cell nucleofection buffer. Ribonucleoproteins (RNPs) were complexed with sgRNAs using 1:2 ratio of protein:sgRNA (IDT). Nucleofection of complexed RNP together with a targeting construct into resuspended iPSCs was accomplished using LONZA 4D Nucleofector. The Nucleofected cells were then plated and assessed for targeting events.

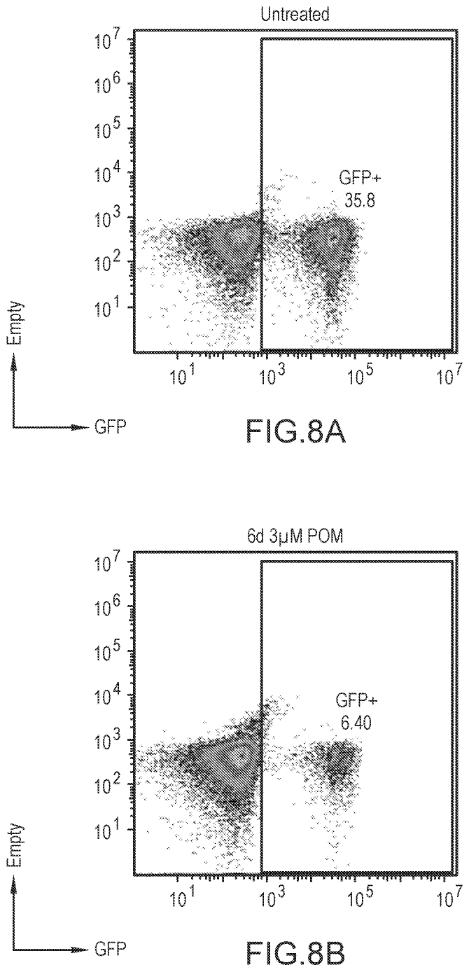

A heterogenous mixture of unedited cells (GFP-negative) and cells edited with a construct depicted in FIG. 7G, comprising a degron fused to GAPDH as well as GFP (GFP-positive) were treated with 3 μM pomalidomide (POM) for 6d or fed with complete medium without POM (untreated). Treatment of cells with POM did not affect unedited cells (FIG. 8A) but depleted the population of edited GFP-positive cells (FIG. 8B), suggesting that inducible degradation of degron-fused GAPDH protein killed gene-edited cells.

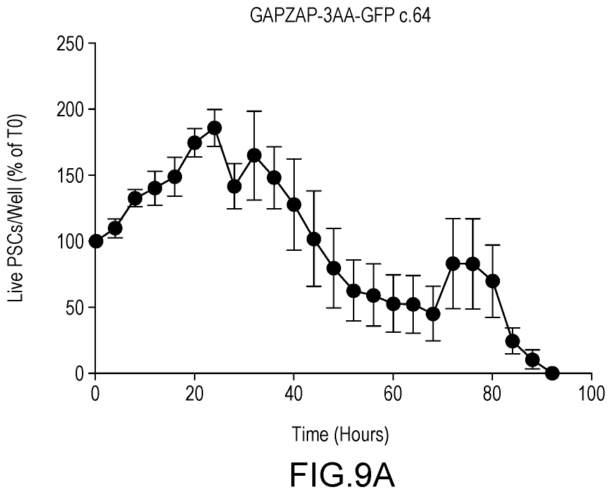

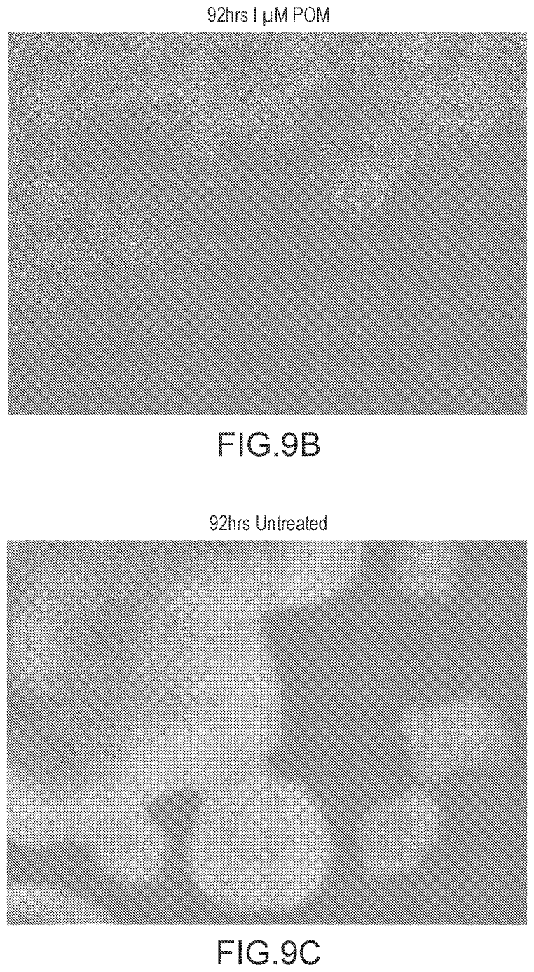

In the next set of assessments, a clone bi-allelically modified with a degron fused to GAPDH and IRES-GFP, as depicted in FIG. 7G, was treated with 1 μM POM for 92 hours and imaged using the Incucyte. The percentage of live cells/well was quantified at several timepoints using the Incucyte software and normalized for the number of live cells/well at the first image timepoint. As depicted in FIG. 9A, cell death rate of gene-edited cells surpassed their proliferation rate around 24 hours after the addition of POM. Furthermore, gene edited cells were completely eliminated at 92 hours after the addition of POM (FIGS. 9A and 9B). Untreated gene-edited cells proliferated normally over the same duration (FIG. 9C).

### 8.3. Example 3: Effect of Targeting Construct Linker Length on Survival of Gene-Edited iPSCs

Two sets of targeting constructs were generated as described in Section 8.1. The first set of targeting constructs were designed to comprise a linker, a degron or superdegron sequence, an IRES sequence, and a GFP transgene sequence flanked by GAPDH homology arms as depicted in FIGS. 7G-7J, wherein the linker lengths were 3 aa (linker 1, GGS), 15 aa (linker 2, SEQ ID NO:23), 27 aa (linker 3, SEQ ID NO:103) and 10 aa (linker 4, SEQ ID NO:15). The second set of targeting constructs were designed to comprise a linker and a degron or superdegron sequence flanked by GAPDH homology arms as depicted in FIGS. 7A-7D. For both sets, the homology arms were designed to enable integration of the construct in the endogenous GAPDH locus, immediately 5′ of the GAPDH endogenous STOP codon. iPSCs were transfected with each construct individually.

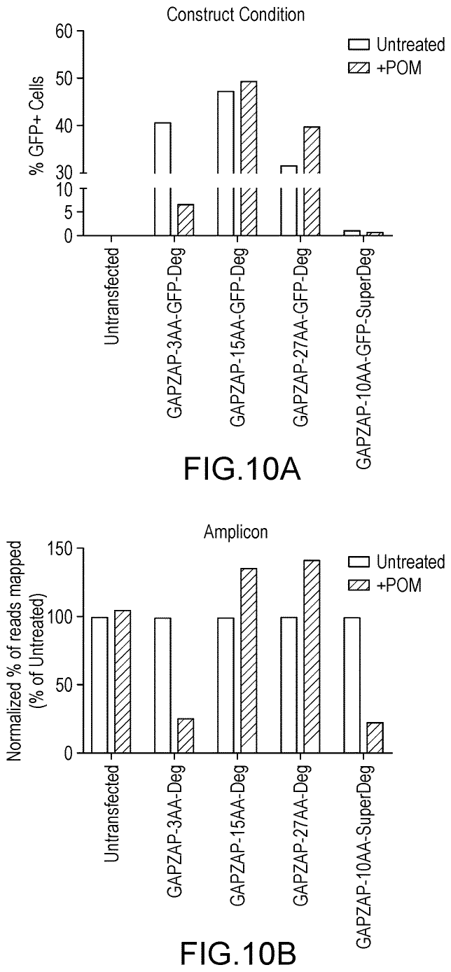

A pool of iPSCs transfected with the first set of targeting constructs, potentially comprising both mono-allelically and bi-allelically gene edited iPSCs, were evaluated using flow cytometry, wherein the depletion of GFP signal was assessed following treatment with 3 μM POM for 4 days. Among gene-edited iPSCs, those that were transfected with the superdegron-comprising targeting construct had the lowest percentage of GFP-positive cells, whereas approximately ⅓ to ½ of untreated cells transfected with one of the degron-comprising targeting constructs were GFP-positive. However, treatment with POM depleted GFP-positive cells only if they were transfected with the degron-comprising targeting construct with the shortest linker or the superdegron-comprising targeting construct (FIG. 10A).

Next, the iPSCs transfected with the second set of targeting constructs were evaluated, wherein amplicon depletion was assessed following POM treatment relative to untreated cells. Consistent with the results of the first set, the targeting construct comprising a 3 aa linker and a degron was associated with amplicon depletion. A similar level of depletion was observed in cells transfected with the superdegron-comprising targeting construct. Taken together, targeting construct linker length and degron type might be key factors to trigger cell death in gene-edited cells.

### 8.4. Example 4: Activation of Targeting Constructs in Gene-Edited iPSCs

To determine whether gene-editing of iPSCs with a targeting construct, wherein the homology arms of which enable its integration in the endogenous GAPDH locus, affects the expression of GAPDH, three clones bi-allelically modified with a targeting construct comprising a 3 aa linker, GGS and a degron (as depicted in FIG. 7G) and one clone bi-allelically modified with superdegron-comprising targeting construct (as depicted in FIG. 7J) were treated with 3 μM POM for up to 3 days.

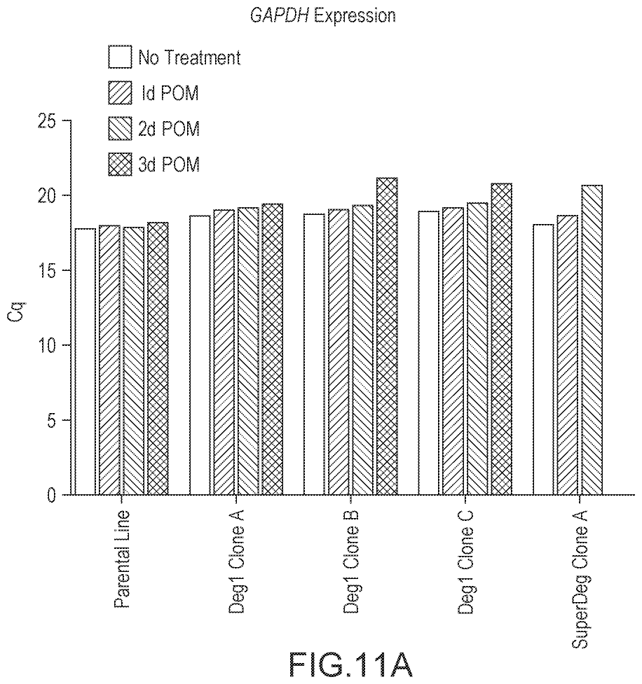

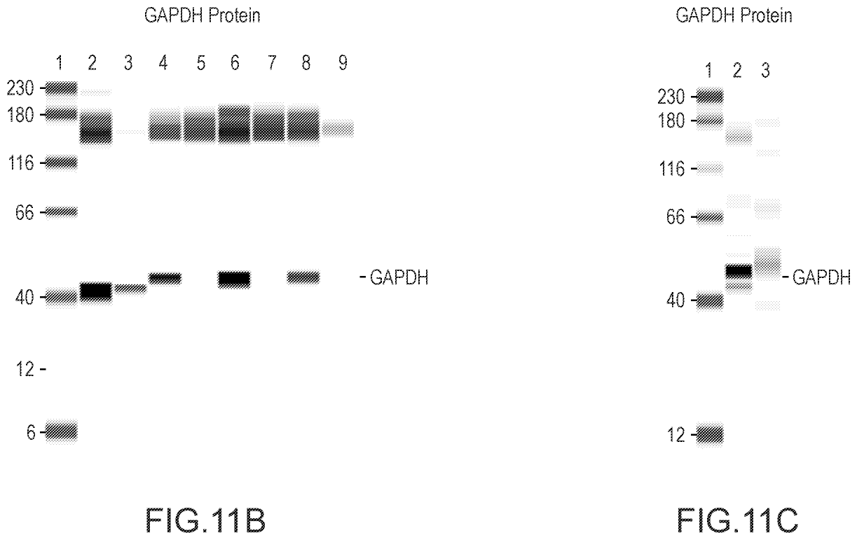

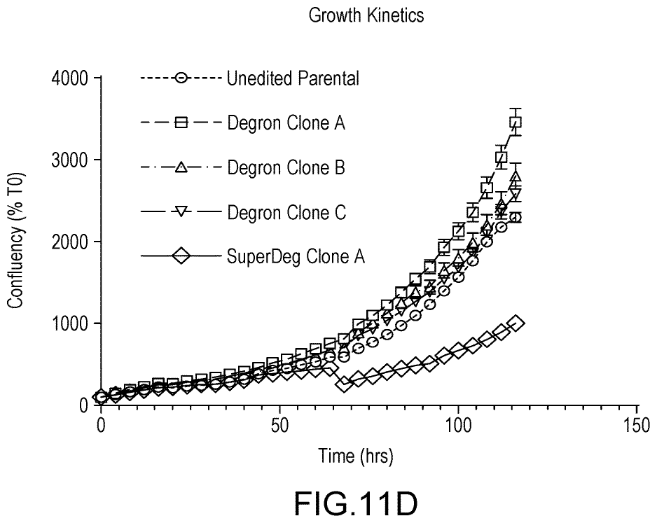

No large differences in GAPDH expression were detected with qPCR between untreated and POM-treated gene-edited cells, all of which resembled the expression in untransfected parental line (FIG. 11A). Assessment of protein expression with western blot revealed that GAPDH protein was completely depleted following one day treatment with 3 μM POM in the same iPSCs clones (FIGS. 11B and 11C). Taken together, these results suggest that none of the targeting constructs altered GAPDH mRNA levels but enabled POM-induced degradation of GAPDH protein.

Next, the unedited parental cells and the gene-edited iPSCs were seeded at equal densities and confluency was tracked over time using an Incucyte imager to assess the effect of gene-editing with the targeting constructs on growth kinetics. The results revealed that all three clones that were gene-edited with a degron-comprising targeting construct grew at comparable rates to the unedited parental cells, whereas the iPSCs gene-edited with a superdegron-comprising targeting construct grew at a slower rate (FIG. 11C).

### 8.5. Example 5: Effect of POM Concentration on Gene-Edited iPSC Death

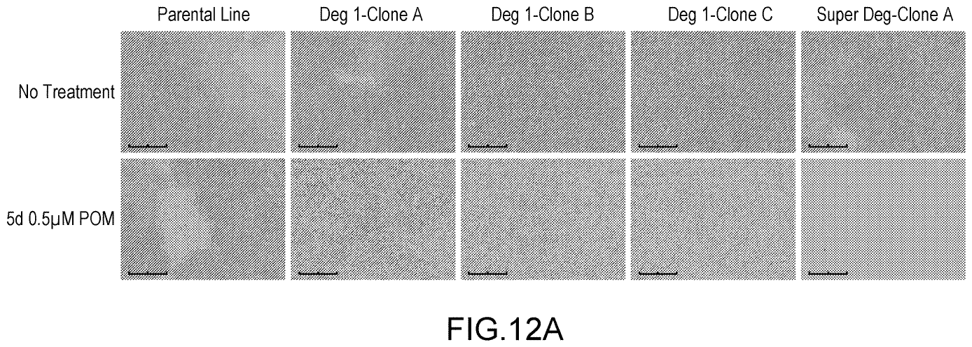

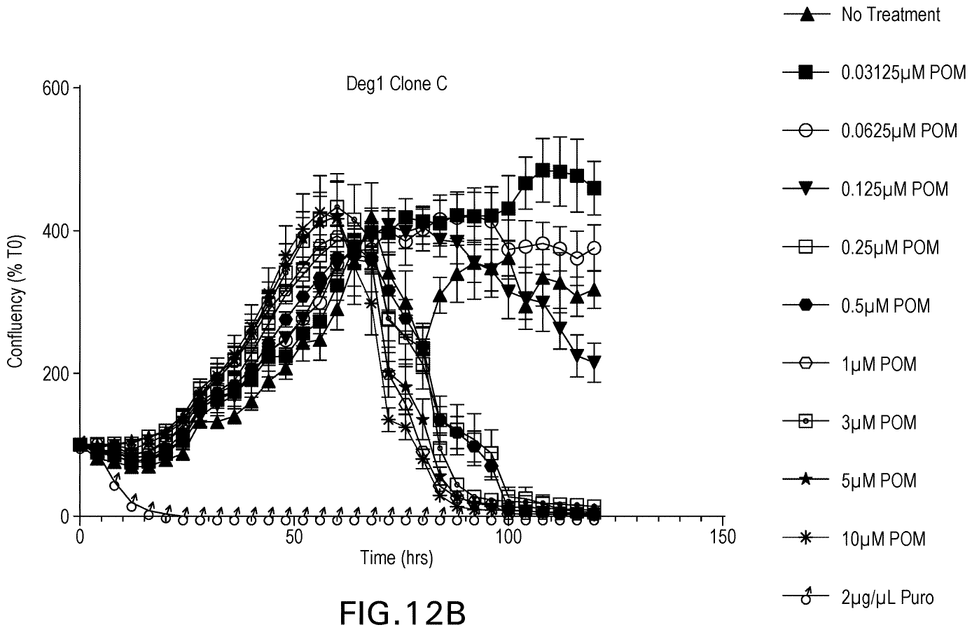

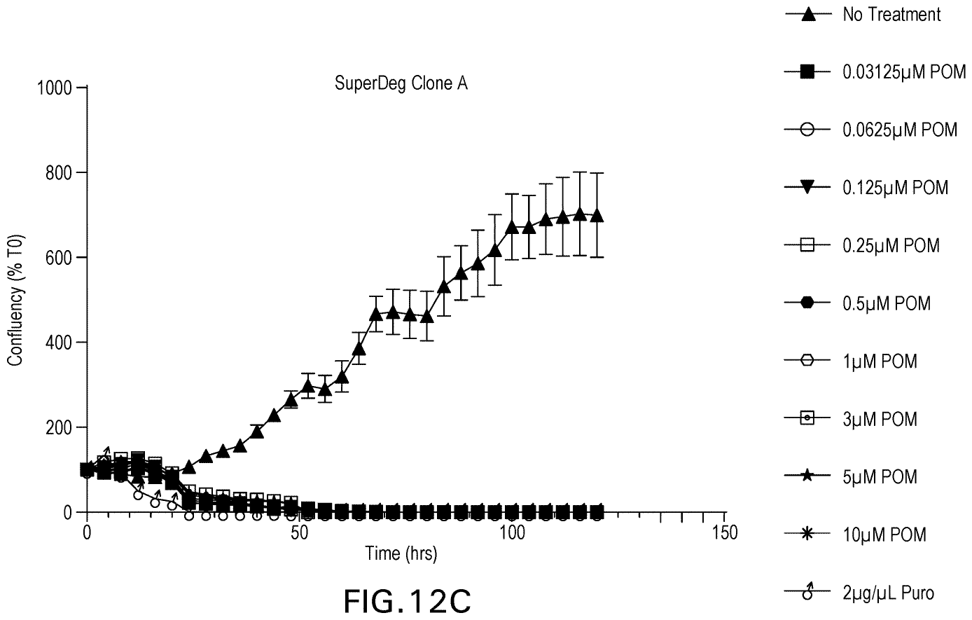

The same gene-edited iPSC clones in Example 4 were treated with 0.5 μM POM for 5 days to determine if there were any differences between the survival rates. This POM treatment resulted in complete killing of all gene-edited iPSCs but did not affect the growth of unedited parental cells (FIG. 12A).

To determine the optimum POM concentration to induce apoptosis, the confluency of gene-edited iPSC clones treated with different concentrations of POM ranging between 0.03125 μM and 10 μM was monitored for 5 days using the Incucyte. The results revealed that 0.25 μM POM was able to trigger apoptosis (not shown) and 0.5 μM POM was sufficient to achieve complete killing of gene-edited iPSCs with a degron-comprising targeting construct (FIG. 12B). This concentration was even lower for the iPSC cells that were gene-edited with a superdegron-comprising targeting construct as the lowest POM concentration evaluated (0.03125 μM) was associated with apoptosis of all cells.

### 8.6. Example 6: Activation of Targeting Constructs in Dopaminergic Neurons Differentiated from Gene-Edited iPSCs

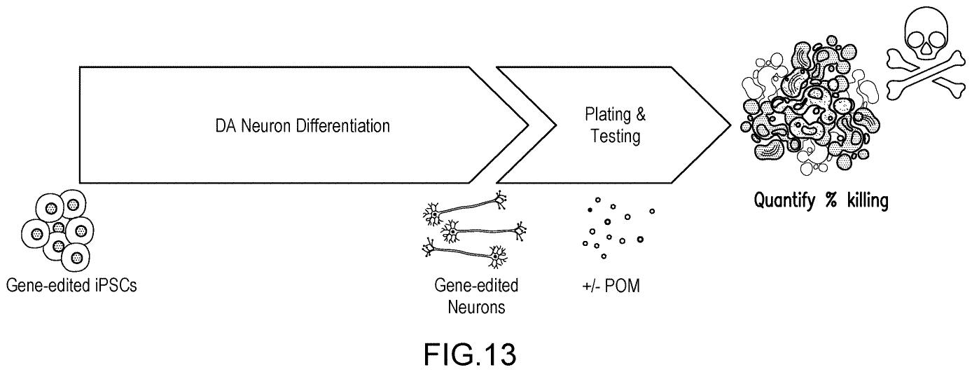

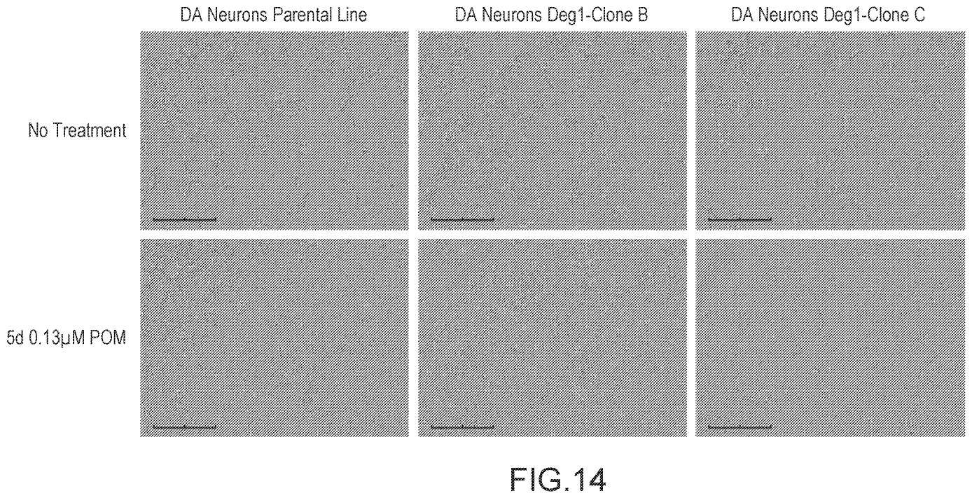

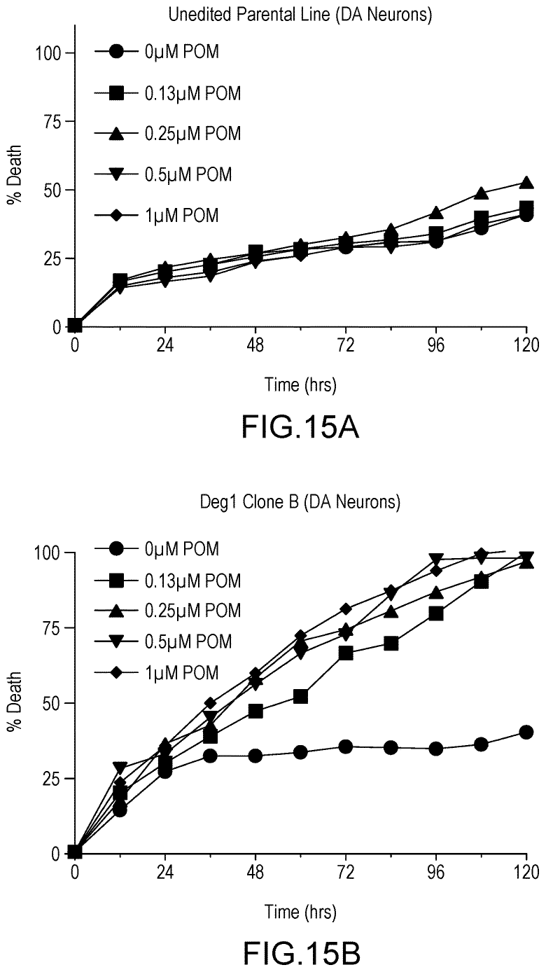

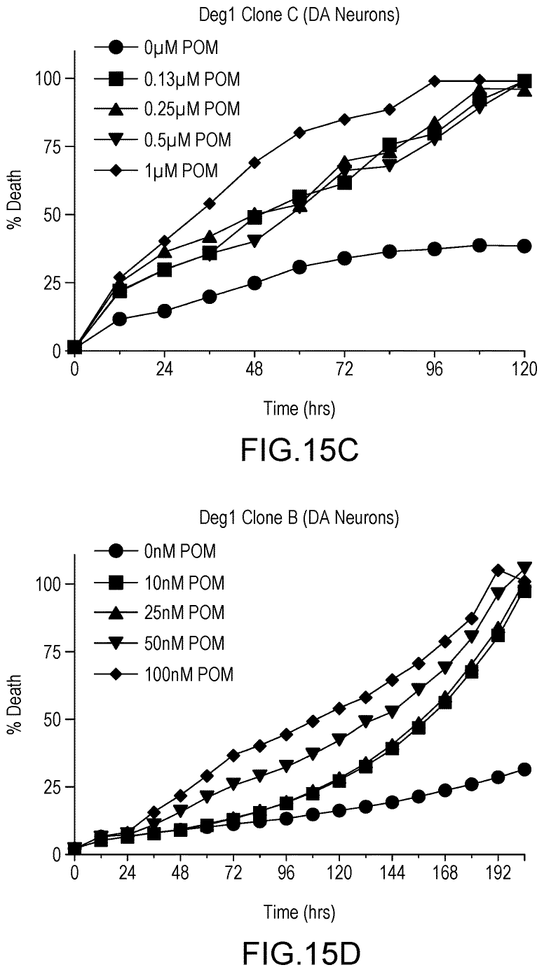

In order to determine the applicability of the degron-comprising targeting constructs to differentiated cells, unedited parental iPSCs and gene-edited iPSC clones B and C were differentiated into dopaminergic (DA) neurons as depicted in FIG. 13. The differentiation protocol was adapted from Kriks et al., 2011, Nature 480 (7378): 547-551 and U.S. Pat. No. 10,711,243, which are hereby incorporated by reference in their entireties. Cell death was assessed post-thaw after cells were plated down in the presence and absence of various concentrations of POM. Annexin V dye was used to quantify cell death in an Incucyte imager over a period of 5 days. No differences in neuronal death were detected in DA neurons derived from unedited parental iPSCs (FIGS. 14 and 15A). All POM concentrations that were evaluated achieved complete death of all neurons differentiated from both clone B and clone C iPSCs by day 5 (FIGS. 14 and 15B-15C). Further assessment with DA neurons derived from clone B revealed complete killing of these cells even with 10 nM POM (FIG. 15C), which was the lowest concentration evaluated. Taken together, these results suggest that DA neurons differentiated from iPSCs that were gene-edited with degron-comprising targeting constructs display nanomolar sensitivity to POM.

### 8.7. Example 7: Extended Testing of POM Concentrations for Activation of Targeting Constructs in Dopaminergic Neurons Differentiated from Gene-Edited iPSCs

Expanding upon the work presented in Example 6, a wider range of POM concentrations was tested in differentiated DA neurons from the unedited parental line, and two clones bi-allelically modified with a targeting construct comprising a linker that is three amino acids in length (3-aa linker) and a degron: gene-edited Clone A, or gene-edited Clone C. Cells were differentiated from iPSCs into DA neurons using the same protocol as depicted in FIG. 13. DA neurons were plated and stained with Annexin V dye and cell death was measured using an Incucyte Imager over a period of 144 hours.

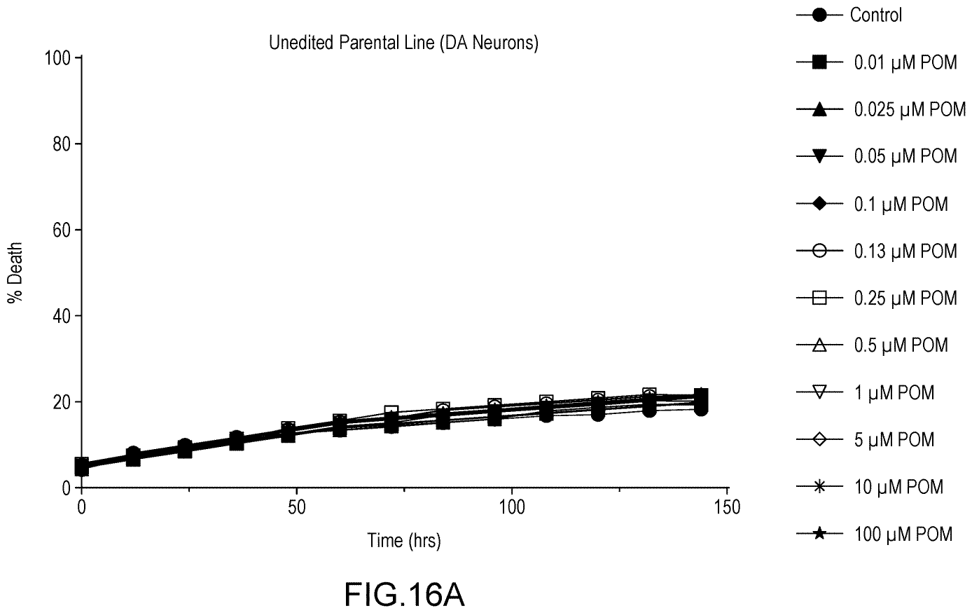

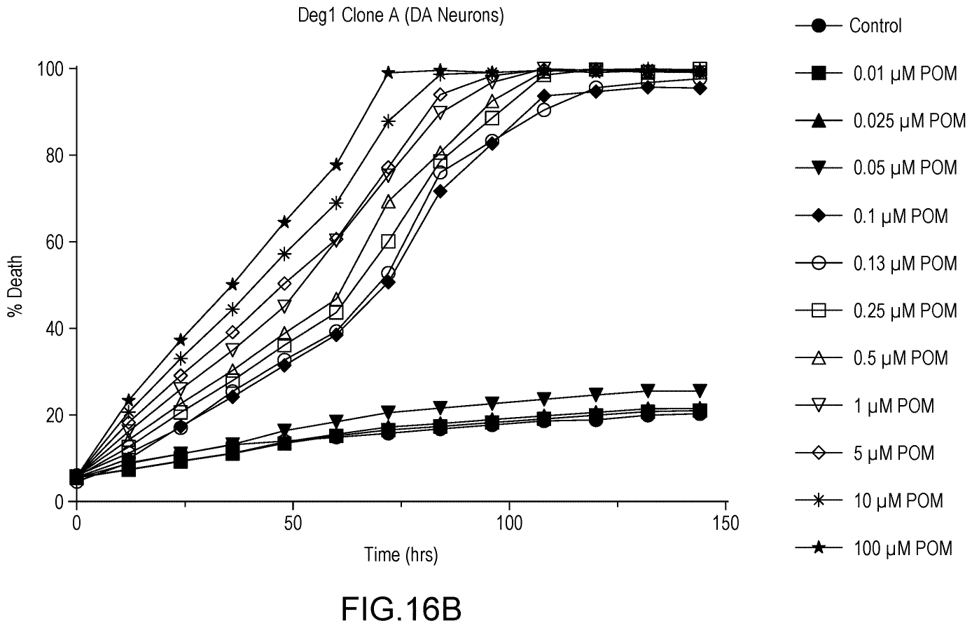

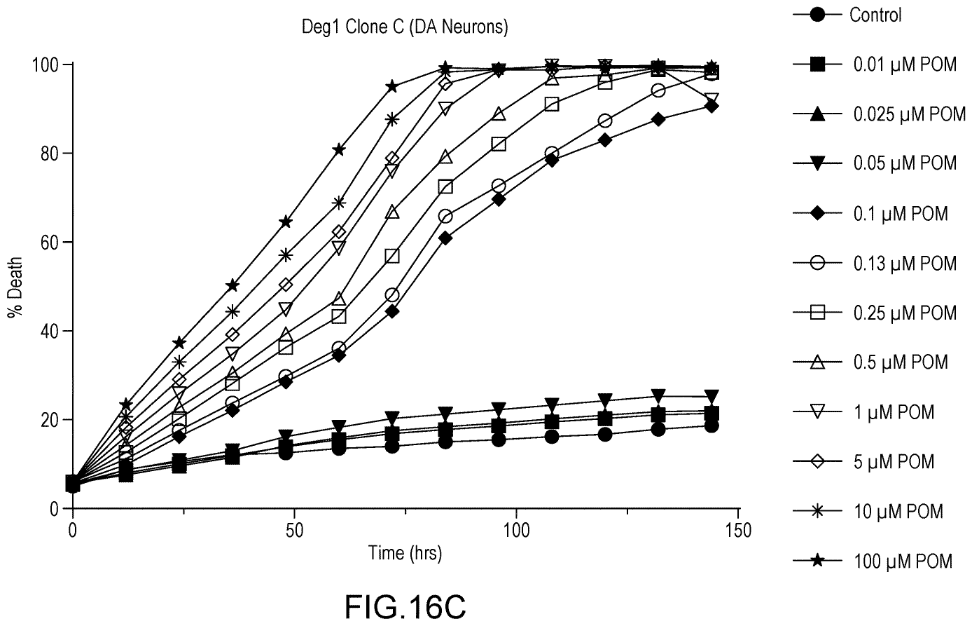

No differences in cell death were observed in DA neurons derived from unedited parental iPSCs at any concentration of POM from the range tested (0.01 μM to 100 μM) as depicted in FIG. 16A. DA Neurons derived from Clone A or Clone C reached nearly complete cell death by 144 hours with as little as 0.1 μM POM (FIGS. 16B and 16C). These results indicate that POM does not affect unedited neurons, but is able to promote apoptosis in neurons that have been modified to include a degron tag on an essential gene.

### 8.8. Example 8: Activation of Essential Gene Targeting Constructs in Myeloid Progenitor Cells Differentiated from Gene-Edited iPSCs

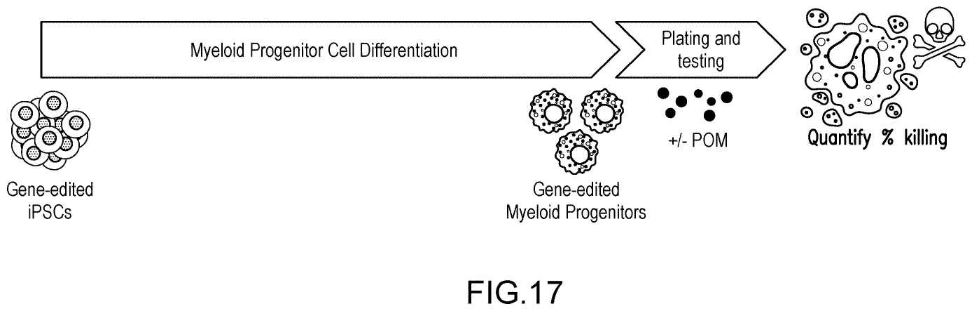

To expand the applicability of the degron-comprising constructs to additional differentiated cell types, unedited parental iPSCs and biallelically gene-edited iPSC clones A, B, and C comprising targeting construct as depicted in FIG. 7G were differentiated into myeloid progenitor (MP) cells as depicted in FIG. 17. The differentiation protocol was adapted from Douvaras et al., 2017 Jun. 6; 8 (6): 1516-1524 and PCT publication nos. WO 2023/150089 A1 and WO 2017/152081 A1, which are hereby incorporated by reference in their entireties. Cell death was assessed post-thaw after MP cells were plated down in the presence and absence of POM. Acridine Orange/Propidium Iodine (AO/PI) dye was used to quantify cell death in an Incucyte Imager over a period of 108 hours.

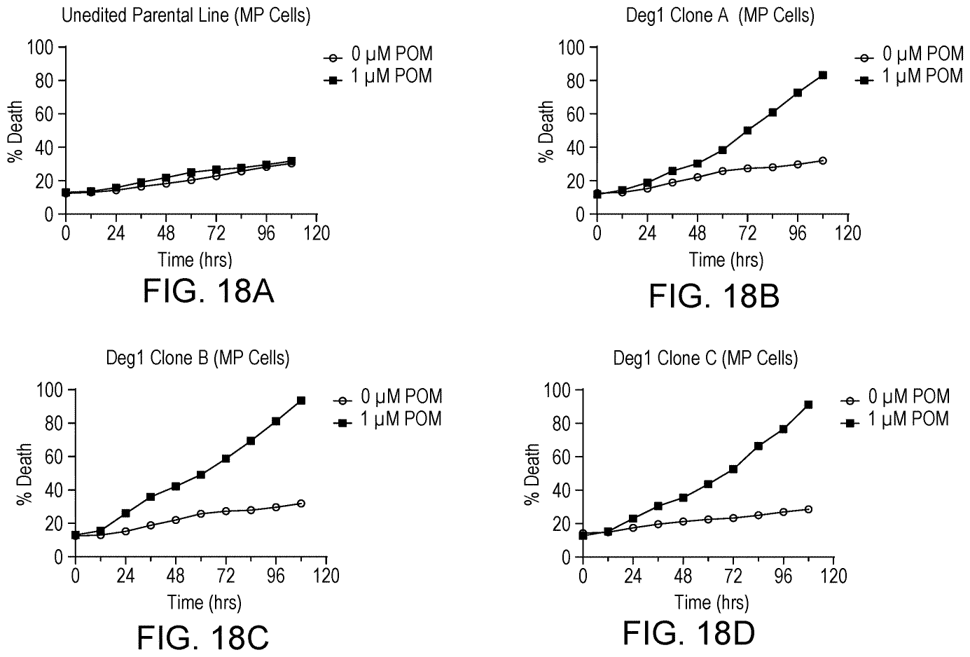

Analysis revealed that all four lines survived comparably well in the absence of POM and the unedited parental line showed no increase in cell death in the presence of POM. All three gene-edited clones died in response to POM over the treatment duration (FIGS. 18A-18D). These results suggest that myeloid progenitors differentiated from iPSCs that were gene-edited with degron-comprising targeting constructs display sensitivity to POM.

### 8.9. Example 9. Targeting of 3AA-Degron and Superdegron Constructs at the RPL13A Locus and Activation of Targeting Constructs in Gene-Edited iPSCs and Differentiated Dopaminergic Neurons and Myeloid Progenitor Cells

To demonstrate functionality of the degron-based kill switch when linked with another essential gene, a targeting construct is designed to comprise a linker that is three amino acids in length (linker 1; GGS) and a degron sequence flanked by RPL13A homology arms as depicted in FIG. 7K and SEQ ID NO:14, and a second targeting construct is designed to comprise a linker that is 10 amino acids in length (linker 4; SEQ ID NO:15) and a superdegron sequence flanked by RPL13A homology arms as depicted in FIG. 7L and SEQ ID NO: 17. Both targeting constructs are transfected into iPSCs, and amplicon depletion assessed following POM treatment relative to untreated cells. Clonal lines generated from these transfected pools are then assessed to determine the optimal POM concentration required to induce apoptosis in iPSCs and in dopaminergic neurons and myeloid progenitor cells differentiated from these gene-edited iPSCs.

### 9. SEQUENCE LISTING

Exemplary sequences of the present disclosure are provided in Table 10 below (where “SEQ” refers to the SEQ ID NO).

TABLE 10

| SEQ | Description | Sequence |
|---|---|---|
| 1. | GAPDH C-terminal LHA- | TTGGTATCGTGGAAGGACTCATGGTATGAGAGCTGGGGA |
| | linker 1-degron- | ATGGGACTGAGGCTCCCACCTTTCTCATCCAAGACTGGC |
| | stop codon-GAPDH C- | TCCTCCCTGCCGGGGCTGCGTGCAACCCTGGGGTTGG |
| | terminal RHA nucleic | GGGTTCTGGGGACTGGCTTTCCCATAATTTCCTTTCAAG |
| | acid sequence | GTGGGGAGGGAGGTAGAGGGGTGATGTGGGGAGTACG |
| | | CTGCAGGGCCTCACTCCTTTTGCAGACCACAGTCCATGC |
| | | CATCACTGCCACCCAGAAGACTGTGGATGGCCCCTCCG |
| | | GGAAACTGTGGCGTGATGGCCGCGGGGCTCTCCAGAAC |
| | | ATCATCCCTGCCTCTACTGGCGCTGCCAAGGCTGTGGG |
| | | CAAGGTCATCCCTGAGCTGAACGGGAAGCTCACTGGCA |
| | | TGGCCTTCCGTGTCCCCACTGCCAACGTGTCAGTGGTG |
| | | GACCTGACCTGCCGTCTAGAAAAACCTGCCAAATATGAT |
| | | GACATCAAGAAGGTGGTGAAGCAGGCGTCGGAGGGCC |
| | | CCCTCAAGGGCATCCTGGGCTACACTGAGCACCAGGTG |
| | | GTCTCCTCTGACTTCAACAGCGACACCCACTCCTCCACC |
| | | TTTGACGCTGGGGCTGGCATTGCCCTCAACGACCACTTT |
| | | GTCAAGCTCATTTCCTGGTATGTGGCTGGGGCCAGAGAC |
| | | TGGCTCTTAAAAAGTGCAGGGTCTGGCGCCCTCTGGTG |
| | | GCTGGCTCAGAAAAAGGGCCCTGACAACTCTTTACATCT |
| | | TCTAGGTATGACAACGAATTTGGCTACAGCAACAGGGTG |
| | | GTGGACCTCATGGCCCACATGGCCTCCAAGGAGGGCGG |
| | | GTCTCGCCCATTCCAGTGTAATCAGTGTGGGGCATCTTT |
| | | TACTCAGAAAGGTAACCTCCTCCGCCACATTAAACTGCA |
| | | CTAAGACCCCTGGACCACCAGCCAAAGCAAGAGCACAA |
| | | GAGGAAGAGAGAGACCCTCACTGCTGGGGAGTCCCTGC |
| | | CACACTCAGTCCCCCACCACACTGAATCTCCCCTCCTCA |
| | | CAGTTGCCATGTAGACCCCTTGAAGAGGGGAGGGGCCT |
| | | AGGGAGCCGCACCTTGTCATGTACCATCAATAAAGTACC |
| | | CTGTGCTCAACCAGTTACTTGTCCTGTCTTATTCTAGGGT |
| | | CTGGGGCAGAGGGGAGGGAAGCTGGGCTTGTGTCAAG |
| | | GTGAGACATTCTTGCTGGGGAGGGACCTGGTATGTTCTC |
| | | CTCAGACTGAGGGTAGGGCCTCCAAACAGCCTTGCTTG |
| | | CTTCGAGAACCATTTGCTTCCCGCTCAGACGTCTTGAGT |
| | | GCTACAGGAAGCTGGCACCACTACTTCAGAGAACAAGG |
| | | CCTTTTCCTCTCCTCGCTCCAGTCCTAGGCTATCTGCTGT |
| | | TGGCCAAACATGGAAGAAGCTATTCTGTGGGCAGCCCCA |
| | | GGGAGGCTGACAGGTGGAGGAAGTCAGGGCTCGCACT |
| | | GGGCTCTGACGCTGACTGGTTAGTGGAGCTCAGCCTGG |
| | | AGCTGAGCTGCAGCGGGCAATTCCAGCTTGGCCTCCGC |
| | | AGCTGTGAGGTCTTGAGCACGTGCTCTATTGCTTTCTGT |
| | | GCCCTCGTGTCTTATCTGAGGACATCGTGGCCAGCCCCT |
| | | AAGGTCTTCAAGCAGGATTCATCTAGGTAAACCAAGTACC |
| | | TAAAACCATGCCCAAGGCGGTAAGGACTATATAATGTTTA |
| | | AAAATCGGTAAAAATGCCCACCTCGCATAGT |
| 2. | GAPDH C-terminal LHA- | TTGGTATCGTGGAAGGACTCATGGTATGAGAGCTGGGGA |
| | linker 4-superdegron- | ATGGGACTGAGGCTCCCACCTTTCTCATCCAAGACTGGC |
| | stop codon-GAPDH | TCCTCCCTGCCGGGGCTGCGTGCAACCCTGGGGTTGG |
| | C-terminal RHA nucleic | GGGTTCTGGGGACTGGCTTTCCCATAATTTCCTTTCAAG |
| | acid sequence | GTGGGGAGGGAGGTAGAGGGGTGATGTGGGGAGTACG |
| | | CTGCAGGGCCTCACTCCTTTTGCAGACCACAGTCCATGC |
| | | CATCACTGCCACCCAGAAGACTGTGGATGGCCCCTCCG |
| | | GGAAACTGTGGCGTGATGGCCGCGGGGCTCTCCAGAAC |
| | | ATCATCCCTGCCTCTACTGGCGCTGCCAAGGCTGTGGG |
| | | CAAGGTCATCCCTGAGCTGAACGGGAAGCTCACTGGCA |
| | | TGGCCTTCCGTGTCCCCACTGCCAACGTGTCAGTGGTG |
| | | GACCTGACCTGCCGTCTAGAAAAACCTGCCAAATATGAT |
| | | GACATCAAGAAGGTGGTGAAGCAGGCGTCGGAGGGCC |
| | | CCCTCAAGGGCATCCTGGGCTACACTGAGCACCAGGTG |
| | | GTCTCCTCTGACTTCAACAGCGACACCCACTCCTCCACC |
| | | TTTGACGCTGGGGCTGGCATTGCCCTCAACGACCACTTT |
| | | GTCAAGCTCATTTCCTGGTATGTGGCTGGGGCCAGAGAC |
| | | TGGCTCTTAAAAAGTGCAGGGTCTGGCGCCCTCTGGTG |
| | | GCTGGCTCAGAAAAAGGGCCCTGACAACTCTTTACATCT |
| | | TCTAGGTATGACAACGAATTTGGCTACAGCAACAGGGTG |
| | | GTGGACCTCATGGCCCACATGGCCTCCAAGGAGGGCTC |
| | | AGGTAGCGGAAGCGGATCAGGTGGATTCAATGTACTGA |
| | | TGGTCCATAAACGGAGTCACACTGGCGAGCGCCCGCTC |
| | | CAATGTGAAATCTGCGGGTTCACGTGTCGGCAGAAGGG |
| | | CAACCTCCTCCGGCATATCAAGCTGCACACGGGTGAAAA |
| | | ACCGTTTAAGTGCCATCTCTGCAATTACGCCTGTCAGAG |
| | | AAGAGATGCTTTGTAAGACCCCTGGACCACCAGCCAAAG |
| | | CAAGAGCACAAGAGGAAGAGAGAGACCCTCACTGCTGG |
| | | GGAGTCCCTGCCACACTCAGTCCCCCACCACACTGAAT |
| | | CTCCCCTCCTCACAGTTGCCATGTAGACCCCTTGAAGAG |
| | | GGGAGGGGCCTAGGGAGCCGCACCTTGTCATGTACCAT |
| | | CAATAAAGTACCCTGTGCTCAACCAGTTACTTGTCCTGTC |
| | | TTATTCTAGGGTCTGGGGCAGAGGGGAGGGAAGCTGGG |
| | | CTTGTGTCAAGGTGAGACATTCTTGCTGGGGAGGGACC |
| | | TGGTATGTTCTCCTCAGACTGAGGGTAGGGCCTCCAAAC |
| | | AGCCTTGCTTGCTTCGAGAACCATTTGCTTCCCGCTCAG |
| | | ACGTCTTGAGTGCTACAGGAAGCTGGCACCACTACTTCA |
| | | GAGAACAAGGCCTTTTCCTCTCCTCGCTCCAGTCCTAGG |
| | | CTATCTGCTGTTGGCCAAACATGGAAGAAGCTATTCTGTG |
| | | GGCAGCCCCAGGGAGGCTGACAGGTGGAGGAAGTCAG |
| | | GGCTCGCACTGGGCTCTGACGCTGACTGGTTAGTGGAG |
| | | CTCAGCCTGGAGCTGAGCTGCAGCGGGCAATTCCAGCT |
| | | TGGCCTCCGCAGCTGTGAGGTCTTGAGCACGTGCTCTAT |
| | | TGCTTTCTGTGCCCTCGTGTCTTATCTGAGGACATCGTG |
| | | GCCAGCCCCTAAGGTCTTCAAGCAGGATTCATCTAGGTA |
| | | AACCAAGTACCTAAAACCATGCCCAAGGCGGTAAGGACT |
| | | ATATAATGTTTAAAAATCGGTAAAAATGCCCACCTCGCATA |
| | | GT |
| 3. | Degron (IKZF3 (144- | RPFQCNQCGASFTQKGNLLRHIKLH |
| | 168)) amino acid | |
| | sequence | |
| 4. | Superdegron amino acid | FNVLMVHKRSHTGERPLQCEICGFTCRQKGNLLRHIKLHT |
| | sequence | GEKPFKCHLCNYACQRRDAL |
| 5. | Alternative degron 1 | FNVLMVHKRSHTGERP |
| | amino acid sequence | |
| 6. | Alternative degron 2 | FNVLMVHRRSHTGERP |
| | amino acid sequence | |
| 7. | Alternative degron 3 | TGEKPFKCHLCNYACQRRDAL |
| | amino acid sequence | |
| 8. | Alternative degron 4 | TGERPFRCHLCNYACQRRDAL |
| | amino acid sequence | |
| 9 | Alternative degron 5 | FQCNQCGASFT |
| | amino acid sequence | |
| 10. | Alternative degron 6 | FQCPICGLVIK |
| | amino acid sequence | |
| 11. | Alternative degron 7 | LQCEICGFTCR |
| | amino acid sequence | |
| 12. | Alternative degron 8 | LQCEICGYQCR |
| | amino acid sequence | |
| 13. | Alternative degron 9 | LQCEVCGFQCR |
| | amino acid sequence | |
| N/A | Linker 1 | GGS |
| 14. | RPL13A C-terminal LHA- | TCTTAAGCCCCTCTCTTTCTCTAACAGAAAAAGCGGATGG |
| | linker 1-degron- | TGGTTCCTGCTGCCCTCAAGGTCGTGCGTCTGAAGCCTA |
| | stop codon-RPL13A C- | CAAGAAAGGTGAGTCCCAGCTTACGCTGCACCATCTACT |
| | terminal RHA | TGGGAGATTTCAGGCCTGCTGAGGGACCTGGGGACCTG |
| | | GAGCCTGGCAGATGATGTCCTTATCTCACGATGGTCTGC |
| | | GGATGTCCCTGTGGGAATGGCGACAATGCCAATGGCTTA |
| | | GCTGATGCCAGGAGGCTTGGGTGGGTGCTTTTCTAACA |
| | | GGCCTGCAGAGAACAGTTGCATTATGATATGCCCAGCTG |
| | | TCAGTCACCTCCCAGCTCTCAACAGCTCCGGCTCTTCAG |
| | | GGTGTGGGGGCTTAGATATCCTTACAACTTCATTTGTTCA |
| | | CCCCCCCCCCCCCCCCCCGCAGTTTGCCTATCTGGGGC |
| | | GCCTGGCTCACGAGGTTGGCTGGAAGTACCAGGCAGTG |
| | | ACAGCCACCCTGGAGGAGAAGAGGAAAGAGAAAGCCAA |
| | | GATCCACTACCGGAAGAAGAAACAGCTCATGGTGAGGC |
| | | CAGGGGCTGGTGCTGAGGGGGGCATCTCACTCCTGGAC |
| | | AGGCCTGGCAGGTGCCTTGCTCACAGAGTACTCTTAACT |
| | | GGCAAAGGACCAGCCGGGGTTGGGGTGGGATGCAGTC |
| | | CATGTAATGAGGGCAATGCAACCCCTCCTGACCACCACC |
| | | ACCTGCACTTATTCTTGGCAGAGGCTACGCAAACAAGCC |
| | | GAGAAGAACGTGGAGAAGAAAATTGACAAATACACAGAG |
| | | GTCCTCAAGACCCACGGACTCCTGGTCGGCGGGTCTCG |
| | | CCCATTCCAGTGTAATCAGTGTGGGGCATCTTTTACTCAG |
| | | AAAGGTAACCTCCTCCGCCACATTAAACTGCACTAAGCC |
| | | CAATAAAGACTGTTAATTCCTCATGCGTTGCCTGCCCTTC |
| | | CTCCATTGTTGCCCTGGAATGTACGGGACCCAGGGGCA |
| | | GCAGCAGTCCAGGTGCCACAGGCAGCCCTGGGACATAG |
| | | GAAGCTGGGAGCAAGGAAAGGGTCTTAGTCACTGCCTC |
| | | CCGAAGTTGCTTGAAAGCACTCGGAGAATTGTGCAGGT |
| | | GTCATTTATCTATGACCAATAGGAAGAGCAACCAGTTACT |
| | | ATGAGTGAAAGGGAGCCAGAAGACTGATTGGAGGGCCC |
| | | TATCTTGTGAGTGGGGCATCTGTTGGACTTTCCACCTGG |
| | | TCATATACTCTGCAGCTGTTAGAATGTGCAAGCACTTGGG |
| | | GACAGCATGAGCTTGCTGTTGTACACAGGGTATTTCTAGA |
| | | AGCAGAAATAGACTGGGAAGATGCACAACCAAGGGGTTA |
| | | CAGGCATCGCCCATGCTCCTCACCTGTATTTTGTAATCAG |
| | | AAATAAATTGCTTTTAAAGAAATCTGGCGTCTTTGCACTGT |
| | | GTCTGCTGTGGAGGCAGGCCCCTGGCAAATGGGGGGT |
| | | GAGGAGCTTGAAGAGGGTAGAATGGGCTGTGCTAATATA |
| | | CAGAATATATGTAACTTGCTATAAATTGAATGATCCTTTATA |
| | | GACACCGTTTACAAACCAAAGACATAAAATGTGGCCAGC |
| | | AGTGCCTGGTGCTTCCTAGTTAATGTAAAGCTGTCTCATT |
| | | CTAATTCAGCTGCAAAGTATGGACCCATGCCCTGCTGCC |
| | | AGGCTGCTGTAGTCCCGGCGGTCTGTAGAGACTAGCATT |
| | | TTGCAAATGATAA |
| 15. | Linker 4 | GSGSGSGSGG |
| 16. | Exemplary Linker A | GGGS |
| N/A | Exemplary Linker B | GSG |
| 17. | RPL13A C-terminal LHA- | TCTTAAGCCCCTCTCTTTCTCTAACAGAAAAAGCGGATGG |
| | linker 4-superdegron- | TGGTTCCTGCTGCCCTCAAGGTCGTGCGTCTGAAGCCTA |
| | stop codon-RPL13A | CAAGAAAGGTGAGTCCCAGCTTACGCTGCACCATCTACT |
| | C-terminal RHA | TGGGAGATTTCAGGCCTGCTGAGGGACCTGGGGACCTG |
| | | GAGCCTGGCAGATGATGTCCTTATCTCACGATGGTCTGC |
| | | GGATGTCCCTGTGGGAATGGCGACAATGCCAATGGCTTA |
| | | GCTGATGCCAGGAGGCTTGGGTGGGTGCTTTTCTAACA |
| | | GGCCTGCAGAGAACAGTTGCATTATGATATGCCCAGCTG |
| | | TCAGTCACCTCCCAGCTCTCAACAGCTCCGGCTCTTCAG |
| | | GGTGTGGGGGCTTAGATATCCTTACAACTTCATTTGTTCA |
| | | CCCCCCCCCCCCCCCCCCGCAGTTTGCCTATCTGGGGC |
| | | GCCTGGCTCACGAGGTTGGCTGGAAGTACCAGGCAGTG |
| | | ACAGCCACCCTGGAGGAGAAGAGGAAAGAGAAAGCCAA |
| | | GATCCACTACCGGAAGAAGAAACAGCTCATGGTGAGGC |
| | | CAGGGGCTGGTGCTGAGGGGGGCATCTCACTCCTGGAC |
| | | AGGCCTGGCAGGTGCCTTGCTCACAGAGTACTCTTAACT |
| | | GGCAAAGGACCAGCCGGGGTTGGGGTGGGATGCAGTC |
| | | CATGTAATGAGGGCAATGCAACCCCTCCTGACCACCACC |
| | | ACCTGCACTTATTCTTGGCAGAGGCTACGCAAACAAGCC |
| | | GAGAAGAACGTGGAGAAGAAAATTGACAAATACACAGAG |
| | | GTCCTCAAGACCCACGGACTCCTGGTCGGCTCAGGTAG |
| | | CGGAAGCGGATCAGGTGGATTCAATGTACTGATGGTCC |
| | | ATAAACGGAGTCACACTGGCGAGCGCCCGCTCCAATGT |
| | | GAAATCTGCGGGTTCACGTGTCGGCAGAAGGGCAACCT |
| | | CCTCCGGCATATCAAGCTGCACACGGGTGAAAAACCGTT |
| | | TAAGTGCCATCTCTGCAATTACGCCTGTCAGAGAAGAGAT |
| | | GCTTTGTAAGCCCAATAAAGACTGTTAATTCCTCATGCGT |
| | | TGCCTGCCCTTCCTCCATTGTTGCCCTGGAATGTACGGG |
| | | ACCCAGGGGCAGCAGCAGTCCAGGTGCCACAGGCAGC |
| | | CCTGGGACATAGGAAGCTGGGAGCAAGGAAAGGGTCTT |
| | | AGTCACTGCCTCCCGAAGTTGCTTGAAAGCACTCGGAG |
| | | AATTGTGCAGGTGTCATTTATCTATGACCAATAGGAAGAG |
| | | CAACCAGTTACTATGAGTGAAAGGGAGCCAGAAGACTGA |
| | | TTGGAGGGCCCTATCTTGTGAGTGGGGCATCTGTTGGAC |
| | | TTTCCACCTGGTCATATACTCTGCAGCTGTTAGAATGTGC |
| | | AAGCACTTGGGGACAGCATGAGCTTGCTGTTGTACACAG |
| | | GGTATTTCTAGAAGCAGAAATAGACTGGGAAGATGCACA |
| | | ACCAAGGGGTTACAGGCATCGCCCATGCTCCTCACCTGT |
| | | ATTTTGTAATCAGAAATAAATTGCTTTTAAAGAAATCTGGC |
| | | GTCTTTGCACTGTGTCTGCTGTGGAGGCAGGCCCCTGG |
| | | CAAATGGGGGGTGAGGAGCTTGAAGAGGGTAGAATGGG |
| | | CTGTGCTAATATACAGAATATATGTAACTTGCTATAAATTGA |
| | | ATGATCCTTTATAGACACCGTTTACAAACCAAAGACATAAA |
| | | ATGTGGCCAGCAGTGCCTGGTGCTTCCTAGTTAATGTAA |
| | | AGCTGTCTCATTCTAATTCAGCTGCAAAGTATGGACCCAT |
| | | GCCCTGCTGCCAGGCTGCTGTAGTCCCGGCGGTCTGTA |
| | | GAGACTAGCATTTTGCAAATGATAA |
| 18. | Exemplary Linker C | GGSG |
| 19 | Exemplary Linker D | GGGS |
| 20. | Exemplary Linker E | GGGGS |
| 21. | Exemplary Linker F | (GGS)3 |
| 22 | Exemplary Linker G | (GGGGS)2 |
| 23. | Exemplary Linker H | (GGGGS)3 |
| | (Linker 2) | |
| 24. | Cas12a amino acid | MTQFEGFTNLYQVSKTLRFELIPQGKTLKHIQEQGFIEEDKA |
| | sequence | RNDHYKELKPIIDRIYKTYADQCLQLVQLDWENLSAAIDSYR |
| | | KEKTEETRNALIEEQATYRNAIHDYFIGRTDNLTDAINKRHA |
| | | EIYKGLFKAELFNGKVLKQLGTVTTTEHENALLRSFDKFTTY |
| | | FSGFYENRKNVFSAEDISTAIPHRIVQDNFPKFKENCHIFTR |
| | | LITAVPSLREHFENVKKAIGIFVSTSIEEVFSFPFYNQLLTQT |
| | | QIDLYNQLLGGISREAGTEKIKGLNEVLNLAIQKNDETAHIIA |
| | | SLPHRFIPLFKQILSDRNTLSFILEEFKSDEEVIQSFCKYKTLL |
| | | RNENVLETAEALFNELNSIDLTHIFISHKKLETISSALCDHWD |
| | | TLRNALYERRISELTGKITKSAKEKVQRSLKHEDINLQEIISA |
| | | AGKELSEAFKQKTSEILSHAHAALDQPLPTTLKKQEEKEILK |
| | | SQLDSLLGLYHLLDWFAVDESNEVDPEFSARLTGIKLEMEP |
| | | SLSFYNKARNYATKKPYSVEKFKLNFQMPTLASGWDVNKE |
| | | KNNGAILFVKNGLYYLGIMPKQKGRYKALSFEPTEKTSEGF |
| | | DKMYYDYFPDAAKMIPKCSTQLKAVTAHFQTHTTPILLSNN |
| | | FIEPLEITKEIYDLNNPEKEPKKFQTAYAKKTGDQKGYREAL |
| | | CKWIDFTRDFLSKYTKTTSIDLSSLRPSSQYKDLGEYYAELN |
| | | PLLYHISFQRIAEKEIMDAVETGKLYLFQIYNKDFAKGHHGK |
| | | PNLHTLYWTGLFSPENLAKTSIKLNGQAELFYRPKSRMKR |
| | | MAHRLGEKMLNKKLKDQKTPIPDTLYQELYDYVNHRLSHDL |
| | | SDEARALLPNVITKEVSHEIIKDRRFTSDKFFFHVPITLNYQA |
| | | ANSPSKFNQRVNAYLKEHPETPIIGIDRGERNLIYITVIDSTG |
| | | KILEQRSLNTIQQFDYQKKLDNREKERVAARQAWSVVGTIK |
| | | DLKQGYLSQVIHEIVDLMIHYQAVVVLENLNFGFKSKRTGIA |
| | | EKAVYQQFEKMLIDKLNCLVLKDYPAEKVGGVLNPYQLTDQ |
| | | FTSFAKMGTQSGFLFYVPAPYTSKIDPLTGFVDPFVWKTIKN |
| | | HESRKHFLEGFDFLHYDVKTGDFILHFKMNRNLSFQRGLP |
| | | GFMPAWDIVFEKNETQFDAKGTPFIAGKRIVPVIENHRFTG |
| | | RYRDLYPANELIALLEEKGIVFRDGSNILPKLLENDDSHAIDT |
| | | MVALIRSVLQMRNSNAATGEDYINSPVRDLNGVCFDSRFQ |
| | | NPEWPMDADANGAYHIALKGQLLLNHLKESKDLKLQNGIS |
| | | NQDWLAYIQELRN |
| 25. | Cas12a variant 1 amino | MTQFEGFTNLYQVSKTLRFELIPQGKTLKHIQEQGFIEEDKA |
| | acid sequence | RNDHYKELKPIIDRIYKTYADQCLQLVQLDWENLSAAIDSYR |
| | | KEKTEETRNALIEEQATYRNAIHDYFIGRTDNLTDAINKRHA |
| | | EIYKGLFKAELFNGKVLKQLGTVTTTEHENALLRSFDKFTTY |
| | | FSGFYENRKNVFSAEDISTAIPHRIVQDNFPKFKENCHIFTR |
| | | LITAVPSLREHFENVKKAIGIFVSTSIEEVFSFPFYNQLLTQT |
| | | QIDLYNQLLGGISREAGTEKIKGLNEVLNLAIQKNDETAHIIA |
| | | SLPHRFIPLFKQILSDRNTLSFILEEFKSDEEVIQSFCKYKTLL |
| | | RNENVLETAEALFNELNSIDLTHIFISHKKLETISSALCDHWD |
| | | TLRNALYERRISELTGKITKSAKEKVQRSLKHEDINLQEIISA |
| | | AGKELSEAFKQKTSEILSHAHAALDQPLPTTLKKQEEKEILK |
| | | SQLDSLLGLYHLLDWFAVDESNEVDPEFSARLTGIKLEMEP |
| | | SLSFYNKARNYATKKPYSVEKFKLNFQRPTLASGWDVNKE |
| | | KNNGAILFVKNGLYYLGIMPKQKGRYKALSFEPTEKTSEGF |
| | | DKMYYDYFPDAAKMIPKCSTQLKAVTAHFQTHTTPILLSNN |
| | | FIEPLEITKEIYDLNNPEKEPKKFQTAYAKKTGDQKGYREAL |
| | | CKWIDFTRDFLSKYTKTTSIDLSSLRPSSQYKDLGEYYAELN |
| | | PLLYHISFQRIAEKEIMDAVETGKLYLFQIYNKDFAKGHHGK |
| | | PNLHTLYWTGLFSPENLAKTSIKLNGQAELFYRPKSRMKR |
| | | MAARLGEKMLNKKLKDQKTPIPDTLYQELYDYVNHRLSHDL |
| | | SDEARALLPNVITKEVSHEIIKDRRFTSDKFLFHVPITLNYQA |
| | | ANSPSKFNQRVNAYLKEHPETPIIGIDRGERNLIYITVIDSTG |
| | | KILEQRSLNTIQQFDYQKKLDNREKERVAARQAWSVVGTIK |
| | | DLKQGYLSQVIHEIVDLMIHYQAVVVLENLNFGFKSKRTGIA |
| | | EKAVYQQFEKMLIDKLNCLVLKDYPAEKVGGVLNPYQLTDQ |
| | | FTSFAKMGTQSGFLFYVPAPYTSKIDPLTGFVDPFVWKTIKN |
| | | HESRKHFLEGFDFLHYDVKTGDFILHFKMNRNLSFQRGLP |
| | | GFMPAWDIVFEKNETQFDAKGTPFIAGKRIVPVIENHRFTG |
| | | RYRDLYPANELIALLEEKGIVFRDGSNILPKLLENDDSHAIDT |
| | | MVALIRSVLQMRNSNAATGEDYINSPVRDLNGVCFDSRFQ |
| | | NPEWPMDADANGAYHIALKGQLLLNHLKESKDLKLQNGIS |
| | | NQDWLAYIQELRNGRSSDDEATADSQHAAPPKKKRKV |
| 26. | B-GEn.1 amino acid | MTIRSMKLKLKIYSGRSAPQLRQGLWRLHRLLNEGTAYYMD |
| | sequence | WLVHMRQEALPGKSKEEIRAELERRVRQQQEKNGVQNDQ |
| | | VPMDEVLSALRQLYELLVPSAVNNSGDAQTLSRKFLSPLVD |
| | | PNSEGGKGTSNAGAKPGWRKKQEAGDPSWEKDYERWLK |
| | | RKQADPTAEILGKLETAGLKPLFPLYTNEVKDIRWMPLTSKQ |
| | | YVRNWDRDMFQQAIEHLLSWETWNRKVNEERAKLKETVR |
| | | RFEEQHLANGKDWLSPLQAYEANREQALRDMAISPSDRFR |
| | | ITRRQIKGWSELYERWNKLAPTASVEAYMQEVRHVQKKLG |
| | | GTFGDADLYRFLAKPENVHIWRDHQERLHYYAAYNDLHKR |
| | | LMSAKEQAAFTLPDPVAHPLWVRFDARDGNLFTYILQADSS |
| | | KQRSRRYVNFSRFLWPVEDGYFEETENVKVELALSKQFYR |
| | | QVIVHDNPTGKQKITFQDYSSKEILEGHLGGAKLQLDRNFL |
| | | RKSGRDFETGDFGPAFLNVVLDLKPKQEVKNGRLQSPLGQ |
| | | ALLVKSRPNDIPKVYGYKPDALAAWLEQASGEESLGSESLR |
| | | QGFRVMSIDLGVRSAAAISVFSVKGEKTREGDKVCYPVGE |
| | | TGLFAVHDRSFLLRLPGESSEKRVNVERDKRKTERMQIRY |
| | | HIRTLARVLRLANKATPMDRIKAVQDVLNDIESTRFMNDHD |
| | | HHVYNHALETLRTYAPDHQGIWEEQVIAAHRQLEHHVGVIV |
| | | GEWRKNWGKDRRGTVGLSMDNIEELDEMRRLLISWSRRA |
| | | RYPREAKPFQVNESNPVHLLRHLQNLKEDRLKQLANLIVMT |
| | | ALGYVYDSKEKKWKAAYPACQLILFEDLQRYRFHLDRSARE |
| | | NSQLMKWAHRSIPKYVWMQGEPYGLQIGDVWAGFTSRYH |
| | | AKTGAPGIRCKALTEKDFQQGRLLESLVAEGMFTLQEVGTL |
| | | KPGDIVPAEGGELFVTLADDSGDRIVITHADINAAQNVQKRF |
| | | WLANSERFRVACRSVQIASQECFIPSSESVAKKMGKGVFV |
| | | RDFSFHKDMEVYHWNNQVKLTAKNVPTDHSDDLQDLQDY |
| | | QAILEEARESSSSYKTLFRDPSGFFFPDDVWVPQNIYWRE |
| | | VKKTITALLRKRIMST |
| 27. | B-GEn.2 amino acid | MPIRSFKLKLVTHNGDSTYMDKLRRGLWKTHVIINRGIAYYM |
| | sequence | NTLALMRQEPYGSKSREEVRLDLLSTLREQQRRNNWSEQ |
| | | TGTDDELLSLSRRVYELLVPSAIGEKGDAQMLSRKFLSPLV |
| | | DPNSEGGRGTAKSGRKPRWKKMMEEGHPDWEKEKEKDA |
| | | AKKAEDPTASILADLEAVGLLPLFPLFSDEQKEIRWLPKKKR |
| | | QFVRTWDRDMFQQALERMLSWESWNRRVAEEYLKLQAQ |
| | | RDEVYAKYLEDAGSWLNDLQTFEKQREEELAEVSFEPNSE |
| | | YLITRRQIRGWKEVYEKWSKTSENASQEQLWRMVADVQTA |
| | | MAGAFGDPKVYQFLSQPKHHHIWREHPNRLFYYSKYNEV |
| | | REKLNRAKKQAAFTLPDPVEHPLWTRFDARGGNIHDYEISK |
| | | VGKQYHVTFSSLILPEAQSWVEIENVTVGIGNSLQLKRQIRL |
| | | DGYADKKQKVKYYDYSSRFELTGVLGGAKIQFDRKHLKKA |
| | | AHRLAEGETGPIFLNVVVDVEPFLEVKNGRLRTPLGQVLQV |
| | | NTRDWPKVVDYKAKELSVLMENTQIGNENGVSTIEAGMRI |
| | | MSIDLGQRTAAAVSIFEVISKKPDEKETKLFYPIADTDLYAVH |
| | | RRSLLLRLPGEEISSKKMIEKRKERARIRSLVRYQIRLLSEVL |
| | | RLHTQGTAEQRRFKLDELLVSIQKKLELDQSEWISELEKLFD |
| | | YIDESAEKWKEALVVAHRTLEPIVVEAVRNWKKSLSKENKD |
| | | RRRIAGISIWSIEELEETRKLLIAWSKHSREPGIPKRLEKEET |
| | | FAPEHLQHIQNVKDDRLKQMANLFVMTALGYKYDEGNKR |
| | | WVEAYPACQVILFEDLSRYRFALDRPRRENNRLMKWAHRS |
| | | IPRLTYMQAELFGIQVGDVYSAYTSRFHAKTGAPGIRCHALT |
| | | EADLQSNSYVVNQLIKDKFIQDNQTEILKAGQIVPWQGGEL |
| | | FVTFADRSGASLAVIHADINAAQNLQKRFWQHNSEVFRVP |
| | | CKVVKGGLVPVYEKMRKLFGKGLFVNIDDPESKEVYRWEH |
| | | STKMKSKTTPVDLESEDIDHEELSDEWEDMQEGYKTLLRD |
| | | PSGFFWSSDSWIPQKDFWIRVKSRIGKSLREQIR |
| 28. | B-GEn.1.2 amino acid | MPIRSFKLKLVTHNGDSTYMDKLRRGLWKTHVIINRGIAYYM |
| | sequence | NTLALMRQEPYGSKSREEVRLDLLSTLREQQRRNNWSEQ |
| | | TGTDDELLSLSRRVYELLVPSAIGEKGDAQMLSRKFLSPLV |
| | | DPNSEGGRGTAKSGRKPRWKKMMEEGHPDWEKEKEKDA |
| | | AKKAEDPTASILADLEAVGLLPLFPLFSDEQKEIRWLPKKKR |
| | | QFVRTWDRDMFQQALERMLSWESWNRRVAEEYQKLQAQ |
| | | RDEVYAKYLEDAGSWLNDLQTFEKQREEELAEVSFEPNSE |
| | | YLITRRQIRGWKEVYEKWSKTSENASQEQLWRMVADVQTA |
| | | MAGAFGDPKVYQFLSQPKHHHIWREHPNRLFYYSKYNEV |
| | | REKLNRAKKQAAFTLPDPVEHPLWTRFDARGGNIHDYEISK |
| | | VGKQYHVTFSSLILPEAQSWVEIENVTVGIGNSLQLKRQIRL |
| | | DGYADKKQKVKYYDYSSRFELTGVLGGAKIQFDRKHLKKA |
| | | AHRLAEGETGPIFLNVVVDVEPFLEVKNGRLRTPLGQVLQV |
| | | NTRDWPKVVDYKAKELSVLMENTQIGNENGVSTIEAGMRI |
| | | MSIDLGQRTAAAVSIFEVISKKPDEKETKLFYPIADTDLYAVH |
| | | RRSLLLRLPGEEISSKKMIEKRKERARIRSLVRYQIRLLSEVL |
| | | RLHTQGTAEQRRFKLDELLVSIQRKLELDQSEWISELEKLF |
| | | DYIDESAEKWKEALVVAHRTLEPIVVEAVRNWKKSLSKENK |
| | | DRRRIAGISIWSIEELEETRKLLIAWSKHSREPGIPKRLEKEE |
| | | TFAPEHLQHIQNVKDDRLKQMANLFVMTALGYKYDEGNKR |
| | | WVEAYPACQVILFEDLSRYRFALDRPRRENNRLMKWAHRS |
| | | IPRLTYMQAELFGIQVGDVYSAYTSRFHAKTGAPGIRCHALT |
| | | EADLQSNSYVVNQLIKDKFIQDNQTEILKAGQIVPWQGGEL |
| | | FVTFADRSGASLAVIHADINAAQNLQKRFWQHNSEVFRVP |
| | | CKVVKGGLVPVYEKMRKLFGKGLFVNIDDPESKEVYRWEH |
| | | STKMKSKTTPVDLESEDIEHEELSDEWEDMQEGYKTLLRD |
| | | PSGFFWSSDSWIPQKDFWIRVKSRIGKSLREQIR |
| 29. | GAPDH N-terminal LHA- | GGAGAAGTTCCCCAACTTTCCCGCCTCTCAGCCTTTGAA |
| | start codon-degron- | AGAAAGAAAGGGGAGGGGGCAGGCCGCGTGCAGCCGC |
| | linker 1-GAPDH N- | GAGCGGTGCTGGGCTCCGGCTCCAATTCCCCATCTCAG |
| | terminal RHA nucleic | TCGTTCCCAAAGTCCTCCTGTTTCATCCAAGCGTGTAAG |
| | acid sequence | GGTCCCCGTCCTTGACTCCCTAGTGTCCTGCTGCCCACA |
| | | GTCCAGTCCTGGGAACCAGCACCGATCACCTCCCATCG |
| | | GGCCAATCTCAGTCCCTTCCCCCCTACGTCGGGGCCCA |
| | | CACGCTCGGTGCGTGCCCAGTTGAACCAGGCGGCTGC |
| | | GGAAAAAAAAAAGCGGGGAGAAAGTAGGGCCCGGCTAC |
| | | TAGCGGTTTTACGGGCGCACGTAGCTCAGGCCTCAAGA |
| | | CCTTGGGCTGGGACTGGCTGAGCCTGGCGGGAGGCGG |
| | | GGTCCGAGTCACCGCCTGCCGCCGCGCCCCCGGTTTCT |
| | | ATAAATTGAGCCCGCAGCCTCCCGCTTCGCTCTCTGCTC |
| | | CTCCTGTTCGACAGTCAGCCGCATCTTCTTTTGCGTCGC |
| | | CAGGTGAAGACGGGCGGAGAGAAACCCGGGAGGCTAG |
| | | GGACGGCCTGAAGGCGGCAGGGGGGGGCGCAGGCCG |
| | | GATGTGTTCGCGCCGCTGCGGGGTGGGCCCGGGCGGC |
| | | CTCCGCATTGCAGGGGGGGGCGGAGGACGTGATGCGG |
| | | CGCGGGCTGGGCATGGAGGCCTGGTGGGGGAGGGGA |
| | | GGGGAGGCGTGTGTGTCGGCCGGGGCCACTAGGCGCT |
| | | CACTGTTCTCTCCCTCCGCGCAGCCGAGCCACATCGCT |
| | | CAGACACCATGCGCCCATTCCAGTGTAATCAGTGTGGGG |
| | | CATCTTTTACTCAGAAAGGTAACCTCCTCCGCCACATTAA |
| | | ACTGCACGGCGGGTCTGGGAAGGTGAAGGTCGGAGTC |
| | | AACGGGTGAGTTCGCGAATGGCTGGGGGGCCCTGGGC |
| | | TGCGACCGCCCCCGAACCGCGTCTACGAGCCTTGCGGG |
| | | CTCCGGGTCTTAGCAGTCGTATGGGGGCAGGGTAGCTG |
| | | TTCCCCGCAAGGAGAGCTCAAGGTCAGCGCTCGGACCT |
| | | GGCGGAGCCCCGCACCCAGGCTGTGGCGCCCTGTGCA |
| | | GCTCCGCCCTTGCGGCGCCATCTGCCCGGAGCCTCCTT |
| | | CCCCTAGTCCCCAGAAACAGGAGGTCCCTACTCCCGCC |
| | | CGAGATCCCGACCCGGACCCCTAGGTGGGGGACGCTTT |
| | | CTTTCCTTTCGCGCTCTGCGGGGTCACGTGTCGCAGAG |
| | | GAGCCCCTCCCCCACGGCCTCCGGCACCGCAGGCCCC |
| | | GGGATGCTAGTGCGCAGCGGGTGCATCCCTGTCCGGAT |
| | | GCTGCGCCTGCGGTAGAGCGGCCGCCATGTTGCAACCG |
| | | GGAAGGAAATGAATGGGCAGCCGTTAGGAAAGCCTGCC |
| | | GGTGACTAACCCTGCGCTCCTGCCTCGATGGGTGGAGT |
| | | CGCGTGTGGCGGGGAAGTCAGGTGGAGCGAGGCTAGC |
| | | TGGCCCGATTTCTCCTCCGGGTGATGCTTTTCCTAGATTA |
| | | TTCTCTGGTAAATCAAAGAAGTGGGTTTATGGAGGTCCTC |
| | | TTGTGTCCCCTCCCCGCAGAGGTGTGGTGGCTGTGGCA |
| | | TGGTGCCAAGCCGGGAGAAGCTGAGTCATGGGTAGTTG |
| | | GAAAAGGACATTTCCACCGCAAAATGGCCCCTCTGGTGG |
| | | TGGCCCCTTCCTGCAGCG |
| 30. | GAPDH N-terminal LHA- | GGAGAAGTTCCCCAACTTTCCCGCCTCTCAGCCTTTGAA |
| | start codon- | AGAAAGAAAGGGGAGGGGGCAGGCCGCGTGCAGCCGC |
| | superdegron-linker 4- | GAGCGGTGCTGGGCTCCGGCTCCAATTCCCCATCTCAG |
| | GAPDH N-terminal RHA | TCGTTCCCAAAGTCCTCCTGTTTCATCCAAGCGTGTAAG |
| | nucleic acid sequence | GGTCCCCGTCCTTGACTCCCTAGTGTCCTGCTGCCCACA |
| | | GTCCAGTCCTGGGAACCAGCACCGATCACCTCCCATCG |
| | | GGCCAATCTCAGTCCCTTCCCCCCTACGTCGGGGCCCA |
| | | CACGCTCGGTGCGTGCCCAGTTGAACCAGGCGGCTGC |
| | | GGAAAAAAAAAAGCGGGGAGAAAGTAGGGCCCGGCTAC |
| | | TAGCGGTTTTACGGGCGCACGTAGCTCAGGCCTCAAGA |
| | | CCTTGGGCTGGGACTGGCTGAGCCTGGGGGGAGGCGG |
| | | GGTCCGAGTCACCGCCTGCCGCCGCGCCCCCGGTTTCT |
| | | ATAAATTGAGCCCGCAGCCTCCCGCTTCGCTCTCTGCTC |
| | | CTCCTGTTCGACAGTCAGCCGCATCTTCTTTTGCGTCGC |
| | | CAGGTGAAGACGGGCGGAGAGAAACCCGGGAGGCTAG |
| | | GGACGGCCTGAAGGCGGCAGGGGGGGGCGCAGGCCG |
| | | GATGTGTTCGCGCCGCTGCGGGGTGGGCCCGGGCGGC |
| | | CTCCGCATTGCAGGGGGGGGCGGAGGACGTGATGCGG |
| | | CGCGGGCTGGGCATGGAGGCCTGGTGGGGGAGGGGA |
| | | GGGGAGGCGTGTGTGTCGGCCGGGGCCACTAGGCGCT |
| | | CACTGTTCTCTCCCTCCGCGCAGCCGAGCCACATCGCT |
| | | CAGACACCATGTTCAATGTACTGATGGTCCATAAACGGAG |
| | | TCACACTGGCGAGCGCCCGCTCCAATGTGAAATCTGCG |
| | | GGTTCACGTGTCGGCAGAAGGGCAACCTCCTCCGGCAT |
| | | ATCAAGCTGCACACGGGTGAAAAACCGTTTAAGTGCCAT |
| | | CTCTGCAATTACGCCTGTCAGAGAAGAGATGCTTTGGGC |
| | | TCAGGTAGCGGAAGCGGATCAGGTGGAGGGAAGGTGA |
| | | AGGTCGGAGTCAACGGGTGAGTTCGCGAATGGCTGGGG |
| | | GGCCCTGGGCTGCGACCGCCCCCGAACCGCGTCTACG |
| | | AGCCTTGCGGGCTCCGGGTCTTAGCAGTCGTATGGGGG |
| | | CAGGGTAGCTGTTCCCCGCAAGGAGAGCTCAAGGTCAG |
| | | CGCTCGGACCTGGCGGAGCCCCGCACCCAGGCTGTGG |
| | | CGCCCTGTGCAGCTCCGCCCTTGCGGCGCCATCTGCCC |
| | | GGAGCCTCCTTCCCCTAGTCCCCAGAAACAGGAGGTCC |
| | | CTACTCCCGCCCGAGATCCCGACCCGGACCCCTAGGTG |
| | | GGGGACGCTTTCTTTCCTTTCGCGCTCTGCGGGGTCAC |
| | | GTGTCGCAGAGGAGCCCCTCCCCCACGGCCTCCGGCA |
| | | CCGCAGGCCCCGGGATGCTAGTGCGCAGCGGGTGCATC |
| | | CCTGTCCGGATGCTGCGCCTGCGGTAGAGCGGCCGCCA |
| | | TGTTGCAACCGGGAAGGAAATGAATGGGCAGCCGTTAG |
| | | GAAAGCCTGCCGGTGACTAACCCTGCGCTCCTGCCTCG |
| | | ATGGGTGGAGTCGCGTGTGGCGGGGAAGTCAGGTGGA |
| | | GCGAGGCTAGCTGGCCCGATTTCTCCTCCGGGTGATGC |
| | | TTTTCCTAGATTATTCTCTGGTAAATCAAAGAAGTGGGTTT |
| | | ATGGAGGTCCTCTTGTGTCCCCTCCCCGCAGAGGTGTG |
| | | GTGGCTGTGGCATGGTGCCAAGCCGGGAGAAGCTGAGT |
| | | CATGGGTAGTTGGAAAAGGACATTTCCACCGCAAAATGG |
| | | CCCCTCTGGTGGTGGCCCCTTCCTGCAGCG |
| 31 | RPL13A N-terminal LHA- | CACAGGAATCCAAATCTCTAAGCCTAGAATGGGCAAGGC |
| | start codon-degron- | TCCGGGGCTCACGCCGGTAATCCCAGCACTTTGGGAGG |
| | linker 1-RPL13A N- | CCGAAACGGGAGGATCGCTTGAGCCCAGGAGTTCGAGA |
| | terminal RHA nucleic | TCAGCCTGGCCAACATGTCAGACTCCCCCCGTTCCCGG |
| | acid sequence | GCCACAACAAAATATATATATATATATATATTAGCCAGGCAT |
| | | GGTGGCGCGCCCCTGTAGTCCAAGTTACTCGAGAGGCT |
| | | GAGGCAGGAGGATCCCTTGAGCCCAGGAAGTGGAGGCT |
| | | GCGGTGAGCCATGATCGCCCCACAGCACTCTGGTGTGG |
| | | ACAACACAGCGAGACCCTGTCTCACAAAATAAAGTAAGC |
| | | CCGGACTGAGTGCGGAAAGGCGGGCCTGGCGGGTCTG |
| | | GTCTCCCCATGCGGGCCACCAGAGGCCCTGCAGCCTTC |
| | | AGTCGCTTGAAGGGGTAATGGCGCTTCCACTCACAAACA |
| | | TGGCGGACAGAGCGTGTGAACGAGATGAACAGCCCCTC |
| | | AAAAATATGGCCGCCGAGGCTGGACGGCCGTGCCCCAG |
| | | CAGCACCGCCTCCGCGCCCCACGTGATCTCTCGCCGGG |
| | | CACAGCGCTGACCGCGGAGGTCCAACCGGAAGAATGTC |
| | | CGGATTGGACATTCGGAAGAGGGCCCGCCTTCCCTGGG |
| | | GAATCTCTGCGCACGCGCAGAACGCTTCGACCAATGAAA |
| | | ACACAGGAAGCCGTCCGCGCAACCGCGTTGCGTCACTT |
| | | CTGCCGCCCCTGTTTCAAGGGATAAGAAACCCTGCGACA |
| | | AAACCTCCTCCTTTTCCAAGCGGCTGCCGAAGATGCGCC |
| | | CATTCCAGTGTAATCAGTGTGGGGCATCTTTTACTCAGAA |
| | | AGGTAACCTCCTCCGCCACATTAAACTGCACGGCGGGTC |
| | | TGCGGAGGTGCAGGTATGGGCTCCGCGCGGGCCGGGG |
| | | CGGCAAGGGGCCGGGTGGGATCCAGGCCGGAATGGGG |
| | | TCGCACCCTCTCTGTCTTGCATGTTTTGCGGAGCTTAAC |
| | | ATCCATAATGAAGCAAAATGGAAGTCTTTTGGGATAAGGG |
| | | GAATCTGTAGGAAATGCCGCGGCTCTCTTTAGGCCTAGG |
| | | GTTGGCTACAATGTGGCATTTCCTTCTCGAGGCGAGGGT |
| | | GATAGAGCTTCCAGCACAGGACAGGTATTTTGCAACTAG |
| | | ATTTTGCTTACCTTGAACTCGGGTAGTGGGTGGGGGCCC |
| | | TGGGGTAGAGTCTTGGCCGAATTGGACCCTTGTGTGTCC |
| | | TCAGCGATGAGGAACACGCGGAAGTGTGGCCGGGCGG |
| | | TCTCGGGGCAATGAGAAACTGAGTTTTGGCCAAAATGGA |
| | | AGGTATTTTTATCCCGCTCAGTGCGCGGGCTACTTTGATT |
| | | TCACGTGGAGGTGTTAATCGGGGTCCCCTGCAGCTCCG |
| | | GTTCTTTTAATTCTCAATGCCTTTCTGCGGGGGGTCGAG |
| | | AGCTGTTGGCTGCGGGGCACTCTGGAATGAACTCGTTTT |
| | | CCGTCGTTTTGGGGATTGGCGATTGTCCTGAGGCGTGTT |
| | | ACTGGAAGTCGAGAGCTGTGATGAATGGTTCCACAAGGA |
| | | TGGGACATCTTTTGGGCCTGACACCTGGGGGCCCTTTC |
| | | CCTGGCTCCTGGTGTAGCTACTTAAGTATAAAGGAGGGA |
| | | TTGGCCCTGGAAAGGTTTCTTTGTGAGTAGTCTGTTGTT |
| | | GGGGGGGGTGACATTTCAGAGCCAGGC |
| 32. | RPL13A N-terminal LHA- | CACAGGAATCCAAATCTCTAAGCCTAGAATGGGCAAGGC |
| | start codon- | TCCGGGGCTCACGCCGGTAATCCCAGCACTTTGGGAGG |
| | superdegron-linker 4- | CCGAAACGGGAGGATCGCTTGAGCCCAGGAGTTCGAGA |
| | RPL13A N-terminal RHA | TCAGCCTGGCCAACATGTCAGACTCCCCCCGTTCCCGG |
| | nucleic acid sequence | GCCACAACAAAATATATATATATATATATATTAGCCAGGCAT |
| | | GGTGGCGCGCCCCTGTAGTCCAAGTTACTCGAGAGGCT |
| | | GAGGCAGGAGGATCCCTTGAGCCCAGGAAGTGGAGGCT |
| | | GCGGTGAGCCATGATCGCCCCACAGCACTCTGGTGTGG |
| | | ACAACACAGCGAGACCCTGTCTCACAAAATAAAGTAAGC |
| | | CCGGACTGAGTGCGGAAAGGCGGGCCTGGCGGGTCTG |
| | | GTCTCCCCATGCGGGCCACCAGAGGCCCTGCAGCCTTC |
| | | AGTCGCTTGAAGGGGTAATGGCGCTTCCACTCACAAACA |
| | | TGGCGGACAGAGCGTGTGAACGAGATGAACAGCCCCTC |
| | | AAAAATATGGCCGCCGAGGCTGGACGGCCGTGCCCCAG |
| | | CAGCACCGCCTCCGCGCCCCACGTGATCTCTCGCCGGG |
| | | CACAGCGCTGACCGCGGAGGTCCAACCGGAAGAATGTC |
| | | CGGATTGGACATTCGGAAGAGGGCCCGCCTTCCCTGGG |
| | | GAATCTCTGCGCACGCGCAGAACGCTTCGACCAATGAAA |
| | | ACACAGGAAGCCGTCCGCGCAACCGCGTTGCGTCACTT |
| | | CTGCCGCCCCTGTTTCAAGGGATAAGAAACCCTGCGACA |
| | | AAACCTCCTCCTTTTCCAAGCGGCTGCCGAAGATGTTCA |
| | | ATGTACTGATGGTCCATAAACGGAGTCACACTGGCGAGC |
| | | GCCCGCTCCAATGTGAAATCTGCGGGTTCACGTGTCGG |
| | | CAGAAGGGCAACCTCCTCCGGCATATCAAGCTGCACAC |
| | | GGGTGAAAAACCGTTTAAGTGCCATCTCTGCAATTACGC |
| | | CTGTCAGAGAAGAGATGCTTTGGGCTCAGGTAGCGGAA |
| | | GCGGATCAGGTGGAGCGGAGGTGCAGGTATGGGCTCC |
| | | GCGCGGGCCGGGGCGGCAAGGGGCCGGGTGGGATCC |
| | | AGGCCGGAATGGGGTCGCACCCTCTCTGTCTTGCATGTT |
| | | TTGCGGAGCTTAACATCCATAATGAAGCAAAATGGAAGTC |
| | | TTTTGGGATAAGGGGAATCTGTAGGAAATGCCGCGGCTC |
| | | TCTTTAGGCCTAGGGTTGGCTACAATGTGGCATTTCCTTC |
| | | TCGAGGCGAGGGTGATAGAGCTTCCAGCACAGGACAGG |
| | | TATTTTGCAACTAGATTTTGCTTACCTTGAACTCGGGTAGT |
| | | GGGTGGGGGCCCTGGGGTAGAGTCTTGGCCGAATTGGA |
| | | CCCTTGTGTGTCCTCAGCGATGAGGAACACGCGGAAGT |
| | | GTGGCCGGGCGGTCTCGGGGCAATGAGAAACTGAGTTT |
| | | TGGCCAAAATGGAAGGTATTTTTATCCCGCTCAGTGCGC |
| | | GGGCTACTTTGATTTCACGTGGAGGTGTTAATCGGGGTC |
| | | CCCTGCAGCTCCGGTTCTTTTAATTCTCAATGCCTTTCTG |
| | | CGGGGGGTCGAGAGCTGTTGGCTGCGGGGCACTCTGG |
| | | AATGAACTCGTTTTCCGTCGTTTTGGGGATTGGCGATTGT |
| | | CCTGAGGCGTGTTACTGGAAGTCGAGAGCTGTGATGAAT |
| | | GGTTCCACAAGGATGGGACATCTTTTGGGCCTGACACCT |
| | | GGGGGCCCTTTCCCTGGCTCCTGGTGTAGCTACTTAAGT |
| | | ATAAAGGAGGGATTGGCCCTGGAAAGGTTTCTTTGTGAG |
| | | TAGTCTGTTGTTGGGGGGGGTGACATTTCAGAGCCAGG |
| | | C |
| 33 | RPLP0 N-terminal LHA- | GCGTGAACCCGGGAGGCGGAGCTTGCAGTCAGCCGAG |
| | start codon-degron- | ATGGCGCCACTGCGCTCCAGCCTGGGCGACAGAGCGA |
| | linker 1-RPLP0 N- | CACTCCGTCTCAAAAAAAAAAAAAAAAAGAGAAACCATTC |
| | terminal RHA nucleic | TGTTTCTAAGCATGTGCAATTATGTCTCAGCTCCACGTCA |
| | acid sequence | TACCTTTTAGTTTGCTGAGCTCGCCAGGTGGCACAGGGA |
| | | ACCGGGACTCCGAATTCGCAGCTCCAGCTCCAGCTCCA |
| | | GCCCAGCAGGTGGCAGCAGCTCAGAGCAAGCTCCTCCA |
| | | ACGCGAGGCAGCGCCTTCCTTCGCGACCCTACTTAAAG |
| | | GCGGCTTTCCGTTGGCTGGAGTGGCAGCGATATTATCCA |
| | | ATGGTTGCCTGTATTCGTTCAGCTTTGTCTGACGGGCGA |
| | | TGGCGCAGCCAATAGACAGGAGCGCTATCCGCGGTTTCT |
| | | GATTGGCTACTTTGTTCGCATTATAAAAGGCACGCGCGG |
| | | GCGCGAGGCCCTTCTCTCGCCAGGCGTCCTCGTGGAAG |
| | | GTTCGTGTGCTAGTTAGATGGGCGCCAGGGGTCGCCGG |
| | | CGGGAAGCATGGAGGGGTCTTTGGGGGCCTTTGGGAAC |
| | | ATGGAGTCCTATTCTGTTCCGCCTGGGGCCTCGGTGGC |
| | | GGCTTGCACGCCCCGAGATGACGGCCGCTGCCCTAGGC |
| | | AGGGCCGGCGGGCGATTGCGCGTGTCCTGCTCCTCTTA |
| | | GGCCCGGGACCGCGGGATGGGTGTCGGCGTGACCAGG |
| | | CCTGAGCTCCCTGTCTCTCCTCAGTGACATCGTCTTTAAA |
| | | CCCTGCGTGGCAATCCCTGACGCACCGCCGTGATGCGC |
| | | CCATTCCAGTGTAATCAGTGTGGGGCATCTTTTACTCAGA |
| | | AAGGTAACCTCCTCCGCCACATTAAACTGCACGGCGGGT |
| | | CTCCCAGGGAAGACAGGGCGACCTGGAAGTCCAACTAC |
| | | TTCCTTAAGATCATCGTAAGTGCAGGGTGGGTCGCCTCT |
| | | GCTCTTCATGTTGCCCCAGCGCAAATAGGGACAGTCAGC |
| | | TGCTATGTGCTGAGGGTCTACTCACACCGGCTACTGAAT |
| | | TAGGCCATTTTTGGGAAAATACTGTTTAGTTAACAATTTCC |
| | | TGAGATAGGTCCCTTCTGTTGCAGATAAACGGGCTCAGG |
| | | CAAGTTAAGTGGGTCCTAAGATGACAGCATTCGTATCCAG |
| | | GTCTGTCTGGCTTCTAAAAGAGCGCGCTTTATACTTTTTT |
| | | TTTTTTAAACGGAGTCTCGCTCTGTTGTCCAGGCTGGAG |
| | | TGCAGTGGCTTGATCTTGGCTTACTGCAACCTCCGCTTC |
| | | CCAGGTTCAAGCGATTCTCCTGCTTCAGCCTCCCGAGTA |
| | | GCTGGGATTACAGGGGCGCGACACCACACCCAGCTAAT |
| | | TTTTGTATTTTCAATAGAGACAGGGATTCACCATGTTGGC |
| | | CAGGATGGTCTCGATCTCTTGACTTCATGATCCACCTTCC |
| | | TCGGCCTCCCTAAGTGCTAGGATTACAGGTGTGAGCCAC |
| | | CGCGCCCGGCCGCACATTACAGTTTTACTCCATTTTTGA |
| | | GAAGGTCTGAGGTCAGGAATGACTCATTGTAAGGAAGCA |
| | | AGCTGTTGCATATTTAGGACCTGTCAGCCAAGAAACTTAA |
| | | GTGTCCAATGACTTTTGGATGGCCTTTGAGGTGGGTCTC |
| | | CCTTGCCCAGAAATGCTGACTGTACTGCGCTTAAGCAAT |
| | | TAGGCAGCATTGTAGAG |
| 34 | RPLPO N-terminal LHA- | GCGTGAACCCGGGAGGCGGAGCTTGCAGTCAGCCGAG |
| | start codon- | ATGGCGCCACTGCGCTCCAGCCTGGGCGACAGAGCGA |
| | superdegron-linker 4- | CACTCCGTCTCAAAAAAAAAAAAAAAAAGAGAAACCATTC |
| | RPLPO N-terminal RHA | TGTTTCTAAGCATGTGCAATTATGTCTCAGCTCCACGTCA |
| | nucleic acid sequence | TACCTTTTAGTTTGCTGAGCTCGCCAGGTGGCACAGGGA |
| | | ACCGGGACTCCGAATTCGCAGCTCCAGCTCCAGCTCCA |
| | | GCCCAGCAGGTGGCAGCAGCTCAGAGCAAGCTCCTCCA |
| | | ACGCGAGGCAGCGCCTTCCTTCGCGACCCTACTTAAAG |
| | | GCGGCTTTCCGTTGGCTGGAGTGGCAGCGATATTATCCA |
| | | ATGGTTGCCTGTATTCGTTCAGCTTTGTCTGACGGGCGA |
| | | TGGCGCAGCCAATAGACAGGAGCGCTATCCGCGGTTTCT |
| | | GATTGGCTACTTTGTTCGCATTATAAAAGGCACGCGCGG |
| | | GCGCGAGGCCCTTCTCTCGCCAGGCGTCCTCGTGGAAG |
| | | GTTCGTGTGCTAGTTAGATGGGCGCCAGGGGTCGCCGG |
| | | CGGGAAGCATGGAGGGGTCTTTGGGGGCCTTTGGGAAC |
| | | ATGGAGTCCTATTCTGTTCCGCCTGGGGCCTCGGTGGC |
| | | GGCTTGCACGCCCCGAGATGACGGCCGCTGCCCTAGGC |
| | | AGGGCCGGCGGGCGATTGCGCGTGTCCTGCTCCTCTTA |
| | | GGCCCGGGACCGCGGGATGGGTGTCGGCGTGACCAGG |
| | | CCTGAGCTCCCTGTCTCTCCTCAGTGACATCGTCTTTAAA |
| | | CCCTGCGTGGCAATCCCTGACGCACCGCCGTGATGTTC |
| | | AATGTACTGATGGTCCATAAACGGAGTCACACTGGCGAG |
| | | CGCCCGCTCCAATGTGAAATCTGCGGGTTCACGTGTCG |
| | | GCAGAAGGGCAACCTCCTCCGGCATATCAAGCTGCACA |
| | | CGGGTGAAAAACCGTTTAAGTGCCATCTCTGCAATTACG |
| | | CCTGTCAGAGAAGAGATGCTTTGGGCTCAGGTAGCGGA |
| | | AGCGGATCAGGTGGACCCAGGGAAGACAGGGCGACCT |
| | | GGAAGTCCAACTACTTCCTTAAGATCATCGTAAGTGCAGG |
| | | GTGGGTCGCCTCTGCTCTTCATGTTGCCCCAGCGCAAAT |
| | | AGGGACAGTCAGCTGCTATGTGCTGAGGGTCTACTCACA |
| | | CCGGCTACTGAATTAGGCCATTTTTGGGAAAATACTGTTT |
| | | AGTTAACAATTTCCTGAGATAGGTCCCTTCTGTTGCAGAT |
| | | AAACGGGCTCAGGCAAGTTAAGTGGGTCCTAAGATGACA |
| | | GCATTCGTATCCAGGTCTGTCTGGCTTCTAAAAGAGCGC |
| | | GCTTTATACTTTTTTTTTTTTAAACGGAGTCTCGCTCTGTT |
| | | GTCCAGGCTGGAGTGCAGTGGCTTGATCTTGGCTTACTG |
| | | CAACCTCCGCTTCCCAGGTTCAAGCGATTCTCCTGCTTC |
| | | AGCCTCCCGAGTAGCTGGGATTACAGGGGCGCGACACC |
| | | ACACCCAGCTAATTTTTGTATTTTCAATAGAGACAGGGATT |
| | | CACCATGTTGGCCAGGATGGTCTCGATCTCTTGACTTCA |
| | | TGATCCACCTTCCTCGGCCTCCCTAAGTGCTAGGATTACA |
| | | GGTGTGAGCCACCGCGCCCGGCCGCACATTACAGTTTT |
| | | ACTCCATTTTTGAGAAGGTCTGAGGTCAGGAATGACTCA |
| | | TTGTAAGGAAGCAAGCTGTTGCATATTTAGGACCTGTCAG |
| | | CCAAGAAACTTAAGTGTCCAATGACTTTTGGATGGCCTTT |
| | | GAGGTGGGTCTCCCTTGCCCAGAAATGCTGACTGTACTG |
| | | CGCTTAAGCAATTAGGCAGCATTGTAGAG |
| 35. | Rigid linker 1 | PAPAP |
| 36. | Rigid linker 2 | AEAAAKEAAAKA |
| 37. | Rigid linker 3 | A(EAAAK)4ALEA(EAAAK)4A |
| 38. | GAPDH-targeting Cpf1 | UGAGCCAGCCACCAGAGGGCG |
| | gRNA sequence 1 | |
| 39. | GAPDH-targeting Cpf1 | AUCUUCUAGGUAUGACAACGA |
| | gRNA sequence 2 | |
| 40. | GAPDH-targeting Cpf1 | GCUACAGCAACAGGGUGGUGG |
| | gRNA sequence 3 | |
| 41. | GAPDH-targeting Cpf1 | CCAUAAUUUCCUUUCAAGGUG |
| | gRNA sequence 4 | |
| 42. | GAPDH-targeting Cpf1 | CUUUCAAGGUGGGGAGGGAGG |
| | gRNA sequence 5 | |
| 43. | GAPDH-targeting Cpf1 | AAGGUGGGGAGGGAGGUAGAG |
| | gRNA sequence 6 | |
| 44. | GAPDH-targeting Cpf1 | GCAGACCACAGUCCAUGCCAU |
| | gRNA sequence 7 | |
| 45. | GAPDH-targeting Cpf1 | CAGACCACAGUCCAUGCCAUC |
| | gRNA sequence 8 | |
| 46. | GAPDH-targeting Cpf1 | CCGGAGGGGCCAUCCACAGUC |
| | gRNA sequence 9 | |
| 47 | GAPDH-targeting Cpf1 | UAGACGGCAGGUCAGGUCCAC |
| | gRNA sequence 10 | |
| 48. | GAPDH-targeting Cpf1 | CUAGACGGCAGGUCAGGUCCA |
| | gRNA sequence 11 | |
| 49. | GAPDH-targeting Cpf1 | UCUAGACGGCAGGUCAGGUCC |
| | gRNA sequence 12 | |
| 50. | GAPDH-targeting Cpf1 | GCAGGUUUUUCUAGACGGCAG |
| | gRNA sequence 13 | |
| 51. | GAPDH-targeting Cpf1 | UCAAGCUCAUUUCCUGGUAUG |
| | gRNA sequence 14 | |
| 52. | GAPDH-targeting Cpf1 | CUGGUAUGUGGCUGGGGCCAG |
| | gRNA sequence 15 | |
| 53. | GAPDH-targeting Cpf1 | AGAGCCAGUCUCUGGCCCCAG |
| | gRNA sequence 16 | |
| 54. | GAPDH-targeting Cpf1 | AAGAGCCAGUCUCUGGCCCCA |
| | gRNA sequence 17 | |
| 55. | GAPDH-targeting Cpf1 | UAAGAGCCAGUCUCUGGCCCC |
| | gRNA sequence 18 | |
| 56. | GAPDH-targeting Cpf1 | CUGAGCCAGCCACCAGAGGGC |
| | gRNA sequence 19 | |
| 57. | GAPDH-targeting Cpf1 | UCUGAGCCAGCCACCAGAGGG |
| | gRNA sequence 20 | |
| 58. | GAPDH-targeting Cpf1 | CAUCUUCUAGGUAUGACAACG |
| | gRNA sequence 21 | |
| 59. | GAPDH-targeting Cpf1 | UUGAUGGUACAUGACAAGGUG |
| | gRNA sequence 22 | |
| 60. | GAPDH-targeting Cpf1 | GAGGCCCUACCCUCAGUCUGA |
| | gRNA sequence 23 | |
| 61. | GAPDH-targeting Cpf1 | CCUCUCCUCGCUCCAGUCCUA |
| | gRNA sequence 24 | |
| 62. | GAPDH-targeting Cpf1 | CUCUCCUCGCUCCAGUCCUAG |
| | gRNA sequence 25 | |
| 63. | GAPDH-targeting Cpf1 | GCCAACAGCAGAUAGCCUAGG |
| | gRNA sequence 26 | |
| 64. | GAPDH-targeting Cpf1 | UGUGCCCUCGUGUCUUAUCUG |
| | gRNA sequence 27 | |
| 65. | GAPDH-targeting Cpf1 | CCUAGAUGAAUCCUGCUUGAA |
| | gRNA sequence 28 | |
| 66. | GAPDH-targeting Cpf1 | GGUACUUGGUUUACCUAGAUG |
| | gRNA sequence 29 | |
| 67. | GAPDH-targeting Cpf1 | AGGUACUUGGUUUACCUAGAU |
| | gRNA sequence 30 | |
| 68. | GAPDH-targeting Cpf1 | AAACAUUAUAUAGUCCUUACC |
| | gRNA sequence 31 | |
| 69. | GAPDH-targeting Cpf1 | UAAACAUUAUAUAGUCCUUAC |
| | gRNA sequence 32 | |
| 70. | GAPDH-targeting Cpf1 | CCGAUUUUUAAACAUUAUAUA |
| | gRNA sequence 33 | |
| 71. | GAPDH-targeting Cpf1 | ACCGAUUUUUAAACAUUAUAU |
| | gRNA sequence 34 | |
| 72. | GAPDH-targeting Cpf1 | UACCGAUUUUUAAACAUUAUA |
| | gRNA sequence 35 | |
| 73. | GAPDH-targeting Cpf1 | AAAAUCGGUAAAAAUGCCCAC |
| | gRNA sequence 36 | |
| 74. | GAPDH-targeting Cpf1 | GAGGAAGAUGAACUGAGAUGU |
| | gRNA sequence 37 | |
| 75. | GAPDH-targeting Cpf1 | AGGAAGAUGAACUGAGAUGUG |
| | gRNA sequence 38 | |
| 76. | GAPDH Cas9 gRNA 1 | CUUCCUCUUGUGCUCUUGCU |
| 77. | GAPDH Cas9 gRNA 2 | CCUCCAAGGAGUAAGACCCC |
| 78. | GAPDH Cas9 gRNA 3 | CAUGGCCCACAUGGCCUCCA |
| 79. | GAPDH Cas9 gRNA 4 | AGCCCCAGCAAGAGCACAAG |
| 80. | RPL13A Cas9 sgRNA 1 | GGAAGGGCAGGCAACGCAUG |
| 81. | RPL13A Cas9 sgRNA 2 | GGCUCAGACCAGGAGUCCGU |
| 82. | RPL13A Cas9 sgRNA 3 | CCUCAAGACCCACGGACUCC |
| 83. | RPL13A Cas9 sgRNA 4 | GUCUUUAUUGGGCUCAGACC |
| 84. | RPL13A Cas9 sgRNA 5 | ACAUUCCAGGGCAACAAUGG |
| 85. | RPL13A Cas9 sgRNA 6 | UUGGCAGAGGCUACGGAAAC |
| 86. | RPL13A Cas9 sgRNA 7 | CUUAUUCUUGGCAGAGGCUA |
| 87. | RPLP0 Cas9 sgRNA 1 | GCAAAUAAAACUGGCUAAGU |
| 88. | RPLP0 Cas9 sgRNA 2 | UUACUUCUUUAAAAAGUCUC |
| 89. | RPLP0 Cas9 sgRNA 3 | UUUGCAAAACAAGGAAAUAA |
| 90. | RPLP0 Cpf1 sgRNA 1 | UUUCCUUGUUUUGCAAAUAAAAC |
| 91. | RPLP0 Cpf1 sgRNA 2 | CAAAUAAAACUGGCUAAGUUGGU |
| 92. | RPLP0 Cpf1 sgRNA 3 | CUUGUUUUGCAAAUAAAACUGGC |
| 93. | RPLP0 Cpf1 sgRNA 4 | GUGAUUAGUCAAAGAGACCAAAU |
| 94. | RPLP0 Cpf1 sgRNA 5 | ACUAAUCACCAAAAAGCAACCAA |
| 95. | RPLP0 Cpf1 sgRNA 6 | AGGUCAAGGCCUUCUUGGCUG |
| 96. | RPLP0 Cpf1 sgRNA 7 | CAAAUAAAACUGGCUAAGUUG |
| 97. | RPL7 Cas9 sgRNA 1 | ACUCAACCUUUCUCAGGAUG |
| 98. | RPL7 Cas9 sgRNA 2 | ACAUUAACUCAACCUUUCUC |
| 99. | RPL7 Cas9 sgRNA 3 | AGUUAAUGUUAGAGUAUAAG |
| 100. | GAPDH amino acid | MGKVKVGVNGFGRIGRLVTRAAFNSGKVDIVAINDPFIDLN |
| | sequence | YMVYMFQYDSTHGKFHGTVKAENGKLVINGNPITIFQERDP |
| | (UniProt #P04406) | SKIKWGDAGAEYVVESTGVFTTMEKAGAHLQGGAKRVIISA |
| | | PSADAPMFVMGVNHEKYDNSLKIISNASCTTNCLAPLAKVI |
| | | HDNFGIVEGLMTTVHAITATQKTVDGPSGKLWRDGRGALQ |
| | | NIIPASTGAAKAVGKVIPELNGKLTGMAFRVPTANVSVVDLT |
| | | CRLEKPAKYDDIKKVVKQASEGPLKGILGYTEHQVVSSDFN |
| | | SDTHSSTFDAGAGIALNDHFVKLISWYDNEFGYSNRVVDL |
| | | MAHMASKE |
| 101. | RPL13A amino acid | MAEVQVLVLDGRGHLLGRLAAIVAKQVLLGRKVVVVRCEGI |
| | sequence | NISGNFYRNKLKYLAFLRKRMNTNPSRGPYHFRAPSRIFW |
| | (UniProt #P40429) | RTVRGMLPHKTKRGQAALDRLKVFDGIPPPYDKKKRMVVP |
| | | AALKVVRLKPTRKFAYLGRLAHEVGWKYQAVTATLEEKRK |
| | | EKAKIHYRKKKQLMRLRKQAEKNVEKKIDKYTEVLKTHGLL |
| | | V |
| 102. | RPLP0 amino acid | MPREDRATWKSNYFLKIIQLLDDYPKCFIVGADNVGSKQM |
| | sequence | QQIRMSLRGKAVVLMGKNTMMRKAIRGHLENNPALEKLLP |
| | (UniProt #P05388) | HIRGNVGFVFTKEDLTEIRDMLLANKVPAAARAGAIAPCEVT |
| | | VPAQNTGLGPEKTSFFQALGITTKISRGTIEILSDVQLIKTGD |
| | | KVGASEATLLNMLNISPFSFGLVIQQVFDNGSIYNPEVLDIT |
| | | EETLHSRFLEGVRNVASVCLQIGYPTVASVPHSIINGYKRVL |
| | | ALSVETDYTFPLAEKVKAFLADPSAFVAAAPVAAATTAAPA |
| | | AAAAPAKVEAKEESEESDEDMGFGLFD |
| 103. | Exemplary linker I | (GGS)9 |
| | (Linker 3) | |
| 104. | RPLPO C-terminal LHA- | CTGAGCTGCCAACCTGGCAATTATTGTCTGCTAAGGGTT |
| | linker 1-degron-stop | CTCTTTATTCACCCTTACTTGGACTTCCTTTCCTGTAGGG |
| | codon-RPLP0 C- | AATCTCACGTAAAATGAAATCTTCCCTCCCCCAGGGTGT |
| | terminal RHA | CCGCAATGTTGCCAGTGTCTGTCTGCAGATTGGCTACCC |
| | | AACTGTTGCATCAGTACCCCATTCTATCATCAACGGGTA |
| | | CAAACGAGTCCTGGCCTTGTCTGTGGAGACGGATTACA |
| | | CCTTCCCACTTGCTGAAAAGGTAAAAGGATCCCACCAGG |
| | | ACCACAGTGGGCCTGACTGTGACAAATTAGCAGGGTGA |
| | | TGTGGCCTTCTACCTTACTGCTTTTATAGTTGTATTTTATA |
| | | TAGCAGATAATTTTGTGAGGGGATATTTGAGAGGTTGGG |
| | | AGGCAGGGAAGGCGTTTCTCACTTGAGAAATGACAAGA |
| | | GACCCAAAGAGGGGGTTAATGGGCAAGAGCTGGGCCTT |
| | | AGGAACCCTGCCTCACTAGGCCATACCCAAGCTGTCCT |
| | | GCTTGGGCTGCTTCTGACAGGAAAGGCTTCACACGGAC |
| | | TTTGATATTGTTGGTCCTTAAACTCTACCAAGGCAGGAG |
| | | GGTGGTGGGTAATAGAGGAGTGTGGATGACCATTTTGA |
| | | CCACTTCCCCCCTCCTTTCAGGTCAAGGCCTTCTTGGCT |
| | | GATCCATCTGCCTTTGTGGCTGCTGCCCCTGTGGCTGCT |
| | | GCCACCACAGCTGCTCCTGCTGCTGCTGCAGCCCCAGC |
| | | TAAGGTTGAAGCCAAGGAAGAGTCGGAGGAGTCGGACG |
| | | AGGATATGGGATTTGGTCTCTTTGACGGCGGGTCTCGC |
| | | CCATTCCAGTGTAATCAGTGTGGGGCATCTTTTACTCAG |
| | | AAAGGTAACCTCCTCCGCCACATTAAACTGCACTAATCA |
| | | CCAAAAAGCAACGAACTTAGCCAGTTTTATTTGCAAAACA |
| | | AGGAAATAAATGCTTACTTCTTTAAAAAGTCTCTTGACTC |
| | | TTAATTTTGTAATTTTTTTTCCTTTTTGACACAGGGTCTGG |
| | | CTGTTGCCCAGGCTGGAGTGTGGTGGTGTAATCATAACT |
| | | CACTGCACCCTTGAACTCCTGGGATCAAGGGATCCTCGT |
| | | ATCTCAGCCTCCCAAGTAGCTGGGACTACAGGCACACA |
| | | CCATGACACTCAGCTACTAATTTTTAAATTTTTTTTTTGTA |
| | | GAGATGTTGCACAAGCTGGTCTCAAATTCCTGGCCTCAA |
| | | GGAATCCTGCCTCAGCCTCCCAAAGTGCTAGGATTACAG |
| | | GCTTGAGCCACCATGTGCCTGGCCCTTAATTTTGAGGTT |
| | | TATAGTGCCATATGCTAGAAACGAAAGCCATGGTAAAAC |
| | | CAGAGCTTTGTATTTAGGTGTTGATGTTTGGGTATCTAAA |
| | | TGAAGCTACCAATCAAACATCCTATACAGTTTTCTAGACA |
| | | CAGTTGTAACTATTACACTAGAATTACTGTTTCTATGGCT |
| | | GCTGCATACTTGGAGTAGGTTTAGTGTCAGCTGAGATAG |
| | | GCACCTGGTGGATGCTGGGGCCAGTCCCCTAGAGTAAA |
| | | GTTTTTCAAACTGGGTGGTGCTCCAACTCGGTGGTAACC |
| | | AATTTATATTTTCGAGATAGTCTCAAATATATTTGAGACT |
| | | GGGGTGCAGTGGCTTGGACTTGGCTCACTGCAACCTCC |
| | | GCCTCCTGGGTTCAAGTGATTCTCCTGCCTCAGCCTCCC |
| | | AAGTAGCTGC |
| 105. | RPLPO C-terminal LHA- | CTGAGCTGCCAACCTGGCAATTATTGTCTGCTAAGGGTT |
| | linker 1-superdegron- | CTCTTTATTCACCCTTACTTGGACTTCCTTTCCTGTAGGG |
| | stop codon-RPLP0 C- | AATCTCACGTAAAATGAAATCTTCCCTCCCCCAGGGTGT |
| | terminal RHA | CCGCAATGTTGCCAGTGTCTGTCTGCAGATTGGCTACCC |
| | | AACTGTTGCATCAGTACCCCATTCTATCATCAACGGGTA |
| | | CAAACGAGTCCTGGCCTTGTCTGTGGAGACGGATTACA |
| | | CCTTCCCACTTGCTGAAAAGGTAAAAGGATCCCACCAGG |
| | | ACCACAGTGGGCCTGACTGTGACAAATTAGCAGGGTGA |
| | | TGTGGCCTTCTACCTTACTGCTTTTATAGTTGTATTTTATA |
| | | TAGCAGATAATTTTGTGAGGGGATATTTGAGAGGTTGGG |
| | | AGGCAGGGAAGGCGTTTCTCACTTGAGAAATGACAAGA |
| | | GACCCAAAGAGGGGGTTAATGGGCAAGAGCTGGGCCTT |
| | | AGGAACCCTGCCTCACTAGGCCATACCCAAGCTGTCCT |
| | | GCTTGGGCTGCTTCTGACAGGAAAGGCTTCACACGGAC |
| | | TTTGATATTGTTGGTCCTTAAACTCTACCAAGGCAGGAG |
| | | GGTGGTGGGTAATAGAGGAGTGTGGATGACCATTTTGA |
| | | CCACTTCCCCCCTCCTTTCAGGTCAAGGCCTTCTTGGCT |
| | | GATCCATCTGCCTTTGTGGCTGCTGCCCCTGTGGCTGCT |
| | | GCCACCACAGCTGCTCCTGCTGCTGCTGCAGCCCCAGC |
| | | TAAGGTTGAAGCCAAGGAAGAGTCGGAGGAGTCGGACG |
| | | AGGATATGGGATTTGGTCTCTTTGACGGCTCAGGTAGC |
| | | GGAAGCGGATCAGGTGGATTCAATGTACTGATGGTCCA |
| | | TAAACGGAGTCACACTGGCGAGCGCCCGCTCCAATGTG |
| | | AAATCTGCGGGTTCACGTGTCGGCAGAAGGGCAACCTC |
| | | CTCCGGCATATCAAGCTGCACACGGGTGAAAAACCGTTT |
| | | AAGTGCCATCTCTGCAATTACGCCTGTCAGAGAAGAGAT |
| | | GCTTTGTAATCACCAAAAAGCAACGAACTTAGCCAGTTTT |
| | | ATTTGCAAAACAAGGAAATAAATGCTTACTTCTTTAAAAA |
| | | GTCTCTTGACTCTTAATTTTGTAATTTTTTTTCCTTTTTGA |
| | | CACAGGGTCTGGCTGTTGCCCAGGCTGGAGTGTGGTGG |
| | | TGTAATCATAACTCACTGCACCCTTGAACTCCTGGGATC |
| | | AAGGGATCCTCGTATCTCAGCCTCCCAAGTAGCTGGGA |
| | | CTACAGGCACACACCATGACACTCAGCTACTAATTTTTAA |
| | | ATTTTTTTTTTGTAGAGATGTTGCACAAGCTGGTCTCAAA |
| | | TTCCTGGCCTCAAGGAATCCTGCCTCAGCCTCCCAAAGT |
| | | GCTAGGATTACAGGCTTGAGCCACCATGTGCCTGGCCC |
| | | TTAATTTTGAGGTTTATAGTGCCATATGCTAGAAACGAAA |
| | | GCCATGGTAAAACCAGAGCTTTGTATTTAGGTGTTGATG |
| | | TTTGGGTATCTAAATGAAGCTACCAATCAAACATCCTATA |
| | | CAGTTTTCTAGACACAGTTGTAACTATTACACTAGAATTA |
| | | CTGTTTCTATGGCTGCTGCATACTTGGAGTAGGTTTAGT |
| | | GTCAGCTGAGATAGGCACCTGGTGGATGCTGGGGCCAG |
| | | TCCCCTAGAGTAAAGTTTTTCAAACTGGGTGGTGCTCCA |
| | | ACTCGGTGGTAACCAATTTATATTTTCGAGATAGTCTCAA |
| | | ATATATTTGAGACTGGGGTGCAGTGGCTTGGACTTGGCT |
| | | CACTGCAACCTCCGCCTCCTGGGTTCAAGTGATTCTCCT |
| | | GCCTCAGCCTCCCAAGTAGCTGC |

### 10. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present specification are intended.

What is claimed is:

1. A fusion protein comprising:
(a) an essential polypeptide;
(b) a degron; and
(c) optionally; a linker.

2. The fusion protein of claim 1, wherein the degron is C-terminal to the essential polypeptide.

3. The fusion protein of any one of claims 1 to 2, wherein the degron is an inducible degron.

4. The fusion protein of claim 3, wherein the degron is a drug-inducible degron.

5. The fusion protein of claim 4, wherein the drug is an immunomodulatory drug (IMiD).

6. The fusion protein of any one of claims 1 to 5, wherein the degron comprises or consists of the amino acid sequence RPFQCNQCGASFTQKGNLLRHIKLH (SEQ ID NO: 3), FNVLMVHKRSHTGERPLQCEICGFTCRQKGNLLRHIKLHTGEKPFKCHLCNYACQRRDAL (SEQ ID NO:4), FNVLMVHKRSHTGERP (SEQ ID NO:5), FNVLMVHRRSHTGERP (SEQ ID NO: 6), TGEKPFKCHLCNYACQRRDAL (SEQ ID NO:7), TGERPFRCHLCNYACQRRDAL (SEQ ID NO:8), FQCNQCGASFT (SEQ ID NO:9), FQCPICGLVIK (SEQ ID NO:10), LQCEICGFTCR (SEQ ID NO: 11), LQCEICGYQCR (SEQ ID NO: 12), or LQCEVCGFQCR (SEQ ID NO:13).

7. The fusion protein of any one of claims 1 to 6, wherein the degron is a superdegron.

8. The fusion protein of any one of claims 1 to 7, which comprises a linker sequence between the essential polypeptide and the degron.

9. The fusion protein of any one of claims 1 to 8, comprising two or more degrons, optionally wherein the degrons are in tandem.

10. The fusion protein of any one of claims 1 to 9, wherein the essential polypeptide is a STEL polypeptide, optionally wherein the STEL polypeptide is GAPDH.

11. A targeting construct comprising:
(a) a first homology arm corresponding to a 5′ target sequence comprising a first region of homology to an essential gene encoding an essential polypeptide in a target genomic locus;
(b) a nucleotide sequence encoding a degron (“degron coding sequence”);
(c) a second homology arm corresponding to a 3′ target sequence comprising second region of homology to the essential gene in the target genomic locus,
wherein the targeting construct is configured such that upon its recombination with the target genomic locus, the essential gene is modified such to encode a fusion protein comprising the essential polypeptide and the degron, optionally wherein the fusion protein has one or more features as defined in any one of claims 1 to 10.

12. The targeting construct of claim 11, wherein the first homology arm and second homology arm are each 500 to 1,500 nucleotides in length and/or wherein the length difference between the first and second homology arms, if any, is less than 75 nucleotides.

13. The targeting construct of claim 11 or claim 12, wherein the targeting construct further comprises a transgene between the degron coding sequence and the second homology arm.

14. The targeting construct of claim 13, wherein the transgene is linked to the nucleotide sequence encoding the fusion protein (“fusion protein coding sequence”).

15. The targeting construct of any one of claims 11 to 14, which is a vector, optionally wherein the vector is a DNA vector or an RNA vector.

16. A system comprising:
(a) the targeting construct of any one of claims 11 to 15;
(b) a CRISPR-associated endonuclease (“Cas polypeptide”) or a nucleic acid encoding a Cas polypeptide; and
(c) a guide RNA (“gRNA”) comprising a scaffold for binding the Cas polypeptide and a spacer sequence corresponding to the essential gene, or a nucleic acid encoding the gRNA.

17. The system of claim 16, wherein the guide RNA is a single guide RNA (“sgRNA”).

18. The system of claim 16 or claim 17, which is in the form of a ribonucleoprotein particle (“RNP”).

19. A method of producing a gene-edited target cell, comprising:
(a) introducing the system of any one of claims 16 to 18 into a target cell; and
(b) culturing the target cell under conditions in which gene editing occurs, thereby producing gene-edited target cell.

20. The method of claim 19, wherein the target cell is a stem cell or a cell differentiated from a stem cell.

21. The method of claim 20, wherein the cell is (a) a human embryonic stem cell, (b) an induced pluripotent stem cell (“iPSC”) or (c) a cell differentiated from (a) or (b).

22. The method of any one of claims 19 to 20, wherein the target cell is:
(a) a human immune cell, optionally selected from a T cell, a T cell expressing a chimeric antigen receptor (CAR) or recombinant TCR, a regulatory T cell, a myeloid cell, a dendritic cell, and a macrophage (e.g., an immunosuppressive macrophage);
(b) a cell in the human nervous system, optionally selected from dopaminergic neuron, a microglial cell, an oligodendrocyte, an astrocyte, a cortical neuron, a spinal or oculomotor neuron, an enteric neuron, a Placode-derived cell, a Schwann cell, and a trigeminal or sensory neuron;
(c) a cell in the human cardiovascular system, optionally selected from a cardiomyocyte, an endothelial cell, and a nodal cell;
(d) a cell in the human metabolic system, optionally selected from a hepatocyte, a cholangiocyte, and a pancreatic beta cell,
(e) a cell in the human ocular system, optionally selected from a retinal pigment epithelial cell, a photoreceptor cone cell, a photoreceptor rod cell, a bipolar cell, or a ganglion cell, or
(f) a progenitor or precursor of any one of the aforementioned cells.

23. A gene-edited target cell obtained or obtainable by the method of any one of claims 19 to 22.

24. A gene-edited target cell comprising an essential gene that encodes a fusion protein comprising:
(a) a degron;
(b) an essential polypeptide encoded by an essential gene as defined in claim 10.

25. A recombinant cell engineered to express the fusion protein of any one of claims 1 to 10, optionally wherein the recombinant cell is a gene-edited target cell according to any one of claim 23 or claim 24.

26. The recombinant cell of claim 25, which is further engineered to express a transgene from a locus other than the essential gene.

27. The recombinant cell of claim 25, which comprises an expression vector comprising a nucleotide sequence encoding the fusion protein of any one of claims 1 to 10.

28. A pharmaceutical composition comprising the gene-edited target cell of claim 23 or claim 24, the recombinant cell of any one of claims 25 to 27 and a pharmaceutically acceptable excipient.

29. Use of the gene-edited target cell claim 23 or claim 24 or the recombinant cell of any one of claims 25 to 27, for the manufacture of a medicament for treating a patient in need thereof, optionally wherein the gene-edited target cell or the recombinant cell is (a) autologous to the patient or (b) allogeneic to the patient.

30. The gene-edited target cell of claim 23 or claim 24, the recombinant cell of any one of claims 25 to 27, or the pharmaceutical composition of claim 28, for use in treating a patient in need thereof, optionally wherein the gene-edited target cell or the recombinant cell is, or the pharmaceutical comprises cells that are: (a) autologous to the patient or (b) allogeneic to the patient.

31. A method of treating a subject with a cell therapy, comprising administering to a subject in need thereof the gene-edited target cell of claim 23 or claim 24, the recombinant cell of any one of claims 25 to 27, or the pharmaceutical composition of claim 28, optionally wherein the gene-edited target cell or the recombinant cell is, or the pharmaceutical comprises cells that are: (a) autologous to the patient or (b) allogeneic to the patient.

32. The method of claim 31, which further comprises administering to the subject an inducer of the degron.

33. The method of claim 31 or claim 32, wherein the degron is a drug-inducible degron.

34. The method of claim 33, wherein the drug is an immunomodulatory drug (IMiD).

35. A method of reducing engineered cells in, or eliminating engineered cells from, a subject who previously received cell therapy with a gene-edited target cell of claim 23 or claim 24, the recombinant cell of any one of claims 25 to 27, or the pharmaceutical composition of claim 28, comprising administering to the subject an inducer of the degron.


SEQUENCE LISTING (105 sequences)

SEQ ID NO: 1 - DNA, 1687 bp, synthetic construct
  source 1..1687: mol_type = other DNA
ttggtatcgtggaaggactcatggtatgagagctggggaatgggactgaggctcccacctttctcatccaagactggctcctccctgccggggctgcgtgcaaccctggggttgggggttctggggactggctttcccataatttcctttcaaggtggggagggaggtagaggggtgatgtggggagtacgctgcagggcctcactccttttgcagaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgtggctggggccagagactggctcttaaaaagtgcagggtctggcgccctctggtggctggctcagaaaaagggccctgacaactctttacatcttctaggtatgacaacgaatttggctacagcaacagggtggtggacctcatggcccacatggcctccaaggagggcgggtctcgcccattccagtgtaatcagtgtggggcatcttttactcagaaaggtaacctcctccgccacattaaactgcactaagacccctggaccaccagccaaagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttacttgtcctgtcttattctagggtctggggcagaggggagggaagctgggcttgtgtcaaggtgagacattcttgctggggagggacctggtatgttctcctcagactgagggtagggcctccaaacagccttgcttgcttcgagaaccatttgcttcccgctcagacgtcttgagtgctacaggaagctggcaccactacttcagagaacaaggccttttcctctcctcgctccagtcctaggctatctgctgttggccaaacatggaagaagctattctgtgggcagccccagggaggctgacaggtggaggaagtcagggctcgcactgggctctgacgctgactggttagtggagctcagcctggagctgagctgcagcgggcaattccagcttggcctccgcagctgtgaggtcttgagcacgtgctctattgctttctgtgccctcgtgtcttatctgaggacatcgtggccagcccctaaggtcttcaagcaggattcatctaggtaaaccaagtacctaaaaccatgcccaaggcggtaaggactatataatgtttaaaaatcggtaaaaatgcccacctcgcatagt

SEQ ID NO: 2 - DNA, 1813 bp, synthetic construct
  source 1..1813: mol_type = other DNA
ttggtatcgtggaaggactcatggtatgagagctggggaatgggactgaggctcccacctttctcatccaagactggctcctccctgccggggctgcgtgcaaccctggggttgggggttctggggactggctttcccataatttcctttcaaggtggggagggaggtagaggggtgatgtggggagtacgctgcagggcctcactccttttgcagaccacagtccatgccatcactgccacccagaagactgtggatggcccctccgggaaactgtggcgtgatggccgcggggctctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgtggctggggccagagactggctcttaaaaagtgcagggtctggcgccctctggtggctggctcagaaaaagggccctgacaactctttacatcttctaggtatgacaacgaatttggctacagcaacagggtggtggacctcatggcccacatggcctccaaggagggctcaggtagcggaagcggatcaggtggattcaatgtactgatggtccataaacggagtcacactggcgagcgcccgctccaatgtgaaatctgcgggttcacgtgtcggcagaagggcaacctcctccggcatatcaagctgcacacgggtgaaaaaccgtttaagtgccatctctgcaattacgcctgtcagagaagagatgctttgtaagacccctggaccaccagccaaagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttacttgtcctgtcttattctagggtctggggcagaggggagggaagctgggcttgtgtcaaggtgagacattcttgctggggagggacctggtatgttctcctcagactgagggtagggcctccaaacagccttgcttgcttcgagaaccatttgcttcccgctcagacgtcttgagtgctacaggaagctggcaccactacttcagagaacaaggccttttcctctcctcgctccagtcctaggctatctgctgttggccaaacatggaagaagctattctgtgggcagccccagggaggctgacaggtggaggaagtcagggctcgcactgggctctgacgctgactggttagtggagctcagcctggagctgagctgcagcgggcaattccagcttggcctccgcagctgtgaggtcttgagcacgtgctctattgctttctgtgccctcgtgtcttatctgaggacatcgtggccagcccctaaggtcttcaagcaggattcatctaggtaaaccaagtacctaaaaccatgcccaaggcggtaaggactatataatgtttaaaaatcggtaaaaatgcccacctcgcatagt

SEQ ID NO: 3 - protein, 25 aa, synthetic construct
RPFQCNQCGASFTQKGNLLRHIKLH

SEQ ID NO: 4 - protein, 60 aa, synthetic construct
FNVLMVHKRSHTGERPLQCEICGFTCRQKGNLLRHIKLHTGEKPFKCHLCNYACQRRDAL

SEQ ID NO: 5 - protein, 16 aa, synthetic construct
FNVLMVHKRSHTGERP

SEQ ID NO: 6 - protein, 16 aa, synthetic construct
FNVLMVHRRSHTGERP

SEQ ID NO: 7 - protein, 21 aa, synthetic construct
TGEKPFKCHLCNYACQRRDAL

SEQ ID NO: 8 - protein, 21 aa, synthetic construct
TGERPFRCHLCNYACQRRDAL

SEQ ID NO: 9 - protein, 11 aa, synthetic construct
FQCNQCGASFT

SEQ ID NO: 10 - protein, 11 aa, synthetic construct
FQCPICGLVIK

SEQ ID NO: 11 - protein, 11 aa, synthetic construct
LQCEICGFTCR

SEQ ID NO: 12 - protein, 11 aa, synthetic construct
LQCEICGYQCR

SEQ ID NO: 13 - protein, 11 aa, synthetic construct
LQCEVCGFQCR

SEQ ID NO: 14 - DNA, 1687 bp, synthetic construct
  source 1..1687: mol_type = other DNA
tcttaagcccctctctttctctaacagaaaaagcggatggtggttcctgctgccctcaaggtcgtgcgtctgaagcctacaagaaaggtgagtcccagcttacgctgcaccatctacttgggagatttcaggcctgctgagggacctggggacctggagcctggcagatgatgtccttatctcacgatggtctgcggatgtccctgtgggaatggcgacaatgccaatggcttagctgatgccaggaggcttgggtgggtgcttttctaacaggcctgcagagaacagttgcattatgatatgcccagctgtcagtcacctcccagctctcaacagctccggctcttcagggtgtgggggcttagatatccttacaacttcatttgttcaccccccccccccccccccgcagtttgcctatctggggcgcctggctcacgaggttggctggaagtaccaggcagtgacagccaccctggaggagaagaggaaagagaaagccaagatccactaccggaagaagaaacagctcatggtgaggccaggggctggtgctgaggggggcatctcactcctggacaggcctggcaggtgccttgctcacagagtactcttaactggcaaaggaccagccggggttggggtgggatgcagtccatgtaatgagggcaatgcaacccctcctgaccaccaccacctgcacttattcttggcagaggctacgcaaacaagccgagaagaacgtggagaagaaaattgacaaatacacagaggtcctcaagacccacggactcctggtcggcgggtctcgcccattccagtgtaatcagtgtggggcatcttttactcagaaaggtaacctcctccgccacattaaactgcactaagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaatctggcgtctttgcactgtgtctgctgtggaggcaggcccctggcaaatggggggtgaggagcttgaagagggtagaatgggctgtgctaatatacagaatatatgtaacttgctataaattgaatgatcctttatagacaccgtttacaaaccaaagacataaaatgtggccagcagtgcctggtgcttcctagttaatgtaaagctgtctcattctaattcagctgcaaagtatggacccatgccctgctgccaggctgctgtagtcccggcggtctgtagagactagcattttgcaaatgataa

SEQ ID NO: 15 - protein, 10 aa, synthetic construct
GSGSGSGSGG

SEQ ID NO: 16 - protein, 4 aa, synthetic construct
GGGS

SEQ ID NO: 17 - DNA, 1813 bp, synthetic construct
  source 1..1813: mol_type = other DNA
tcttaagcccctctctttctctaacagaaaaagcggatggtggttcctgctgccctcaaggtcgtgcgtctgaagcctacaagaaaggtgagtcccagcttacgctgcaccatctacttgggagatttcaggcctgctgagggacctggggacctggagcctggcagatgatgtccttatctcacgatggtctgcggatgtccctgtgggaatggcgacaatgccaatggcttagctgatgccaggaggcttgggtgggtgcttttctaacaggcctgcagagaacagttgcattatgatatgcccagctgtcagtcacctcccagctctcaacagctccggctcttcagggtgtgggggcttagatatccttacaacttcatttgttcaccccccccccccccccccgcagtttgcctatctggggcgcctggctcacgaggttggctggaagtaccaggcagtgacagccaccctggaggagaagaggaaagagaaagccaagatccactaccggaagaagaaacagctcatggtgaggccaggggctggtgctgaggggggcatctcactcctggacaggcctggcaggtgccttgctcacagagtactcttaactggcaaaggaccagccggggttggggtgggatgcagtccatgtaatgagggcaatgcaacccctcctgaccaccaccacctgcacttattcttggcagaggctacgcaaacaagccgagaagaacgtggagaagaaaattgacaaatacacagaggtcctcaagacccacggactcctggtcggctcaggtagcggaagcggatcaggtggattcaatgtactgatggtccataaacggagtcacactggcgagcgcccgctccaatgtgaaatctgcgggttcacgtgtcggcagaagggcaacctcctccggcatatcaagctgcacacgggtgaaaaaccgtttaagtgccatctctgcaattacgcctgtcagagaagagatgctttgtaagcccaataaagactgttaattcctcatgcgttgcctgcccttcctccattgttgccctggaatgtacgggacccaggggcagcagcagtccaggtgccacaggcagccctgggacataggaagctgggagcaaggaaagggtcttagtcactgcctcccgaagttgcttgaaagcactcggagaattgtgcaggtgtcatttatctatgaccaataggaagagcaaccagttactatgagtgaaagggagccagaagactgattggagggccctatcttgtgagtggggcatctgttggactttccacctggtcatatactctgcagctgttagaatgtgcaagcacttggggacagcatgagcttgctgttgtacacagggtatttctagaagcagaaatagactgggaagatgcacaaccaaggggttacaggcatcgcccatgctcctcacctgtattttgtaatcagaaataaattgcttttaaagaaatctggcgtctttgcactgtgtctgctgtggaggcaggcccctggcaaatggggggtgaggagcttgaagagggtagaatgggctgtgctaatatacagaatatatgtaacttgctataaattgaatgatcctttatagacaccgtttacaaaccaaagacataaaatgtggccagcagtgcctggtgcttcctagttaatgtaaagctgtctcattctaattcagctgcaaagtatggacccatgccctgctgccaggctgctgtagtcccggcggtctgtagagactagcattttgcaaatgataa

SEQ ID NO: 18 - protein, 4 aa, synthetic construct
GGSG

SEQ ID NO: 19 - protein, 4 aa, synthetic construct
GGGS

SEQ ID NO: 20 - protein, 5 aa, synthetic construct
GGGGS

SEQ ID NO: 21 - protein, 9 aa, synthetic construct
GGSGGSGGS

SEQ ID NO: 22 - protein, 10 aa, synthetic construct
GGGGSGGGGS

SEQ ID NO: 23 - protein, 15 aa, synthetic construct
GGGGSGGGGSGGGGS

SEQ ID NO: 24 - protein, 1307 aa, unidentified
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 25 - protein, 1331 aa, unidentified
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 26 - protein, 1103 aa, unidentified
MTIRSMKLKLKIYSGRSAPQLRQGLWRLHRLLNEGTAYYMDWLVHMRQEALPGKSKEEIRAELERRVRQQQEKNGVQNDQVPMDEVLSALRQLYELLVPSAVNNSGDAQTLSRKFLSPLVDPNSEGGKGTSNAGAKPGWRKKQEAGDPSWEKDYERWLKRKQADPTAEILGKLETAGLKPLFPLYTNEVKDIRWMPLTSKQYVRNWDRDMFQQAIEHLLSWETWNRKVNEERAKLKETVRRFEEQHLANGKDWLSPLQAYEANREQALRDMAISPSDRFRITRRQIKGWSELYERWNKLAPTASVEAYMQEVRHVQKKLGGTFGDADLYRFLAKPENVHIWRDHQERLHYYAAYNDLHKRLMSAKEQAAFTLPDPVAHPLWVRFDARDGNLFTYILQADSSKQRSRRYVNFSRFLWPVEDGYFEETENVKVELALSKQFYRQVIVHDNPTGKQKITFQDYSSKEILEGHLGGAKLQLDRNFLRKSGRDFETGDFGPAFLNVVLDLKPKQEVKNGRLQSPLGQALLVKSRPNDIPKVYGYKPDALAAWLEQASGEESLGSESLRQGFRVMSIDLGVRSAAAISVFSVKGEKTREGDKVCYPVGETGLFAVHDRSFLLRLPGESSEKRVNVERDKRKTERMQIRYHIRTLARVLRLANKATPMDRIKAVQDVLNDIESTRFMNDHDHHVYNHALETLRTYAPDHQGIWEEQVIAAHRQLEHHVGVIVGEWRKNWGKDRRGTVGLSMDNIEELDEMRRLLISWSRRARYPREAKPFQVNESNPVHLLRHLQNLKEDRLKQLANLIVMTALGYVYDSKEKKWKAAYPACQLILFEDLQRYRFHLDRSARENSQLMKWAHRSIPKYVWMQGEPYGLQIGDVWAGFTSRYHAKTGAPGIRCKALTEKDFQQGRLLESLVAEGMFTLQEVGTLKPGDIVPAEGGELFVTLADDSGDRIVITHADINAAQNVQKRFWLANSERFRVACRSVQIASQECFIPSSESVAKKMGKGVFVRDFSFHKDMEVYHWNNQVKLTAKNVPTDHSDDLQDLQDYQAILEEARESSSSYKTLFRDPSGFFFPDDVWVPQNIYWREVKKTITALLRKRIMST

SEQ ID NO: 27 - protein, 1091 aa, unidentified
MPIRSFKLKLVTHNGDSTYMDKLRRGLWKTHVIINRGIAYYMNTLALMRQEPYGSKSREEVRLDLLSTLREQQRRNNWSEQTGTDDELLSLSRRVYELLVPSAIGEKGDAQMLSRKFLSPLVDPNSEGGRGTAKSGRKPRWKKMMEEGHPDWEKEKEKDAAKKAEDPTASILADLEAVGLLPLFPLFSDEQKEIRWLPKKKRQFVRTWDRDMFQQALERMLSWESWNRRVAEEYLKLQAQRDEVYAKYLEDAGSWLNDLQTFEKQREEELAEVSFEPNSEYLITRRQIRGWKEVYEKWSKTSENASQEQLWRMVADVQTAMAGAFGDPKVYQFLSQPKHHHIWREHPNRLFYYSKYNEVREKLNRAKKQAAFTLPDPVEHPLWTRFDARGGNIHDYEISKVGKQYHVTFSSLILPEAQSWVEIENVTVGIGNSLQLKRQIRLDGYADKKQKVKYYDYSSRFELTGVLGGAKIQFDRKHLKKAAHRLAEGETGPIFLNVVVDVEPFLEVKNGRLRTPLGQVLQVNTRDWPKVVDYKAKELSVLMENTQIGNENGVSTIEAGMRIMSIDLGQRTAAAVSIFEVISKKPDEKETKLFYPIADTDLYAVHRRSLLLRLPGEEISSKKMIEKRKERARIRSLVRYQIRLLSEVLRLHTQGTAEQRRFKLDELLVSIQKKLELDQSEWISELEKLFDYIDESAEKWKEALVVAHRTLEPIVVEAVRNWKKSLSKENKDRRRIAGISIWSIEELEETRKLLIAWSKHSREPGIPKRLEKEETFAPEHLQHIQNVKDDRLKQMANLFVMTALGYKYDEGNKRWVEAYPACQVILFEDLSRYRFALDRPRRENNRLMKWAHRSIPRLTYMQAELFGIQVGDVYSAYTSRFHAKTGAPGIRCHALTEADLQSNSYVVNQLIKDKFIQDNQTEILKAGQIVPWQGGELFVTFADRSGASLAVIHADINAAQNLQKRFWQHNSEVFRVPCKVVKGGLVPVYEKMRKLFGKGLFVNIDDPESKEVYRWEHSTKMKSKTTPVDLESEDIDHEELSDEWEDMQEGYKTLLRDPSGFFWSSDSWIPQKDFWIRVKSRIGKSLREQIR

SEQ ID NO: 28 - protein, 1091 aa, unidentified
MPIRSFKLKLVTHNGDSTYMDKLRRGLWKTHVIINRGIAYYMNTLALMRQEPYGSKSREEVRLDLLSTLREQQRRNNWSEQTGTDDELLSLSRRVYELLVPSAIGEKGDAQMLSRKFLSPLVDPNSEGGRGTAKSGRKPRWKKMMEEGHPDWEKEKEKDAAKKAEDPTASILADLEAVGLLPLFPLFSDEQKEIRWLPKKKRQFVRTWDRDMFQQALERMLSWESWNRRVAEEYQKLQAQRDEVYAKYLEDAGSWLNDLQTFEKQREEELAEVSFEPNSEYLITRRQIRGWKEVYEKWSKTSENASQEQLWRMVADVQTAMAGAFGDPKVYQFLSQPKHHHIWREHPNRLFYYSKYNEVREKLNRAKKQAAFTLPDPVEHPLWTRFDARGGNIHDYEISKVGKQYHVTFSSLILPEAQSWVEIENVTVGIGNSLQLKRQIRLDGYADKKQKVKYYDYSSRFELTGVLGGAKIQFDRKHLKKAAHRLAEGETGPIFLNVVVDVEPFLEVKNGRLRTPLGQVLQVNTRDWPKVVDYKAKELSVLMENTQIGNENGVSTIEAGMRIMSIDLGQRTAAAVSIFEVISKKPDEKETKLFYPIADTDLYAVHRRSLLLRLPGEEISSKKMIEKRKERARIRSLVRYQIRLLSEVLRLHTQGTAEQRRFKLDELLVSIQRKLELDQSEWISELEKLFDYIDESAEKWKEALVVAHRTLEPIVVEAVRNWKKSLSKENKDRRRIAGISIWSIEELEETRKLLIAWSKHSREPGIPKRLEKEETFAPEHLQHIQNVKDDRLKQMANLFVMTALGYKYDEGNKRWVEAYPACQVILFEDLSRYRFALDRPRRENNRLMKWAHRSIPRLTYMQAELFGIQVGDVYSAYTSRFHAKTGAPGIRCHALTEADLQSNSYVVNQLIKDKFIQDNQTEILKAGQIVPWQGGELFVTFADRSGASLAVIHADINAAQNLQKRFWQHNSEVFRVPCKVVKGGLVPVYEKMRKLFGKGLFVNIDDPESKEVYRWEHSTKMKSKTTPVDLESEDIEHEELSDEWEDMQEGYKTLLRDPSGFFWSSDSWIPQKDFWIRVKSRIGKSLREQIR

SEQ ID NO: 29 - DNA, 1687 bp, synthetic construct
  source 1..1687: mol_type = other DNA
ggagaagttccccaactttcccgcctctcagcctttgaaagaaagaaaggggagggggcaggccgcgtgcagccgcgagcggtgctgggctccggctccaattccccatctcagtcgttcccaaagtcctcctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgtgtgtcggccggggccactaggcgctcactgttctctccctccgcgcagccgagccacatcgctcagacaccatgcgcccattccagtgtaatcagtgtggggcatcttttactcagaaaggtaacctcctccgccacattaaactgcacggcgggtctgggaaggtgaaggtcggagtcaacgggtgagttcgcgaatggctggggggccctgggctgcgaccgcccccgaaccgcgtctacgagccttgcgggctccgggtcttagcagtcgtatgggggcagggtagctgttccccgcaaggagagctcaaggtcagcgctcggacctggcggagccccgcacccaggctgtggcgccctgtgcagctccgcccttgcggcgccatctgcccggagcctccttcccctagtccccagaaacaggaggtccctactcccgcccgagatcccgacccggacccctaggtgggggacgctttctttcctttcgcgctctgcggggtcacgtgtcgcagaggagcccctcccccacggcctccggcaccgcaggccccgggatgctagtgcgcagcgggtgcatccctgtccggatgctgcgcctgcggtagagcggccgccatgttgcaaccgggaaggaaatgaatgggcagccgttaggaaagcctgccggtgactaaccctgcgctcctgcctcgatgggtggagtcgcgtgtggcggggaagtcaggtggagcgaggctagctggcccgatttctcctccgggtgatgcttttcctagattattctctggtaaatcaaagaagtgggtttatggaggtcctcttgtgtcccctccccgcagaggtgtggtggctgtggcatggtgccaagccgggagaagctgagtcatgggtagttggaaaaggacatttccaccgcaaaatggcccctctggtggtggccccttcctgcagcg

SEQ ID NO: 30 - DNA, 1813 bp, synthetic construct
  source 1..1813: mol_type = other DNA
ggagaagttccccaactttcccgcctctcagcctttgaaagaaagaaaggggagggggcaggccgcgtgcagccgcgagcggtgctgggctccggctccaattccccatctcagtcgttcccaaagtcctcctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgtgtgtcggccggggccactaggcgctcactgttctctccctccgcgcagccgagccacatcgctcagacaccatgttcaatgtactgatggtccataaacggagtcacactggcgagcgcccgctccaatgtgaaatctgcgggttcacgtgtcggcagaagggcaacctcctccggcatatcaagctgcacacgggtgaaaaaccgtttaagtgccatctctgcaattacgcctgtcagagaagagatgctttgggctcaggtagcggaagcggatcaggtggagggaaggtgaaggtcggagtcaacgggtgagttcgcgaatggctggggggccctgggctgcgaccgcccccgaaccgcgtctacgagccttgcgggctccgggtcttagcagtcgtatgggggcagggtagctgttccccgcaaggagagctcaaggtcagcgctcggacctggcggagccccgcacccaggctgtggcgccctgtgcagctccgcccttgcggcgccatctgcccggagcctccttcccctagtccccagaaacaggaggtccctactcccgcccgagatcccgacccggacccctaggtgggggacgctttctttcctttcgcgctctgcggggtcacgtgtcgcagaggagcccctcccccacggcctccggcaccgcaggccccgggatgctagtgcgcagcgggtgcatccctgtccggatgctgcgcctgcggtagagcggccgccatgttgcaaccgggaaggaaatgaatgggcagccgttaggaaagcctgccggtgactaaccctgcgctcctgcctcgatgggtggagtcgcgtgtggcggggaagtcaggtggagcgaggctagctggcccgatttctcctccgggtgatgcttttcctagattattctctggtaaatcaaagaagtgggtttatggaggtcctcttgtgtcccctccccgcagaggtgtggtggctgtggcatggtgccaagccgggagaagctgagtcatgggtagttggaaaaggacatttccaccgcaaaatggcccctctggtggtggccccttcctgcagcg

SEQ ID NO: 31 - DNA, 1687 bp, synthetic construct
  source 1..1687: mol_type = other DNA
cacaggaatccaaatctctaagcctagaatgggcaaggctccggggctcacgccggtaatcccagcactttgggaggccgaaacgggaggatcgcttgagcccaggagttcgagatcagcctggccaacatgtcagactccccccgttcccgggccacaacaaaatatatatatatatatatattagccaggcatggtggcgcgcccctgtagtccaagttactcgagaggctgaggcaggaggatcccttgagcccaggaagtggaggctgcggtgagccatgatcgccccacagcactctggtgtggacaacacagcgagaccctgtctcacaaaataaagtaagcccggactgagtgcggaaaggcgggcctggcgggtctggtctccccatgcgggccaccagaggccctgcagccttcagtcgcttgaaggggtaatggcgcttccactcacaaacatggcggacagagcgtgtgaacgagatgaacagcccctcaaaaatatggccgccgaggctggacggccgtgccccagcagcaccgcctccgcgccccacgtgatctctcgccgggcacagcgctgaccgcggaggtccaaccggaagaatgtccggattggacattcggaagagggcccgccttccctggggaatctctgcgcacgcgcagaacgcttcgaccaatgaaaacacaggaagccgtccgcgcaaccgcgttgcgtcacttctgccgcccctgtttcaagggataagaaaccctgcgacaaaacctcctccttttccaagcggctgccgaagatgcgcccattccagtgtaatcagtgtggggcatcttttactcagaaaggtaacctcctccgccacattaaactgcacggcgggtctgcggaggtgcaggtatgggctccgcgcgggccggggcggcaaggggccgggtgggatccaggccggaatggggtcgcaccctctctgtcttgcatgttttgcggagcttaacatccataatgaagcaaaatggaagtcttttgggataaggggaatctgtaggaaatgccgcggctctctttaggcctagggttggctacaatgtggcatttccttctcgaggcgagggtgatagagcttccagcacaggacaggtattttgcaactagattttgcttaccttgaactcgggtagtgggtgggggccctggggtagagtcttggccgaattggacccttgtgtgtcctcagcgatgaggaacacgcggaagtgtggccgggcggtctcggggcaatgagaaactgagttttggccaaaatggaaggtatttttatcccgctcagtgcgcgggctactttgatttcacgtggaggtgttaatcggggtcccctgcagctccggttcttttaattctcaatgcctttctgcggggggtcgagagctgttggctgcggggcactctggaatgaactcgttttccgtcgttttggggattggcgattgtcctgaggcgtgttactggaagtcgagagctgtgatgaatggttccacaaggatgggacatcttttgggcctgacacctgggggccctttccctggctcctggtgtagctacttaagtataaaggagggattggccctggaaaggtttctttgtgagtagtctgttgttggggcgggtgacatttcagagccaggc

SEQ ID NO: 32 - DNA, 1813 bp, synthetic construct
  source 1..1813: mol_type = other DNA
cacaggaatccaaatctctaagcctagaatgggcaaggctccggggctcacgccggtaatcccagcactttgggaggccgaaacgggaggatcgcttgagcccaggagttcgagatcagcctggccaacatgtcagactccccccgttcccgggccacaacaaaatatatatatatatatatattagccaggcatggtggcgcgcccctgtagtccaagttactcgagaggctgaggcaggaggatcccttgagcccaggaagtggaggctgcggtgagccatgatcgccccacagcactctggtgtggacaacacagcgagaccctgtctcacaaaataaagtaagcccggactgagtgcggaaaggcgggcctggcgggtctggtctccccatgcgggccaccagaggccctgcagccttcagtcgcttgaaggggtaatggcgcttccactcacaaacatggcggacagagcgtgtgaacgagatgaacagcccctcaaaaatatggccgccgaggctggacggccgtgccccagcagcaccgcctccgcgccccacgtgatctctcgccgggcacagcgctgaccgcggaggtccaaccggaagaatgtccggattggacattcggaagagggcccgccttccctggggaatctctgcgcacgcgcagaacgcttcgaccaatgaaaacacaggaagccgtccgcgcaaccgcgttgcgtcacttctgccgcccctgtttcaagggataagaaaccctgcgacaaaacctcctccttttccaagcggctgccgaagatgttcaatgtactgatggtccataaacggagtcacactggcgagcgcccgctccaatgtgaaatctgcgggttcacgtgtcggcagaagggcaacctcctccggcatatcaagctgcacacgggtgaaaaaccgtttaagtgccatctctgcaattacgcctgtcagagaagagatgctttgggctcaggtagcggaagcggatcaggtggagcggaggtgcaggtatgggctccgcgcgggccggggcggcaaggggccgggtgggatccaggccggaatggggtcgcaccctctctgtcttgcatgttttgcggagcttaacatccataatgaagcaaaatggaagtcttttgggataaggggaatctgtaggaaatgccgcggctctctttaggcctagggttggctacaatgtggcatttccttctcgaggcgagggtgatagagcttccagcacaggacaggtattttgcaactagattttgcttaccttgaactcgggtagtgggtgggggccctggggtagagtcttggccgaattggacccttgtgtgtcctcagcgatgaggaacacgcggaagtgtggccgggcggtctcggggcaatgagaaactgagttttggccaaaatggaaggtatttttatcccgctcagtgcgcgggctactttgatttcacgtggaggtgttaatcggggtcccctgcagctccggttcttttaattctcaatgcctttctgcggggggtcgagagctgttggctgcggggcactctggaatgaactcgttttccgtcgttttggggattggcgattgtcctgaggcgtgttactggaagtcgagagctgtgatgaatggttccacaaggatgggacatcttttgggcctgacacctgggggccctttccctggctcctggtgtagctacttaagtataaaggagggattggccctggaaaggtttctttgtgagtagtctgttgttggggcgggtgacatttcagagccaggc

SEQ ID NO: 33 - DNA, 1687 bp, synthetic construct
  source 1..1687: mol_type = other DNA
gcgtgaacccgggaggcggagcttgcagtcagccgagatggcgccactgcgctccagcctgggcgacagagcgacactccgtctcaaaaaaaaaaaaaaaaagagaaaccattctgtttctaagcatgtgcaattatgtctcagctccacgtcataccttttagtttgctgagctcgccaggtggcacagggaaccgggactccgaattcgcagctccagctccagctccagcccagcaggtggcagcagctcagagcaagctcctccaacgcgaggcagcgccttccttcgcgaccctacttaaaggcggctttccgttggctggagtggcagcgatattatccaatggttgcctgtattcgttcagctttgtctgacgggcgatggcgcagccaatagacaggagcgctatccgcggtttctgattggctactttgttcgcattataaaaggcacgcgcgggcgcgaggcccttctctcgccaggcgtcctcgtggaaggttcgtgtgctagttagatgggcgccaggggtcgccggcgggaagcatggaggggtctttgggggcctttgggaacatggagtcctattctgttccgcctggggcctcggtggcggcttgcacgccccgagatgacggccgctgccctaggcagggccggcgggcgattgcgcgtgtcctgctcctcttaggcccgggaccgcgggatgggtgtcggcgtgaccaggcctgagctccctgtctctcctcagtgacatcgtctttaaaccctgcgtggcaatccctgacgcaccgccgtgatgcgcccattccagtgtaatcagtgtggggcatcttttactcagaaaggtaacctcctccgccacattaaactgcacggcgggtctcccagggaagacagggcgacctggaagtccaactacttccttaagatcatcgtaagtgcagggtgggtcgcctctgctcttcatgttgccccagcgcaaatagggacagtcagctgctatgtgctgagggtctactcacaccggctactgaattaggccatttttgggaaaatactgtttagttaacaatttcctgagataggtcccttctgttgcagataaacgggctcaggcaagttaagtgggtcctaagatgacagcattcgtatccaggtctgtctggcttctaaaagagcgcgctttatacttttttttttttaaacggagtctcgctctgttgtccaggctggagtgcagtggcttgatcttggcttactgcaacctccgcttcccaggttcaagcgattctcctgcttcagcctcccgagtagctgggattacaggggcgcgacaccacacccagctaatttttgtattttcaatagagacagggattcaccatgttggccaggatggtctcgatctcttgacttcatgatccaccttcctcggcctccctaagtgctaggattacaggtgtgagccaccgcgcccggccgcacattacagttttactccatttttgagaaggtctgaggtcaggaatgactcattgtaaggaagcaagctgttgcatatttaggacctgtcagccaagaaacttaagtgtccaatgacttttggatggcctttgaggtgggtctcccttgcccagaaatgctgactgtactgcgcttaagcaattaggcagcattgtagag

SEQ ID NO: 34 - DNA, 1813 bp, synthetic construct
  source 1..1813: mol_type = other DNA
gcgtgaacccgggaggcggagcttgcagtcagccgagatggcgccactgcgctccagcctgggcgacagagcgacactccgtctcaaaaaaaaaaaaaaaaagagaaaccattctgtttctaagcatgtgcaattatgtctcagctccacgtcataccttttagtttgctgagctcgccaggtggcacagggaaccgggactccgaattcgcagctccagctccagctccagcccagcaggtggcagcagctcagagcaagctcctccaacgcgaggcagcgccttccttcgcgaccctacttaaaggcggctttccgttggctggagtggcagcgatattatccaatggttgcctgtattcgttcagctttgtctgacgggcgatggcgcagccaatagacaggagcgctatccgcggtttctgattggctactttgttcgcattataaaaggcacgcgcgggcgcgaggcccttctctcgccaggcgtcctcgtggaaggttcgtgtgctagttagatgggcgccaggggtcgccggcgggaagcatggaggggtctttgggggcctttgggaacatggagtcctattctgttccgcctggggcctcggtggcggcttgcacgccccgagatgacggccgctgccctaggcagggccggcgggcgattgcgcgtgtcctgctcctcttaggcccgggaccgcgggatgggtgtcggcgtgaccaggcctgagctccctgtctctcctcagtgacatcgtctttaaaccctgcgtggcaatccctgacgcaccgccgtgatgttcaatgtactgatggtccataaacggagtcacactggcgagcgcccgctccaatgtgaaatctgcgggttcacgtgtcggcagaagggcaacctcctccggcatatcaagctgcacacgggtgaaaaaccgtttaagtgccatctctgcaattacgcctgtcagagaagagatgctttgggctcaggtagcggaagcggatcaggtggacccagggaagacagggcgacctggaagtccaactacttccttaagatcatcgtaagtgcagggtgggtcgcctctgctcttcatgttgccccagcgcaaatagggacagtcagctgctatgtgctgagggtctactcacaccggctactgaattaggccatttttgggaaaatactgtttagttaacaatttcctgagataggtcccttctgttgcagataaacgggctcaggcaagttaagtgggtcctaagatgacagcattcgtatccaggtctgtctggcttctaaaagagcgcgctttatacttttttttttttaaacggagtctcgctctgttgtccaggctggagtgcagtggcttgatcttggcttactgcaacctccgcttcccaggttcaagcgattctcctgcttcagcctcccgagtagctgggattacaggggcgcgacaccacacccagctaatttttgtattttcaatagagacagggattcaccatgttggccaggatggtctcgatctcttgacttcatgatccaccttcctcggcctccctaagtgctaggattacaggtgtgagccaccgcgcccggccgcacattacagttttactccatttttgagaaggtctgaggtcaggaatgactcattgtaaggaagcaagctgttgcatatttaggacctgtcagccaagaaacttaagtgtccaatgacttttggatggcctttgaggtgggtctcccttgcccagaaatgctgactgtactgcgcttaagcaattaggcagcattgtagag

SEQ ID NO: 35 - protein, 5 aa, synthetic construct
PAPAP

SEQ ID NO: 36 - protein, 12 aa, synthetic construct
AEAAAKEAAAKA

SEQ ID NO: 37 - protein, 46 aa, synthetic construct
AEAAAKEAAAKEAAAKEAAAKALEAEAAAKEAAAKEAAAKEAAAKA

SEQ ID NO: 38 - RNA, 21 nt, synthetic construct
  source 1..21: mol_type = other RNA
tgagccagccaccagagggcg

SEQ ID NO: 39 - RNA, 21 nt, synthetic construct
  source 1..21: mol_type = other RNA
atcttctaggtatgacaacga

SEQ ID NO: 40 - RNA, 21 nt, synthetic construct
  source 1..21: mol_type = other RNA
gctacagcaacagggtggtgg

SEQ ID NO: 41 - RNA, 21 nt, synthetic construct
  source 1..21: mol_type = other RNA
ccataatttcctttcaaggtg

SEQ ID NO: 42 - RNA, 21 nt, synthetic construct
  source 1..21: mol_type = other RNA
ctttcaaggtggggagggagg

SEQ ID NO: 43 - RNA, 21 nt, synthetic construct
  source 1..21: mol_type = other RNA
aaggtggggagggaggtagag

SEQ ID NO: 44 - RNA, 21 nt, synthetic construct
  source 1..21: mol_type = other RNA
gcagaccacagtccatgccat

SEQ ID NO: 45 - RNA, 21 nt, synthetic construct
  source 1..21: mol_type = other RNA
cagaccacagtccatgccatc

SEQ ID NO: 46 - RNA, 21 nt, synthetic construct
  source 1..21: mol_type = other RNA
ccggaggggccatccacagtc

SEQ ID NO: 47 - RNA, 21 nt, synthetic construct
  source 1..21: mol_type = other RNA
tagacggcaggtcaggtccac

SEQ ID NO: 48 - RNA, 21 nt, synthetic construct
  source 1..21: mol_type = other RNA
ctagacggcaggtcaggtcca

SEQ ID NO: 49 - RNA, 21 nt, synthetic construct
  source 1..21: mol_type = other RNA
tctagacggcaggtcaggtcc

SEQ ID NO: 50 - RNA, 21 nt, synthetic construct
  source 1..21: mol_type = other RNA
gcaggtttttctagacggcag

SEQ ID NO: 51 - RNA, 21 nt, synthetic construct
  source 1..21: mol_type = other RNA
tcaagctcatttcctggtatg

SEQ ID NO: 52 - RNA, 21 nt, synthetic construct
  source 1..21: mol_type = other RNA
ctggtatgtggctggggccag

SEQ ID NO: 53 - RNA, 21 nt, synthetic construct
  source 1..21: mol_type = other RNA
agagccagtctctggccccag

SEQ ID NO: 54 - RNA, 21 nt, synthetic construct
  source 1..21: mol_type = other RNA
aagagccagtctctggcccca

SEQ ID NO: 55 - RNA, 21 nt, synthetic construct
  source 1..21: mol_type = other RNA
taagagccagtctctggcccc

SEQ ID NO: 56 - RNA, 21 nt, synthetic construct
  source 1..21: mol_type = other RNA
ctgagccagccaccagagggc

SEQ ID NO: 57 - RNA, 21 nt, synthetic construct
  source 1..21: mol_type = other RNA
tctgagccagccaccagaggg

SEQ ID NO: 58 - RNA, 21 nt, synthetic construct
  source 1..21: mol_type = other RNA
catcttctaggtatgacaacg

SEQ ID NO: 59 - RNA, 21 nt, synthetic construct
  source 1..21: mol_type = other RNA
ttgatggtacatgacaaggtg

SEQ ID NO: 60 - RNA, 21 nt, synthetic construct
  source 1..21: mol_type = other RNA
gaggccctaccctcagtctga

SEQ ID NO: 61 - RNA, 21 nt, synthetic construct
  source 1..21: mol_type = other RNA
cctctcctcgctccagtccta

SEQ ID NO: 62 - RNA, 21 nt, synthetic construct
  source 1..21: mol_type = other RNA
ctctcctcgctccagtcctag

SEQ ID NO: 63 - RNA, 21 nt, synthetic construct
  source 1..21: mol_type = other RNA
gccaacagcagatagcctagg

SEQ ID NO: 64 - RNA, 21 nt, synthetic construct
  source 1..21: mol_type = other RNA
tgtgccctcgtgtcttatctg

SEQ ID NO: 65 - RNA, 21 nt, synthetic construct
  source 1..21: mol_type = other RNA
cctagatgaatcctgcttgaa

SEQ ID NO: 66 - RNA, 21 nt, synthetic construct
  source 1..21: mol_type = other RNA
ggtacttggtttacctagatg

SEQ ID NO: 67 - RNA, 21 nt, synthetic construct
  source 1..21: mol_type = other RNA
aggtacttggtttacctagat

SEQ ID NO: 68 - RNA, 21 nt, synthetic construct
  source 1..21: mol_type = other RNA
aaacattatatagtccttacc

SEQ ID NO: 69 - RNA, 21 nt, synthetic construct
  source 1..21: mol_type = other RNA
taaacattatatagtccttac

SEQ ID NO: 70 - RNA, 21 nt, synthetic construct
  source 1..21: mol_type = other RNA
ccgatttttaaacattatata

SEQ ID NO: 71 - RNA, 21 nt, synthetic construct
  source 1..21: mol_type = other RNA
accgatttttaaacattatat

SEQ ID NO: 72 - RNA, 21 nt, synthetic construct
  source 1..21: mol_type = other RNA
taccgatttttaaacattata

SEQ ID NO: 73 - RNA, 21 nt, synthetic construct
  source 1..21: mol_type = other RNA
aaaatcggtaaaaatgcccac

SEQ ID NO: 74 - RNA, 21 nt, synthetic construct
  source 1..21: mol_type = other RNA
gaggaagatgaactgagatgt

SEQ ID NO: 75 - RNA, 21 nt, synthetic construct
  source 1..21: mol_type = other RNA
aggaagatgaactgagatgtg

SEQ ID NO: 76 - RNA, 20 nt, synthetic construct
  source 1..20: mol_type = other RNA
cttcctcttgtgctcttgct

SEQ ID NO: 77 - RNA, 20 nt, synthetic construct
  source 1..20: mol_type = other RNA
cctccaaggagtaagacccc

SEQ ID NO: 78 - RNA, 20 nt, synthetic construct
  source 1..20: mol_type = other RNA
catggcccacatggcctcca

SEQ ID NO: 79 - RNA, 20 nt, synthetic construct
  source 1..20: mol_type = other RNA
agccccagcaagagcacaag

SEQ ID NO: 80 - RNA, 20 nt, synthetic construct
  source 1..20: mol_type = other RNA
ggaagggcaggcaacgcatg

SEQ ID NO: 81 - RNA, 20 nt, synthetic construct
  source 1..20: mol_type = other RNA
ggctcagaccaggagtccgt

SEQ ID NO: 82 - RNA, 20 nt, synthetic construct
  source 1..20: mol_type = other RNA
cctcaagacccacggactcc

SEQ ID NO: 83 - RNA, 20 nt, synthetic construct
  source 1..20: mol_type = other RNA
gtctttattgggctcagacc

SEQ ID NO: 84 - RNA, 20 nt, synthetic construct
  source 1..20: mol_type = other RNA
acattccagggcaacaatgg

SEQ ID NO: 85 - RNA, 20 nt, synthetic construct
  source 1..20: mol_type = other RNA
ttggcagaggctacggaaac

SEQ ID NO: 86 - RNA, 20 nt, synthetic construct
  source 1..20: mol_type = other RNA
cttattcttggcagaggcta

SEQ ID NO: 87 - RNA, 20 nt, synthetic construct
  source 1..20: mol_type = other RNA
gcaaataaaactggctaagt

SEQ ID NO: 88 - RNA, 20 nt, synthetic construct
  source 1..20: mol_type = other RNA
ttacttctttaaaaagtctc

SEQ ID NO: 89 - RNA, 20 nt, synthetic construct
  source 1..20: mol_type = other RNA
tttgcaaaacaaggaaataa

SEQ ID NO: 90 - RNA, 23 nt, synthetic construct
  source 1..23: mol_type = other RNA
tttccttgttttgcaaataaaac

SEQ ID NO: 91 - RNA, 23 nt, synthetic construct
  source 1..23: mol_type = other RNA
caaataaaactggctaagttggt

SEQ ID NO: 92 - RNA, 23 nt, synthetic construct
  source 1..23: mol_type = other RNA
cttgttttgcaaataaaactggc

SEQ ID NO: 93 - RNA, 23 nt, synthetic construct
  source 1..23: mol_type = other RNA
gtgattagtcaaagagaccaaat

SEQ ID NO: 94 - RNA, 23 nt, synthetic construct
  source 1..23: mol_type = other RNA
actaatcaccaaaaagcaaccaa

SEQ ID NO: 95 - RNA, 21 nt, synthetic construct
  source 1..21: mol_type = other RNA
aggtcaaggccttcttggctg

SEQ ID NO: 96 - RNA, 21 nt, synthetic construct
  source 1..21: mol_type = other RNA
caaataaaactggctaagttg

SEQ ID NO: 97 - RNA, 20 nt, synthetic construct
  source 1..20: mol_type = other RNA
actcaacctttctcaggatg

SEQ ID NO: 98 - RNA, 20 nt, synthetic construct
  source 1..20: mol_type = other RNA
acattaactcaacctttctc

SEQ ID NO: 99 - RNA, 20 nt, synthetic construct
  source 1..20: mol_type = other RNA
agttaatgttagagtataag

SEQ ID NO: 100 - protein, 335 aa, Homo sapiens
MGKVKVGVNGFGRIGRLVTRAAFNSGKVDIVAINDPFIDLNYMVYMFQYDSTHGKFHGTVKAENGKLVINGNPITIFQERDPSKIKWGDAGAEYVVESTGVFTTMEKAGAHLQGGAKRVIISAPSADAPMFVMGVNHEKYDNSLKIISNASCTTNCLAPLAKVIHDNFGIVEGLMTTVHAITATQKTVDGPSGKLWRDGRGALQNIIPASTGAAKAVGKVIPELNGKLTGMAFRVPTANVSVVDLTCRLEKPAKYDDIKKVVKQASEGPLKGILGYTEHQVVSSDFNSDTHSSTFDAGAGIALNDHFVKLISWYDNEFGYSNRVVDLMAHMASKE

SEQ ID NO: 101 - protein, 203 aa, Homo sapiens [sequence omitted]

SEQ ID NO: 102 - protein, 317 aa, Homo sapiens
MPREDRATWKSNYFLKIIQLLDDYPKCFIVGADNVGSKQMQQIRMSLRGKAVVLMGKNTMMRKAIRGHLENNPALEKLLPHIRGNVGFVFTKEDLTEIRDMLLANKVPAAARAGAIAPCEVTVPAQNTGLGPEKTSFFQALGITTKISRGTIEILSDVQLIKTGDKVGASEATLLNMLNISPFSFGLVIQQVFDNGSIYNPEVLDITEETLHSRFLEGVRNVASVCLQIGYPTVASVPHSIINGYKRVLALSVETDYTFPLAEKVKAFLADPSAFVAAAPVAAATTAAPAAAAAPAKVEAKEESEESDEDMGFGLFD

SEQ ID NO: 103 - protein, 27 aa, synthetic construct
GGSGGSGGSGGSGGSGGSGGSGGSGGS

SEQ ID NO: 104 - DNA, 1687 bp, synthetic construct
  source 1..1687: mol_type = other DNA
ctgagctgccaacctggcaattattgtctgctaagggttctctttattcacccttacttggacttcctttcctgtagggaatctcacgtaaaatgaaatcttccctcccccagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtaaaaggatcccaccaggaccacagtgggcctgactgtgacaaattagcagggtgatgtggccttctaccttactgcttttatagttgtattttatatagcagataattttgtgaggggatatttgagaggttgggaggcagggaaggcgtttctcacttgagaaatgacaagagacccaaagagggggttaatgggcaagagctgggccttaggaaccctgcctcactaggccatacccaagctgtcctgcttgggctgcttctgacaggaaaggcttcacacggactttgatattgttggtccttaaactctaccaaggcaggagggtggtgggtaatagaggagtgtggatgaccattttgaccacttcccccctcctttcaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgacggcgggtctcgcccattccagtgtaatcagtgtggggcatcttttactcagaaaggtaacctcctccgccacattaaactgcactaatcaccaaaaagcaacgaacttagccagttttatttgcaaaacaaggaaataaatgcttacttctttaaaaagtctcttgactcttaattttgtaattttttttcctttttgacacagggtctggctgttgcccaggctggagtgtggtggtgtaatcataactcactgcacccttgaactcctgggatcaagggatcctcgtatctcagcctcccaagtagctgggactacaggcacacaccatgacactcagctactaatttttaaattttttttttgtagagatgttgcacaagctggtctcaaattcctggcctcaaggaatcctgcctcagcctcccaaagtgctaggattacaggcttgagccaccatgtgcctggcccttaattttgaggtttatagtgccatatgctagaaacgaaagccatggtaaaaccagagctttgtatttaggtgttgatgtttgggtatctaaatgaagctaccaatcaaacatcctatacagttttctagacacagttgtaactattacactagaattactgtttctatggctgctgcatacttggagtaggtttagtgtcagctgagataggcacctggtggatgctggggccagtcccctagagtaaagtttttcaaactgggtggtgctccaactcggtggtaaccaatttatattttcgagatagtctcaaatatatttgagactggggtgcagtggcttggacttggctcactgcaacctccgcctcctgggttcaagtgattctcctgcctcagcctcccaagtagctgc

SEQ ID NO: 105 - DNA, 1813 bp, synthetic construct
  source 1..1813: mol_type = other DNA
ctgagctgccaacctggcaattattgtctgctaagggttctctttattcacccttacttggacttcctttcctgtagggaatctcacgtaaaatgaaatcttccctcccccagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtaaaaggatcccaccaggaccacagtgggcctgactgtgacaaattagcagggtgatgtggccttctaccttactgcttttatagttgtattttatatagcagataattttgtgaggggatatttgagaggttgggaggcagggaaggcgtttctcacttgagaaatgacaagagacccaaagagggggttaatgggcaagagctgggccttaggaaccctgcctcactaggccatacccaagctgtcctgcttgggctgcttctgacaggaaaggcttcacacggactttgatattgttggtccttaaactctaccaaggcaggagggtggtgggtaatagaggagtgtggatgaccattttgaccacttcccccctcctttcaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgacggctcaggtagcggaagcggatcaggtggattcaatgtactgatggtccataaacggagtcacactggcgagcgcccgctccaatgtgaaatctgcgggttcacgtgtcggcagaagggcaacctcctccggcatatcaagctgcacacgggtgaaaaaccgtttaagtgccatctctgcaattacgcctgtcagagaagagatgctttgtaatcaccaaaaagcaacgaacttagccagttttatttgcaaaacaaggaaataaatgcttacttctttaaaaagtctcttgactcttaattttgtaattttttttcctttttgacacagggtctggctgttgcccaggctggagtgtggtggtgtaatcataactcactgcacccttgaactcctgggatcaagggatcctcgtatctcagcctcccaagtagctgggactacaggcacacaccatgacactcagctactaatttttaaattttttttttgtagagatgttgcacaagctggtctcaaattcctggcctcaaggaatcctgcctcagcctcccaaagtgctaggattacaggcttgagccaccatgtgcctggcccttaattttgaggtttatagtgccatatgctagaaacgaaagccatggtaaaaccagagctttgtatttaggtgttgatgtttgggtatctaaatgaagctaccaatcaaacatcctatacagttttctagacacagttgtaactattacactagaattactgtttctatggctgctgcatacttggagtaggtttagtgtcagctgagataggcacctggtggatgctggggccagtcccctagagtaaagtttttcaaactgggtggtgctccaactcggtggtaaccaatttatattttcgagatagtctcaaatatatttgagactggggtgcagtggcttggacttggctcactgcaacctccgcctcctgggttcaagtgattctcctgcctcagcctcccaagtagctgc